(19) United States
(12) Patent Application Publication
(10) Pub. No.: US 2025/0179057 A1
(43) Pub. Date: Jun. 5, 2025

(54) MEK IMMUNE ONCOLOGY INHIBITORS AND THERAPEUTIC USES THEREOF

(71) Applicant: Immuneering Corporation, Cambridge, MA (US)

(72) Inventors: Brett Matthew Hall, San Diego, CA (US); Bart Lieven DeCorte, Southampton, PA (US); Peter John King, San Diego, CA (US); Ruben Leenders, Cambridge, MA (US); Anita Wegert, Cambridge, MA (US); Kevin Fowler, Cambridge, MA (US); Sarah Kolitz, Cambridge, MA (US); Robin Doodeman, Cambridge, MA (US); Jarno Poelakker, Cambridge, MA (US); Rutger Henk Adriaan Folmer, Cambridge, MA (US)

(21) Appl. No.: 18/726,908

(22) PCT Filed: Jan. 5, 2023
(86) PCT No.: PCT/US2023/060181

§ 371 Date: Jul. 5, 2024

Related U.S. Application Data
(60) Provisional application No. 63/297,116, filed on Jan. 6, 2022.

(51) Int. Cl. C07D 405/06; A61K 31/37; A61K 31/397; A61K 31/496; A61P 35/00; C07D 311/16; C07D 311/18

(52) U.S. Cl. CPC C07D 405/06; A61K 31/37; A61K 31/397; A61K 31/496; A61P 35/00; C07D 311/16; C07D 311/18

(57) ABSTRACT

The present disclosure provides compounds, compositions containing such compounds, and methods of designing, developing, producing and preparing compounds represented by general Formula (III), including pharmaceutically acceptable salts thereof or a synthetic intermediate thereof:

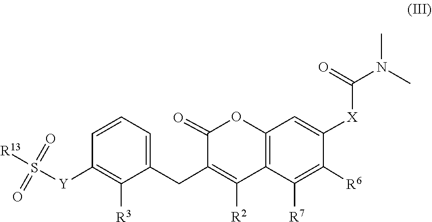

The compounds act as MEK inhibitors and are capable of displaying one or more beneficial therapeutic effects, including treating cancer.

### RELATED APPLICATIONS

This patent application is a U.S. National Phase of International Application No. PCT/US2023/060181, filed Jan. 5, 2023, which claims the benefit of U.S. Provisional Application No. 63/297,116, filed Jan. 6, 2022. All of the foregoing applications are fully incorporated herein by reference in their entireties for all purposes.

### BACKGROUND

### Field

The present invention relates to the fields of chemistry and medicine. More particularly, the present invention relates to MEK inhibitors, MEK immune oncology inhibitors, techniques for designing and synthesizing such MEK inhibitors, MEK immune oncology compositions comprising MEK inhibitors, MEK immune oncology inhibitors, and methods of treating disease comprising administering MEK inhibitors.

### Description of the Related Technology

In healthy cells, the Mitogen Activated Protein Kinase (MAPK) pathway utilizes parallel signaling streams to decipher complex extracellular stimuli and drive cellular programs that promote proliferation, differentiation, survival, motility, apoptosis, and stress response. The RAS-RAF-MEK-ERK cascade is one of three distinct MAPK pathways and is the one most often exploited in cancer. Gain of function mutations in RAS (KRAS, NRAS, HRAS) or RAF (ARAF, BRAF, CRAF/RAF1) are common in cancers. RAS mutations alone represent up to 95% of pancreatic cancer (KRAS), 20-30% of melanoma (NRAS), 40% of non-small cell lung cancer (KRAS) and 45% of colorectal cancer (KRAS). Patients with these tumor types often face poor prognoses and limited therapeutic options, which has led to intense research on how to create active drugs against this pathway. As a central node in the MAPK signaling pathway, MEK has been an attractive drug target for over two decades, but in the clinic, MEK inhibitors have suffered from class-effect toxicities and acquired resistance. Additionally, regulatory approvals have been restricted mainly to RAF mutant disease. Clinical setbacks in the RAS mutant setting likely stem from a mechanistic blind spot of first generation MEK inhibitors that leads to increased pathway reactivation, which in turn, necessitates sustained target engagement that limits drug tolerability. Therefore, new ways to target MEK with greater tolerability and broader activity are urgently needed.

MEK1 and MEK2 (MEK) are closely related dual-specificity kinases that are activated by upstream mediators including RAF (ARAF, BRAF, RAF1[also known as CRAF]), KSR (KSR1, KSR2) and RAS (KRAS, NRAS, HRAS). When activated by phosphorylation on two serine residues, pMEK in turn facilitates phosphorylation of ERK1 and ERK2 (pERK), which leads to regulation of multiple downstream targets. Inappropriate activation of this pathway is associated with multiple oncogenic cell processes including proliferation, survival, growth, tumor metabolism, migration and immune evasion. Multiple targeted agents have been, and continue to be, developed with the goal of reducing MAPK pathway activity at each level from RAS to ERK, and clinical proof-of-concept has been achieved for several drugs in this area, including those that target KRAS^{G12C}, BRAF^{V600E/K} and MEK. Common challenges for drugs that block this core homeostatic pathway include clinically limiting toxicities stemming from sustained on target inhibition, narrow subsets of addressable patients based on tumors that display specific target mutations (e.g., KRAS^{G12C} or BRAFV^{600E/K}), and acquired or adaptive resistance that limits clinical utility for emergent drugs.

Previous MEK inhibitors suffer from one or more critical shortcomings. First generation MEK inhibitors counteract pathway reactivation by sustaining drug occupancy in the allosteric pocket of MEK throughout the dosing cycle (i.e., chronic inhibition). This is generally achieved by endowing drugs with long half-lives and dosing at regular intervals or creating drugs with moderate half-lives and dosing at higher frequencies. Both approaches lead to chronic suppression of the MAPK pathway by sustaining active steady state drug trough levels. Second generation MEK inhibitors resist pathway reactivation by engaging the allosteric pocket in MEK in a unique way that prevents RAF activation of MEK itself but still have long half-lives, leading to chronic pathway ablation. This continuous disruption of this core biologic pathway creates at least three well-documented challenges for first and second generation MEK inhibitors: (1) tolerability: clinically limiting, class effect safety issues (Heinzerling 2019), (2) acquired/adaptive resistance: selective pressure for escape mutations (Corcoran 2011), and (3) clinical utility: reduced drug-drug combination potential due to limitations on drug-related safety and toxicity. Accordingly, first generation MEK inhibitors suffer from multiple shortcomings: (1) sustained on-target occupancy drives acquired and/or adaptive resistance and dose-limiting toxicities, (2) mechanistic drug-target interaction fails to effectively control pathway reactivation (e.g., CRAF-bypass) and (3) limited clinical utility for drug combinations due to high baseline drug-related toxicity.

A common feature of nearly all MEK inhibitors, is their allosteric target engagement, which is highly selective for MEK and non-ATP competitive, commonly referred to as a Type-III allosteric inhibitor. First generation MEK inhibitors, exemplified by trametinib, cobimetinib, binimetinib and selumetinib, sustainably suppress MAPK pathway activity through chronic occupancy of MEK1 and MEK2. Yet, they display dose-limiting class effect toxicities and are sensitive to pathway reactivation events. While second generation MEK inhibitors, exemplified by VS-6766 (CH5126766, that has a mean terminal half-life of 53.6-hours, Guo et al Lancet Oncol. 2020 November; 21 (11): 1478-1488), display mechanistic resistance to pathway reactivation, they also sustainably suppress MAPK pathway activity through chronic occupancy of MEK1 and MEK2 and, as such, share similar class effect toxicities as first-generation inhibitors. Table 1 is a summary of certain characteristics of MEK inhibitors for RAS mutant disease treatment.

TABLE 1
Characteristics of MEK Inhibitors by Generation in RAS Mutant Disease

| | | MoA | Pathway | Pathway | Class-effect |
|---|---|---|---|---|---|
| MEKi Type | Examples | Engagement | Bypass | Suppression | Toxicities |
| First | trametinib, | Type-III non- | Sensitive | Chronic | Yes |
| Generation | cobimetinib, | ATP | | | |
| | binimetinib, | competitive | | | |
| | selumetinib | | | | |
| Second | VS-6766 | Type-III non- | Resistant | Chronic | Yes |
| Generation | | ATP | | | |
| | | competitive | | | |

Therefore, there remains an unmet need for improved MEK inhibitor compounds, such as MEK inhibitor compounds with shorter half-life, such compounds having a shorter half-life in mouse and/or human liver microsome stability testing.

### SUMMARY OF THE DISCLOSURE

The compounds disclosed in the present application have been discovered to exhibit surprising and unexpected biological effects. In some embodiments, the chemical compounds of the present application are useful as dual-MEK inhibitors exhibiting surprising and unexpected biological effects.

In some embodiments, the compounds are MEK inhibitor compounds characterized by an unexpectedly short in vivo half-life, including novel dual-MEK inhibitors with unprecedentedly short half-lives, and useful to cyclically inhibit and release MEK and ERK activity. Unlike previous MEK inhibitors, Applicants have discovered compounds that can be used in methods of treatment designed to maximize drug exposures at Cmax while achieving a near-zero drug trough within 12- to 24-hour cycles. In some embodiments, the compounds disclosed herein can be administered on a schedule to drive deep, cyclic inhibition of the MAPK pathway in a subject.

Some embodiments related to compound having the chemical structure of Formula (IV)

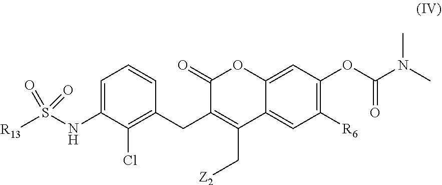

or a pharmaceutically acceptable salt thereof, wherein R₆ is hydrogen, fluoro or chloro, R₁₃ is ethyl or —NR_{A}R_{B} wherein R_{A} is hydrogen and R_{B} is methyl, Z₂ is —NR⁵R^{5′},

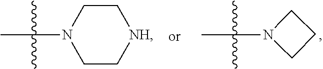 [SMILES: *N1CCNCC1 ; *N1CCC1]

R⁵ is C₁ to C₆ alkyl, and R^{5′} is C₁ to C₆ alkyl. In some embodiments, R⁵ is methyl. In some embodiments, R^{5′} is methyl. In some embodiments, R⁵, is ethyl. In some embodiments, Z₂ is —NR⁵R^{5′}. In some embodiments, R₁₃ is —NR_{A}R_{B}. In some embodiments, the compound is

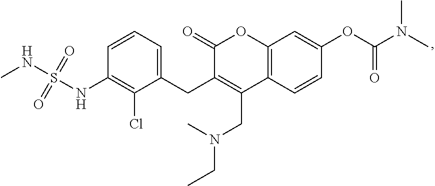

or a pharmaceutically acceptable salt thereof. In some embodiments, the compound is

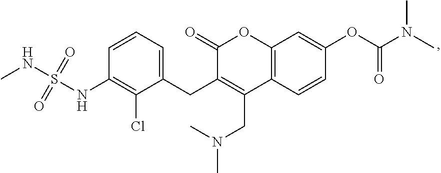

or a pharmaceutically acceptable salt thereof. In some embodiments, the compound is

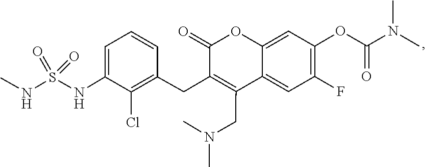

or a pharmaceutically acceptable salt thereof. In some embodiments, the compound is

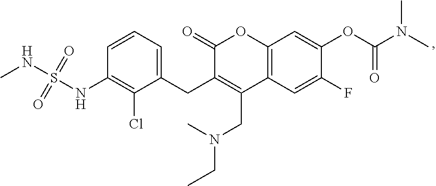

or a pharmaceutically acceptable salt thereof. In some embodiments, the compound is

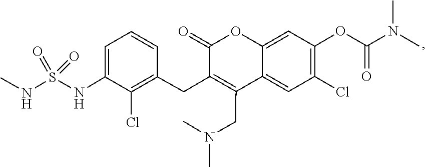

or, or a pharmaceutically acceptable salt thereof. In some embodiments, the compound is

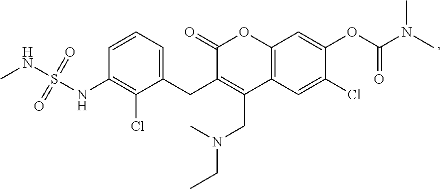

or a pharmaceutically acceptable salt thereof. In some embodiments, R₁₃ is ethyl. In some embodiments, the compound is

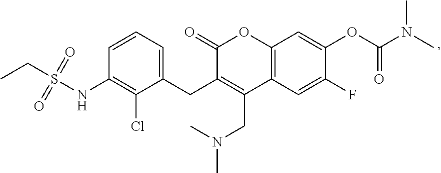 [SMILES: CCS(=O)(=O)Nc1cccc(Cc2c(CN(C)C)c3cc(F)c(OC(=O)N(C)C)cc3oc2=O)c1Cl]

or a pharmaceutically acceptable salt thereof. In some embodiments, the compound is

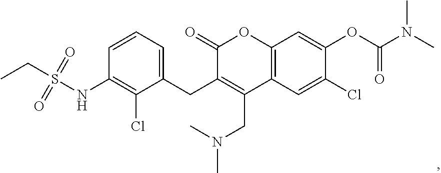 [SMILES: CCS(=O)(=O)Nc1cccc(Cc2c(CN(C)C)c3cc(Cl)c(OC(=O)N(C)C)cc3oc2=O)c1Cl]

or a pharmaceutically acceptable salt thereof. In some embodiments, the compound is

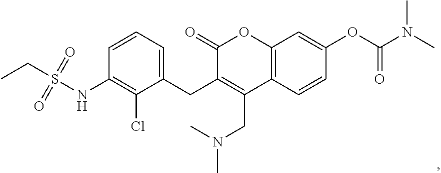 [SMILES: CCS(=O)(=O)Nc1cccc(Cc2c(CN(C)C)c3ccc(OC(=O)N(C)C)cc3oc2=O)c1Cl]

or a pharmaceutically acceptable salt thereof. In some embodiments, Z₂ is

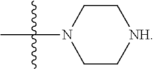 [SMILES: *N1CCNCC1]

In some embodiments, R₁₃ is ethyl. In some embodiments, the compound is

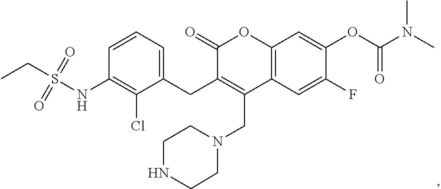 [SMILES: CCS(=O)(=O)Nc1cccc(Cc2c(CN3CCNCC3)c3cc(F)c(OC(=O)N(C)C)cc3oc2=O)c1Cl]

or a pharmaceutically acceptable salt thereof. In some embodiments, the compound is

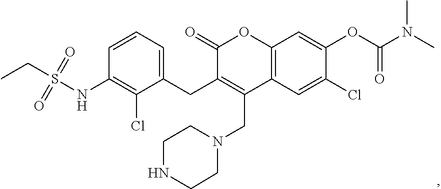 [SMILES: CCS(=O)(=O)Nc1cccc(Cc2c(CN3CCNCC3)c3cc(Cl)c(OC(=O)N(C)C)cc3oc2=O)c1Cl]

or a pharmaceutically acceptable salt thereof. In some embodiments, the compound is

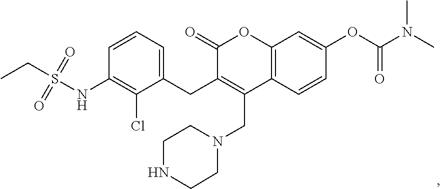 [SMILES: CCS(=O)(=O)Nc1cccc(Cc2c(CN3CCNCC3)c3ccc(OC(=O)N(C)C)cc3oc2=O)c1Cl]

or a pharmaceutically acceptable salt thereof. In some embodiments, R₁₃ is —NR_{A}R_{B}. In some embodiments, the compound is

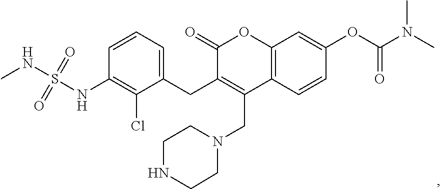

or a pharmaceutically acceptable salt thereof. In some embodiments, the compound is

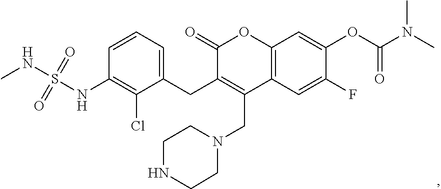

or a pharmaceutically acceptable salt thereof. In some embodiments, the compound is

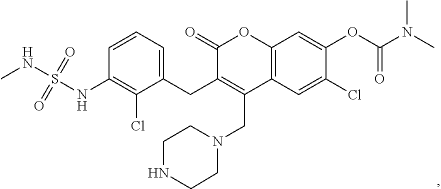

or a pharmaceutically acceptable salt thereof. In some embodiments, Z₂ is

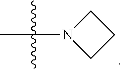 [SMILES: *N1CCC1]

In some embodiments, R₁₃ is ethyl. In some embodiments, R₁₃ is —NR_{A}R_{B}. In some embodiments, the compound is

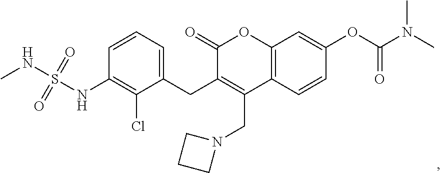

or a pharmaceutically acceptable salt thereof. In some embodiments, the compound is

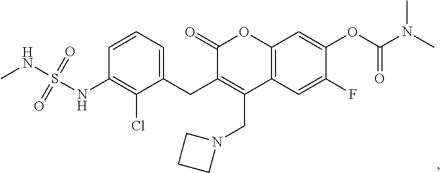

or a pharmaceutically acceptable salt thereof. In some embodiments, the compound is

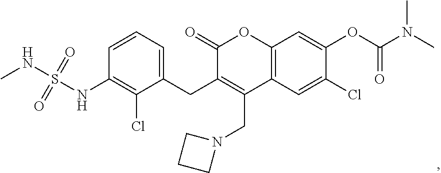

or a pharmaceutically acceptable salt thereof.

Some embodiments relate to a compound having the structure of Formula (III):

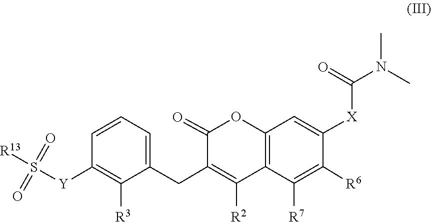

including pharmaceutically acceptable salts thereof, wherein, R² is L; R⁶ is selected from the group consisting of H or fluoro, chloro or bromo; R⁷ is H; R¹³ is selected from the group consisting of optionally substituted optionally substituted amin, C₁ to C₆ alkyl, H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L; R³ is a chloro, X is —O—, Y is

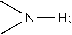

L is —Z₁-Z₂, Z₁ is —CH₂—; and Z₂, is selected from the group consisting of —NR⁵R^{5′}, optionally substituted C₃ to C₈ heterocyclyl, —CH₂—, —O—, —S—, S═O, —SO₂—, C═O, —CO₂—, —NO₂, —NH—, —CH₂CCH, —CH₂CN, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₁₀ heteroaryl, —CH₂-(optionally substituted aryl), —CH₂— (optionally substituted C₃ to C₈ cycloalkyl), and —CH₂-(optionally substituted C₃ to C₁₀ heteroaryl); and each R⁵ and R^{5′} are independently selected optionally substituted C₁ to C₆ alkyl. In some embodiments, Z₂, is —NR⁵R^{5′}. In some embodiments, R⁵ is methyl. In some embodiments, R^{5′} is methyl. In some embodiments, R^{5′} is ethyl. In some embodiments, Z₂ is

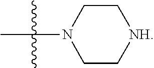 [SMILES: *N1CCNCC1]

In some embodiments, Z₂ is optionally substituted

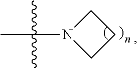

wherein n is 1, 2, 3 or 4. In some embodiments, n is 1. In some embodiments, R¹³ is —NR_{A}R_{B} wherein R_{A} and R_{B} are each independently selected from hydrogen, or C₁₋₆ alkyl. In some embodiments, R_{A} is hydrogen and R_{B} is methyl. In some embodiments, R¹³ is C₁ to C₆ alkyl. In some embodiments, R¹³ is ethyl. In some embodiments, R⁶ is fluoro. In some embodiments, R⁶ is chloro. In some embodiments, R⁶ is H.

Some embodiments relate to a compound having the structure of Formula (III):

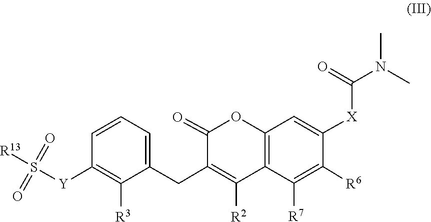

including pharmaceutically acceptable salts thereof, wherein, R² is L; R⁶ is selected from the group consisting of H or fluoro, chloro or bromo; R⁷ is H; R¹³ is C₁ to C₆ alkyl; R³ is a chloro; X is —O—; Y is

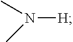

L is —Z₁-Z₂; Z₁ is —CH₂—; Z₂, is —NR⁵R^{5′}; and each R⁵ and R^{5′} are independently selected from optionally substituted C₁ to C₆ alkyl.

Some embodiments relate to a compound having the structure of Formula (III):

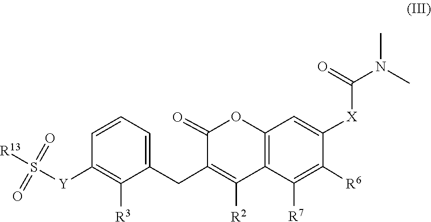

including pharmaceutically acceptable salts thereof, wherein, R² is selected from the group consisting of halogen, H, deuterium, hydroxyl, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L, R⁶ is selected from the group consisting of H, halogen, deuterium, hydroxyl, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L; R⁷ is selected from the group consisting of deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L; R¹³ is selected from the group consisting of optionally substituted C₁ to C₆ alkyl, optionally substituted amino, H, deuterium, hydroxyl, halogen, cyano, nitro, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L; R³ is a chloro, bromo, or iodo; X is —O—, C(R⁵)₂, CH(R⁵), CH₂,

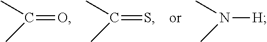

### Y is

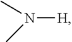

C(R⁵)₂, CH(R⁵), CH₂, —O—,

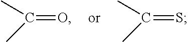

L is —Z₁-Z₂ or —Z₁-Z₂—Z₃; Z₁ is selected from the group consisting of —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl); Z₂, is selected from the group consisting of —NR⁵R^{5′}, —CH₂—, —O—, —S—, S═O, —SO₂—, C═O, —CO₂—, —NO₂, —NH—, —CH₂CCH, —CH₂CN, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl); Z₃ is selected from the group consisting of —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl); and each R⁵ and R^{5′} are independently selected from optionally substituted C₁ to C₆ alkyl, H, deuterium, optionally substituted C₂ to C₆ alkenyl, optionally substituted C₂ to C₆ alkynyl, optionally substituted C₃ to C₈ carbocyclyl, optionally substituted C₆ to C₁₀ aryl, optionally substituted C₃ to C₈ heterocyclyl, and optionally substituted C₃ to C₁₀ heteroaryl.

In some embodiments, the design of certain compounds was based in part on the Applicant's discovery that a specific halogen atom change from a fluorine (F) to chlorine (Cl) at the R³ position of Formula (III) is a critical site substitution that was unexpectedly instrumental in further decreasing metabolic stability when exposed to mouse or human microsomes. This change from F to Cl at R³ in Formula (III) was not obvious, as exemplified by a key example within the same target drug class of MEK inhibitors. Binimetinib and selumetinib are U.S. FDA registered MEK inhibitors that differ by a single halogen, where the F— in binimetinib is altered to a Cl in selumetinib. Selumetinib (at ≥6.2 hours—National Center for Biotechnology Information (2023). PubChem Compound Summary for CID 10127622, Selumetinib. Retrieved Jan. 5, 2023 from hips://pubchem.ncbi.nlm.nih.gov/compoundSelumetinib) displays a human plasma drug half-life that is approximately two times longer than binimetinib (at 3.5 hours—National Center for Biotechnology Information (2023). PubChem Compound Summary for CID 10288191, Binimetinib. Retrieved Jan. 5, 2023 from https://pubchem.ncbi.nlm.nih.gov/compound/Binimetinib). Although many drug-like properties varied unpredictably amongst newly synthesized analogues of Formula (III) having Cl at R³, we consistently observed lower microsome metabolic stability, which was a key and desirable attribute for a short-lived, dual-MEK inhibitor designed to enable BID dosing.

Some embodiments relate to a pharmaceutical composition comprising a compound as described herein and a pharmaceutically acceptable salt thereof. Some embodiments relate to a method of treating cancer. In some embodiments, the method includes administering to a subject in need thereof an effective amount of a compound as described herein or a pharmaceutical composition thereof. Some embodiments relate to a use of a compound as described herein for the treatment of cancer. Some embodiments relate to a compound selected from the group consisting of: 4-((dimethylamino)methyl)-5-fluoro-3-(2-fluoro-3-((N-methylsulfamoyl)amino)benzyl)-2-oxo-2H-chromen-7-yl dimethylcarbamate (compound 244); 4-((dimethylamino)methyl)-3-(2-fluoro-3-((N-methylsulfamoyl)amino)benzyl)-5-methoxy-2-oxo-2H-chromen-7-yl dimethylcarbamate (compound 252); 4-((dimethylamino)methyl)-3-(2-fluoro-3-((N-methylsulfamoyl)amino)benzyl)-5-methyl-2-oxo-2H-chromen-7-yl dimethylcarbamate (compound 253); and 4-((dimethylamino)methyl)-3-(3-(ethylsulfonamido)-2-fluorobenzyl)-5-methoxy-2-oxo-2H-chromen-7-yl dimethylcarbamate (compound 269). In some embodiments, the compound is 4-((dimethylamino)methyl)-5-fluoro-3-(2-fluoro-3-((N-methylsulfamoyl)amino)benzyl)-2-oxo-2H-chromen-7-yl dimethylcarbamate (compound 244). In some embodiments, the compound is 4-((dimethylamino)methyl)-3-(2-fluoro-3-((N-methylsulfamoyl)amino)benzyl)-5-methyl-2-oxo-2H-chromen-7-yl dimethylcarbamate (compound 253). In some embodiments, the compound is 4-((dimethylamino)methyl)-3-(3-(ethylsulfonamido)-2-fluorobenzyl)-5-methoxy-2-oxo-2H-chromen-7-yl dimethylcarbamate (compound 269).

Some embodiments relate to a compound selected from the group consisting of: 3-(2-chloro-3-((N-methylsulfamoyl)amino)benzyl)-4-((ethyl(methyl)amino)methyl)-2-oxo-2H-chromen-7-yl dimethylcarbamate (compound 245); 3-(2-chloro-3-((N-methylsulfamoyl)amino)benzyl)-2-oxo-4-(piperazin-1-ylmethyl)-2H-chromen-7-yl dimethylcarbamate (compound 246); 6-chloro-3-(2-fluoro-3-((2,2,2-trifluoroethyl)sulfonamido)benzyl)-2-oxo-4-(piperazin-1-ylmethyl)-2H-chromen-7-yl dimethylcarbamate (compound 247); 3-(2-chloro-3-(ethylsulfonamido)benzyl)-4-((dimethylamino)methyl)-2-oxo-2H-chromen-7-yl dimethylcarbamate (compound 249); 3-(2-chloro-3-((N-methylsulfamoyl)amino)benzyl)-6-fluoro-2-oxo-4-(piperazin-1-ylmethyl)-2H-chromen-7-yl dimethylcarbamate (compound 254); 3-(2-chloro-3-(ethylsulfonamido)benzyl)-6-fluoro-2-oxo-4-(piperazin-1-ylmethyl)-2H-chromen-7-yl dimethylcarbamate (compound 255); 6-chloro-3-(2-chloro-3-((N-methylsulfamoyl)amino)benzyl)-2-oxo-4-(piperazin-1-ylmethyl)-2H-chromen-7-yl dimethylcarbamate (compound 256); 6-chloro-3-(2-chloro-3-(ethylsulfonamido)benzyl)-2-oxo-4-(piperazin-1-ylmethyl)-2H-chromen-7-yl dimethylcarbamate (compound 257); 3-(2-chloro-3-(ethylsulfonamido)benzyl)-2-oxo-4-(piperazin-1-ylmethyl)-2H-chromen-7-yl dimethylcarbamate (compound 258); and 3-(2-chloro-3-((N-methylsulfamoyl)amino)benzyl)-4-((dimethylamino)methyl)-2-oxo-2H-chromen-7-yl dimethylcarbamate (compound 274). In some embodiments, the compound is 3-(2-chloro-3-((N-methylsulfamoyl)amino)benzyl)-4-((ethyl(methyl)amino)methyl)-2-oxo-2H-chromen-7-yl dimethylcarbamate(compound 245). In some embodiments, the compound is 3-(2-chloro-3-((N-methylsulfamoyl)amino)benzyl)-2-oxo-4-(piperazin-1-ylmethyl)-2H-chromen-7-yl dimethylcarbamate (compound 246). In some embodiments, the compound is 3-(2-chloro-3-(ethylsulfonamido)benzyl)-4-((dimethylamino)methyl)-2-oxo-2H-chromen-7-yl dimethylcarbamate (compound 249). In some embodiments, the compound is 3-(2-chloro-3-((N-methylsulfamoyl)amino)benzyl)-6-fluoro-2-oxo-4-(piperazin-1-ylmethyl)-2H-chromen-7-yl dimethylcarbamate (compound 254). In some embodiments, the compound is 3-(2-chloro-3-(ethylsulfonamido)benzyl)-6-fluoro-2-oxo-4-(piperazin-1-ylmethyl)-2H-chromen-7-yl dimethylcarbamate (compound 245). In some embodiments, the compound is 6-chloro-3-(2-chloro-3-((N-methylsulfamoyl)amino)benzyl)-2-oxo-4-(piperazin-1-ylmethyl)-2H-chromen-7-yl dimethylcarbamate (compound 256). In some embodiments, the compound is 6-chloro-3-(2-chloro-3-(ethylsulfonamido)benzyl)-2-oxo-4-(piperazin-1-ylmethyl)-2H-chromen-7-yl dimethylcarbamate (compound 257). In some embodiments, the compound is 3-(2-chloro-3-(ethylsulfonamido)benzyl)-2-oxo-4-(piperazin-1-ylmethyl)-2H-chromen-7-yl dimethylcarbamate. In some embodiments, the compound is 3-(2-chloro-3-((N-methylsulfamoyl)amino)benzyl)-4-((dimethylamino)methyl)-2-oxo-2H-chromen-7-yl dimethylcarbamate (compound 274).

Some embodiments relate to a compound selected from the group consisting of: 3-(2-chloro-3-((N-methylsulfamoyl)amino)benzyl)-4-((dimethylamino)methyl)-6-fluoro-2-oxo-2H-chromen-7-yl dimethylcarbamate; 3-(2-chloro-3-(ethylsulfonamido)benzyl)-4-((dimethylamino)methyl)-6-fluoro-2-oxo-2H-chromen-7-yl dimethylcarbamate; 3-(2-chloro-3-((N-methylsulfamoyl)amino)benzyl)-4-((ethyl(methyl)amino)methyl)-6-fluoro-2-oxo-2H-chromen-7-yl dimethylcarbamate; 4-(azetidin-1-ylmethyl)-3-(2-chloro-3-((N-methylsulfamoyl)amino)benzyl)-6-fluoro-2-oxo-2H-chromen-7-yl dimethylcarbamate; 6-chloro-3-(2-chloro-3-((N-methylsulfamoyl)amino)benzyl)-4-((dimethylamino)methyl)-2-oxo-2H-chromen-7-yl dimethylcarbamate; 6-chloro-3-(2-chloro-3-((N-methylsulfamoyl)amino)benzyl)-4-((ethyl(methyl)amino)methyl)-2-oxo-2H-chromen-7-yl dimethylcarbamate; and 4-(azetidin-1-ylmethyl)-6-chloro-3-(2-chloro-3-((N-methylsulfamoyl)amino)benzyl)-2-oxo-2H-chromen-7-yl dimethylcarbamate. In some embodiments, the compound is 3-(2-chloro-3-((N-methylsulfamoyl)amino)benzyl)-4-((dimethylamino)methyl)-6-fluoro-2-oxo-2H-chromen-7-yl dimethylcarbamate. In some embodiments, the compound is 3-(2-chloro-3-(ethylsulfonamido)benzyl)-4-((dimethylamino)methyl)-6-fluoro-2-oxo-2H-chromen-7-yl dimethylcarbamate. In some embodiments, the compound is 3-(2-chloro-3-((N-methylsulfamoyl)amino)benzyl)-4-((ethyl(methyl)amino)methyl)-6-fluoro-2-oxo-2H-chromen-7-yl dimethylcarbamate. In some embodiments, the compound is 4-(azetidin-1-ylmethyl)-3-(2-chloro-3-((N-methylsulfamoyl)amino)benzyl)-6-fluoro-2-oxo-2H-chromen-7-yl dimethylcarbamate. In some embodiments, the compound is 6-chloro-3-(2-chloro-3-((N-methylsulfamoyl)amino)benzyl)-4-((dimethylamino)methyl)-2-oxo-2H-chromen-7-yl dimethylcarbamate. In some embodiments, the compound is 6-chloro-3-(2-chloro-3-((N-methylsulfamoyl)amino)benzyl)-4-((ethyl(methyl)amino)methyl)-2-oxo-2H-chromen-7-yl dimethylcarbamate.

Some embodiments relate to a compound selected from the group consisting of: 3-(2-fluoro-3-((N-methylsulfamoyl)amino)benzyl)-4-(((2-fluoroethyl)(methyl)amino)methyl)-2-oxo-2H-chromen-7-yl dimethylcarbamate; 3-(3-(ethylsulfonamido)-2-fluorobenzyl)-4-(((2-fluoroethyl)(methyl)amino)methyl)-2-oxo-2H-chromen-7-yl dimethylcarbamate; 3-(2-fluoro-3-((N-methylsulfamoyl)amino)benzyl)-4-((methyl(prop-2-yn-1-yl)amino)methyl)-2-oxo-2H-chromen-7-yl dimethylcarbamate; 4-(((2,2-difluoroethyl)(methyl)amino)methyl)-3-(2-fluoro-3-((N-methylsulfamoyl)amino)benzyl)-2-oxo-2H-chromen-7-yl dimethylcarbamate; 4-(((cyanomethyl)(methyl)amino)methyl)-3-(2-fluoro-3-((N-methylsulfamoyl)amino)benzyl)-2-oxo-2H-chromen-7-yl dimethylcarbamate; 4-((dimethylamino)methyl)-3-(2-fluoro-3-(methyl(sulfamoyl)amino)benzyl)-2-oxo-2H-chromen-7-yl dimethylcarbamate; 4-((dimethylamino)methyl)-3-(2-fluoro-3-(hydroxymethyl)benzyl)-2-oxo-2H-chromen-7-yl dimethylcarbamate; 4-((dimethylamino)methyl)-3-(3-((ethylsulfonyl)methyl)-2-fluorobenzyl)-2-oxo-2H-chromen-7-yl dimethylcarbamate; and 3-(3-((tert-butylsulfinyl)amino)-2-fluorobenzyl)-4-((dimethylamino)methyl)-2-oxo-2H-chromen-7-yl dimethylcarbamate. In some embodiments, the compound is 3-(2-fluoro-3-((N-methylsulfamoyl)amino)benzyl)-4-(((2-fluoroethyl)(methyl)amino)methyl)-2-oxo-2H-chromen-7-yl dimethylcarbamate. In some embodiments, the compound is 3-(3-(ethylsulfonamido)-2-fluorobenzyl)-4-(((2-fluoroethyl)(methyl)amino)methyl)-2-oxo-2H-chromen-7-yl dimethylcarbamate. In some embodiments, the compound is 3-(2-fluoro-3-((N-methylsulfamoyl)amino)benzyl)-4-((methyl(prop-2-yn-1-yl)amino)methyl)-2-oxo-2H-chromen-7-yl dimethylcarbamate. In some embodiments, the compound is 4-(((2,2-difluoroethyl)(methyl)amino)methyl)-3-(2-fluoro-3-((N-methylsulfamoyl)amino)benzyl)-2-oxo-2H-chromen-7-yl dimethylcarbamate. In some embodiments, the compound is 4-(((cyanomethyl)(methyl)amino)methyl)-3-(2-fluoro-3-((N-methylsulfamoyl)amino)benzyl)-2-oxo-2H-chromen-7-yl dimethylcarbamate. In some embodiments, the compound is 4-((dimethylamino)methyl)-3-(2-fluoro-3-(methyl(sulfamoyl)amino)benzyl)-2-oxo-2H-chromen-7-yl dimethylcarbamate. In some embodiments, the compound is 4-((dimethylamino)methyl)-3-(2-fluoro-3-(hydroxymethyl)benzyl)-2-oxo-2H-chromen-7-yl dimethylcarbamate. In some embodiments, the compound is 3-(3-((tert-butylsulfinyl)amino)-2-fluorobenzyl)-4-((dimethylamino)methyl)-2-oxo-2H-chromen-7-yl dimethylcarbamate.

### BRIEF DESCRIPTION OF THE DRAWINGS

FIG. 1 illustrates exemplary compounds of the disclosure pharmacokinetics compared to analog compounds.

FIG. 2A illustrates a graph of pERK:total ERK (activation) in A549 lung cancer model; FIG. 2B illustrates a graph of pERK:total ERK (activation) in A375 model; FIG. 2C illustrates a graph of pERK:total ERK in a SK-MEL-2Melanoma model.

FIG. 3 illustrates a graph of a colon-26 syngeneic CRC tumor mouse model (BID) study.

FIG. 4 illustrates a graph of a colon-26 syngeneic CRC tumor mouse model (QD) study.

FIG. 5 illustrates a table of compounds of the disclosure.

### DETAILED DESCRIPTION

In some embodiments, MEK inhibitors are provided. Various embodiments of these compounds include compounds having the structure of Formula I as described herein or pharmaceutically acceptable salts thereof. In some embodiments, prodrugs, metabolites, stereoisomers, hydrates, solvates, polymorphs, and pharmaceutically acceptable salts of the compounds disclosed herein are provided.

In certain aspects, therapeutic methods or uses are providing herein for the treatment, prevention, or amelioration of a disease or condition in a subject, these methods comprising administering at least one compound disclosed herein to the subject. In some embodiments, therapeutic methods or uses are provided for the treatment, prevention or amelioration of cancer comprising administering of a compound having the structures of Formula (I), (Ia), (Ib), (Ic), (Id) (II), (IIa), (IIb), (IIc), (IId), (III), or (IV), as described herein. In some embodiments, therapeutic methods or uses are provided for the treatment of cancer cachexia comprising administering a compound having the structures of Formula (I), (Ia), (Ib), (Ic), (Id) (II), (IIa), (IIb), (IIc), (IId), (III), or (IV), as described herein.

### Definitions

Unless expressly defined otherwise, technical and/or scientific terms used herein have the same meaning as is commonly understood by one of ordinary skill in the art. In the event that there are a plurality of definitions for a term herein, those in this section prevail unless stated otherwise. As used in the specification and the appended claims, the singular forms “a,” “an,” and “the” include plural referents unless the context clearly dictates otherwise. Unless otherwise indicated, conventional methods of mass spectroscopy, NMR, HPLC, protein chemistry, biochemistry, and pharmacology are employed. The use of either the conjunction “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

While the disclosure has been illustrated and described in detail in the foregoing description, such description is to be considered illustrative or exemplary and not restrictive. The disclosure is not limited to the disclosed embodiments. Variations to the disclosed embodiments can be understood and effected by those skilled in the art in practicing the claimed disclosure, from a study of the disclosure and the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

Where a range of values is provided, it is understood that the upper and lower limit, and each intervening value between the upper and lower limit of the range is encompassed within the embodiments.

The term “prodrug,” as used herein, refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility, but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

Metabolites of the compounds disclosed herein include active species that are produced upon introduction of the compounds into the biological milieu.

Compounds disclosed herein having at least one chiral center they may exist as a racemate or as each enantiomer, and may exist as enantiomeric-enriched mixtures of the enantimoers. It should be noted that all such isomers and mixtures thereof are included in the scope of the present invention. Furthermore, the crystalline forms for the compounds disclosed herein may exist as alternative polymorphs. Such polymorphs are included in one embodiment of the present invention. In addition, some of the compounds of the present invention may form solvates with water (i.e., hydrates) or common organic solvents. Such solvates are included in one embodiment of the present invention.

The term “pharmaceutically acceptable salt,” as used herein, refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

If the manufacture of pharmaceutical formulations involves intimate mixing of the pharmaceutical excipients and the active ingredient in its salt form, then it may be desirable to use pharmaceutical excipients which are non-basic, that is, either acidic or neutral excipients.

In various embodiments, the compounds disclosed herein can be used alone, in combination with other compounds disclosed herein, or in combination with one or more other agents active in the therapeutic areas described herein.

The term “halogen atom,” as used herein, means any one of the radio-stable atoms of column 7 of the Periodic Table of the Elements, e.g., fluorine, chlorine, bromine, or iodine, with fluorine and chlorine being preferred.

The term “ester,” as used herein, refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

The term “amide,” as used herein, refers to a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxyl, or carboxyl side chain on the compounds disclosed herein can be esterified or amidified. The procedures and specific groups to be used to achieve this end are known to those of skill in the art and can readily be found in reference sources such as Greene and Wuts, Protective Groups in Organic Synthesis, 3^{rd} Ed., John Wiley & Sons, New York, NY, 1999, which is incorporated herein in its entirety.

The term “aromatic,” as used herein, refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” refers to an aromatic group which contains at least one heterocyclic ring.

As used herein, “Cₐ to C_{b}” in which “a” and “b” are integers refer to the number of carbon atoms in an alkyl, alkenyl or alkynyl group, or the number of carbon atoms in the ring of a cycloalkyl, aryl, heteroaryl or heterocyclyl group. That is, the alkyl, alkenyl, alkynyl, ring of the cycloalkyl, ring of the aryl, ring of the heteroaryl or ring of the heterocyclyl can contain from “a” to “b”, inclusive, carbon atoms. Thus, for example, a “C₁ to C₄ alkyl” group or a “C₁-C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. Likewise, for example, cycloalkyl group may contain from “a” to “b”, inclusive, total atoms, such as a C₃-C₈ cycloalkyl group, 3 to 8 carbon atoms in the ring(s). If no “a” and “b” are designated with regard to an alkyl, cycloalkyl, or cycloalkenyl, the broadest range described in these definitions is to be assumed. Similarly, a “4 to 7 membered heterocyclyl” group refers to all heterocyclyl groups with 4 to 7 total ring atoms, for example, azetidine, oxetane, oxazoline, pyrrolidine, piperidine, piperazine, morpholine, and the like. As used herein, the term “C₁-C₆” includes C₁, C₂, C₃, C₄, C₅ and C₆, and a range defined by any of the two preceding numbers. For example, C₁-C₆ alkyl includes C₁, C₂, C₃, C₄, C₅ and C₆ alkyl, C₂-C₆ alkyl, C₁-C₃ alkyl, etc. Similarly, C₃-C₈ carbocyclyl or cycloalkyl each includes hydrocarbon ring containing 3, 4, 5, 6, 7 and 8 carbon atoms, or a range defined by any of the two numbers, such as C₃-C₇ cycloalkyl or C₅-C₆cycloalkyl. As another example, 3 to 10 membered heterocyclyl includes 3, 4, 5, 6, 7, 8, 9, or 10 ring atoms, or a range defined by any of the two preceding numbers, such as 4 to 6 membered or 5 to 7 membered heterocyclyl.

As used herein, “alkyl” refers to a straight or branched hydrocarbon chain fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Wherever a substituent is described as being “optionally substituted” that substituent may be substituted with one of the above substituents.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group of the compounds may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group of the compounds may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene. An aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. When substituted, substituents on an aryl group may form a non-aromatic ring fused to the aryl group, including a cycloalkyl, cycloalkenyl, cycloalkynyl, and heterocyclyl.

As used herein, “heteroaryl” refers to a monocyclic or multicyclic aromatic ring system (a ring system with fully delocalized pi-electron system), one or two or more fused rings that contain(s) one or more heteroatoms, that is, an element other than carbon, including but not limited to, nitrogen, oxygen and sulfur. Examples of heteroaryl rings include, but are not limited to, furan, thiophene, phthalazine, pyrrole, oxazole, thiazole, imidazole, pyrazole, isoxazole, isothiazole, triazole, thiadiazole, pyridine, pyridazine, pyrimidine, pyrazine and triazine. A heteroaryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. When substituted, substituents on a heteroayl group may form a non-aromatic ring fused to the aryl group, including a cycloalkyl, cycloalkenyl, cycloalkynyl, and heterocyclyl.

As used herein, an “aralkyl” or “arylalkyl” refers to an aryl group connected, as a substituent, via an alkylene group. The alkylene and aryl group of an aralkyl may be substituted or unsubstituted. Examples include but are not limited to benzyl, substituted benzyl, 2-phenylethyl, 3-phenylpropyl, and naphtylalkyl. In some cases, the alkylene group is a lower alkylene group.

As used herein, a “heteroaralkyl” or “heteroarylalkyl” is heteroaryl group connected, as a substituent, via an alkylene group. The alkylene and heteroaryl group of heteroaralkyl may be substituted or unsubstituted. Examples include but are not limited to 2-thienylmethyl, 3-thienylmethyl, furylmethyl, thienylethyl, pyrrolylalkyl, pyridylalkyl, isoxazollylalkyl, and imidazolylalkyl, and their substituted as well as benzo-fused analogs. In some cases, the alkylene group is a lower alkylene group.

As used herein, a “alkylene” refers to a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” refers to a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

As used herein, “alkylidene” refers to a divalent group, such as =CR′R″, which is attached to one carbon of another group, forming a double bond, alkylidene groups include, but are not limited to, methylidene (═CH₂) and ethylidene (═CHCH₃). As used herein, “arylalkylidene” refers to an alkylidene group in which either R′ and R″ is an aryl group. An alkylidene group may be substituted or unsubstituted.

As used herein, “alkoxy” refers to the formula —OR wherein R is an alkyl is defined as above, e.g. methoxy, ethoxy, n-propoxy, 1-methylethoxy (isopropoxy), n-butoxy, iso-butoxy, sec-butoxy, tert-butoxy, amoxy, tert-amoxy and the like. An alkoxy may be substituted or unsubstituted.

As used herein, “alkylthio” refers to the formula —SR wherein R is an alkyl is defined as above, e.g. methylmercapto, ethylmercapto, n-propylmercapto, 1-methylethylmercapto (isopropylmercapto), n-butylmercapto, iso-butylmercapto, sec-butylmercapto, tert-butylmercapto, and the like. An alkylthio may be substituted or unsubstituted.

As used herein, “aryloxy” and “arylthio” refers to RO- and RS-, respectively, in which R is an aryl, such as but not limited to phenyl. Both an aryloxyl and arylthio may be substituted or unsubstituted.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

As used herein, “cycloalkyl” refers to a completely saturated (no double bonds) mono- or multi-cyclic hydrocarbon ring system. When composed of two or more rings, the rings may be joined together in a fused, bridged or spiro-connected fashion. Cycloalkyl groups may range from C₃ to C₁₀, in other embodiments it may range from C₃ to C₆. A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be an alkyl or selected from those indicated above with regard to substitution of an alkyl group unless otherwise indicated. When substituted, substituents on a cycloalkyl group may form an aromatic ring fused to the cycloalkyl group, including an aryl and a heteroaryl.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). When composed of two or more rings, the rings may be connected together in a fused, bridged or spiro-connected fashion. A cycloalkenyl group may be unsubstituted or substituted. When substituted, the substituent(s) may be an alkyl or selected from the groups disclosed above with regard to alkyl group substitution unless otherwise indicated. When substituted, substituents on a cycloalkenyl group may form an aromatic ring fused to the cycloalkenyl group, including an aryl and a heteroaryl.

As used herein, “cycloalkynyl” refers to a cycloalkyl group that contains one or more triple bonds in the ring. When composed of two or more rings, the rings may be joined together in a fused, bridged or spiro-connected fashion. A cycloalkynyl group may be unsubstituted or substituted. When substituted, the substituent(s) may be an alkyl or selected from the groups disclosed above with regard to alkyl group substitution unless otherwise indicated. When substituted, substituents on a cycloalkynyl group may form an aromatic ring fused to the cycloalkynyl group, including an aryl and a heteroaryl.

As used herein, “heteroalicyclic” or “heteroalicyclyl” refers to a stable 3- to 18 membered ring which consists of carbon atoms and from one to five heteroatoms selected from the group consisting of nitrogen, oxygen and sulfur. The “heteroalicyclic” or “heteroalicyclyl” may be monocyclic, bicyclic, tricyclic, or tetracyclic ring system, which may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl groups may be unsubstituted or substituted. When substituted, the substituent(s) may be one or more groups independently selected from the group consisting of alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” include but are not limited to, azepinyl, acridinyl, carbazolyl, cinnolinyl, dioxolanyl, imidazolinyl, morpholinyl, oxiranyl, piperidinyl N-oxide, piperidinyl, piperazinyl, pyrrolidinyl, 4-piperidonyl, pyrazolidinyl, 2-oxopyrrolidinyl, thiamorpholinyl, thiamorpholinyl sulfoxide, and thiamorpholinyl sulfone. When substituted, substituents on a heteroalicyclyl group may form an aromatic ring fused to the heteroalicyclyl group, including an aryl and a heteroaryl.

As used herein, the term “(cycloalkenyl)alkyl” refers to a cycloalkenyl group connected, as a substituent, via an alkylene group. The alkylene and cycloalkenyl of a (cycloalkenyl)alkyl may be substituted or unsubstituted. In some cases, the alkylene group is a lower alkylene group.

As used herein, the term “(cycloalkynyl)alkyl” to a cycloalkynyl group connected, as a substituent, via an alkylene group. The alkylene and cycloalkynyl of a (cycloalkynyl)alkyl may be substituted or unsubstituted. In some cases, the alkylene group is a lower alkylene group.

As used herein, the term “O-carboxy” refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

As used herein, the term “C-carboxy” refers to a “—C(═O)R” group in which R can be the same as defined with respect to O-carboxy. A C-carboxy may be substituted or unsubstituted.

As used herein, the term “trihalomethanesulfonyl” refers to an “X₃CSO₂—” group wherein X is a halogen.

As used herein, the term “cyano” refers to a “—CN” group.

As used herein, the term “cyanato” refers to an “—OCN” group.

As used herein, the term “isocyanato” refers to a “—NCO” group.

As used herein, the term “thiocyanato” refers to a “—SCN” group.

As used herein, the term “isothiocyanato” refers to an “—NCS” group.

As used herein, the term “sulfinyl” refers to a “—S(═O)—R” group in which R can be the same as defined with respect to O-carboxy. A sulfinyl may be substituted or unsubstituted.

As used herein, the term “sulfonyl” refers to an “—SO₂R” group in which R can be the same as defined with respect to O-carboxy. A sulfonyl may be substituted or unsubstituted.

As used herein, the term “S-sulfonamido” refers to a “—SO₂NR_{A}R_{B}” group in which R_{A} and R_{B} can be the same as defined with respect to O-carboxy. An S-sulfonamido may be substituted or unsubstituted.

As used herein, the term “N-sulfonamido” refers to a “—SO₂N(R_{A})(R_{B})” group in which R, R_{A}, and R_{B} can be the same as defined with respect to O-carboxy. A sulfonyl may be substituted or unsubstituted.

As used herein, the term “trihalomethanesulfonamido” refers to an “X₃CSO₂N(R)—” group with X as halogen and R can be the same as defined with respect to O-carboxy. A trihalomethanesulfonamido may be substituted or unsubstituted.

As used herein, the term “O-carbamyl” refers to a “—OC(═O)NR_{A}R_{B}” group in which R_{A} and R_{B} can be the same as defined with respect to O-carboxy. An O-carbamyl may be substituted or unsubstituted.

As used herein, the term “N-carbamyl” refers to an “ROC(═O)NR_{A}—” group in which R and R_{A} can be the same as defined with respect to O-carboxy. An N-carbamyl may be substituted or unsubstituted.

As used herein, the term “O-thiocarbamyl” refers to a “—OC(═S)—NR_{A}R_{B}” group in which R_{A} and R_{B} can be the same as defined with respect to O-carboxy. An O-thiocarbamyl may be substituted or unsubstituted.

As used herein, the term “N-thiocarbamyl” refers to an “ROC(═S)NR_{A}—” group in which R and R_{A} can be the same as defined with respect to O-carboxy. An N-thiocarbamyl may be substituted or unsubstituted.

As used herein, the term “C-amido” refers to a “—C(═O)NR_{A}R_{B}” group in which R_{A} and R_{B} can be the same as defined with respect to O-carboxy. A C-amido may be substituted or unsubstituted.

As used herein, the term “N-amido” refers to a “RC(═O)NR_{A}—” group in which R and R_{A} can be the same as defined with respect to O-carboxy. An N-amido may be substituted or unsubstituted.

As used herein, the term “amino” refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, the term “aminoalkyl” refers to an amino group connected via an alkylene group.

As used herein, the term “ester” refers to a “—C(═O)OR” group in which R can be the same as defined with respect to O-carboxy. An ester may be substituted or unsubstituted.

As used herein, the term “lower aminoalkyl” refers to an amino group connected via a lower alkylene group. A lower aminoalkyl may be substituted or unsubstituted.

As used herein, the term “lower alkoxyalkyl” refers to an alkoxy group connected via a lower alkylene group. A lower alkoxyalkyl may be substituted or unsubstituted.

As used herein, the term “acetyl” refers to a —C(═O)CH₃, group.

As used herein, the term “trihalomethanesulfonyl” refers to a X₃CS(═O)₂-group where X is a halogen.

As used herein, the term “O-carbamyl” refers to a —OC(═O)—NR, in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carbamyl can be substituted or unsubstituted.

As used herein, the term “N-carbamyl” refers to a ROC(═O)NH— group, in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An N-carbamyl can be substituted or unsubstituted.

As used herein, the term “O-thiocarbamyl” refers to a —OC(═S)—NR, in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-thiocarbamyl can be substituted or unsubstituted.

As used herein, the term “N-thiocarbamyl” refers to an ROC(═S)NH-group, in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An N-thiocarbamyl can be substituted or unsubstituted.

As used herein, the term “perhaloalkyl” refers to an alkyl group where all of the hydrogen atoms are replaced by halogen atoms.

As used herein, the term “halogen” or “halo,” refer to any one of the radio-stable atoms of column 7 of the Periodic Table of the Elements, e.g., fluorine, chlorine, bromine, or iodine, with fluorine and chlorine being preferred.

As used herein, the term “carbocyclyl” refers to a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle [2.2.2]octanyl, adamantyl, and spiro[4.4]nonanyl.

As used herein, the term “(cycloalkyl)alkyl” refers to a cycloalkyl group connected, as a substituent, via an alkylene group. The alkylene and cycloalkyl of a (cycloalkyl)alkyl may be substituted or unsubstituted. Examples include but are not limited cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, the term “cycloalkyl” refers to a fully saturated carbocyclyl ring or ring system. Examples include cyclopropyl, cyclobutyl, cyclopentyl, and cyclohexyl.

As used herein, the term “cycloalkenyl” means a carbocyclyl ring or ring system having at least one double bond, wherein no ring in the ring system is aromatic. An example is cyclohexenyl.

As used herein, the term “heterocyclyl” refers to three-, four-, five-, six-, seven-, and eight- or more membered rings wherein carbon atoms together with from 1 to 3 heteroatoms constitute said ring. A heterocyclyl can optionally contain one or more unsaturated bonds situated in such a way, however, that an aromatic pi-electron system does not arise. The heteroatoms are independently selected from oxygen, sulfur, and nitrogen.

A heterocyclyl can further contain one or more carbonyl or thiocarbonyl functionalities, so as to make the definition include oxo-systems and thio-systems such as lactams, lactones, cyclic imides, cyclic thioimides, cyclic carbamates, and the like.

As used herein, “heterocyclyl” refers to a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, the term “(heterocyclyl)alkyl” refers to a heterocyclyl group connected, as a substituent, via an alkylene group. Examples include, but are not limited to, imidazolinylmethyl and indolinylethyl.

The terms “purified,” “substantially purified,” and “isolated” as used herein, refer to compounds disclosed herein being free of other, dissimilar compounds with which the compounds of the invention are normally associated in their natural state, so that the compounds of the invention comprise at least 0.5%, 1%, 5%, 10%, or 20%, and most preferably at least 50% or 75% of the mass, by weight, of a given sample.

Substituted groups are based upon or derived from the unsubstituted parent group in which there has been an exchange of one or more hydrogen atoms for another atom or group. Unless otherwise indicated, when a group is deemed to be “substituted,” the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, a substituted group is substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

Unless otherwise indicated, when a substituent is deemed to be “optionally substituted,” it is meant that the substituent” is a group that may be substituted with one or more group(s) individually and independently selected from alkyl, alkenyl, alkynyl, cycloalkyl, aryl, heteroaryl, heteroalicyclic, hydroxyl, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

The term “agent” or “test agent,” as used herein, includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog,” as used herein, refers to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The term “mammal,” as used herein, is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “microbial infection,” as used herein, refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient,” as used herein, includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “subject,” as used herein, refers to a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “effective amount” or a “therapeutically effective amount.” as used herein, refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

The term “treat,” “treatment,” or “treating,” as used herein, refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject.

It is to be understood that where compounds disclosed herein have unfilled valencies, then the valencies are to be filled with hydrogens and/or deuteriums.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

The term “immune checkpoint inhibitor” as used herein refers to a molecule (e.g., small molecule, peptide, polypeptide, protein, antibody, antibody fragment and the like) that acts as an inhibitor (antagonist) of an immune checkpoint pathway. Inhibition of a pathway can include blockade of the pathway through binding to a receptor or signaling molecule that is part of the immune checkpoint pathway.

The term “about,” as used herein, refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. When a value is preceded by the term about, the component is not intended to be limited strictly to that value, but it is intended to include amounts that vary from the value.

### Compounds

Some embodiments provide a compound of Formula (I):

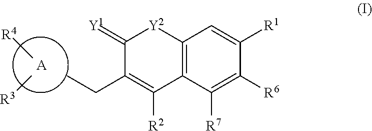

In some embodiments, Formula (I) is a pharmaceutically acceptable salt as described herein.

In some embodiments, Formula (I) is represented by Formula (Ia), Formula (Ib), Formula (Ic), or Formula (Id):

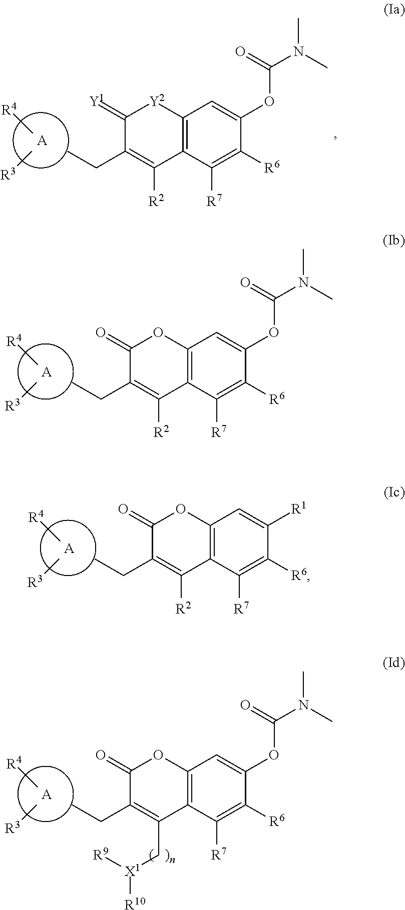

In some embodiments, Formula (Ia), Formula (Ib), Formula (Ic), or Formula (Id) are a pharmaceutically acceptable salt as described herein.

Some embodiments provide a compound of Formula (II):

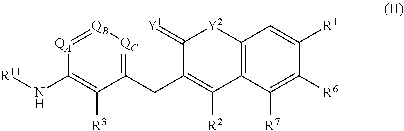

In some embodiments, Formula (II) is a pharmaceutically acceptable salt as described herein.

In some embodiments, Formula (II) is represented by Formula (IIa), Formula (IIb), Formula (IIc):

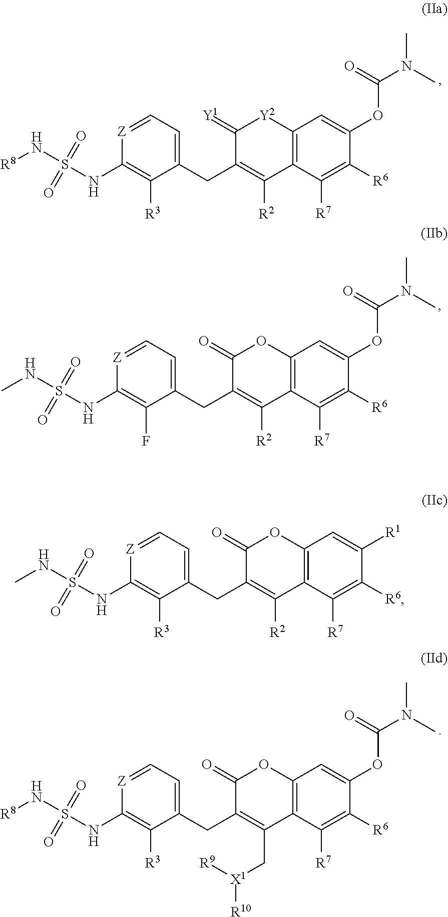

In some embodiments, Formula (IIa), Formula (IIb), Formula (IIc), Formula (IId) may be a pharmaceutically acceptable salt as described herein.

Some embodiments provide a compound of Formula (III):

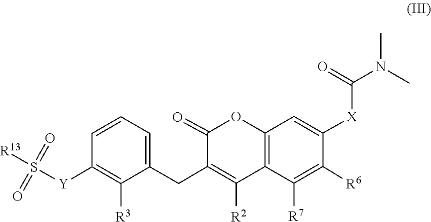

In some embodiments, Formula (III) is a pharmaceutically acceptable salt as described herein.

In some embodiments, Ring A is

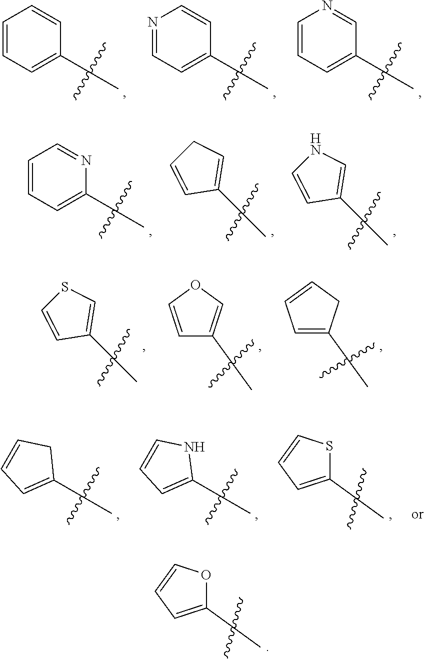

In some embodiments of the compounds of Formula (I) or (Ic), R¹ may be selected from H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, O-aryl, O-heteroaryl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, or L.

In some embodiments, R¹ is not O-pyrimidinyl. In some embodiments, R¹ is not an ether-linked pyrimidyl.

In some embodiments of the compounds of Formula (I), (Ia), (Ib), (II), (IIa), (IIb), or (III), R² may be selected from H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, O-aryl, O-heteroaryl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, or L. In some embodiments, R² is L. In some further embodiments, R² is —CH₃.

In some embodiments of the compounds of Formula (I), (Ia), (Ib), (Ic), (Id), (II), (IIa), (IId), or (III), R³ may be selected from H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, or L. In some further embodiments, R² is L. In some further embodiments, R² is —CH₃.

In some embodiments of the compounds of Formula (I), (Ia), (Tb), (Ic), (Id), (II), or (IIa), R⁴ may be selected from H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, or L.

In some embodiments of the compounds of Formula (I), (Ia), (Tb), (Ic), (II), (IIa), (IIb), or (IIc), R⁵ may be selected from H, deuterium, hydroxyl, halogen, cyano, nitro, optionally substituted amino, optionally substituted C₁ to C₆ alkoxy, optionally substituted C₁ to C₆ alkyl, optionally substituted C₂ to C₆ alkenyl, optionally substituted C₂ to C₆ alkynyl. In some embodiments, R⁵ is H, deuterium, halo, or an optionally substituted C₁ to C₆ alkyl.

In some embodiments of the compounds of Formula (I), (Ia), (Ib), (Ic), (II), (IIa), (IIb), or (IIc), R^{5′} may be selected from H, deuterium, hydroxyl, halogen, cyano, nitro, optionally substituted amino, optionally substituted C₁ to C₆ alkoxy, optionally substituted C₁ to C₆ alkyl, optionally substituted C₂ to C₆ alkenyl, optionally substituted C₂ to C₆ alkynyl. In some embodiments, R^{5′} is H, deuterium, halo, or an optionally substituted C₁ to C₆ alkyl.

In some embodiments of the compounds Formula (I), (Ia), (Ib), (Ic), (II), (IIa), (IIb), (IIc), (IId), or (III), R⁶ may be selected from H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, or L. In some embodiments, R⁶ is selected from the group consisting of H or fluoro, chloro or bromo.

In some embodiments of the compounds of Formula (I), (Ia), (Ib), (Ic), (Id), (II), (IIa), (IIb), (IIc), (IId), or (III), R⁷ may be selected from H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, or L. In some embodiments, R⁷ is F, Cl, or Br. In some embodiments, R⁷ is Cl. In some embodiments, R⁷ may be selected from H, deuterium, hydroxyl, halogen, cyano, nitro, optionally substituted amino, optionally substituted C₁ to C₆ alkoxy, optionally substituted C₁ to C₆ alkyl, optionally substituted C₂ to C₆ alkenyl, optionally substituted C₂ to C₆ alkynyl. In some embodiments, R⁷ is halo. In some embodiments, R⁷ is H. In some embodiments, R⁷ is selected from the group consisting of H, F, methyl, or methoxy. In some embodiments, R⁷ is H or F.

In some embodiments of the compounds of Formula (IIa), R⁸ selected from H, deuterium, optionally substituted C₁ to C₆ alkyl, optionally substituted C₂ to C₆ alkenyl, optionally substituted C₂ to C₆ alkynyl, optionally substituted C₃ to C₈ carbocyclyl, optionally substituted C₆ to C₁₀ aryl, optionally substituted C₃ to C₈ heterocyclyl, optionally substituted C₃ to C₁₀ heteroaryl. In some further embodiments, R⁸ is selected from halo, H, deuterium, or CH₃.

In some embodiments of the compounds of Formula (Id), R⁹ may be selected from hydrogen, deuterium, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl) or —CH₂-(optionally substituted C₃ to C₁₀ heteroaryl). In some further embodiments, Z₂ is C₃ to C₈ cycloalkyl, optionally substituted C₃ to C₈ heterocyclyl, optionally substituted C₃ to C₈ heteroaryl, —NR⁵R^{5′}, —CH₂CH, or —CH₂CN.

In some embodiments of the compounds of Formula (Id), R¹⁰ may be selected from hydrogen, deuterium, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl) or —CH₂-(optionally substituted C₃ to C₁₀ heteroaryl). In some further embodiments, Z₂ is C₃ to C₈ cycloalkyl, optionally substituted C₃ to C₈ heterocyclyl, optionally substituted C₃ to C₈ heteroaryl, —NR⁵R^{5′}, —CH₂CH, or —CH₂CN.

In some embodiments of the compounds of Formula (II), R¹¹ is independently H, deuterium, optionally substituted C₁ to C₆ alkyl, optionally substituted C₂ to C₆ alkenyl, optionally substituted C₂ to C₆ alkynyl, optionally substituted C₃ to C₈ carbocyclyl, optionally substituted C₆ to C₁₀ aryl, optionally substituted C₃ to C₈ heterocyclyl, optionally substituted C₃ to C₁₀ heteroaryl, or L. In some further embodiments, R¹¹ is selected from halo, H or CH₃.

In some embodiments of the compounds of Formula (III), R¹³ may be selected from H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, or L. In some embodiments, R¹³ is C₁ to C₆ alkyl.

In some embodiments of the compounds of Formula (III), X may be selected from C(R⁵)₂, CH(R⁵), CH₂, —O—,

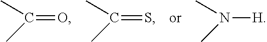

In some further embodiments, X is CH₂ or —O—. In some further embodiments, X is —O—.

In some embodiments of the compounds of Formula (Id) or (IId), X¹ is N or CH.

In some embodiments of the compounds of Formula (Id), n is 1, 2, 3, or 4.

In some embodiments of the compounds of Formula (I), (Ia), (II), or (IIa), Y¹ may be selected from C(R⁵)₂, CH(R⁵), CH₂, —O—,

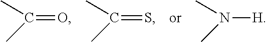

In some further embodiments, Y¹ is CH₂ or —O—. In some further embodiments, Y¹ is —O—. In some embodiments, Y¹ is

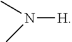

In some embodiments of the compounds of Formula (I), (Ia), (II), or (IIa), Y² may be selected from C(R⁵)₂, CH(R⁵), CH₂, —O—,

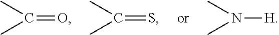

In some embodiments, Y² is —O—. In some embodiments, Y² is

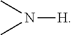

In some embodiments of the compounds of Formula (I), (Ia), (Ib), (Ic), (Id), (II), (IIa), (IIb), (IIc), (IId), or (III), L may be selected from —Z₁-Z₂. In some embodiments of the compounds of Formula (III), L may be selected from —Z₁-Z₂—Z₃.

In some embodiments of the compounds of Formula (I), (Ia), (Ib), (Ic), (Id), (II), (IIa), (IIb), (IIc), (IId), or (III), Z₁ may be selected from —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —NHCH₂CO—, —R⁵(CO)NH—, —R⁵NH—SO₂—, —R⁵SO₂—NH—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₁ heteroaryl, —CH₂-(optionally substituted aryl), —CH₂-(optionally substituted C₃ to C₈ cycloalkyl) or —CH₂-(optionally substituted C₃ to C₁₀ heteroaryl). In some further embodiments, Z₁ is —CH₂—.

In some embodiments of the compounds of Formula (I), (Ia), (Ib), (Ic), (Id), (II), (IIa), (IIb), (IIc), (IId), or (III), Z₂ may be selected from hydrogen, deuterium, halo, —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NHCH₂CO—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —CH₂R⁵—, —OR⁵—, —SR⁵—, S═O—R⁵, —SO₂R⁵, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl) or —CH₂-(optionally substituted C₃ to C₁₀ heteroaryl). In some further embodiments, Z₂ is C₃ to C₈ cycloalkyl, optionally substituted C₃ to C₈ heterocyclyl, optionally substituted C₃ to C₈ heteroaryl, —NR⁵R⁵, —CH₂CH, or —CH₂CN. In some further embodiments, Z₂ is optionally substituted C₃ to C₈ heterocyclyl. In some embodiments, Z₂ is —NR⁵R^{5′}. In some embodiments, Z₁ is —CH₂— and Z₂ is —NR⁵R^{5′}.

In some embodiments of the compounds of Formula (I), (Ia), (Ib), (Ic), (Id), (II), (IIa), (IIb), (IIc), (IId), or (III), Z₃ may be selected from hydrogen, deuterium, halo, —COH, —CO₂H, —NO₂, —CH₂CCH, —CH₂CN, —NR⁵R^{5′}, —(CO)NH₂, —(CO)NR⁵R^{5′}, —SO₂—NH₂, —R⁵CH₃, —R⁵—COH, —R⁵CO₂H, —R⁵NH₂, —R⁵NH(COH), —R⁵(CO)NH₂, —R⁵NH—SO₂H, —R⁵SO₂—NH₂, —CH₂R⁵, —OR⁵, —SO₂R⁵—, —CO₂R⁵, —NHR⁵, —NH(CO)R⁵, —(CO)NHR⁵, —NH—SO₂R⁵, —SO₂—NHR⁵, optionally substituted amino, optionally substituted C₁ to C₄ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl) or —CH₂-(optionally substituted C₃ to C₁₀ heteroaryl).

In some embodiments, Formula (I), (Ia), (Ib), (Ic), (Id), (II), (IIa), (IIb), (IIc), (IId), or (III) is a compound of a disclosed formula, for example Formula (I), (Ia), (Ib), (Ic), (Id), (II), (IIa), (IIb), (IIc), (IId), or (III), but excluding the compounds:

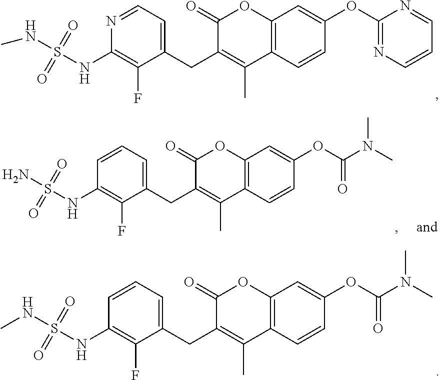

Some embodiments provide a compound of Formula (IV):

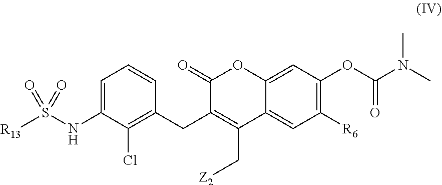

In some embodiments, Formula (IV) is a pharmaceutically acceptable salt as described herein.

In some embodiments of the compounds of Formula (IV), R₆ is hydrogen, fluoro or chloro.

In some embodiments of the compounds of Formula (IV), R₁₃ is ethyl or —NR_{A}R_{B} wherein R_{A} is hydrogen and R_{B} is methyl.

In some embodiments of the compounds of Formula (IV), Z₂ is —NR⁵R^{5′},

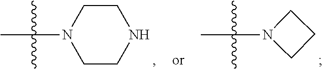 [SMILES: *N1CCNCC1 ; *N1CCC1]

In some embodiments of the compounds of Formula (IV), R⁵ is C₁ to C₆ alkyl.

In some embodiments of the compounds of Formula (IV), R^{5′} is C₁ to C₆ alkyl.

In some embodiments of the compounds of Formula (I), (Ia), (Ib), (Ic), (II), (IIa), (IIb), (IIc), (IId), (III), or (IV) are selected from Compounds of Tables B, C, D, E, and pharmaceutically acceptable salts thereof.

TABLE B
Exemplary compounds of the disclosure.

| No. | Structure |
|---|---|
| 236 | 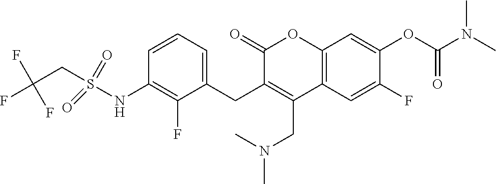 [SMILES: CN(C)Cc1c(Cc2cccc(NS(=O)(=O)CC(F)(F)F)c2F)c(=O)oc2cc(OC(=O)N(C)C)c(F)cc12] |
| 237 | 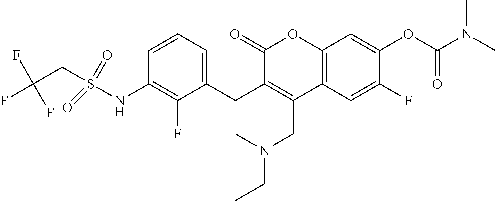 [SMILES: CCN(C)Cc1c(Cc2cccc(NS(=O)(=O)CC(F)(F)F)c2F)c(=O)oc2cc(OC(=O)N(C)C)c(F)cc12] |
| 238 | 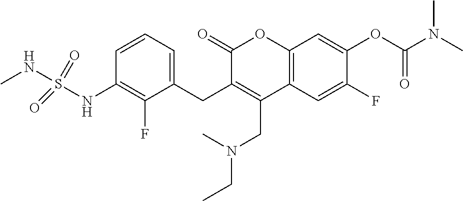 |
| 239 | 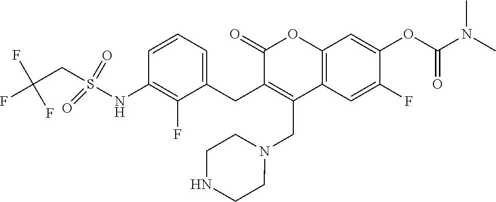 [SMILES: CN(C)C(=O)Oc1cc2oc(=O)c(Cc3cccc(NS(=O)(=O)CC(F)(F)F)c3F)c(CN3CCNCC3)c2cc1F] |
| 240 | 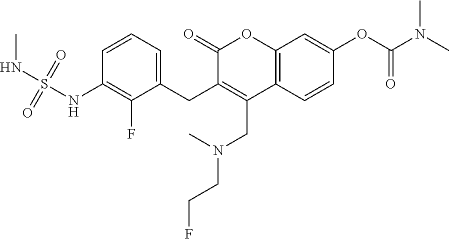 [SMILES: CNS(=O)(=O)Nc1cccc(Cc2c(CN(C)CCF)c3ccc(OC(=O)N(C)C)cc3oc2=O)c1F] |
| 241 | 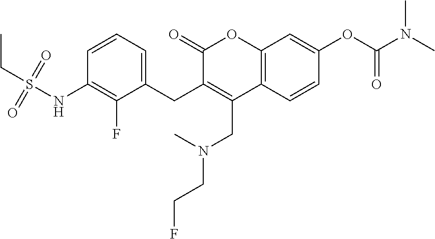 [SMILES: CCS(=O)(=O)Nc1cccc(Cc2c(CN(C)CCF)c3ccc(OC(=O)N(C)C)cc3oc2=O)c1F] |
| 242 | 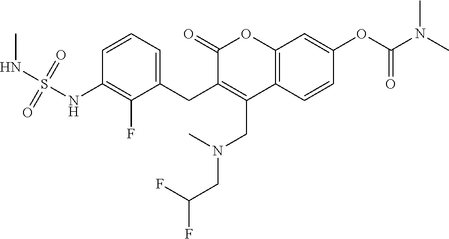 |
| 243 | 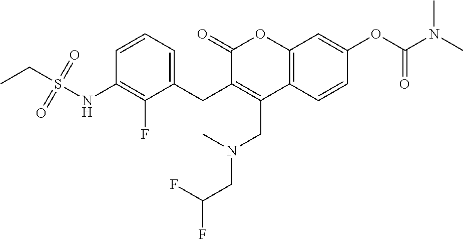 [SMILES: CCS(=O)(=O)Nc1cccc(Cc2c(CN(C)CC(F)F)c3ccc(OC(=O)N(C)C)cc3oc2=O)c1F] |
| 244 | 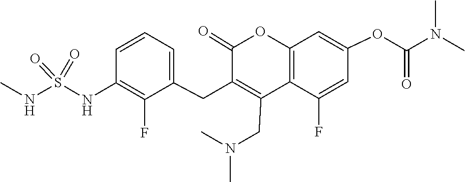 |
| 245 | 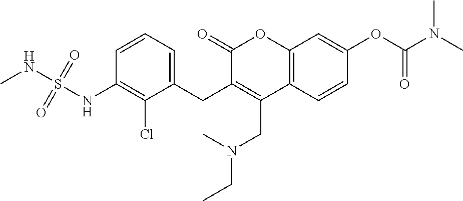 |
| 246 | 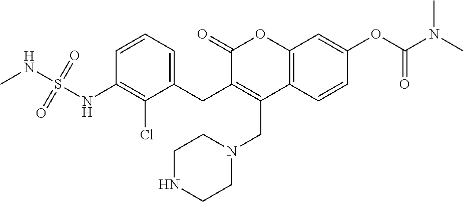 |
| 247 | 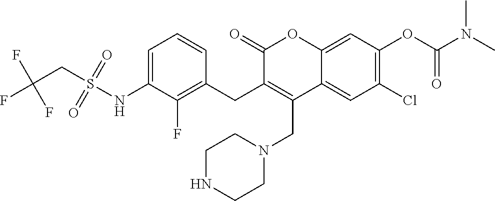 [SMILES: CN(C)C(=O)Oc1cc2oc(=O)c(Cc3cccc(NS(=O)(=O)CC(F)(F)F)c3F)c(CN3CCNCC3)c2cc1Cl] |
| 248 | 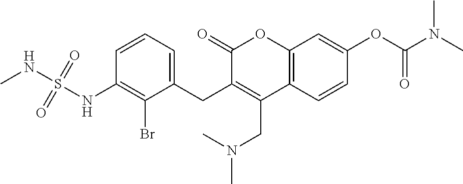 |
| 249 | 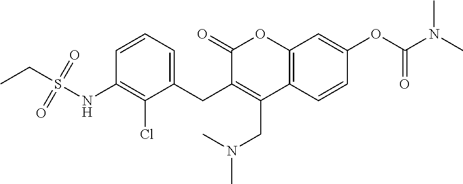 [SMILES: CCS(=O)(=O)Nc1cccc(Cc2c(CN(C)C)c3ccc(OC(=O)N(C)C)cc3oc2=O)c1Cl] |
| 250 | 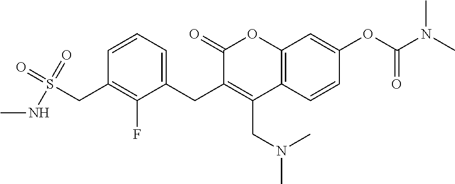 |
| 251 | 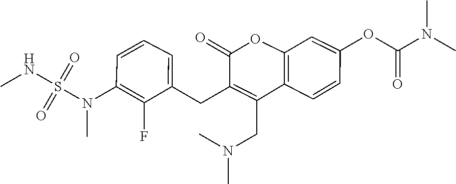 |
| 252 | 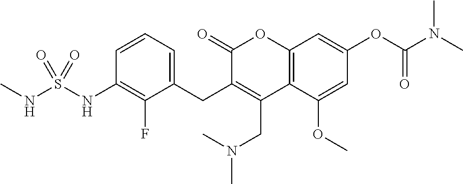 |
| 253 | 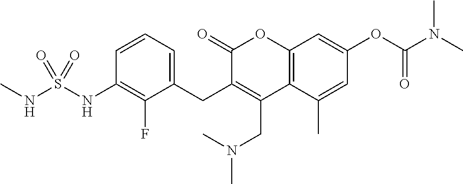 |
| 254 | 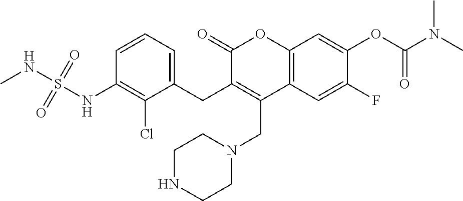 |
| 255 | 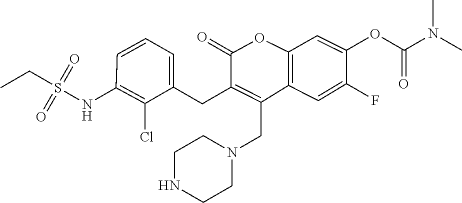 [SMILES: CCS(=O)(=O)Nc1cccc(Cc2c(CN3CCNCC3)c3cc(F)c(OC(=O)N(C)C)cc3oc2=O)c1Cl] |
| 256 | 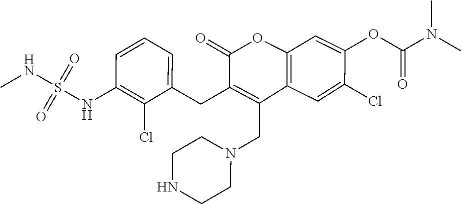 |
| 257 | 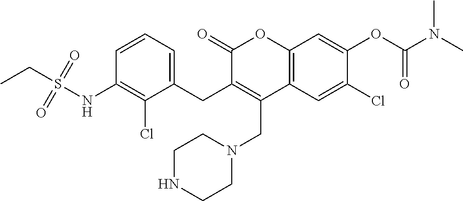 [SMILES: CCS(=O)(=O)Nc1cccc(Cc2c(CN3CCNCC3)c3cc(Cl)c(OC(=O)N(C)C)cc3oc2=O)c1Cl] |
| 258 | 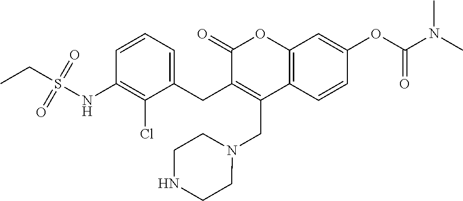 [SMILES: CCS(=O)(=O)Nc1cccc(Cc2c(CN3CCNCC3)c3ccc(OC(=O)N(C)C)cc3oc2=O)c1Cl] |
| 259 | 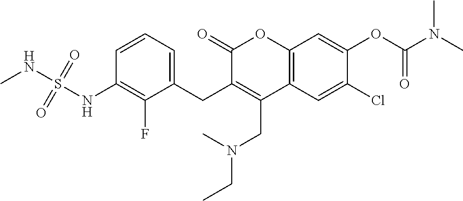 |
| 260 | 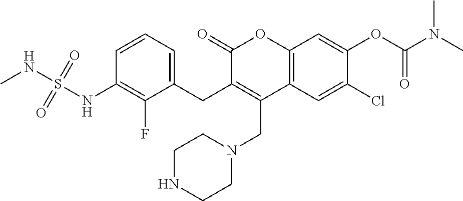 |
| 261 | 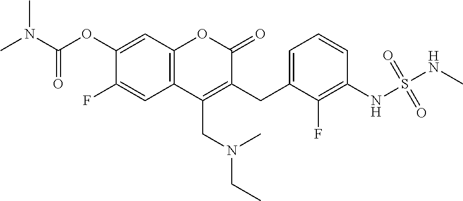 |
| 262 | 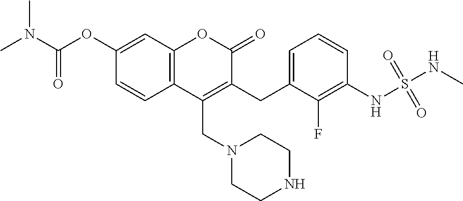 |
| 263 | 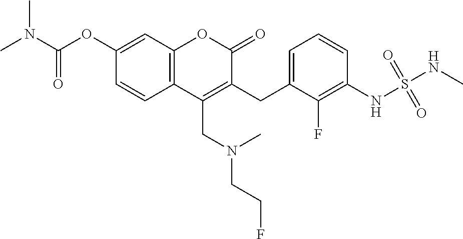 |
| 264 | 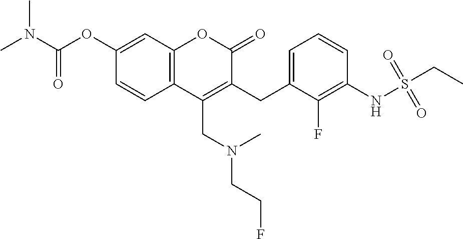 [SMILES: CCS(=O)(=O)Nc1cccc(Cc2c(CN(C)CCF)c3ccc(OC(=O)N(C)C)cc3oc2=O)c1F] |
| 265 | 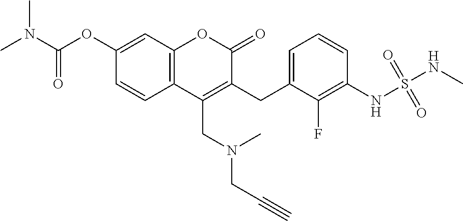 |
| 266 | 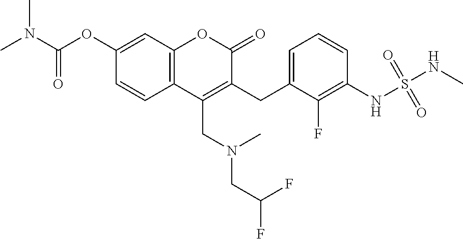 |
| 267 | 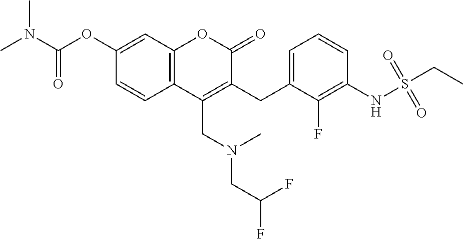 [SMILES: CCS(=O)(=O)Nc1cccc(Cc2c(CN(C)CC(F)F)c3ccc(OC(=O)N(C)C)cc3oc2=O)c1F] |
| 268 | 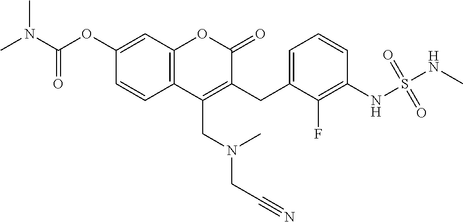 |
| 269 | 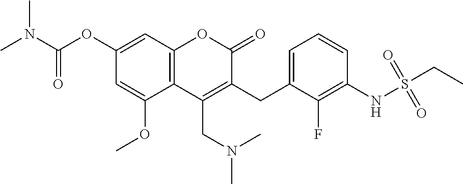 [SMILES: CCS(=O)(=O)Nc1cccc(Cc2c(CN(C)C)c3c(OC)cc(OC(=O)N(C)C)cc3oc2=O)c1F] |
| 270 | 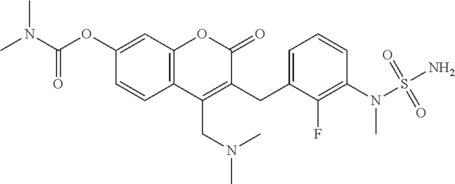 [SMILES: CN(C)Cc1c(Cc2cccc(N(C)S(N)(=O)=O)c2F)c(=O)oc2cc(OC(=O)N(C)C)ccc12] |
| 271 | 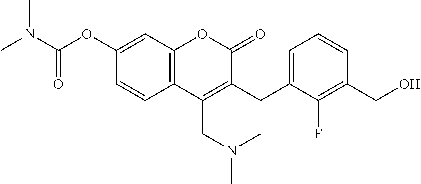 [SMILES: CN(C)Cc1c(Cc2cccc(CO)c2F)c(=O)oc2cc(OC(=O)N(C)C)ccc12] |
| 272 | 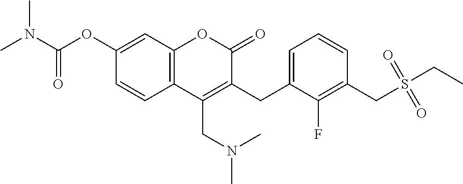 [SMILES: CCS(=O)(=O)Cc1cccc(Cc2c(CN(C)C)c3ccc(OC(=O)N(C)C)cc3oc2=O)c1F] |
| 273 | 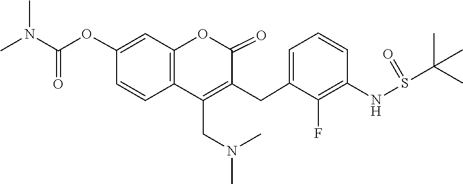 [SMILES: CN(C)Cc1c(Cc2cccc(NS(=O)C(C)(C)C)c2F)c(=O)oc2cc(OC(=O)N(C)C)ccc12] |
| 274 | 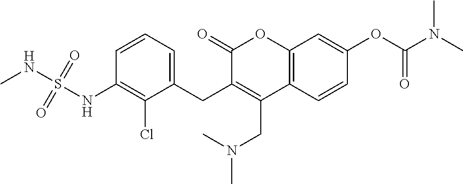 |
| 275 | 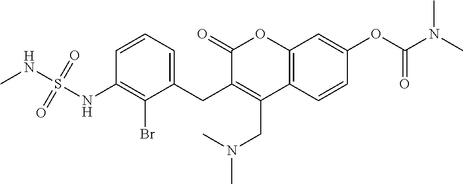 |
| 276 | 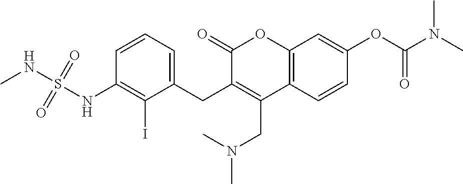 |
| 277 | 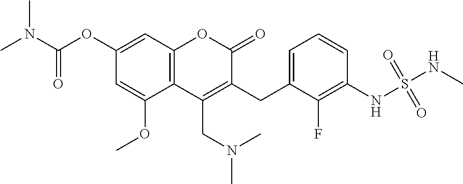 |

TABLE C
Exemplary Compounds of the disclosure.

| No. | Structure |
|---|---|
| 244 | 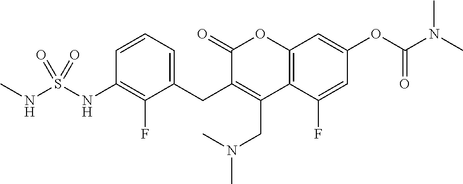 |
| 252 | 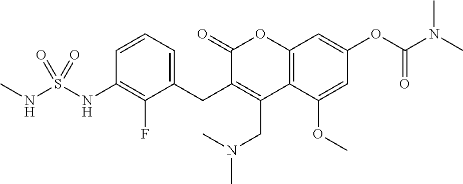 |
| 253 | 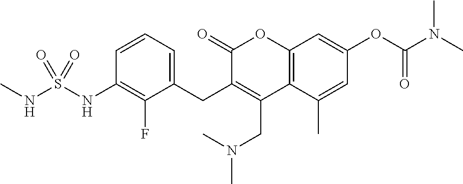 |
| 261 | 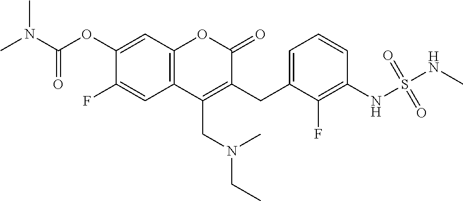 |
| 262 | 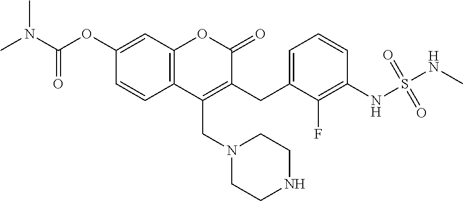 |
| 263 | 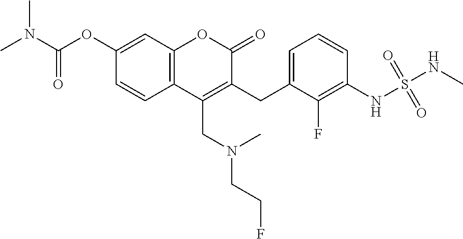 |
| 264 | 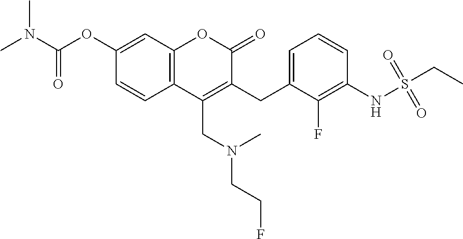 [SMILES: CCS(=O)(=O)Nc1cccc(Cc2c(CN(C)CCF)c3ccc(OC(=O)N(C)C)cc3oc2=O)c1F] |
| 265 | 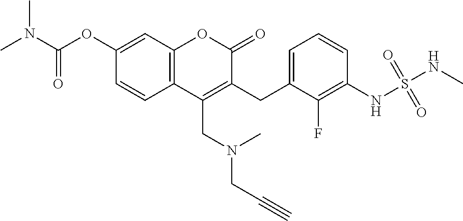 |
| 266 | 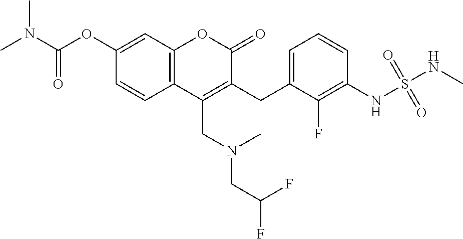 |
| 267 | 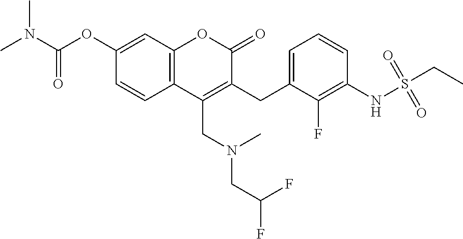 [SMILES: CCS(=O)(=O)Nc1cccc(Cc2c(CN(C)CC(F)F)c3ccc(OC(=O)N(C)C)cc3oc2=O)c1F] |
| 268 | 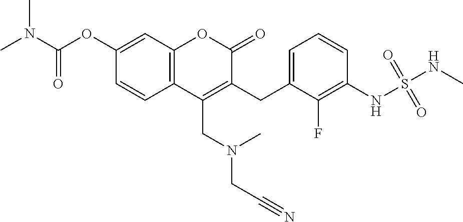 |
| 269 | 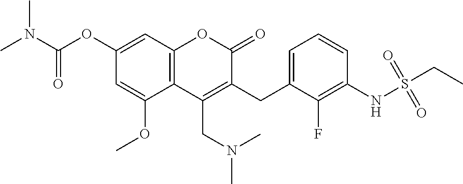 [SMILES: CCS(=O)(=O)Nc1cccc(Cc2c(CN(C)C)c3c(OC)cc(OC(=O)N(C)C)cc3oc2=O)c1F] |
| 270 | 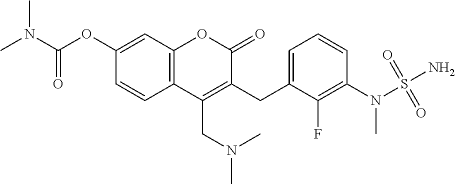 [SMILES: CN(C)Cc1c(Cc2cccc(N(C)S(N)(=O)=O)c2F)c(=O)oc2cc(OC(=O)N(C)C)ccc12] |
| 271 | 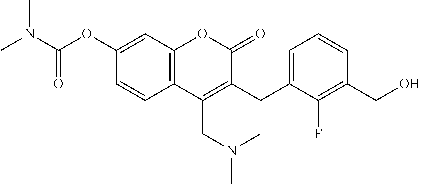 [SMILES: CN(C)Cc1c(Cc2cccc(CO)c2F)c(=O)oc2cc(OC(=O)N(C)C)ccc12] |
| 272 | 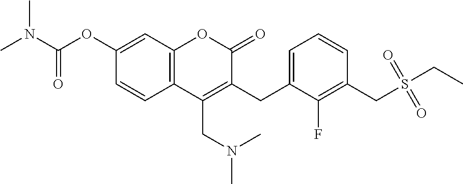 [SMILES: CCS(=O)(=O)Cc1cccc(Cc2c(CN(C)C)c3ccc(OC(=O)N(C)C)cc3oc2=O)c1F] |
| 273 | 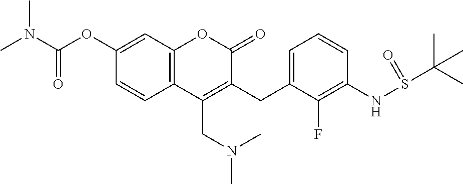 [SMILES: CN(C)Cc1c(Cc2cccc(NS(=O)C(C)(C)C)c2F)c(=O)oc2cc(OC(=O)N(C)C)ccc12] |

TABLE D
Exemplary Compounds of the disclosure.

| No. | Structure |
|---|---|
| 245 | 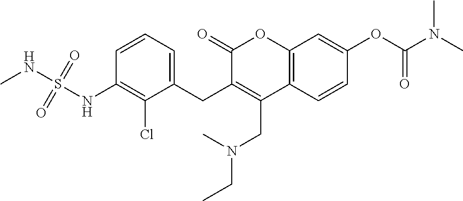 |
| 246 | 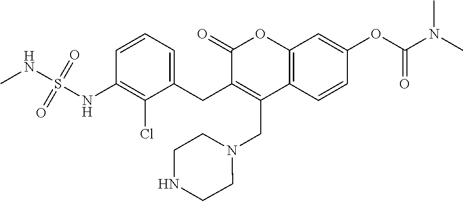 |
| 249 | 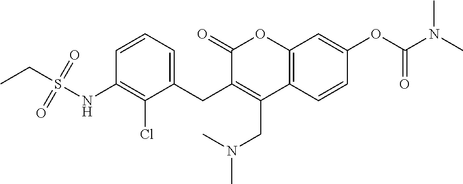 [SMILES: CCS(=O)(=O)Nc1cccc(Cc2c(CN(C)C)c3ccc(OC(=O)N(C)C)cc3oc2=O)c1Cl] |
| 254 | 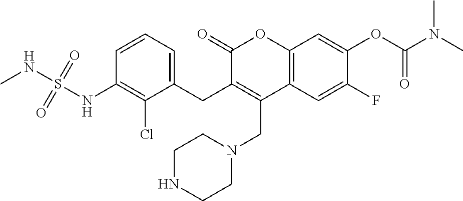 |
| 255 | 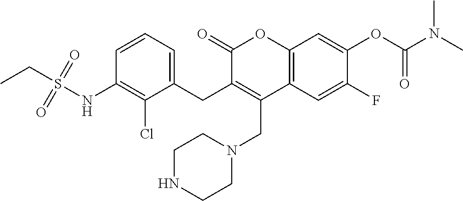 [SMILES: CCS(=O)(=O)Nc1cccc(Cc2c(CN3CCNCC3)c3cc(F)c(OC(=O)N(C)C)cc3oc2=O)c1Cl] |
| 256 | 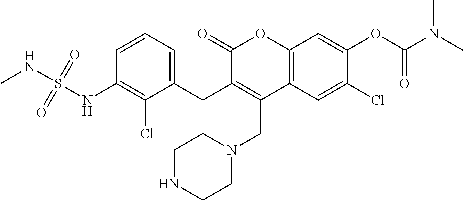 |
| 257 | 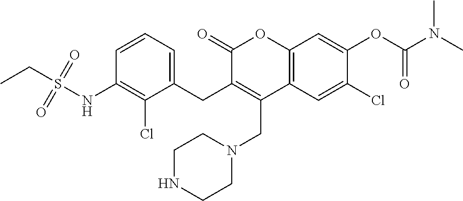 [SMILES: CCS(=O)(=O)Nc1cccc(Cc2c(CN3CCNCC3)c3cc(Cl)c(OC(=O)N(C)C)cc3oc2=O)c1Cl] |
| 258 | 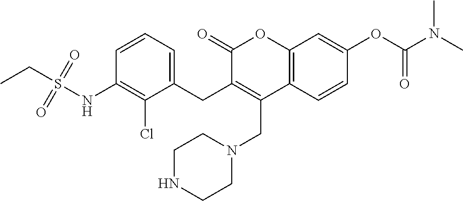 [SMILES: CCS(=O)(=O)Nc1cccc(Cc2c(CN3CCNCC3)c3ccc(OC(=O)N(C)C)cc3oc2=O)c1Cl] |
| 274 | 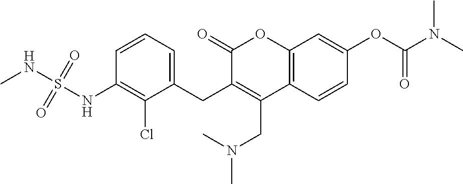 |

TABLE E
Exemplary compounds of the disclosure.

| No. | Structure |
|---|---|
| 278 | 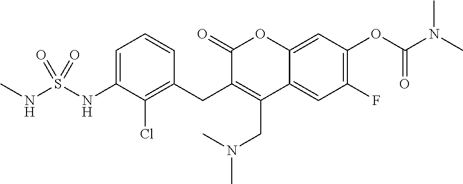 |
| 279 | 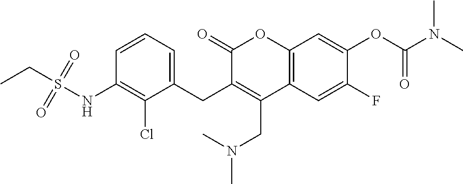 [SMILES: CCS(=O)(=O)Nc1cccc(Cc2c(CN(C)C)c3cc(F)c(OC(=O)N(C)C)cc3oc2=O)c1Cl] |
| 280 | 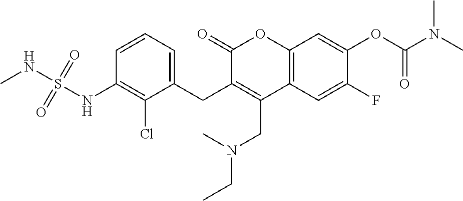 |
| 281 | 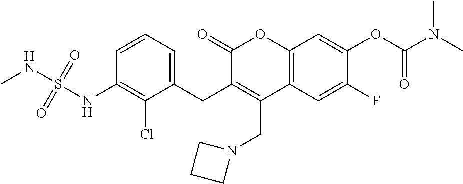 |
| 282 | 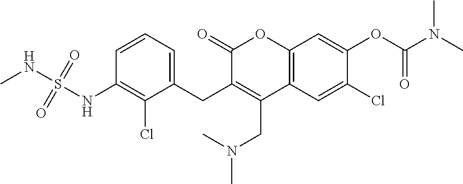 |
| 283 | 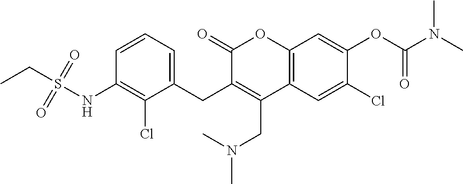 |
| 284 | 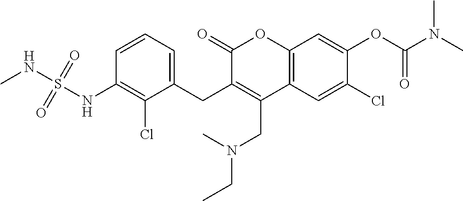 |
| 285 | 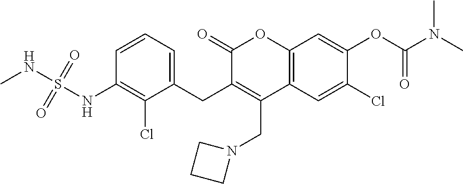 |
| 286 | 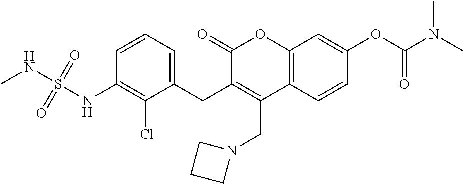 |

In some embodiments, the pharmaceutically acceptable salt can be an alkaline metal salt. In some embodiments, the pharmaceutically acceptable salt can be an alkali metal salt. In some embodiments, the pharmaceutically acceptable salt can be an alkali earth metal salt. In some embodiments, the pharmaceutically acceptable salt can be an ammonium salt.

### Syntheses

Compounds of Formula (I), (Ia), (Ib), (Ic), (Id), (II), (IIa), (IIb), (IIc), (IId), (III), (IV), or pharmaceutically acceptable salts thereof, described herein may be prepared in various ways, including those known to those skilled in the art. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims. Examples of methods are described in the Examples below.

### Methods of Preparation

The compounds disclosed herein may be synthesized by methods described below, or by modification of these methods. Ways of modifying the methodology include, among others, temperature, solvent, reagents etc., and will be obvious to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and Greene & Wuts, Protective Groups in Organic Synthesis, John Wiley & Sons, 1991, which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety.

Where the processes for the preparation of the compounds disclosed herein give rise to mixtures of stereoisomers, such isomers may be separated by conventional techniques such as preparative chiral chromatography. The compounds may be prepared in racemic form or individual enantiomers may be prepared by stereoselective synthesis or by resolution. The compounds may be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid, followed by fractional crystallization and regeneration of the free base. The compounds may also be resolved using a chiral auxiliary by formation of diastereomeric derivatives such as esters, amides or ketals followed by chromatographic separation and removal of the chiral auxiliary.

### Pharmaceutical Compositions

In another aspect, pharmaceutical compositions are disclosed that comprise a physiologically acceptable surface active agents, carriers, diluents, excipients, smoothing agents, suspension agents, film forming substances, and coating assistants, or a combination thereof; and a compound disclosed herein. Acceptable carriers or diluents for therapeutic use are well known in the pharmaceutical art, and are described, for example, in Remington's Pharmaceutical Sciences, 18th Ed., Mack Publishing Co., Easton, PA (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The term “pharmaceutical composition,” as used herein, refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier,” as used herein, refers to a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent,” as used herein, refers to chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable,” as used herein, refers to a carrier or diluent that does not abrogate the biological activity and properties of the compound.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

For each of the compounds described herein, and for each genus or sub-genus of compounds described herein, also described are pharmaceutical compositions comprising the compound, alone or in a mixture with other compounds of the genus or sub-genus, or with alternative compounds described herein, or with one or more alternative pharmaceutically active compounds, and one or more pharmaceutically acceptable carrier, diluent, excipient or combination thereof. The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in any manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, PA, 18th edition, 1990.

Suitable routes of administration may, for example, include oral, rectal, transmucosal, topical, or intestinal administration; parenteral delivery, including intramuscular, subcutaneous, intravenous, intramedullary injections, as well as intrathecal, direct intraventricular, intraperitoneal, intranasal, or intraocular injections. The compounds can also be administered in sustained or controlled release dosage forms, including depot injections, osmotic pumps, pills, transdermal (including electrotransport) patches, and the like, for prolonged and/or timed, pulsed administration at a predetermined rate.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Further disclosed herein are various pharmaceutical compositions well known in the pharmaceutical art for uses that include intraocular, intranasal, and intraauricular delivery. Suitable penetrants for these uses are generally known in the art. Pharmaceutical compositions for intraocular delivery include aqueous ophthalmic solutions of the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions for intranasal delivery may also include drops and sprays often prepared to simulate in many respects nasal secretions to ensure maintenance of normal ciliary action. As disclosed in Remington's Pharmaceutical Sciences, 18th Ed., Mack Publishing Co., Easton, PA (1990), which is incorporated herein by reference in its entirety, and well-known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the ear. Common solvents for such aural formulations include glycerin and water.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For hydrophobic compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Agents intended to be administered intracellularly may be administered using techniques well known to those of ordinary skill in the art. For example, such agents may be encapsulated into liposomes. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. Alternatively, small hydrophobic organic molecules may be directly administered intracellularly.

Additional therapeutic or diagnostic agents may be incorporated into the pharmaceutical compositions. Alternatively or additionally, pharmaceutical compositions may be combined with other compositions that contain other therapeutic or diagnostic agents.

### Parenteral Pharmaceutical Composition

To prepare a parenteral pharmaceutical composition suitable for administration by injection (subcutaneous, intravenous, or the like), 0.1 mg to 120 mg of a water-soluble salt/soluble material itself/solubilized complex of a compound of a preferred embodiment is dissolved in sterile water and then mixed with 10 μL of 0.9% sterile saline. The mixture is incorporated into a dosage unit form suitable for administration by injection.

### Injectable Pharmaceutical Composition

To prepare an injectable formulation, 0.1 mg to 100 mg of a compound of Formula (I), (Ia), (Ib), (Ic), (II), (IIa), (IIb), (IIc), (III), or (IV), 2.0 mL of sodium acetate buffer solution (0.4 M), HCl (1 N) or NaOH (1 M) (q.s. to suitable pH), water (distilled, sterile) (q.s. to 20 mL) are mixed. All of the above ingredients, except water, are combined and stirred and if necessary, with slight heating if necessary. A sufficient quantity of water is then added.

### Oral Pharmaceutical Composition

To prepare a pharmaceutical composition for oral delivery, 0.1 mg to 120 mg of a compound of an embodiment is mixed with 750 mg of starch. The mixture is incorporated into an oral dosage unit, such as a hard gelatin capsule, or 0.1 mg to 120 mg of compound is granulated with binder solution such as starch solution along with suitable diluents such as microcrystalline cellulose or like, disintegrants such as croscaramellose sodium, dry the resultant mixture and add lubricant and compress into tablet which is suitable for oral administration.

### Sublingual (Hard Lozenge) Pharmaceutical Composition

To prepare a pharmaceutical composition for buccal delivery, such as a hard lozenge, 0.1 mg to 120 mg of a compound of a preferred embodiment is mixed with 420 mg of powdered sugar/mannitol/xylitol or such sugars that provide negative heat of solution to the system, 1.6 mL of light corn syrup, 2.4 mL distilled water, and 0.42 mL mint extract or other flavorants. The mixture is blended and poured into a mold to form a lozenge suitable for buccal administration.

### Fast-Disintegrating Sublingual Tablet

A fast-disintegrating sublingual tablet is prepared by mixing 48.5% by weight of a compound of a preferred embodiment, 20% by weight of microcrystalline cellulose (KG-802), 24.5% by weight of either mannitol or modified dextrose or combination that help dissolve the compressed tablet faster in the mouth, 5% by weight of low-substituted hydroxypropyl cellulose (50 μm), and 2% by weight of magnesium stearate. Tablets are prepared by direct compression (AAPS PharmSciTech. 2006; 7(2):E41). The total weight of the compressed tablets is maintained at 150 mg. The formulation is prepared by mixing the amount of the compound of a preferred embodiment with the total quantity of microcrystalline cellulose (MCC) and mannitol/modified dextrose or combination, and two-thirds of the quantity of low-substituted hydroxypropyl cellulose (L-HPC) by using a three dimensional manual mixer (Inversina, Bioengineering AG, Switzerland) for 4.5 minutes. All of the magnesium stearate (MS) and the remaining one-third of the quantity of L-HPC are added 30 seconds before the end of mixing.

### Inhalation Pharmaceutical Composition

To prepare a pharmaceutical composition for inhalation delivery, 0.1 mg to 100 mg of a compound of a preferred embodiment is mixed with 50 mg of anhydrous citric acid and 100 mL of 0.9% sodium chloride solution. The mixture is incorporated into an inhalation delivery unit, such as a nebulizer, which is suitable for inhalation administration.

### Nebulizer Suspension Pharmaceutical Composition

In another embodiment, a compound of a preferred embodiment (0.1 mg to 100 mg) is suspended in sterile water (100 mL); Span 85 (1 g) is added followed by addition of dextrose (5.5 g) and ascorbic acid (10 mg). Benzalkonium chloride (3 mL of a 1:750 aqueous solution) is added and the pH is adjusted to 7 with phosphate buffer. The suspension is packaged in sterile nebulizers.

### Transdermal Patch Pharmaceutical Composition

To prepare a pharmaceutical composition for transdermal delivery, 0.1 mg to 100 mg of a compound of a preferred embodiment is embedded in, or deposited on, a patch with a single adhesive face. The resulting patch is then attached to the skin via the adhesive face for transdermal administration.

### Topical Gel Pharmaceutical Composition

To prepare a pharmaceutical topical gel composition, 0.1 mg to 100 mg of a compound of a preferred embodiment is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution

To prepare a pharmaceutical ophthalmic solution composition, 0.1 mg to 100 mg of a compound of a preferred embodiment is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 0.1 mg to 100 mg of a compound of a preferred embodiment is mixed with 30 mL of a 0.05M phosphate buffer solution (pH 4.4). The solution is placed in a nasal administrator designed to deliver 100 μl of spray for each application.

### Immune Checkpoint Inhibitors

In some embodiments, one or more immune checkpoint inhibitor may be co-administered with a compound of Formula (I), (Ia), (Ib), (Ic), (II), (IIa), (IIb), (IIc), (III), or (IV). A review describing immune checkpoint pathways and the blockade of such pathways with immune checkpoint inhibitor compounds is provided by Pardoll in Nature Reviews Cancer (April, 2012), pages 252-264, which is incorporated herein by reference in its entirety. Immune check point inhibitor compounds display anti-tumor activity by blocking one or more of the endogenous immune checkpoint pathways that downregulate an anti-tumor immune response. The inhibition or blockade of an immune checkpoint pathway typically involves inhibiting a checkpoint receptor and ligand interaction with an immune checkpoint inhibitor compound to reduce or eliminate the down regulation signal and resulting diminishment of the anti-tumor response.

In some embodiments of the present disclosure, the immune checkpoint inhibitor compound inhibits the signaling interaction between an immune checkpoint receptor and the corresponding ligand of the immune checkpoint receptor. The immune checkpoint inhibitor compound can act by blocking activation of the immune checkpoint pathway by inhibition (antagonism) of an immune checkpoint receptor (some examples of receptors include CTLA-4, PD-1, LAG-3, TIM-3, BTLA, and KIR) or by inhibition of a ligand of an immune checkpoint receptor (some examples of ligands include PD-L1 and PD-L2). In such embodiments, the effect of the immune checkpoint inhibitor compound is to reduce or eliminate down regulation of certain aspects of the immune system anti-tumor response in the tumor microenvironment.

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8; which are incorporated herein by reference in their entirety). Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes.

The PD-1 gene encodes a 55 kDa type I transmembrane protein (Agata et al. (1996) Int Immunol. 8:765-72, which is incorporated herein by reference in its entirety). Although structurally similar to CTLA-4, PD-1 lacks the MYPPY motif that is important for B7-1 and B7-2 binding. Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43; which are incorporated herein by reference in their entirety). Both PD-L1 and PD-L2 are B7 homologs that bind to PD-1, but do not bind to other CD28 family members. PD-L1 is abundant in a variety of human cancers (Dong et al. (2002) Nat. Med. 8:787-9, which is incorporated herein by reference in its entirety).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745; which are incorporated herein by reference in their entirety). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100; which are incorporated herein by reference in their entirety). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66; which are incorporated herein by reference in their entirety).

The immune checkpoint receptor cytotoxic T-lymphocyte associated antigen 4 (CTLA-4) is expressed on T-cells and is involved in signaling pathways that reduce the level of T-cell activation. It is believed that CTLA-4 can downregulate T-cell activation through competitive binding and sequestration of CD80 and CD86. In addition, CTLA-4 has been shown to be involved in enhancing the immunosuppressive activity of T_{Reg} cells.

The immune checkpoint receptor programmed death 1 (PD-1) is expressed by activated T-cells upon extended exposure to antigen. Engagement of PD-1 with its known binding ligands, PD-L1 and PD-L2, occurs primarily within the tumor microenvironment and results in downregulation of anti-tumor specific T-cell responses. Both PD-L1 and PD-L2 are known to be expressed on tumor cells. The expression of PD-L1 and PD-L2 on tumors has been correlated with decreased survival outcomes.

The immune checkpoint receptor T cell membrane protein 3 (TIM-3) is expressed on Th1 and Tc1 cells, but not other T-cells. Interaction of TIM-3 with its ligand, galectin-9, produces a Th1 cell death signal. TIM-3 has been reported to play a role in maintaining T-cell exhaustion and blockade of TIM-3 has been shown to restore activity to exhausted T-cells.

The immune checkpoint receptor B- and T-lymphocyte attenuator (BTLA) receptor is expressed on both resting and activated B-cells and T-cells. Activation of BTLA when combined with its ligand HVEM (herpes virus entry mediator) results in downregulation of both T-cell activation and proliferation. HVEM is expressed by certain tumors (e.g., melanoma) and tumor-associated endothelial cells.

The immune checkpoint receptors known as killer cell immunoglobulin-like receptors (KIR) are a polymorphic family of receptors expressed on NK cells and some T-cells and function as regulators of immune tolerance associated with natural killer (NK) cells. Blocking certain KIR receptors with inhibitor compounds can facilitate the destruction of tumors through the increased activity of NK cells.

In some embodiments of the present disclosure, the immune checkpoint inhibitor compound is a small organic molecule (molecular weight less than 1000 daltons), a peptide, a polypeptide, a protein, an antibody, an antibody fragment, or an antibody derivative. In some embodiments, the immune checkpoint inhibitor compound is an antibody. In some embodiments, the antibody is a monoclonal antibody, specifically a human or a humanized monoclonal antibody.

Monoclonal antibodies, antibody fragments, and antibody derivatives for blocking immune checkpoint pathways can be prepared by any of several methods known to those of ordinary skill in the art, including but not limited to, somatic cell hybridization techniques and hybridoma, methods. Hybridoma generation is described in Antibodies, A Laboratory Manual, Harlow and Lane, 1988, Cold Spring Harbor Publications, New York, which is incorporated herein by reference in its entirety. Human monoclonal antibodies can be identified and isolated by screening phage display libraries of human immunoglobulin genes by methods described for example in U.S. Pat. Nos. 5,223,409, 5,403,484, 5,571,698, 6,582,915, and 6,593,081, which are incorporated herein by reference in their entirety. Monoclonal antibodies can be prepared using the general methods described in U.S. Pat. No. 6,331,415 (Cabilly), which is incorporated herein by reference in its entirety.

As an example, human monoclonal antibodies can be prepared using a XenoMouse™ (Abgenix, Freemont, Calif.) or hybridomas of B cells from a XenoMouse. A XenoMouse is a murine host having functional human immunoglobulin genes as described in U.S. Pat. No. 6,162,963 (Kucherlapati), which is incorporated herein by reference in its entirety.

Methods for the preparation and use of immune checkpoint antibodies are described in the following illustrative publications. The preparation and therapeutic uses of anti-CTLA-4 antibodies are described in U.S. Pat. No. 7,229,628 (Allison), U.S. Pat. No. 7,311,910 (Linsley), and U.S. Pat. No. 8,017,144 (Korman), which are incorporated herein by reference in their entirety. The preparation and therapeutic uses of anti-PD-1 antibodies are described in U.S. Pat. No. 8,008,449 (Korman) and U.S. Patent Application No. 2011/0271358 (Freeman), which are incorporated herein by reference in their entirety. The preparation and therapeutic uses of anti-PD-L1 antibodies are described in U.S. Pat. No. 7,943,743 (Korman), which is incorporated herein by reference in its entirety. The preparation and therapeutic uses of anti-TIM-3 antibodies are described in U.S. Pat. No. 8,101,176 (Kuchroo) and U.S. Pat. No. 8,552,156 (Tagayanagi), which are incorporated herein by reference in their entirety. The preparation and therapeutic uses of anti-LAG-3 antibodies are described in U.S. Patent Application No. 2011/0150892 (Thudium) and International Publication Number WO2014/008218 (Lonberg), which are incorporated herein by reference in their entirety. The preparation and therapeutic uses of anti-KIR antibodies are described in U.S. Pat. No. 8,119,775 (Moretta), which is incorporated herein by reference in its entirety. The preparation of antibodies that block BTLA regulated inhibitory pathways (anti-BTLA antibodies) are described in U.S. Pat. No. 8,563,694 (Mataraza), which is incorporated herein by reference in its entirety.

In some embodiments, the one or more immune checkpoint inhibitor is an inhibitor of PD-1, PD-L1, or CTLA-4. In some embodiments, the immune checkpoint inhibitor is a PD-1 inhibitor. In some embodiments, the immune checkpoint inhibitor is a binding ligand of PD-L1. In some embodiments, the immune checkpoint inhibitor is a PD-L1 inhibitor. In some embodiments, the immune checkpoint inhibitor is a CTLA-4 inhibitor.

In some embodiments, the one or more immune checkpoint inhibitor as described herein includes a first immune checkpoint inhibitor and a second immune checkpoint inhibitor, wherein the first immune checkpoint inhibitor is different from the second immune checkpoint inhibitor. In some embodiments, the first and the second immune checkpoint inhibitor are independently an inhibitor of PD-1, PD-L1 or CTLA-4. In some embodiments, the first immune checkpoint inhibitor is a PD-1 inhibitor, and the second immune checkpoint inhibitor is a CTLA-4 inhibitor.

In some embodiments, the immune checkpoint inhibitor is pembrolizumab, nivolumab, cemiplimab, atezolizumab, avelumab, pembrolizumab, pidilizumab, ipilimumab, BMS 936559, durvalumab, or any combinations thereof. In some embodiments, the one or more immune checkpoint inhibitor may include an anti-PD-1 HuMAbs can be selected from 17D8, 2D3, 4H1, 5C4 (also referred to herein as nivolumab), 4A1 1, 7D3 and 5F4, all of which are described in U.S. Pat. No. 8,008,449, which is incorporated herein by reference in its entirety. In some embodiments, the anti-PD-1 HuMAbs can be selected from 3G10, 12A4 (also referred to herein as BMS-936559), 10A5, 5F8, 10H10, 1B12, 7H1, 1 1E6, 12B7, and 13G4, all of which are described in U.S. Pat. No. 7,943,743, which is incorporated herein by reference in its entirety.

In some embodiments, the one or more immune checkpoint inhibitor may be incorporated in a pharmaceutically acceptable formulation. In some embodiments, the one or more immune checkpoint inhibitor is incorporated in a pharmaceutically acceptable aqueous formulation. Examples of acceptable aqueous formulations include isotonic buffered and pH 4.5-8 adjusted saline solutions such as Lactated Ringer's Solution and the like.

In some embodiments, the immune checkpoint inhibitor compound is incorporated in a pharmaceutically acceptable liposome formulation, wherein the formulation is a passive or targeted liposome formulation. Examples of methods for the preparation of suitable liposome formulations of antibodies are described U.S. Pat. No. 5,399,331 (Loughrey), U.S. Pat. No. 8,304,565 (Wu) and U.S. Pat. No. 7,780,882 (Chang), which are incorporated herein by reference in their entirety.

In some embodiments, the one or more immune checkpoint inhibitor may be an antibody. In some embodiments, the antibody is a dry, lyophilized solid that is reconstituted with an aqueous reconstitution solvent prior to use. In some embodiments, the antibody is incorporated in a pharmaceutically acceptable formulation and the pharmaceutically acceptable formulation is injected directly into a tumor. In some embodiments, the immune checkpoint inhibitor antibody is incorporated in a pharmaceutically acceptable formulation and the pharmaceutically acceptable formulation is injected into the peritumoral region surrounding a tumor. The peritumoral region may contain antitumor immune cells. In some embodiments, the antibody is incorporated in a pharmaceutically acceptable formulation and the pharmaceutically acceptable formulation is administered by intravenous injection or infusion. In some embodiments, the immune checkpoint inhibitor antibody is incorporated in a pharmaceutically acceptable formulation and the pharmaceutically acceptable formulation is administered by subcutaneous injection or intradermal injection. In some embodiments, the antibody is incorporated in a pharmaceutically acceptable formulation and the pharmaceutically acceptable formulation is administered by intraperitoneal injection or lavage.

The precise amount of immune checkpoint inhibitor compound incorporated in a particular method or therapeutic combination of the disclosure may vary according to factors known in art such as for example, the physical and clinical status of the subject, the method of administration, the content of the formulation, the physical and chemical nature of the immune checkpoint inhibitor compound, the intended dosing regimen or sequence. Those of ordinary skill in the art, however, can readily determine the appropriate amount with due consideration of such factors.

### Methods of Treatment/Uses

Aspects disclosed herein relate to administering to a subject in need an effective amount of a compound of Formula (I), (Ia), (Ib), (Ic), (Id), (II), (IIa), (IIb), (IIc), (IId), (III), (IV), or a pharmaceutically acceptable salt thereof), or a pharmaceutical composition that includes one or more compounds as described herein (such as one or more compounds of Formula (I), (Ia), (Ib), (Ic), (Id), (II), (IIa), (IIb), (IIc), (IId), (III), (IV), or a pharmaceutically acceptable salt thereof).

As disclosed elsewhere herein, some embodiments pertain to treating a disease or condition, such as cancer, through administration of a compound or composition as disclosed herein. A subject in need of receiving a compound or composition as disclosed herein to improve the subject's health need not always be identified prior to receiving a first treatment with the compound or composition. For example, a subject may be predetermined that they will develop a disease or condition, such as cancer, prior to showing any signs of the disease or condition. Alternatively, the subject may receive treatment prophylactically if he or she is at risk or not developing a disease or condition, such as cancer, (e.g., once a patient shows symptoms of another disease or condition associated with a cancer). Accordingly, in some embodiments, the compound or composition may be administered to the subject after the subject receives an early stage diagnosis. In some embodiments, not every subject is a candidate for such administration and identification of treatment subjects may be desirable. It is understood that patient selection depends upon a number of factors within the skill of the ordinarily skilled physician. Thus, some embodiments disclosed herein further comprise identifying a subject as one that will benefit from administering an effective amount of at least one compound or composition to increase longevity, increase survival time or increase life span.

In other aspects, the present disclosure is directed to a method for the treatment, prevention or prophylaxis of cancer can include administering to a subject in need thereof an effective amount of one or more compound described herein (such as a compound of Formula (I), (Ia), (Ib), (Ic), (Id), (II), (IIa), (IIb), (IIc), (IId), (III), (IV), or a pharmaceutically acceptable salt thereof), or a pharmaceutical composition that includes a compound described herein (such as a compound of Formula (I), (Ia), (Ib), (Ic), (Id), (II), (IIa), (IIb), (IIc), (IId), (III), (IV), or a pharmaceutically acceptable salt thereof). In certain embodiments, the cancer may be selected from brain cancer, breast cancer, lung cancer, ovarian cancer, pancreatic cancer, stomach cancer, prostate cancer, renal cancer, colorectal cancer, melanoma, or leukemia. In further or additional embodiments, the cancer is brain cancer or ach-enocortical carcinoma. In further or additional embodiments, the cancer is breast cancer. In further or additional embodiments, the cancer is ovarian cancer. In further or additional embodiments, the cancer is pancreatic cancer. In further or additional embodiments, the cancer is stomach cancer. In further or additional embodiments, the cancer is prostate cancer. In further or additional embodiments, the cancer is renal cancer. In further or additional embodiments, the cancer is colorectal cancer. In further or additional embodiments, the cancer is myeloid leukemia. In further or additional embodiments, the cancer is glioblastoma. In further or additional embodiments, the cancer is follicular lymphoma. In further or additional embodiments, the cancer is pre-B acute leukemia. In further or additional embodiments, the cancer is chronic lymphocytic B-leukemia. In further or additional embodiments, the cancer is mesothelioma. In further or additional embodiments, the cancer is small cell line cancer. In further or additional embodiments, the cancer is melanoma.

Some embodiments relate to a method of inhibiting proliferation of a cell having a RAS mutation, comprising administering a compound of Formula (I), (Ia), (Ib), (Ic), (Id), (II), (IIa), (IIb), (IIc), (IId), (III), (IV), or a pharmaceutically acceptable salt thereof). In some embodiments, the cancer has associated with a RAS mutation. Some embodiments relate to a method of inducing apoptosis in a cell having a RAS mutation, comprising administering a compound of Formula (I), (Ia), (Ib), (Ic), (Id), (II), (IIa), (IIb), (IIc), (IId), (III), (IV), or a pharmaceutically acceptable salt thereof). Some embodiments relate to a method of inhibiting proliferation of a cell having a KRAS mutation, comprising administering a compound of Formula (I), (Ia), (Ib), (Ic), (Id), (II), (IIa), (IIb), (IIc), (IId), (III), (IV), or a pharmaceutically acceptable salt thereof). In some embodiments, the cancer has associated with a KRAS mutation. Some embodiments relate to a method of inducing apoptosis in a cell having a KRAS mutation, comprising administering a compound of Formula (I)), (Ia), (Ib), (Ic), (Id), (II), (IIa), (IIb), (IIc), (IId), (III), (IV), or a pharmaceutically acceptable salt thereof). Some embodiments relate to a method of inhibiting proliferation of a cell having a NRAS mutation, comprising administering a compound of Formula (I), (Ia), (Ib), (Ic), (Id), (II), (IIa), (IIb), (IIc), (IId), (III), (IV), or a pharmaceutically acceptable salt thereof). In some embodiments, the cancer has associated with a NRAS mutation. Some embodiments relate to a method of inducing apoptosis in a cell in a cell having a RAS mutation, comprising administering a compound of Formula (I), (Ia), (Ib), (Ic), (Id), (II), (IIa), (IIb), (IIc), (IId), (III), (IV), or a pharmaceutically acceptable salt thereof). In some embodiments, the cancer has associated with a HRAS mutation. Some embodiments relate to a method of inducing apoptosis in a cell having a RAS mutation, comprising administering a compound of Formula (I), (Ia), (Ib), (Ic), (Id), (II), (IIa), (IIb), (IIc), (IId), (III), (IV), or a pharmaceutically acceptable salt thereof). In some embodiments, the KRAS mutation is at codons 12, 13, 59, 61 and/or 146. In some embodiments, the mutant form of the KRAS protein has one or more amino acid substitutions selected from the group consisting of G12C, G12S, G12R, G12F, G12L, G12N, G12A, G12D, G12V, G13C, G13S, G13D, G13V, G13P, S17G, P34S, A59E, A59G, A59T, Q61K, Q61L, Q61R, and Q61H. In some embodiments, the mutant form of the KRAS protein has one or more amino acid substitutions selected from the group consisting of G12C, G12R, G12S, G12A, G12D, G12V, G13C, G13R, G13S, G13A, G13D, G13V, A59E, A59G, A59T, Q61K, Q61L, Q61R, Q61H, K117N, K117R, K117E, A146P, A146T and A146V.

In certain embodiments, the cancer is resistant to treatment of a MEK protein kinase inhibitor. In other embodiments, the cancer is resistant to treatment of a RAF protein kinase inhibitor. In still further embodiments, the resistance is acquired resistance. In other embodiments, the resistance is de novo resistance. In further or additional embodiments, the cancer is resistant to an anticancer agent.

In some aspects provided herein are a compounds or pharmaceutical compositions and methods for treating cancer comprising a therapeutically effective amount of a dual-MEK protein kinase inhibitor. In some embodiments, the administration of the dual-MEK protein kinase inhibitor provides an increase in the area under the serum concentration time curve (AUC) of the dual-MEK protein kinase inhibitor. In some embodiments, the cancer is resistant to treatment of a RAF protein kinase inhibitor. In further embodiments, the cancer is resistant to a RAF protein kinase inhibitor and the RAF protein kinase inhibitor comprises an A-RAF inhibitor, a B-RAF inhibitor, or a C-RAF inhibitor. In further embodiments, the cancer is resistant to a RAF protein kinase inhibitor, and the RAF protein kinase inhibitor comprises a B-RAF inhibitor.

In some embodiments, the resistant cancer is pancreatic, melanoma, colon, lung, or stomach cancer. In further embodiments, the resistant cancer is pancreatic. In additional embodiments, the resistant cancer is stomach. In alternative embodiments, provided are pharmaceutical combinations and methods for resensitizing cancer cells to treatment in a patient having or suspected of having a cancer resistant to an anticancer agent, comprising the step of administering to the patient a therapeutically effective amount of a dual-MEK inhibitor as disclosed herein.

In some embodiments, a compound of Formula (I), (Ia), (Ib), (Ic), (Id), (II), (IIa), (IIb), (IIc), (IId), (III), (IV), or a pharmaceutically acceptable salt thereof, is co-administered with a CTLA-4 receptor inhibitor compound. In some embodiments, a compound of Formula (I) is co-administered a PD-1 or PD-L1 receptor inhibitor compound.

In some embodiments, the method comprises treating a subject by co-administering a therapeutically effective amount of a compound of Formula (I), (Ia), (Ib), (Ic), (Id), (II), (IIa), (IIb), (IIc), (IId), (III), (IV), or a pharmaceutically acceptable salt thereof, and a LAG-3 receptor inhibitor compound. In some embodiments, the method comprises treating a subject by co-administering a therapeutically effective amount of a compound of Formula (I) and a TIM-3 receptor inhibitor compound. In some embodiments, the method comprises treating a subject by co-administering a therapeutically effective amount of a compound of Formula (I), (Ia), (Ib), (Ic), (Id), (II), (IIa), (IIb), (IIc), (IId), (III), (IV), or a pharmaceutically acceptable salt thereof, and a BTLA receptor inhibitor compound. In some embodiments, the method comprises treating a subject by co-administering a therapeutically effective amount of a compound of Formula (I), (Ia), (Ib), (Ic), (Id), (II), (IIa), (IIb), (IIc), (IId), (III), (IV), or a pharmaceutically acceptable salt thereof, and a KIR receptor inhibitor compound. In some embodiments, the method comprises treating a subject by co-administering a therapeutically effective amount of a compound of Formula (I), (Ia), (Ib), (Ic), (Id), (II), (IIa), (IIb), (IIc), (IId), (III), (IV), or a pharmaceutically acceptable salt thereof, and a PD-L1 inhibitor compound. In some embodiments, the method comprises treating a subject by co-administering a therapeutically effective amount of a compound of Formula (I), (Ia), (Ib), (Ic), (Id), (II), (IIa), (IIb), (IIc), (IId), (III), (IV), or a pharmaceutically acceptable salt thereof, and a PD-L2 inhibitor compound.

In some embodiments of the present disclosure, the method comprises treating a subject by co-administering a therapeutically effective amount of a compound of Formula (I), (Ia), (Ib), (Ic), (Id), (II), (IIa), (IIb), (IIc), (IId), (III), (IV), or a pharmaceutically acceptable salt thereof, and a blocking antibody of an immune checkpoint pathway. In some embodiments, the method comprises treating a subject by co-administering a therapeutically effective amount of a compound of Formula (I), (Ia), (Ib), (Ic), (Id), (II), (IIa), (IIb), (IIc), (IId), (III), (IV), or a pharmaceutically acceptable salt thereof, and an anti-CTLA-4 receptor antibody. In some embodiments, the method comprises treating a subject by co-administering a therapeutically effective amount of a compound of Formula (I), (Ia), (Ib), (Ic), (Id), (II), (IIa), (IIb), (IIc), (IId), (III), (IV), or a pharmaceutically acceptable salt thereof, and an anti-PD-1 receptor antibody.

In some embodiments, the method comprises co-administering to a subject having a tumor a therapeutically effective amount of the compound of Formula (I), (Ia), (Ib), (Ic), (Id), (II), (IIa), (IIb), (IIc), (IId), (III), (IV), or a pharmaceutically acceptable salt thereof, and an anti-LAG-3 receptor antibody. In some embodiments, the method comprises co-administering to a subject having a tumor a therapeutically effective amount of the compound of Formula (I), (Ia), (Ib), (Ic), (Id), (II), (IIa), (IIb), (IIc), (IId), (III), (IV), or a pharmaceutically acceptable salt thereof, and an anti-TIM-3 receptor antibody. In some embodiments, the method comprises co-administering to a subject having a tumor a therapeutically effective amount of the compound of Formula (I), (Ia), (Ib), (Ic), (Id), (II), (IIa), (IIb), (IIc), (IId), (III), (IV), or a pharmaceutically acceptable salt thereof, and an anti-BTLA receptor antibody. In some embodiments, the method comprises co-administering to a subject having a tumor a therapeutically effective amount of the compound of Formula (I), (Ia), (Ib), (Ic), (Id), (II), (IIa), (IIb), (IIc), (IId), (III), (IV), or a pharmaceutically acceptable salt thereof, and an anti-KIR receptor antibody. In some embodiments, the anti-KIR receptor antibody is lirilumab. In some embodiments, the method comprises co-administering to a subject having a tumor a therapeutically effective amount of the compound of Formula (I), (Ia), (Ib), (Ic), (Id), (II), (IIa), (IIb), (IIc), (IId), (III), (IV), or a pharmaceutically acceptable salt thereof, and an anti-PD-1 antibody. In some embodiments the anti-PD-1 antibody is lambrolizumab, pidilizumab, or nivolumab. In some embodiments, the method comprises co-administering to a subject having a tumor a therapeutically effective amount of the compound of Formula (I), (Ia), (Ib), (Ic), (Id), (II), (IIa), (IIb), (IIc), (IId), (III), (IV), or a pharmaceutically acceptable salt thereof, and an anti-PD-L1 antibody. In some embodiments, the method comprises co-administering to a subject having a tumor a therapeutically effective amount of the compound of Formula (I), (Ia), (Ib), (Ic), (Id), (II), (IIa), (IIb), (IIc), (IId), (III), (IV), or a pharmaceutically acceptable salt thereof, and an anti-PD-L2 antibody. In some embodiments, the method comprises co-administering to a subject having a tumor a therapeutically effective amount of the compound of Formula (I), (Ia), (Ib), (Ic), (Id), (II), (IIa), (IIb), (IIc), (IId), (III), (IV), or a pharmaceutically acceptable salt thereof, and an anti-CTLA-4 antibody. In some embodiments, the anti-CTLA-4 antibody is ipilimumab or tremelimumab.

In some embodiments, a method comprises co-administering a compound of Formula (I), (Ia), (Ib), (Ic), (Id), (II), (IIa), (IIb), (IIc), (IId), (III), (IV), or a pharmaceutically acceptable salt thereof, and an immune checkpoint inhibitor for treating, preventing, or ameliorating a cancer or tumor in a subject by administering a compound of Formula (I), or a pharmaceutically acceptable salt thereof, and an immune checkpoint inhibitor. In some embodiments, the subject was resistant to prior treatment with only an immune checkpoint inhibitor.

In some embodiments, a method for treating a subject with a cancer or tumor comprises administering a compound of Formula (I), (Ia), (Ib), (Ic), (Id), (II), (IIa), (IIb), (IIc), (IId), (III), (IV), or a pharmaceutically acceptable salt thereof, and a PD-1 inhibitor. In some embodiments, a method for treating a subject with a cancer or tumor comprises administering a compound of Formula (I), (Ia), (Ib), (Ic), (Id), (II), (IIa), (IIb), (IIc), (IId), (III), (IV), or a pharmaceutically acceptable salt thereof, and a PD-L1 inhibitor. In some embodiments, a method for treating a subject with a cancer or tumor comprises administering a compound of Formula (I), (Ia), (Ib), (Ic), (Id), (II), (IIa), (IIb), (IIc), (IId), (III), (IV), or a pharmaceutically acceptable salt thereof, and a PD-L2 inhibitor. In some embodiments, a method for treating a subject with a cancer or tumor comprises administering a compound of Formula (I), (Ia), (Ib), (Ic), (Id), (II), (IIa), (IIb), (IIc), (IId), (III), (IV), or a pharmaceutically acceptable salt thereof, and a CTLA-4 inhibitor. In some embodiments, a method for treating a subject with a cancer or tumor comprises administering a compound of Formula (I), (Ia), (Ib), (Ic), (Id), (II), (IIa), (IIb), (IIc), (IId), (III), (IV), or a pharmaceutically acceptable salt thereof, a PD-1 inhibitor and a CTLA-4 inhibitor. In some embodiments, a method for treating a subject with a cancer or tumor comprises administering a compound of Formula (I), (Ia), (Ib), (Ic), (Id), (II), (IIa), (IIb), (IIc), (IId), (III), (IV), or a pharmaceutically acceptable salt thereof, and a LAG-3 inhibitor. In some embodiments, a method for treating a subject with a cancer or tumor comprises administering a compound of Formula (I), (Ia), (Ib), (Ic), (Id), (II), (IIa), (IIb), (IIc), (IId), (III), (IV), or a pharmaceutically acceptable salt thereof, and a KIR inhibitor. In some embodiments, a method for treating a subject with a cancer or tumor comprises administering a compound of Formula (I), (Ia), (Ib), (Ic), (Id), (II), (IIa), (IIb), (IIc), (IId), (III), (IV), or a pharmaceutically acceptable salt thereof, and a TIM-3 inhibitor. In some embodiments, a method for treating a subject with a cancer or tumor comprises administering a compound of Formula (I), (Ia), (Ib), (Ic), (Id), (II), (IIa), (IIb), (IIc), (IId), (III), (IV), or a pharmaceutically acceptable salt thereof, and a BTLA inhibitor.

In some embodiments, a method for treating a subject comprises treating a subject having exhibited resistance to a PD-1 inhibitor by administering a therapeutically effective amount of a compound of Formula (I), (Ia), (Ib), (Ic), (Id), (II), (IIa), (IIb), (IIc), (IId), (III), (IV), or a pharmaceutically acceptable salt thereof. In some embodiments, a method comprises treating a subject having exhibited resistance to a PD-L1 inhibitor by administering a therapeutically effective amount of a compound of Formula (I), (Ia), (Ib), (Ic), (Id), (II), (IIa), (IIb), (IIc), (IId), (III), (IV), or a pharmaceutically acceptable salt thereof. In some embodiments, a method comprises treating a subject having exhibited resistance to a PD-L2 inhibitor by administering a therapeutically effective amount of a compound of Formula (I), (Ia), (Ib), (Ic), (Id), (II), (IIa), (IIb), (IIc), (IId), (III), (IV), or a pharmaceutically acceptable salt thereof. In some embodiments, a method comprises treating a subject having exhibited resistance to a CTLA-4 inhibitor by administering a therapeutically effective amount of a compound of Formula (I), (Ia), (Ib), (Ic), (Id), (II), (IIa), (IIb), (IIc), (IId), (III), (IV), or a pharmaceutically acceptable salt thereof. In some embodiments, the method includes administering the compound of Formula (I), (Ia), (Ib), (Ic), (Id), (II), (IIa), (IIb), (IIc), (IId), (III), (IV), or a pharmaceutically acceptable salt thereof when the subject has exhibited resistance to two different immune checkpoint inhibitors. The two different immune inhibitors can be selected from a CTLA-4 receptor inhibitor, a PD-1 receptor inhibitor, a LAG-3 receptor inhibitor, a TIM-3 receptor inhibitor, a BTLA receptor inhibitor, a KIR receptor inhibitor a PD-L1 inhibitor or a PD-L2 inhibitor. In some embodiments, a method comprises treating a subject having exhibited resistance to a PD-1 inhibitor and CTLA-4 inhibitor by administering a therapeutically effective amount of a compound of Formula (I), (Ia), (Ib), (Ic), (Id), (II), (IIa), (IIb), (IIc), (IId), (III), (IV), or a pharmaceutically acceptable salt thereof. In some embodiments, a method comprises treating a subject having exhibited resistance to a PD-L1 inhibitor and a CTLA-4 inhibitor by administering a therapeutically effective amount of a compound of Formula (I), (Ia), (Ib), (Ic), (Id), (II), (IIa), (IIb), (IIc), (IId), (III), (IV), or a pharmaceutically acceptable salt thereof. In some embodiments, a method comprises treating a subject having exhibited resistance to a PD-1 inhibitor, a PD-L1 inhibitor, and a CTLA-4 inhibitor by administering a therapeutically effective amount of a compound of Formula (I) (Ia), (Ib), (Ic), (Id), (II), (IIa), (IIb), (IIc), (IId), (III), (IV), or a pharmaceutically acceptable salt thereof.

Some embodiments disclosed herein relate to a method of treating a mammal having a disease that can include administering to a subject in need thereof an effective amount of one or more compound described herein (such as a compound of Formula (I), (Ia), (Ib), (Ic), (Id), (II), (IIa), (IIb), (IIc), (IId), (III), (IV), or a pharmaceutically acceptable salt thereof), or a pharmaceutical composition that includes a compound described herein (such as a compound of Formula (I), (Ia), (Ib), (Ic), (Id), (II), (IIa), (IIb), (IIc), (IId), (III), (IV), or a pharmaceutically acceptable salt thereof). Other embodiments disclosed herein relate to a method of treating a subject with cancer cachexia that can include administering to a subject an effective amount of one or more compounds described herein (such as a compound of Formula (I), (Ia), (Ib), (Ic), (Id), (II), (IIa), (IIb), (IIc), (IId), (III), (IV), or a pharmaceutically acceptable salt thereof), or a pharmaceutically acceptable salt of any of the foregoing), or a pharmaceutical composition that includes a compound described herein such as a compound of Formula (I), (Ia), (Ib), (Ic), (Id), (II), (IIa), (IIb), (IIc), (IId), (III), (IV), or a pharmaceutically acceptable salt thereof).

Some embodiments described herein relate to using one or more compounds described herein (such as a compound of Formula (I), (Ia), (Ib), (Ic), (Id), (II), (IIa), (IIb), (IIc), (IId), (III), (IV), or a pharmaceutically acceptable salt thereof), in the manufacture of a medicament for ameliorating and/or treating cancer or conditions of cancer, such as cancer cachexia, that can include administering to a subject an effective amount of one or more compounds described herein (such as a compound of Formula (I), (Ia), (Tb), (Ic), (Id), (II), (IIa), (Iib), (Iic), (Iid), (III), (IV), or a pharmaceutically acceptable salt thereof). Still other embodiments described herein relate to one or more compounds described herein (such as a compound of Formula (I), (Ia), (Ib), (Ic), (Id), (II), (IIa), (IIb), (IIc), (IId), (III), (IV), or a pharmaceutically acceptable salt thereof) that can be used for ameliorating and/or treating cancer or conditions of cancer, such as cancer cachexia, by administering to a subject an effective amount of one or more compounds described herein, or a pharmaceutically acceptable salt thereof.

Some embodiments disclosed herein relate to methods of ameliorating and/or treating cancer that can include contacting a cancerous cell an effective amount of one or more compounds described herein (such as a compound of Formula (I), (Ia), (Ib), (Ic), (Id), (II), (IIa), (Iib), (Iic), (Iid), (III), (IV), or a pharmaceutically acceptable salt thereof), or a pharmaceutical composition that includes one or more compounds described herein (such as a compound of Formula (I), (Ia), (Tb), (Ic), (Id), (II), (IIa), (IIb), (IIc), (IId), (III), (IV), or a pharmaceutically acceptable salt thereof). In some embodiments, a compound of Formula (I), (Ia), (Ib), (Ic), (Id), (II), (IIa), (IIb), (IIc), (IId), (III), (IV), or a pharmaceutically acceptable salt thereof, can act as an inhibitor of MEK. In some embodiments, a compound of Formula (I), (Ia), (Ib), (Ic), (Id), (II), (IIa), (IIb), (IIc), (IId), (III), (IV), or a pharmaceutically acceptable salt thereof, can act as an inhibitor of ERK. In some embodiments, a compound of Formula (I), (Ia), (Ib), (Ic), (Id), (II), (IIa), (Iib), (Iic), (Iid), (III), (IV), or a pharmaceutically acceptable salt thereof, may act as a STAT3 (pSER-727) inhibitor. In some embodiments, a compound of Formula (I), (Ia), (Ib), (Ic), (Id), (II), (IIa), (IIb), (IIc), (IId), (III), (IV), or a pharmaceutically acceptable salt thereof, may reduce inflammatory cachexia and muscle wasting.

In some embodiments, the compound of Formula (I), (Ia), (Ib), (Ic), (Id), (II), (IIa), (IIb), (IIc), (IId), (III), (IV), or a pharmaceutically acceptable salt thereof, may be administered in a single dose, once daily. In some embodiments, the compound of Formula (I), (Ia), (Ib), (Ic), (Id), (II), (IIa), (IIb), (IIc), (IId), (III), (IV), or a pharmaceutically acceptable salt thereof, may be administered in multiple doses, more than once per day. In some embodiments, the compound of Formula (I), (Ia), (Ib), (Ic), (Id), (II), (IIa), (IIb), (IIc), (IId), (III), (IV), or a pharmaceutically acceptable salt thereof, may be administered once a day. In some embodiments, the compound of Formula (I), (Ia), (Ib), (Ic), (Id), (II), (IIa), (IIb), (IIc), (IId), (III), (IV), or a pharmaceutically acceptable salt thereof, may be administered twice a day. In some embodiments, the compound of Formula (I), (Ia), (Ib), (Ic), (Id), (II), (IIa), (IIb), (IIc), (IId), (III), (IV), or a pharmaceutically acceptable salt thereof, may be administered trice a day. In some embodiments, the compound of Formula (I), (Ia), (Ib), (Ic), (Id), (II), (IIa), (IIb), (IIc), (IId), (III), (IV), or a pharmaceutically acceptable salt thereof, may be administered four times a day.

In some aspects, a compound of Formula (I), (Ia), (Ib), (Ic), (Id), (II), (IIa), (IIb), (IIc), (IId), (III), (IV), or a pharmaceutically acceptable salt thereof, may inhibit abnormal cell growth. In some embodiments, the abnormal cell growth occurs in a mammal. Methods for inhibiting abnormal cell growth may comprise administering an effective amount of a compound of Formula (I), (Ia), (Ib), (Ic), (Id), (II), (IIa), (Iib), (Iic), (Iid), (III), (IV), or a pharmaceutically acceptable salt thereof, wherein abnormal cell growth is inhibited. Methods for inhibiting abnormal cell growth in a mammal may comprise administering to the mammal a compound of Formula (I), (Ia), (Ib), (Ic), (Id), (II), (IIa), (IIb), (IIc), (IId), (III), (IV), or a pharmaceutically acceptable salt thereof, wherein the amounts of the compound is effective in inhibiting abnormal cell growth in the mammal.

In other aspects, the present invention is directed to a method for degrading, inhibiting the growth of or killing a cancer cell comprising contacting said cell with a compound of Formula (I), (Ia), (Ib), (Ic), (Id), (II), (IIa), (IIb), (IIc), (IId), (III), (IV), or a pharmaceutically acceptable salt thereof, effective to degrade, inhibit the growth of or to kill said cell. In some embodiments, the cancer cells comprise brain, breast, lung, ovarian, pancreatic, stomach, prostate, renal, melanoma or colorectal cancer cells.

In some embodiments, the cancer cells are degraded. In some embodiments, 1% of the cancer cells are degraded. In further or additional embodiments, 2% of the cancer cells are degraded. In further or additional embodiments, 3% of the cancer cells are degraded. In further or additional embodiments, 4% of the cancer cells are degraded. In further or additional embodiments, 5% of the cancer cells are degraded. In further or additional embodiments, 10% of the cancer cells are degraded. In further or additional embodiments, 20% of the cancer cells are degraded. In further or additional embodiments, 25% of the cancer cells are degraded. In further or additional embodiments, 30% of the cancer cells are degraded. In further or additional embodiments, 40% of the cancer cells are degraded. In further or additional embodiments, 50% of the cancer cells are degraded. In further or additional embodiments, 60% of the cancer cells are degraded. In further or additional embodiments, 70% of the cancer cells are degraded. In further or additional embodiments, 75% of the cancer cells are degraded. In further or additional embodiments, 80% of the cancer cells are degraded. In further or additional embodiments, 90% of the cancer cells are degraded. In further or additional embodiments, 100% of the cancer cells are degraded. In further or additional embodiments, essentially all of the cancer cells are degraded.

In some embodiments, the cancer cells are killed. In further or additional embodiments, 1% of the cancer cells are killed. In further or additional embodiments, 2% of the cancer cells are killed. In further or additional embodiments, 3% of the cancer cells are killed. In further or additional embodiments, 4% of the cancer cells are killed. In further or additional embodiments, 5% of the cancer cells are killed. In further or additional embodiments, 1.0% of the cancer cells are killed. In further or additional embodiments, 20% of the cancer cells are killed. In further or additional embodiments, 25% of the cancer cells are killed. In further or additional embodiments, 30% of the cancer cells are killed. In additional embodiments, 40% of the cancer cells are killed. In further or additional embodiments, 50% of the cancer cells are killed. In further or additional embodiments, 60% of the cancer cells are killed. In further or additional embodiments, 70% of the cancer cells are killed. In further or additional embodiments, 75% of the cancer cells are killed. In further or additional embodiments, 80% of the cancer cells are killed. In further or additional embodiments, 90% of the cancer cells are killed. In further or additional embodiments, 100% of the cancer cells are killed. In farther or additional embodiments, essentially all of the cancer cells are killed.

In further or additional embodiments, the growth of the cancer cells is inhibited. In further or additional embodiments, the growth of the cancer cells is about 1% inhibited. In further or additional embodiments, the growth of the cancer cells is about 2% inhibited. In further or additional embodiments, the growth of the cancer cells is about 3% inhibited. In further or additional embodiments, the growth of the cancer cells is about 4% inhibited. In further or additional embodiments, the growth of the cancer cells is about 5% inhibited. In further or additional embodiments, the growth of the cancer cells is about 10% inhibited. In further or additional embodiments, the growth of the cancer cells is about 20% inhibited. In further or additional embodiments, the growth of the cancer cells is about 25% inhibited. In further or additional embodiments, the growth of the cancer cells is about 30% inhibited, hi further or additional embodiments, the growth of the cancer cells is about 40% inhibited. In further or additional embodiments, the growth of the cancer cells is about 50% inhibited. In further or additional embodiments, the growth of the cancer cells is about 60% inhibited. In further or additional embodiments, the growth of the cancer cells is about 70% inhibited. In further or additional embodiments, the growth of the cancer cells is about 75% inhibited. In further or additional embodiments, the growth of the cancer cells is about 80% inhibited. In further or additional embodiments, the growth of the cancer cells is about 90% inhibited. In further or additional embodiments, the growth of the cancer cells is about 100% inhibited.

In some embodiments, the size of a tumor is reduced by administering a therapeutically effective amount of a compound of Formula (I), (Ia), (Ib), (Ic), (Id), (II), (IIa), (IIb), (IIc), (IId), (III), (IV), or a pharmaceutically acceptable salt thereof. In further or additional embodiments, the size of a tumor is reduced by at least 1%. In further or additional embodiments, the size of a tumor is reduced by at least 2%. In further or additional embodiments, the size of a tumor is reduced by at least 3%. In further or additional embodiments, the size of a tumor is reduced by at least 4%. In further or additional embodiments, the size of a tumor is reduced by at least 5%. In further or additional embodiments, the size of a tumor is reduced by at least 10%. In further or additional embodiments, the size of a tumor is reduced by at least 20%. In further or additional embodiments, the size of a tumor is reduced by at least 25%. In further or additional embodiments, the size of a tumor is reduced by at least 30%. In further or additional embodiments, the size of a tumor is reduced by at least 40%. In further or additional embodiments, the size of a tumor is reduced by at least 50%. In further or additional embodiments, the size of a tumor is reduced by at least 60%. In further or additional embodiments, the size of a tumor is reduced by at least 70%. In further or additional embodiments, the size of a tumor is reduced by at least 75%. In further or additional embodiments, the size of a tumor is reduced by at least 80%. In further or additional embodiments, the size of a tumor is reduced by at least 85%. In further or additional embodiments, the size of a tumor is reduced by at least 90%. In further or additional embodiments, the size of a tumor is reduced by at least 95%. In further or additional embodiments, the tumor is eradicated. In some embodiments, the size of a tumor does not increase.

In some embodiments, tumor proliferation is reduced by administering a compound of Formula (I), (Ia), (Ib), (Ic), (Id), (II), (IIa), (IIb), (IIc), (IId), (III), (IV), or a pharmaceutically acceptable salt thereof. In some embodiments, tumor proliferation is reduced by at least 1%. In some embodiments, tumor proliferation is reduced by at least 2%. In some embodiments, tumor proliferation is reduced by at least 3%. In some embodiments, tumor proliferation is reduced by at least 4%. In some embodiments, tumor proliferation is reduced by at least 5%. In some embodiments, tumor proliferation is reduced by at least 10%. In some embodiments, tumor proliferation is reduced by at least 20%. In some embodiments, tumor proliferation is reduced by at least 25%. In some embodiments, tumor proliferation is reduced by at least 30%. In some embodiments, tumor proliferation is reduced by at least 40%. In some embodiments, tumor proliferation is reduced by at least 50%. In some embodiments, tumor proliferation is reduced by at least 60%. In some embodiments, tumor proliferation is reduced by at least 70%. In some embodiments, tumor proliferation is reduced by at least 75%. In some embodiments, tumor proliferation is reduced by at least 75%. In some embodiments, tumor proliferation is reduced by at least 80%. In some embodiments, tumor proliferation is reduced by at least 90%. In some embodiments, tumor proliferation is reduced by at least 95%. In some embodiments, tumor proliferation is prevented.

### Methods of Administration

The compounds or pharmaceutical compositions may be administered to the patient by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; as well as administration topically; as deemed appropriate by those of skill in the art for bringing the compound of the invention into contact with living tissue.

Pharmaceutical compositions suitable for administration include compositions where the active ingredients are contained in an amount effective to achieve its intended purpose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 1 microgram/kg and 200 mg/kg body weight, preferably between about 180 microgram/kg and 10 mg/kg body weight. Alternatively dosages may be based and calculated upon the surface area of the patient, as understood by those of skill in the art.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). Typically, the dose range of the composition administered to the patient can be from about 0.5 to 1000 mg/kg of the patient's body weight. The dosage may be a single one or a series of two or more given in the course of one or more days, as is needed by the patient. In instances where human dosages for compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or TD₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, an oral dose of between 0.1 mg and 2000 mg of each active ingredient, preferably between 1 mg and 500 mg, e.g. 5 to 200 mg. In other embodiments, an intravenous, subcutaneous, or intramuscular dose of each active ingredient of between 0.01 mg and 100 mg, preferably between 0.1 mg and 60 mg, e.g. 1 to 40 mg is used. In cases of administration of a pharmaceutically acceptable salt, dosages may be calculated as the free base. In some embodiments, the composition is administered 1 to 4 times per day. Alternatively the compositions of the invention may be administered by continuous intravenous infusion, preferably at a dose of each active ingredient up to 1000 mg per day. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections. In some embodiments, the compounds will be administered for a period of continuous therapy, for example for a week or more, or for months or years.

In further or additional embodiments the amount of a compound of Formula (I), (Ia), (Ib), (Ic), (Id), (II), (IIa), (IIb), (IIc), (IId), (III), (IV), or a pharmaceutically acceptable salt thereof, may be administered in a range from about 0.001 to about 1000 mg/kg body weight/day. In further or additional embodiments, the amount a compound of Formula (I), (Ia), (Ib), (Ic), (Id), (II), (IIa), (IIb), (IIc), (IId), (III), (IV), or a pharmaceutically acceptable salt thereof, may be administered the range of about 0.5 to about 50 mg/kg/day. In further or additional embodiments the amount a compound of Formula (I), (Ia), (Ib), (Ic), (Id), (II), (IIa), (Iib), (Iic), (Iid), (III), (IV), or a pharmaceutically acceptable salt thereof, may be administered from about 0.001 to about 7 g/day. In further or additional embodiments the amount a compound of Formula (I), (Ia), (Ib), (Ic), (Id), (II), (IIa), (IIb), (IIc), (IId), (III), (IV), or a pharmaceutically acceptable salt thereof, may be administered from about 0.002 to about 6 g/day. In further or additional embodiments, the amount a compound of Formula (I), (Ia), (Ib), (Ic), (Id), (II), (IIa), (IIb), (IIc), (IId), (III), (IV), or a pharmaceutically acceptable salt thereof, may be administered from about 0.005 to about 5 g/day. In further or additional embodiments, the amount a compound of Formula (I), (Ia), (Ib), (Ic), (Id), (II), (IIa), (IIb), (IIc), (IId), (III), (IV), or a pharmaceutically acceptable salt thereof, may be administered from about 0.01 to about 5 g/day. In further or additional embodiments, the amount a compound of Formula (I), (Ia), (Ib), (Ic), (Id), (II), (IIa), (IIb), (IIc), (IId), (III), (IV), or a pharmaceutically acceptable salt thereof, may be administered from about 0.02 to about 5 g/day. In further or additional embodiments, the amount a compound of Formula (I), (Ia), (Ib), (Ic), (Id), (II), (IIa), (IIb), (IIc), (IId), (III), (IV), or a pharmaceutically acceptable salt thereof, may be administered from about 0.05 to about 2.5 g/day. In further or additional embodiments, the amount a compound of Formula (I), (Ia), (Ib), (Ic), (Id), (II), (IIa), (IIb), (IIc), (IId), (III), (IV), or a pharmaceutically acceptable salt thereof, may be administered from about 0.1 to about 1 g/day. In further or additional embodiments, dosage levels below the lower limit of the aforesaid range may be more than adequate.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered may be dependent on the subject being treated, on the subject's weight, the severity of the affliction, the manner of administration and the judgment of the prescribing physician.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

### Administration and Pharmaceutical Compositions

The compounds are administered at a therapeutically effective dosage. While human dosage levels have yet to be specifically identified for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, come embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

### Second (or Other Additional) Agents

In some embodiments, the second therapeutic agent is anti-inflammatory agent. In some embodiments, the second therapeutic agent is a non-steroidal anti-inflammatory agent. In some embodiments, the second therapeutic agent is anti-cancer agent.

In some embodiments, the methods comprise administering an effective amount of a compound of Formula (I), (Ia), (Ib), (Ic), (Id), (II), (IIa), (IIb), (IIc), (IId), (III), (IV), or a pharmaceutically acceptable salt thereof, in combination with an amount of a chemotherapeutic, wherein the amounts of the combination and the chemotherapeutic are together effective in inhibiting abnormal cell growth. Many chemotherapeutics are presently known in the art and can be used in combination. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Also described are methods for inhibiting abnormal cell growth in a mammal comprising administering to the mammal an amount of a MEK protein kinase inhibitor and/or Raf protein kinase inhibitor in combination with radiation therapy, wherein the amounts of the MEK protein kinase inhibitor and/or Raf protein kinase inhibitor in combination with the radiation therapy effective in inhibiting abnormal cell growth or treating the hyperproliferative disorder in the mammal. Techniques for administering radiation therapy are known in the art, and these techniques can be used in the combination therapy described herein.

In some embodiments, the disclosure also relates to a method of inhibiting abnormal cell growth in a mammal which may comprises a compound of Formula (I), (Ia), (Ib), (Ic), (II), (IIa), (IIb), (IIc), (III), (IV), or a pharmaceutically acceptable salt thereof, and an amount of one or more substances selected from anti-angiogenesis agents, signal transduction inhibitors, and antiproliferative agents. Anti-angiogenesis agents, such as MMP-2 (matrix-metalloprotienase 2) inhibitors, MMP-9 (matrix-metalloprotienase 9) inhibitors, and COX-11 (cyclooxygenase 11) inhibitors, can be used in conjunction with a compound of the present invention and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 19911), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997). Some MMP-2 and MMP-9 inhibitors have little or no activity inhibiting MMP-1, while some selectively inhibit MMP-2 and/or AMP-9 relative to the other matrix-motalloproteinases (L e., MAP-1, NEMP-3, MMP-4, M7v1P-5, MMP-6, MMP-7, MMP-8, MMP-10, MMP-11, and MMP-13). Some specific examples of M1v1P inhibitors useful in the present invention are AG-3340, RU 32-3555, and RS 13-0830.

In some embodiments, a compound of Formula (I), (Ia), (Ib), (Ic), (Id), (II), (IIa), (IIb), (IIc), (IId), (III), (IV), or a pharmaceutically acceptable salt thereof, is administered with at least one additional therapeutic agent. In some embodiments, the therapeutic agent is a taxol, bortezornib or both. In further or additional embodiments, the therapeutic agent is selected from the group consisting of cytotoxic agents, anti-angiogenesis agents and anti-neoplastic agents. In further or additional embodiments, the anti-neoplastic agents selected from the group of consisting of alkylating agents, anti-metabolites, epiclophyllotoxims; antineoplastic enzymes, topoisomerase inhibitors, procarbazine, mitoxantrone, platinum coordination complexes, biological response modifiers and growth inhibitors, hormonal/anti-hormonal therapeutic agents, and baematopoietic growth factors.

Many chemotherapeutics are presently known in the art and can be used in combination with the compounds and compositions of the disclosure. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

In some embodiments, the combination is administered in combination with an additional therapy. In further or additional embodiments, the additional therapy is radiation therapy, chemotherapy, surgery or any combination thereof. In further or additional embodiments, the combination is administered in combination with at least one additional therapeutic agent. In further or additional embodiments, the therapeutic agent is selected from the group of cytotoxic agents, anti-angiogenesis agents and anti-neopiastic agents. In further or additional embodiments, the anti-neoplastic agent is selected from the group of consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors.

In some embodiments, the second therapeutic is an agent for co-regulating RAF pathways. In some embodiments, the second therapeutic agent is a RAF inhibitor. In some embodiments, the RAF inhibitor is vemurafenib, dabrafenib, encorafenib, XL-281, LGX-818, CEP-32496, and ARQ-736.

In some embodiments, the second therapeutic agent is selected from aspirin; diflunisal; salsalate; acetaminophen; ibuprofen; dexibuprofen; naproxen; fenoprofen; ketoprofen; dexketoprofen; flurbiprofen; oxaprozin; loxoprofen; indomethacin; tolmetin; sulindac; etodolac; ketorolac; diclofenac; aceclofenac; nabumetone; enolic acid; piroxicam; meloxicam; tenoxicam; droxicam; lornoxicam; isoxicam; mefenamic acid; meclofenamic acid; flufenamic acid; tolfenamic acid; sulfonanilides; clonixin; licofelone; dexamethasone; and prednisone.

In some embodiments, the second therapeutic agent is selected from mechlorethamine; cyclophosphamide; melphalan; chlorambucil; ifosfamide; busulfan; N-nitroso-N-methylurea (MNU); carmustine (BCNU); lomustine (CCNU); semustine (MeCCNU); fotemustine; streptozotocin; dacarbazine; mitozolomide; temozolomide; thiotepa; mytomycin; diaziquone (AZQ); cisplatin; carboplatin; and oxaliplatin.

In some embodiments, the second therapeutic agent is selected from vincristine; vinblastine; vinorelbine; vindesine; vinflunine; paclitaxel; docetaxel; etoposide; teniposide; tofacitinib; ixabepilone; irinotecan; topotecan; camptothecin; doxorubicin; mitoxantrone; and teniposide.

In some embodiments, the second therapeutic agent is selected from actinomycin; bleomycin; plicamycin; mitomycin; daunorubicin; epirubicin; idarubicin; pirarubicin; aclarubicin; mitoxantrone; cyclophosphamide; methotrexate; 5-fluorouracil; prednisolone; folinic acid; methotrexate; melphalan; capecitabine; mechlorethamine; uramustine; melphalan; chlorambucil; ifosfamide; bendamustine; 6-mercaptopurine; and procarbazine.

In some embodiments, the second therapeutic agent is selected from cladribine; pemetrexed; fludarabine; gemcitabine; hydroxyurea; nelarabine; cladribine; clofarabine; ytarabine; decitabine; cytarabine; cytarabine liposomal; pralatrexate; floxuridine; fludarabine; colchicine; thioguanine; cabazitaxel; larotaxel; ortataxel; tesetaxel; aminopterin; pemetrexed; pralatrexate; raltitrexed; pemetrexed; carmofur; and floxuridine.

In some embodiments, the second therapeutic agent is selected from azacitidine; decitabine; hydroxycarbamide; topotecan; irinotecan; belotecan; teniposide; aclarubicin; epirubicin; idarubicin; amrubicin; pirarubicin; valrubicin; zorubicin; mitoxantrone; pixantrone; mechlorethamine; chlorambucil; prednimustine; uramustine; estramustine; carmustine; lomustine; fotemustine; nimustine; ranimustine; carboquone; thioTEPA; triaziquone; and triethylenemelamine.

In some embodiments, the second therapeutic agent is selected from nedaplatin; satraplatin; procarbazine; dacarbazine; temozolomide; altretamine; mitobronitol; pipobroman; actinomycin; bleomycin; plicamycin; aminolevulinic acid; methyl aminolevulinate; efaproxiral; talaporfin; temoporfin; verteporfin; alvocidib; seliciclib; palbociclib; bortezomib; carfilzomib; anagrelide; masoprocol; olaparib; belinostat; panobinostat; romidepsin; vorinosta; idelalisib; atrasentan; bexarotene; testolactone; amsacrine; trabectedin; alitretinoin; tretinoin; demecolcine; elsamitrucin; etoglucid; lonidamine; lucanthone; mitoguazone; mitotane; oblimersen; omacetaxine mepesuccinate; and eribulin.

In some embodiments, the second therapeutic agent is selected from azathioprine; Mycophenolic acid; leflunomide; teriflunomide; tacrolimus; cyclosporin; pimecrolimus; abetimus; gusperimus; lenalidomide; pomalidomide; thalidomide; anakinra; sirolimus; everolimus; ridaforolimus; temsirolimus; umirolimus; zotarolimus; eculizumab; adalimumab; afelimomab; certolizumab pegol; golimumab; infliximab; nerelimomab; mepolizumab; omalizumab; faralimomab; elsilimomab; lebrikizumab; ustekinumab; etanercept; otelixizumab; teplizumab; visilizumab; clenoliximab; keliximab; zanolimumab; efalizumab; erlizumab; obinutuzumab; rituximab; and ocrelizumab.

In some embodiments, the second therapeutic agent is selected from pascolizumab; gomiliximab; lumiliximab; teneliximab; toralizumab; aselizumab; galiximab; gavilimomab; ruplizumab; belimumab; blisibimod; ipilimumab; tremelimumab; bertilimumab; lerdelimumab; metelimumab; natalizumab; tocilizumab; odulimomab; basiliximab; daclizumab; inolimomab; zolimoma; atorolimumab; cedelizumab; fontolizumab; maslimomab; morolimumab; pexelizumab; reslizumab; rovelizumab; siplizumab; talizumab; telimomab; vapaliximab; vepalimomab; abatacept; belatacept; pegsunercept; aflibercept; alefacept; and rilonacept.

Accordingly, some aspects described herein relate to the following numbered alternatives:

1. A compound having the structure of Formula (III):

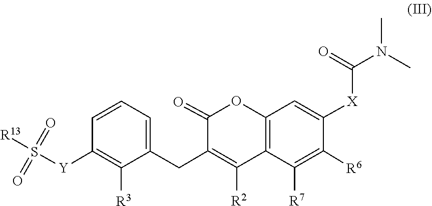

including pharmaceutically acceptable salts thereof, wherein:
R², R⁶, R⁷, and R¹³ are each independently selected from the group consisting of H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L;
R³ is a chloro, bromo, or iodo;
X is C(R⁵)₂, CH(R⁵), CH₂, —O—,

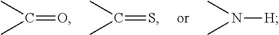

Y is C(R⁵)₂, CH(R⁵), CH₂, —O—,

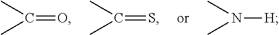

L is —Z₁-Z₂ or —Z₁-Z₂—Z₃;
Z₁, Z₂, and Z₃ are independently selected from the group consisting of —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl); and
each R⁵ and R^{5′} are independently selected from H, deuterium, optionally substituted C₁ to C₆ alkyl, optionally substituted C₂ to C₆ alkenyl, optionally substituted C₂ to C₆ alkynyl, optionally substituted C₃ to C₈ carbocyclyl, optionally substituted C₆ to C₁₀ aryl, optionally substituted C₃ to C₈ heterocyclyl, and optionally substituted C₃ to C₁₀ heteroaryl.

2. The compound of alternative 1, wherein R⁶ is H, deuterium, hydroxyl or halo.

3. The compound of alternative 1 or 2, wherein R⁶ is H or halo.

4. The compound of any one of alternatives 1 to 3, wherein halo is selected from fluoro, chloro or bromo.

5. The compound of any one of alternatives 1 to 4, wherein R⁷ is L.

6. The compound of alternative 5, wherein L is —Z₁-Z₂.

7. The compound of alternative 6, wherein Z₁ is —CH₂—.

8. The compound of alternative 6 or 7, wherein Z₂ is selected from optionally substituted C₃ to C₈ cycloalkyl, optionally substituted C₃ to C₈ heterocyclyl, optionally substituted C₃ to C₈ heteroaryl, —NR⁵R^{5′}, —CH₂CCH, or —CH₂CN.

9. The compound of alternative 5, wherein L is —Z₁-Z₂—Z₃.

10. The compound of alternative 9, wherein Z₁ is —CH₂—, Z₂ is selected from the group consisting of —NR⁵R^{5′}, —NHCH₂CO—, C₃ to C₈ cycloalkyl, optionally substituted C₃ to C₈ heterocyclyl, optionally substituted C₃ to C₈ heteroaryl and Z₃ is selected from the group consisting of H, deuterium, halo, optionally substituted C₁ to C₆ alkyl, optionally substituted C₃ to C₈ cycloalkyl, optionally substituted C₆ to C₁₀ aryl, or —CH₂-(optionally substituted aryl).

11. The compound of any one of alternatives 1 to 10, wherein R³ is chloro.

12. The compound of any one of alternatives 1 to 10, wherein R³ is bromo.

13. The compound of any one of alternatives 1 to 10, wherein R³ is iodo.

14. The compound of any one of alternatives 1 to 13, wherein R² is L.

15. The compound of alternative 14, wherein L is —Z₁-Z₂.

16. The compound of alternative 15, wherein Z₁ is —CH₂— or —NH—.

17. The compound of any one of alternatives 1 to 16, wherein X is CH₂ or

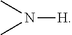

18. The compound of any one of alternatives 1 to 17, wherein Y is CH₂ or

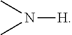

19. The compound of alternative 1, wherein the compound is selected from a compound of Table B, C, D, or E.

20. A pharmaceutical composition comprising a therapeutically effective amount of at least one compound having the structure of Formula (III)

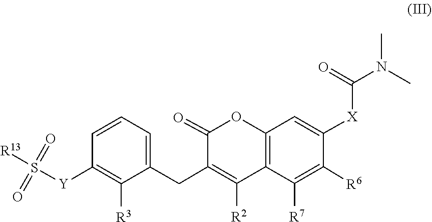

including pharmaceutically acceptable salts thereof, wherein:
R², R⁶, R⁷, and R¹³ are each independently selected from the group consisting of H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L;
R³ is a chloro, bromo or iodo;
X is C(R⁵)₂, CH(R⁵), CH₂, —O—,

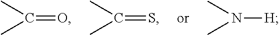

Y is C(R⁵)₂, CH(R⁵), CH₂, —O—,

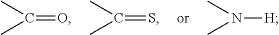

L is —Z₁-Z₂ or —Z₁-Z₂—Z₃;
Z₁, Z₂, and Z₃ are independently selected from the group consisting of —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl); and
each R⁵ and R^{5′} are independently selected from H, deuterium, optionally substituted C₁ to C₆ alkyl, optionally substituted C₂ to C₆ alkenyl, optionally substituted C₂ to C₆ alkynyl, optionally substituted C₃ to C₈ carbocyclyl, optionally substituted C₆ to C₁₀ aryl, optionally substituted C₃ to C₈ heterocyclyl, and optionally substituted C₃ to C₁₀ heteroaryl.

21. The pharmaceutical composition of alternative 20, wherein R⁶ is H, deuterium, hydroxyl or halo.

22. The pharmaceutical composition of alternative 20 or 21, wherein R⁶ is H or halo.

23. The pharmaceutical composition of any one of alternatives 20 to 22, wherein halo is selected from chloro or bromo.

24. The pharmaceutical composition of any one of alternatives 20 to 23, wherein R⁷ is L.

25. The pharmaceutical composition of alternative 24, wherein L is —Z₁-Z₂.

26. The pharmaceutical composition of alternative 25, wherein Z₁ is —CH₂—.

27. The pharmaceutical composition of alternative 25 or 26, wherein Z₂ is selected from optionally substituted C₃ to C₈ cycloalkyl, optionally substituted C₃ to C₈ heterocyclyl, optionally substituted C₃ to C₈ heteroaryl, —NR⁵R^{5′}, —CH₂CCH, or —CH₂CN.

28. The pharmaceutical composition of alternative 24, wherein L is —Z₁-Z₂—Z₃.

29. The pharmaceutical composition of alternative 28, wherein Z₁ is —CH₂—, Z₂ is selected from the group consisting of —NR⁵R^{5′}, —NHCH₂CO—, C₃ to C₈ cycloalkyl, optionally substituted C₃ to C₈ heterocyclyl, optionally substituted C₃ to C₈ heteroaryl and Z₃ is selected from the group consisting of H, deuterium, halo, optionally substituted C₁ to C₆ alkyl, optionally substituted C₃ to C₈ cycloalkyl, optionally substituted C₆ to C₁₀ aryl, or —CH₂-(optionally substituted aryl).

30. The pharmaceutical composition of any one of alternatives 20 to 29, wherein R³ is chloro.

31. The pharmaceutical composition of any one of alternatives 20 to 29, wherein R³ is bromo.

32. The pharmaceutical composition of any one of alternatives 20 to 29, wherein the R³ is iodo.

33. The pharmaceutical composition of any one of alternatives 30 to 32, wherein R¹³ is L.

34. The pharmaceutical composition of alternative 33, wherein L is —Z₁-Z₂.

35. The pharmaceutical composition of alternative 34, wherein Z₁ is —CH₂— or —NH—.

36. The pharmaceutical composition of any one of alternatives 20 to 35, wherein X is CH₂ or

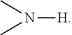

37. The pharmaceutical composition of any one of alternatives 20 to 36, wherein Y is CH₂ or

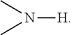

38. The pharmaceutical composition of alternative 20, wherein the compound is selected from a compound of Table B, C, D, or E.

39. The pharmaceutical composition of any one of any one of alternatives 20 to 38, further comprising one or more immune checkpoint inhibitors.

40. The pharmaceutical composition of alternative 40, wherein the immune checkpoint inhibitor is an inhibitor of PD-1, PD-L1, PD-L2, PD-L3, PD-L4, CTLA-4, LAG3, B7-H3, B7-H4, KIR or TIM3.

41. The pharmaceutical composition of alternative 39 or 40, wherein the immune checkpoint inhibitor is a PD-1 inhibitor.

42. The pharmaceutical composition of alternative 39 or 40, wherein the immune checkpoint inhibitor is a PD-L1 inhibitor.

43. The pharmaceutical composition of alternative 39 or 40, wherein the immune checkpoint inhibitor is a PD-L2 inhibitor.

44. The pharmaceutical composition of alternative 39 or 40, wherein the immune checkpoint inhibitor is a CTLA-4 inhibitor.

45. The pharmaceutical composition of alternative 39 or 40, comprising a first immune checkpoint inhibitor and a second immune checkpoint inhibitor, wherein the first immune checkpoint inhibitor is different from the second immune checkpoint inhibitor.

46. The pharmaceutical composition of alternative 39 or 40, wherein the first and the second immune checkpoint inhibitor is independently an inhibitor of PD-1, PD-L1, PD-L2, PD-L3, PD-L4, CTLA-4, LAG3, B7-H3, B7-H4, KIR or TIM3.

47. The pharmaceutical composition of alternative 39 or 40, wherein the first immune checkpoint inhibitor is a PD-1 inhibitor, and the second immune checkpoint inhibitor is a CTLA-4 inhibitor.

48. The pharmaceutical composition of alternative 39 or 40, wherein the first immune checkpoint inhibitor is a PD-L1 inhibitor, and the second immune checkpoint inhibitor is a CTLA-4 inhibitor.

49. The pharmaceutical composition of alternative 39 or 40, wherein the first immune checkpoint inhibitor is a PD-L2 inhibitor, and the second immune checkpoint inhibitor is a CTLA-4 inhibitor.

50. The pharmaceutical composition of alternative 39 or 40, wherein the immune checkpoint inhibitor is an antibody.

51. The pharmaceutical composition of alternative 39 or 40, wherein the immune checkpoint inhibitor is a PD-1 antibody.

52. The pharmaceutical composition of alternative 39 or 40, wherein the immune checkpoint inhibitor is a PD-L1 antibody.

53. The pharmaceutical composition of alternative 39 or 40, wherein the immune checkpoint inhibitor is a PD-L2 antibody.

54. The pharmaceutical composition of alternative 39 or 40, wherein the immune checkpoint inhibitor is a CTLA-4 antibody.

55. The pharmaceutical composition of alternative 39 or 40, wherein the immune checkpoint inhibitor is nivolumab, pembrolizumab, pidilizumab, ipilimumab, BMS 936559, atezolizumab, durvalumab, or any combinations thereof.

56. A method of treating a mammal having a disease or disorder, comprising administering to the mammal a therapeutically effective amount of a compound of any one alternatives 1 to 19 or a pharmaceutical composition of any one of alternatives 20 to 55.

57. A method of treating a disease or disorder, comprising administering to a subject suffering from said disease or disorder an effective amount of a compound of any alternatives 1 to 19 or a pharmaceutical composition of any one of alternatives 20 to 55.

58. A method of treating a disease, comprising administering to a subject suffering from said disease an effective amount of a compound of any one alternatives 1 to 19 or a pharmaceutical composition of any one of alternatives 20 to 55.

59. The method of any one of alternatives 56 to 58, wherein the disease is cancer.

60. A method of treating cancer cachexia in a mammal with cancer comprising administering an effective amount of a compound of any one alternatives 1 to 19 or a pharmaceutical composition of any one of alternatives 20 to 55.

61. The method according to any one of alternatives 56 to 60, wherein the compound of any one alternatives 1 to 19 or a pharmaceutical composition of any one of alternatives 20 to 55.

62. The method according to any one of alternatives 56 to 60, wherein the compound of any one alternatives 1 to 19 or a pharmaceutical composition of any one of alternatives 20 to 55.

63. The method according to any one of alternatives 56 to 60, wherein the compound of any one alternatives 1 to 19 or a pharmaceutical composition of any one of alternatives 20 to 55 is administered in multiple doses, more than once per day.

64. The method of alternative 59, wherein the cancer is selected from the group consisting of brain cancer, breast cancer, lung cancer, non-small cell lung cancer, ovarian cancer, pancreatic cancer, stomach cancer, prostate cancer, renal cancer, colorectal cancer or leukemia. In further or additional embodiments, the fibrogenetic disorder is scleroderma, polymyositis, systemic lupus, rheumatoid arthritis, liver cirrhosis, keloid formation, interstitial nephritis or pulmonary fibrosis.

65. The method alternative 59 or 64, wherein the cancer is associated with a RAS mutation.

66. The method alternative 65, wherein the RAS mutation is a KRAS mutation selected from the group consisting of G12C, G12S, G12R, G12F, G12L, G12N, G12A, G12D, G12V, G13C, G13S, G13D, G13V, G13P, S17G, P34S, A59E, A59G, A59T, Q61K, Q61L, Q61R, and Q61H.

67. A compound having the chemical structure of Formula (IV)

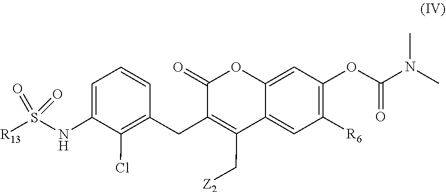

or a pharmaceutically acceptable salt thereof, wherein:
R₆ is hydrogen, fluoro or chloro;
R₁₃ is ethyl or —NR_{A}R_{B} wherein R_{A} is hydrogen and R_{B} is methyl;
Z₂ is —NR⁵R^{5′},

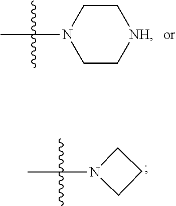 [SMILES: *N1CCNCC1 ; *N1CCC1]

R⁵ is C₁ to C₆ alkyl; and
R^{5′} is C₁ to C₆ alkyl.

68. The compound of alternative 67, wherein R⁵ is methyl.

69. The compound of alternative 68, wherein R^{5′} is methyl.

70. The compound of alternative 68, wherein R^{5′} is ethyl.

71. The compound of any one of alternatives 67 to 70, wherein Z₂ is —NR⁵R^{5′}.

72. The compound of any one of alternatives 67 to 71, wherein R₁₃ is —NR_{A}R_{B}.

73. The compound of alternative 72, wherein the compound is

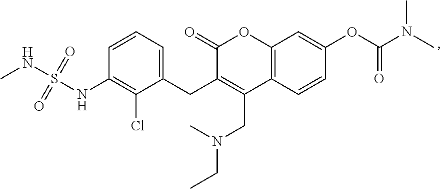

or a pharmaceutically acceptable salt thereof.

74. The compound of alternative 72, wherein the compound is

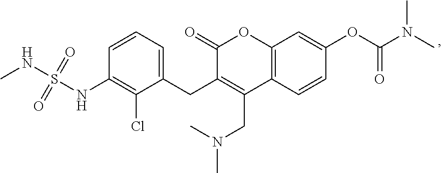

or a pharmaceutically acceptable salt thereof.

75. The compound of alternative 72, wherein the compound is

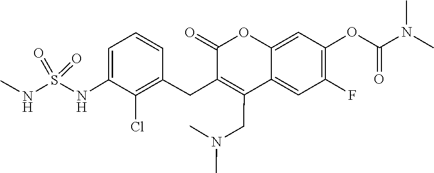

or a pharmaceutically acceptable salt thereof.

76. The compound of alternative 72, wherein the compound is

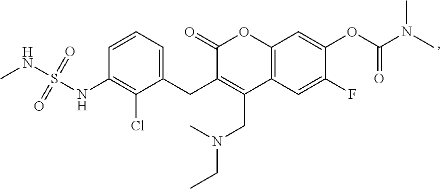

or a pharmaceutically acceptable salt thereof.

77. The compound of alternative 72, wherein the compound is

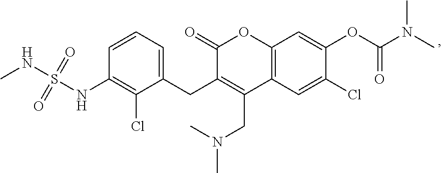

or, or a pharmaceutically acceptable salt thereof.

78. The compound of alternative 72, wherein the compound is

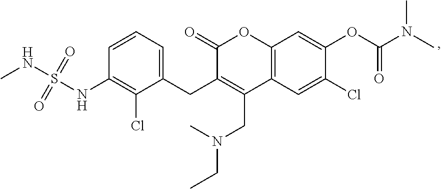

or a pharmaceutically acceptable salt thereof.

79. The compound of any one of alternatives 67 to 71, wherein R₁₃ is ethyl.

80. The compound of alternative 79, wherein the compound is

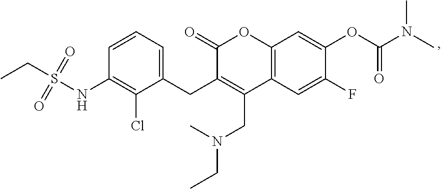 [SMILES: CCN(C)Cc1c(Cc2cccc(NS(=O)(=O)CC)c2Cl)c(=O)oc2cc(OC(=O)N(C)C)c(F)cc12]

or a pharmaceutically acceptable salt thereof.

81. The compound of alternative 79, wherein the compound is

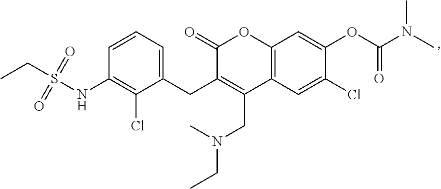 [SMILES: CCN(C)Cc1c(Cc2cccc(NS(=O)(=O)CC)c2Cl)c(=O)oc2cc(OC(=O)N(C)C)c(Cl)cc12]

or a pharmaceutically acceptable salt thereof.

82. The compound of alternative 79, wherein the compound is

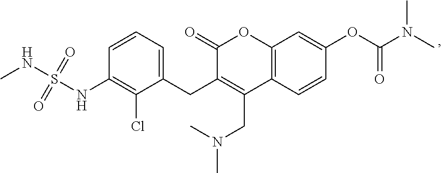

for a pharmaceutically acceptable salt thereof.

83 The compound of any one of alternatives 67 to 70, wherein Z₂ is

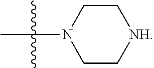 [SMILES: *N1CCNCC1]

84. The compound of alternative 83, wherein R₁₃ is ethyl.

85. The compound of alternative 84, wherein the compound is

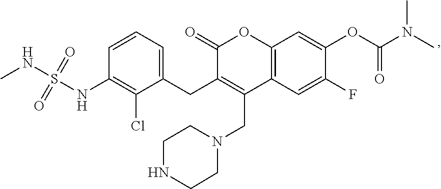

or a pharmaceutically acceptable salt thereof.

86. The compound of alternative 84, wherein the compound is

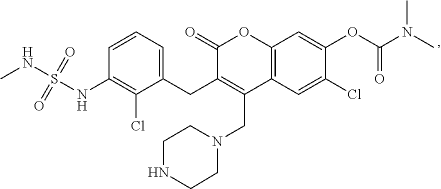

or a pharmaceutically acceptable salt thereof.

87. The compound of alternative 84, wherein the compound is

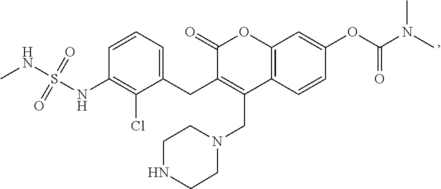

or a pharmaceutically acceptable salt thereof.

88. The compound of alternative 83, wherein R₁₃ is —NR_{A}R_{B}.

89. The compound of alternative 88, wherein the compound is

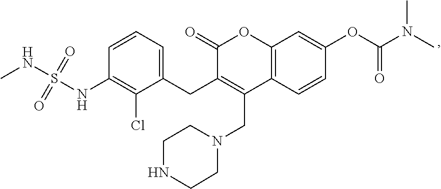

or a pharmaceutically acceptable salt thereof.

90. The compound of alternative 88, wherein the compound is

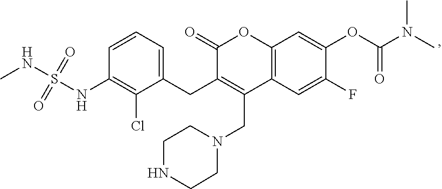

or a pharmaceutically acceptable salt thereof.

91. The compound of alternative 88, wherein the compound is

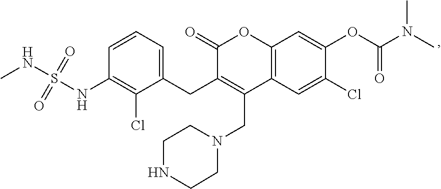

or a pharmaceutically acceptable salt thereof.

92. The compound of any one of alternatives 67 to 70, wherein Z₂ is

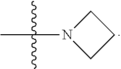 [SMILES: *N1CCC1]

93. The compound of alternative 92, wherein R₁₃ is ethyl.

94. The compound of alternative 92, wherein R₁₃ is —NR_{A}R_{B}.

95. The compound of alternative 92 or 94, wherein the compound is

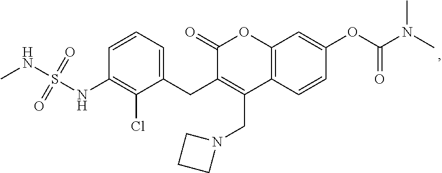

or a pharmaceutically acceptable salt thereof.

96. The compound of alternative 92 or 94, wherein the compound is

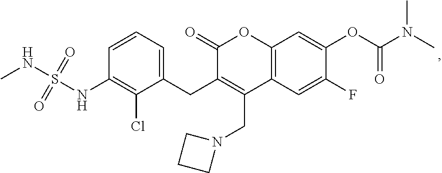

or a pharmaceutically acceptable salt thereof.

97. The compound of alternative 92 or 94, wherein the compound is

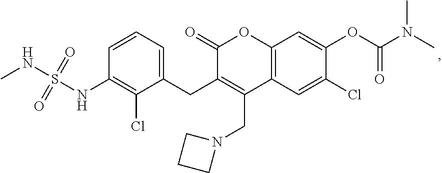

or a pharmaceutically acceptable salt thereof.

98. A compound having the structure of Formula (III):

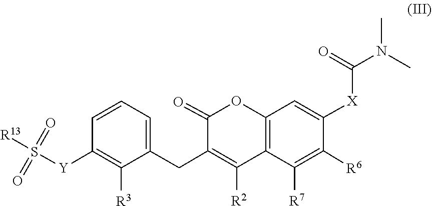

including pharmaceutically acceptable salts thereof, wherein:
R² is L;
R⁶ is selected from the group consisting of H or fluoro, chloro or bromo;
R⁷ is H;
R¹³ is selected from the group consisting of optionally substituted optionally substituted amin, C₁ to C₆ alkyl, H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L;
R³ is a chloro;
X is —O—;
Y is

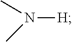

L is —Z₁-Z₂;
Z₁ is —CH₂—; and
Z₂, is selected from the group consisting of —NR⁵R^{5′}, optionally substituted C₃ to C₈ heterocyclyl, —CH₂—, —O—, —S—, S═O, —SO₂—, C═O, —CO₂—, —NO₂, —NH—, —CH₂CCH, —CH₂CN, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl); and each R⁵ and R^{5′} are independently selected optionally substituted C₁ to C₆ alkyl.

99. The compound of alternative 98, wherein Z₂, is —NR⁵R^{5′}.

100. The compound of alternative 99, wherein R⁵ is methyl.

101. The compound of alternative 99, wherein R^{5′} is methyl.

102. The compound of alternative 99, wherein R^{5′} is ethyl.

103. The compound of alternative 98, wherein Z₂ is

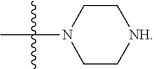 [SMILES: *N1CCNCC1]

104. The compound of alternative 98, wherein Z₂ is optionally substituted

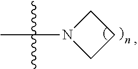

wherein n is 1, 2, 3 or 4.

105. The compound of alternative 104, wherein n is 1.

106. The compound of any one of alternatives 98 to 105, wherein R¹³ is —NR_{A}R_{B} wherein R_{A} and R_{B} are each independently selected from hydrogen, or C₁₋₆ alkyl.

107. The compound of alternative 106, wherein R_{A} is hydrogen and R_{B} is methyl.

108. The compound of any one of alternatives 98 to 105, wherein R¹³ is C₁ to C₆ alkyl.

109. The compound of alternative 108, wherein R¹³ is ethyl.

110. The compound of any one of alternatives 98 to 109, wherein R⁶ is fluoro.

111. The compound of any one of alternatives 98 to 109, wherein R⁶ is chloro.

112. The compound of any one of alternatives 98 to 109, wherein R⁶ is H.

113. A compound having the structure of Formula (III):

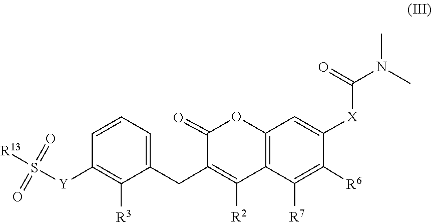

including pharmaceutically acceptable salts thereof, wherein,
R² is L;
R⁶ is selected from the group consisting of H or fluoro, chloro or bromo;
R⁷ is H;
R¹³ is C₁ to C₆ alkyl;
R³ is a chloro;
X is —O—;
Y is

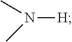

L is —Z₁-Z₂;
Z₁ is —CH₂—;
Z₂ is —NR⁵R^{5′}; and
each R⁵ and R^{5′} are independently selected from optionally substituted C₁ to C₆ alkyl.

114. A compound having the structure of Formula (III):

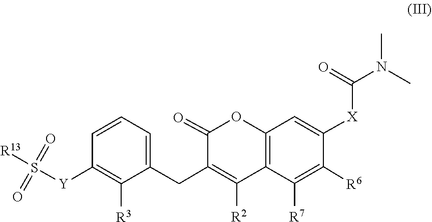

including pharmaceutically acceptable salts thereof, wherein,
R² is selected from the group consisting of halogen, H, deuterium, hydroxyl, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L;
R⁶ is selected from the group consisting of H, halogen, deuterium, hydroxyl, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L;
R⁷ is selected from the group consisting of deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L;
R¹³ is selected from the group consisting of optionally substituted C₁ to C₆ alkyl, optionally substituted amino, H, deuterium, hydroxyl, halogen, cyano, nitro, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L;
R³ is a chloro, bromo, or iodo;
X is —O—, C(R⁵)₂, CH(R⁵), CH₂,

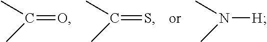

Y is

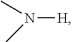

C(R⁵)₂, CH(R⁵), CH₂, —O—,

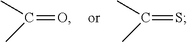

L is —Z₁-Z₂ or —Z₁-Z₂—Z₃;
Z₁ is selected from the group consisting of —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl); Z₂, is selected from the group consisting of —NR⁵R^{5′}, —CH₂—, —O—, —S—, S═O, —SO₂—, C═O, —CO₂—, —NO₂, —NH—, —CH₂CCH, —CH₂CN, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl);
Z₃ is selected from the group consisting of —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R'S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl); and
each R⁵ and R^{5′} are independently selected from optionally substituted C₁ to C₆ alkyl, H, deuterium, optionally substituted C₂ to C₆ alkenyl, optionally substituted C₂ to C₆ alkynyl, optionally substituted C₃ to C₈ carbocyclyl, optionally substituted C₆ to C₁₀ aryl, optionally substituted C₃ to C₈ heterocyclyl, and optionally substituted C₃ to C₁₀ heteroaryl.

115. A compound selected from the list consisting of:

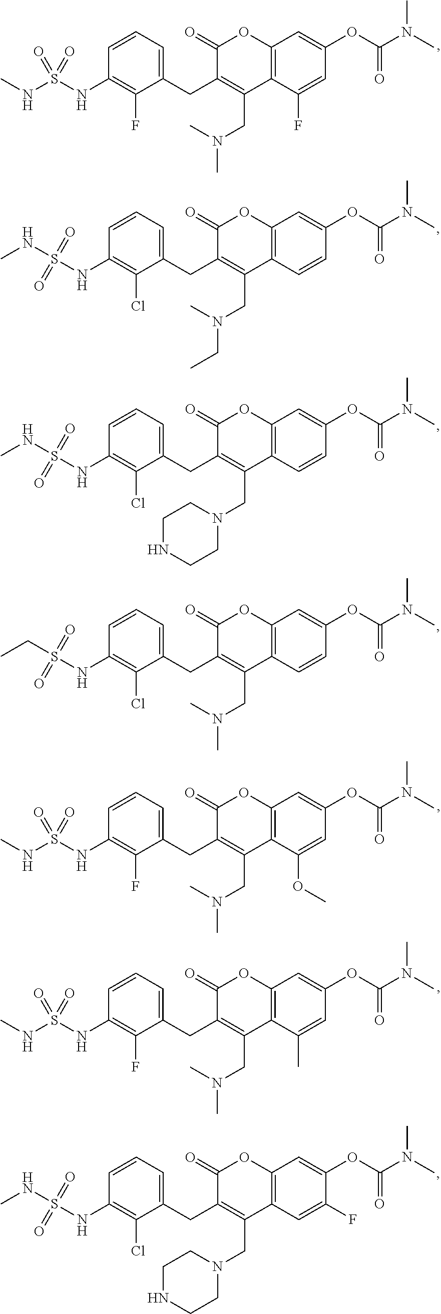

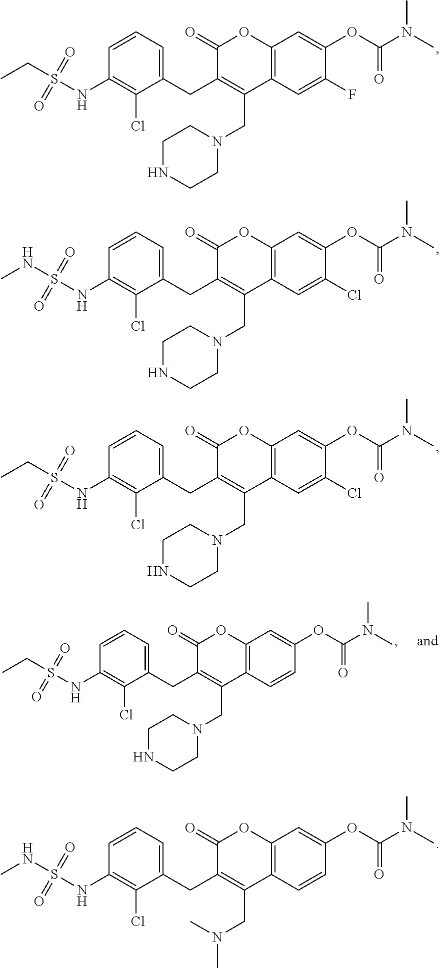

116. A compound selected from the list consisting of:

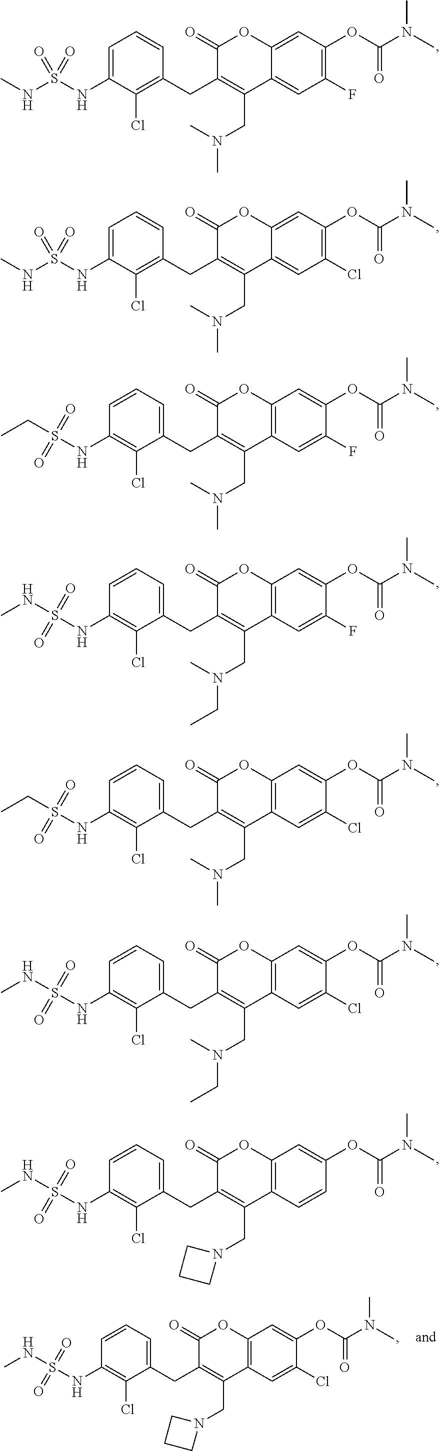

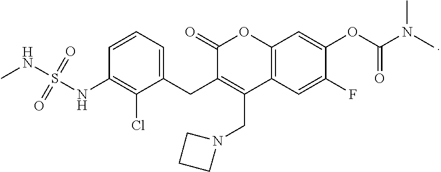

117. A pharmaceutical composition comprising a compound of any one of alternatives 67 to 116, or a pharmaceutically acceptable salt thereof.

118. A method of treating cancer, comprising administering to a subject in need thereof an effective amount of a compound of any one alternatives 67 to 116 or a pharmaceutical composition thereof.

119. A use of a compound of any one of alternatives 67 to 116 for the treatment of cancer.

120. A compound selected from the group consisting of:
4-((dimethylamino)methyl)-5-fluoro-3-(2-fluoro-3-((N-methylsulfamoyl)amino)benzyl)-2-oxo-2H-chromen-7-yl dimethylcarbamate;
4-((dimethylamino)methyl)-3-(2-fluoro-3-((N-methylsulfamoyl)amino)benzyl)-5-methoxy-2-oxo-2H-chromen-7-yl dimethylcarbamate;
4-((dimethylamino)methyl)-3-(2-fluoro-3-((N-methylsulfamoyl)amino)benzyl)-5-methyl-2-oxo-2H-chromen-7-yl dimethylcarbamate; and
4-((dimethylamino)methyl)-3-(2-fluoro-3-((N-methylsulfamoyl)amino)benzyl)-5-methoxy-2-oxo-2H-chromen-7-yl dimethylcarbamate.

121. The compound of alternative 120, wherein the compound is 4-((dimethylamino)methyl)-5-fluoro-3-(2-fluoro-3-((N-methylsulfamoyl)amino)benzyl)-2-oxo-2H-chromen-7-yl dimethylcarbamate.

122. The compound of alternative 120, wherein the compound is 4-((dimethylamino)methyl)-3-(2-fluoro-3-((N-methylsulfamoyl)amino)benzyl)-5-methyl-2-oxo-2H-chromen-7-yl dimethylcarbamate.

123. The compound of alternative 120, wherein the compound is 4-((dimethylamino)methyl)-3-(2-fluoro-3-((N-methylsulfamoyl)amino)benzyl)-5-methoxy-2-oxo-2H-chromen-7-yl dimethylcarbamate.

124. A pharmaceutical composition comprising a compound of alternatives 1 and a pharmaceutically acceptable salt.

125. A compound selected from the group consisting of:
3-(2-chloro-3-((N-methylsulfamoyl)amino)benzyl)-4-((ethyl(methyl)amino)methyl)-2-oxo-2H-chromen-7-yl dimethylcarbamate;
3-(2-chloro-3-((N-methylsulfamoyl)amino)benzyl)-2-oxo-4-(piperazin-1-ylmethyl)-2H-chromen-7-yl dimethylcarbamate;
3-(2-chloro-3-((N-methylsulfamoyl)amino)benzyl)-2-oxo-4-(piperazin-1-ylmethyl)-2H-chromen-7-yl dimethylcarbamate;
3-(2-chloro-3-(ethylsulfonamido)benzyl)-4-((dimethylamino)methyl)-2-oxo-2H-chromen-7-yl dimethylcarbamate;
3-(2-chloro-3-((N-methylsulfamoyl)amino)benzyl)-6-fluoro-2-oxo-4-(piperazin-1-ylmethyl)-2H-chromen-7-yl dimethylcarbamate;
3-(2-chloro-3-(ethylsulfonamido)benzyl)-6-fluoro-2-oxo-4-(piperazin-1-ylmethyl)-2H-chromen-7-yl dimethylcarbamate;
6-chloro-3-(2-chloro-3-((N-methylsulfamoyl)amino)benzyl)-2-oxo-4-(piperazin-1-ylmethyl)-2H-chromen-7-yl dimethylcarbamate;
6-chloro-3-(2-chloro-3-(ethylsulfonamido)benzyl)-2-oxo-4-(piperazin-1-ylmethyl)-2H-chromen-7-yl dimethylcarbamate;
3-(2-chloro-3-(ethylsulfonamido)benzyl)-2-oxo-4-(piperazin-1-ylmethyl)-2H-chromen-7-yl dimethylcarbamate; and
3-(2-chloro-3-((N-methylsulfamoyl)amino)benzyl)-4-((dimethylamino)methyl)-2-oxo-2H-chromen-7-yl dimethylcarbamate.

126. The compound of alternative 126, wherein the compound is 3-(2-chloro-3-((N-methylsulfamoyl)amino)benzyl)-4-((ethyl(methyl)amino)methyl)-2-oxo-2H-chromen-7-yl dimethylcarbamate.

127. The compound of alternative 126, wherein the compound is 3-(2-chloro-3-((N-methylsulfamoyl)amino)benzyl)-2-oxo-4-(piperazin-1-ylmethyl)-2H-chromen-7-yl dimethylcarbamate.

128. The compound of alternative 126, wherein the compound is 3-(2-chloro-3-(ethylsulfonamido)benzyl)-4-((dimethylamino)methyl)-2-oxo-2H-chromen-7-yl dimethylcarbamate.

129. The compound of alternative 126, wherein the compound is 3-(2-chloro-3-((N-methylsulfamoyl)amino)benzyl)-6-fluoro-2-oxo-4-(piperazin-1-ylmethyl)-2H-chromen-7-yl dimethylcarbamate.

130. The compound of alternative 126, wherein the compound is 3-(2-chloro-3-(ethylsulfonamido)benzyl)-6-fluoro-2-oxo-4-(piperazin-1-ylmethyl)-2H-chromen-7-yl dimethylcarbamate.

131. The compound of alternative 126, wherein the compound is 6-chloro-3-(2-chloro-3-((N-methylsulfamoyl)amino)benzyl)-2-oxo-4-(piperazin-1-ylmethyl)-2H-chromen-7-yl dimethylcarbamate.

132. The compound of alternative 126, wherein the compound is 6-chloro-3-(2-chloro-3-(ethylsulfonamido)benzyl)-2-oxo-4-(piperazin-1-ylmethyl)-2H-chromen-7-yl dimethylcarbamate.

133. The compound of alternative 126, wherein the compound is 3-(2-chloro-3-(ethylsulfonamido)benzyl)-2-oxo-4-(piperazin-1-ylmethyl)-2H-chromen-7-yl dimethylcarbamate.

134. The compound of alternative 126, wherein the compound is 3-(2-chloro-3-((N-methylsulfamoyl)amino)benzyl)-4-((dimethylamino)methyl)-2-oxo-2H-chromen-7-yl dimethylcarbamate.

135. A pharmaceutical composition comprising a compound of any one of alternatives 126 to 134 and a pharmaceutically acceptable salt

136. A compound selected from the group consisting of:
3-(2-chloro-3-((N-methylsulfamoyl)amino)benzyl)-4-((dimethylamino)methyl)-6-fluoro-2-oxo-2H-chromen-7-yl dimethylcarbamate;
3-(2-chloro-3-(ethylsulfonamido)benzyl)-4-((dimethylamino)methyl)-6-fluoro-2-oxo-2H-chromen-7-yl dimethylcarbamate;
3-(2-chloro-3-((N-methylsulfamoyl)amino)benzyl)-4-((ethyl(methyl)amino)methyl)-6-fluoro-2-oxo-2H-chromen-7-yl dimethylcarbamate;
4-(azetidin-1-ylmethyl)-3-(2-chloro-3-((N-methylsulfamoyl)amino)benzyl)-6-fluoro-2-oxo-2H-chromen-7-yl dimethylcarbamate;
6-chloro-3-(2-chloro-3-((N-methylsulfamoyl)amino)benzyl)-4-((dimethylamino)methyl)-2-oxo-2H-chromen-7-yl dimethylcarbamate;
6-chloro-3-(2-chloro-3-((N-methylsulfamoyl)amino)benzyl)-4-((ethyl(methyl)amino)methyl)-2-oxo-2H-chromen-7-yl dimethylcarbamate; and
4-(azetidin-1-ylmethyl)-6-chloro-3-(2-chloro-3-((N-methylsulfamoyl)amino)benzyl)-2-oxo-2H-chromen-7-yl dimethylcarbamate.

137. The compound of alternative 136, wherein the compound is 3-(2-chloro-3-((N-methylsulfamoyl)amino)benzyl)-4-((dimethylamino)methyl)-6-fluoro-2-oxo-2H-chromen-7-yl dimethylcarbamate.

138. The compound of alternative 136, wherein the compound is 3-(2-chloro-3-(ethylsulfonamido)benzyl)-4-((dimethylamino)methyl)-6-fluoro-2-oxo-2H-chromen-7-yl dimethylcarbamate.

139. The compound of alternative 136, wherein the compound is 3-(2-chloro-3-((N-methylsulfamoyl)amino)benzyl)-4-((ethyl(methyl)amino)methyl)-6-fluoro-2-oxo-2H-chromen-7-yl dimethylcarbamate.

140. The compound of alternative 136, wherein the compound is 4-(azetidin-1-ylmethyl)-3-(2-chloro-3-((N-methylsulfamoyl)amino)benzyl)-6-fluoro-2-oxo-2H-chromen-7-yl dimethylcarbamate.

141. The compound of alternative 136, wherein the compound is 6-chloro-3-(2-chloro-3-((N-methylsulfamoyl)amino)benzyl)-4-((dimethylamino)methyl)-2-oxo-2H-chromen-7-yl dimethylcarbamate.

142. The compound of alternative 136, wherein the compound is 6-chloro-3-(2-chloro-3-((N-methylsulfamoyl)amino)benzyl)-4-((ethyl(methyl)amino)methyl)-2-oxo-2H-chromen-7-yl dimethylcarbamate.

143. A pharmaceutical composition comprising a compound of any one of alternatives 136 to 142 and a pharmaceutically acceptable salt.

144. A compound selected from the group consisting of:
3-(2-fluoro-3-((N-methylsulfamoyl)amino)benzyl)-4-(((2-fluoroethyl)(methyl)amino)methyl)-2-oxo-2H-chromen-7-yl dimethylcarbamate;
3-(3-(ethylsulfonamido)-2-fluorobenzyl)-4-(((2-fluoroethyl)(methyl)amino)methyl)-2-oxo-2H-chromen-7-yl dimethylcarbamate;
3-(2-fluoro-3-((N-methylsulfamoyl)amino)benzyl)-4-((methyl(prop-2-yn-1-yl)amino)methyl)-2-oxo-2H-chromen-7-yl dimethylcarbamate;
4-(((2,2-difluoroethyl)(methyl)amino)methyl)-3-(2-fluoro-3-((N-methylsulfamoyl)amino)benzyl)-2-oxo-2H-chromen-7-yl dimethylcarbamate;
4-(((cyanomethyl)(methyl)amino)methyl)-3-(2-fluoro-3-((N-methylsulfamoyl)amino)benzyl)-2-oxo-2H-chromen-7-yl dimethylcarbamate;
4-((dimethylamino)methyl)-3-(2-fluoro-3-(methyl(sulfamoyl)amino)benzyl)-2-oxo-2H-chromen-7-yl dimethylcarbamate;
4-((dimethylamino)methyl)-3-(2-fluoro-3-(hydroxymethyl)benzyl)-2-oxo-2H-chromen-7-yl dimethylcarbamate;
4-((dimethylamino)methyl)-3-(3-((ethylsulfonyl)methyl)-2-fluorobenzyl)-2-oxo-2H-chromen-7-yl dimethylcarbamate; and
3-(3-((tert-butylsulfinyl)amino)-2-fluorobenzyl)-4-((dimethylamino)methyl)-2-oxo-2H-chromen-7-yl dimethylcarbamate.

145. The compound of alternative 144, wherein the compound is 3-(2-fluoro-3-((N-methylsulfamoyl)amino)benzyl)-4-(((2-fluoroethyl)(methyl)amino)methyl)-2-oxo-2H-chromen-7-yl dimethylcarbamate.

146. The compound of alternative 144, wherein the compound is 3-(3-(ethylsulfonamido)-2-fluorobenzyl)-4-(((2-fluoroethyl)(methyl)amino)methyl)-2-oxo-2H-chromen-7-yl dimethylcarbamate;

147. The compound of alternative 144, wherein the compound is 3-(2-fluoro-3-((N-methylsulfamoyl)amino)benzyl)-4-((methyl(prop-2-yn-1-yl)amino)methyl)-2-oxo-2H-chromen-7-yl dimethylcarbamate.

148. The compound of alternative 144, wherein the compound is 4-(((2,2-difluoroethyl)(methyl)amino)methyl)-3-(2-fluoro-3-((N-methylsulfamoyl)amino)benzyl)-2-oxo-2H-chromen-7-yl dimethylcarbamate.

149. The compound of alternative 144, wherein the compound is 4-(((cyanomethyl)(methyl)amino)methyl)-3-(2-fluoro-3-((N-methylsulfamoyl)amino)benzyl)-2-oxo-2H-chromen-7-yl dimethylcarbamate.

150. The compound of alternative 144, wherein the compound is 4-((dimethylamino)methyl)-3-(2-fluoro-3-(methyl(sulfamoyl)amino)benzyl)-2-oxo-2H-chromen-7-yl dimethylcarbamate.

151. The compound of alternative 144, wherein the compound is 4-((dimethylamino)methyl)-3-(2-fluoro-3-(hydroxymethyl)benzyl)-2-oxo-2H-chromen-7-yl dimethylcarbamate.

152. The compound of alternative 144, wherein the compound is 3-(3-((tert-butylsulfinyl)amino)-2-fluorobenzyl)-4-((dimethylamino)methyl)-2-oxo-2H-chromen-7-yl dimethylcarbamate.

153. A pharmaceutical composition comprising a compound of any one of alternatives 144 to 152 and a pharmaceutically acceptable salt.

154. A compound having the structure of Formula (III):

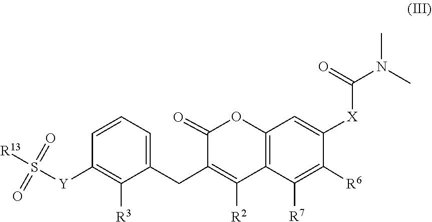

including pharmaceutically acceptable salts thereof,
wherein,
R², R⁶, R⁷, and R¹³ are each independently selected from the group consisting of H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C₁ to C₆ alkoxy, optionally substituted C₁ to C₆ alkyl, optionally substituted C₂ to C₆ alkenyl, optionally substituted C₂ to C₆ alkynyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, and L;
R³ is a chloro;
X is C(R⁵)₂, CH(R⁵), CH₂, —O—,

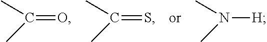

Y is C(R⁵)₂, CH(R⁵), CH₂, —O—,

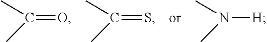

L is —Z₁-Z₂ or —Z₁-Z₂—Z₃;
Z₁, Z₂, and Z₃ are independently selected from the group consisting of —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C₁ to C₆ alkyl, optionally substituted C₃ to C₈ cycloalkyl, optionally substituted C₆ to C₁₀ aryl, optionally substituted C₃ to C₈ heterocyclyl, optionally substituted C₃ to C₁₀ heteroaryl, —CH₂-(optionally substituted aryl), —CH₂-(optionally substituted C₃ to C₈ cycloalkyl), and —CH₂-(optionally substituted C₃ to C₁₀ heteroaryl); and
each R⁵ and R^{5′} are independently selected from H, deuterium, optionally substituted C₁ to C₆ alkyl, optionally substituted C₂ to C₆ alkenyl, optionally substituted C₂ to C₆ alkynyl, optionally substituted C₃ to C₈ carbocyclyl, optionally substituted C₆ to C₁₀ aryl, optionally substituted C₃ to C₈ heterocyclyl, and optionally substituted C₃ to C₁₀ heteroaryl.

155. The compound or pharmaceutically acceptable salt according to alternative 154, wherein R² is CH₂—Z₂.

156. The compound or pharmaceutically acceptable salt according to alternative 155, wherein Z₂ is —NR⁵R^{5′} or optionally substituted C₃ to C₈ heterocyclyl.

157. The compound or pharmaceutically acceptable salt according to alternative 155 or alternative 156, wherein Z₂ is —NR⁵R^{5′} and each of R⁵ and R^{5′} is optionally substituted C₁ to C₆ alkyl, optionally wherein R⁵ and R^{5′} are each Me, optionally wherein R⁵ is Me and R^{5′} is Et.

158. The compound or pharmaceutically acceptable salt according alternative 155 or alternative 156, wherein Z₂ is optionally substituted C₃ to C₈ heterocyclyl and the heterocycle is a nitrogen-containing heterocyclyl, optionally wherein Z₂ is

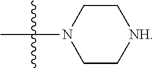 [SMILES: *N1CCNCC1]

159. The compound or pharmaceutically acceptable salt according to any one of alternatives 154 to 158, wherein Y is CH₂ or

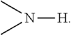

160. The compound or pharmaceutically acceptable salt according to alternative 159, wherein Y is

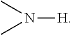

161. The compound or pharmaceutically acceptable salt according to any one of alternatives 154 to 160, wherein R⁶ is H or halo, optionally wherein halo is selected from fluoro, chloro or bromo, optionally wherein halo is fluoro or chloro, optionally wherein halo is chloro.

162. The compound or pharmaceutically acceptable salt according to any one of alternatives 154 to 161, wherein R⁷ is H.

163. The compound or pharmaceutically acceptable salt according to any one of alternatives 154 to 162, wherein X is O.

164. The compound or pharmaceutically acceptable salt according to any one of alternatives 154 to 163, wherein R¹³ is optionally substituted amino or optionally substituted C₁ to C₆ alkyl.

165. The compound or pharmaceutically acceptable salt according to any one of alternatives to 164, wherein R¹³ is NHMe or Et, optionally wherein R¹³ is NHMe.

166. The compound or pharmaceutically acceptable salt according to alternative 154, wherein the compound is selected from the group consisting of:

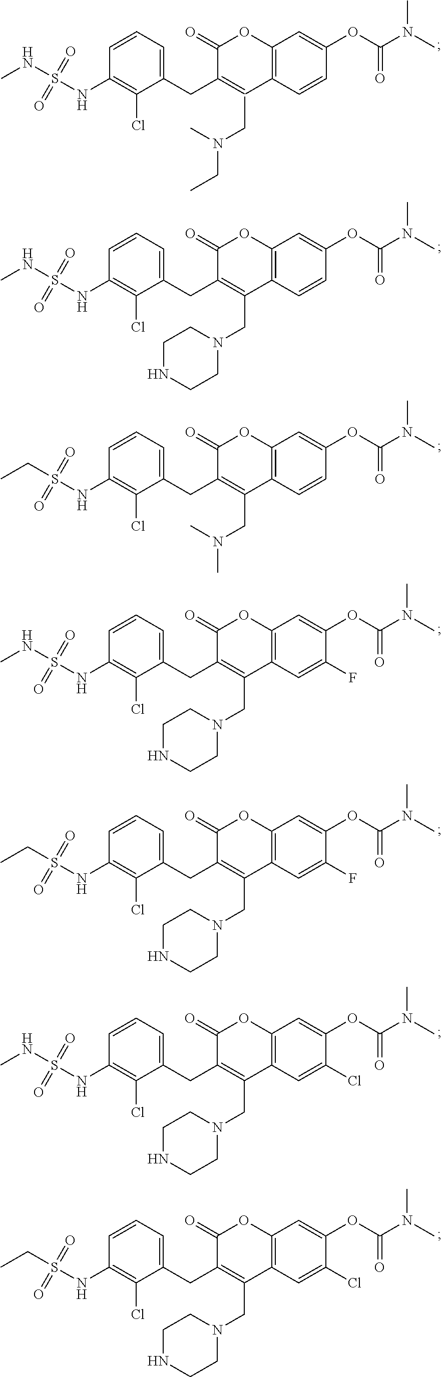

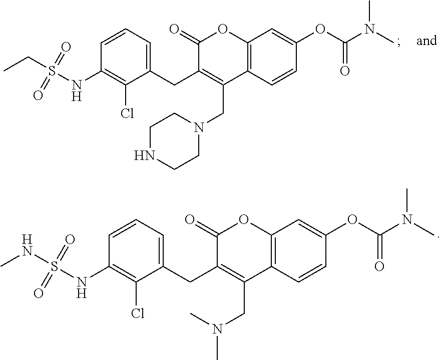

167. The compound or pharmaceutically acceptable salt according to alternative 154, wherein the compound is:

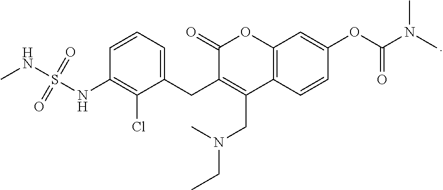

168. The compound or pharmaceutically acceptable salt according to alternative 154, wherein the compound is:

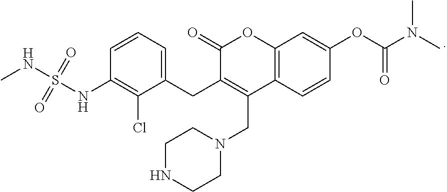

169. The compound or pharmaceutically acceptable salt according to alternative 154, wherein the compound is:

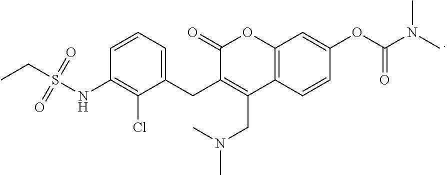 [SMILES: CCS(=O)(=O)Nc1cccc(Cc2c(CN(C)C)c3ccc(OC(=O)N(C)C)cc3oc2=O)c1Cl]

170. The compound or pharmaceutically acceptable salt according to alternative 154, wherein the compound is:

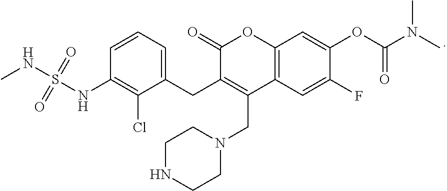

171. The compound or pharmaceutically acceptable salt according to alternative 154, wherein the compound is:

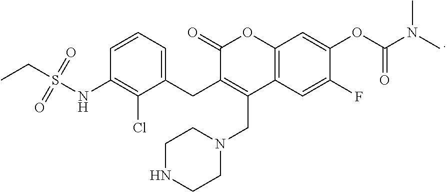 [SMILES: CCS(=O)(=O)Nc1cccc(Cc2c(CN3CCNCC3)c3cc(F)c(OC(=O)N(C)C)cc3oc2=O)c1Cl]

172. The compound or pharmaceutically acceptable salt according to alternative 154, wherein the compound is:

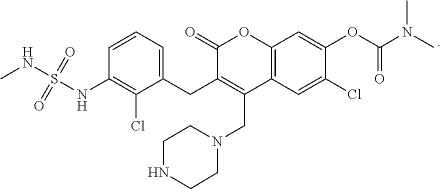

173. The compound or pharmaceutically acceptable salt according to alternative 154, wherein the compound is:

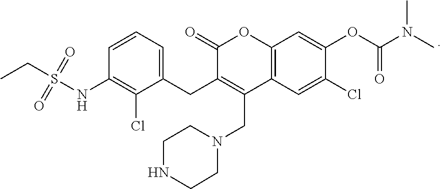 [SMILES: CCS(=O)(=O)Nc1cccc(Cc2c(CN3CCNCC3)c3cc(Cl)c(OC(=O)N(C)C)cc3oc2=O)c1Cl]

174. The compound or pharmaceutically acceptable salt according to alternative 154, wherein the compound is:

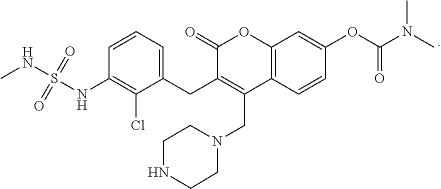

175. The compound or pharmaceutically acceptable salt according to alternative 154, wherein the compound is:

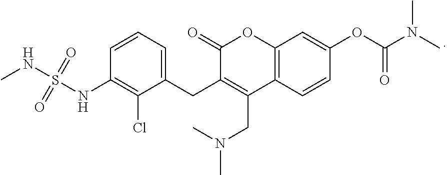

176. A pharmaceutical composition comprising a therapeutically effective amount of at least one compound or pharmaceutically acceptable salt thereof as defined in any one of alternative 154 to 175.

177. A compound or pharmaceutically acceptable salt thereof as defined in any one of alternatives 154 to 175, for use in the treatment of cancer.

### EXAMPLES

### General Procedures

Additional embodiments are disclosed in further detail in the following examples, which are not in any way intended to limit the scope of the claims.

Materials used in preparing compounds of Formula (I), (Ia), (Ib), or (Ic), described herein may be made by known methods or are commercially available. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry (Wiley), Carey and Sundberg, Advanced Organic Chemistry (incorporated herein by reference in their entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1

### General Synthesis A

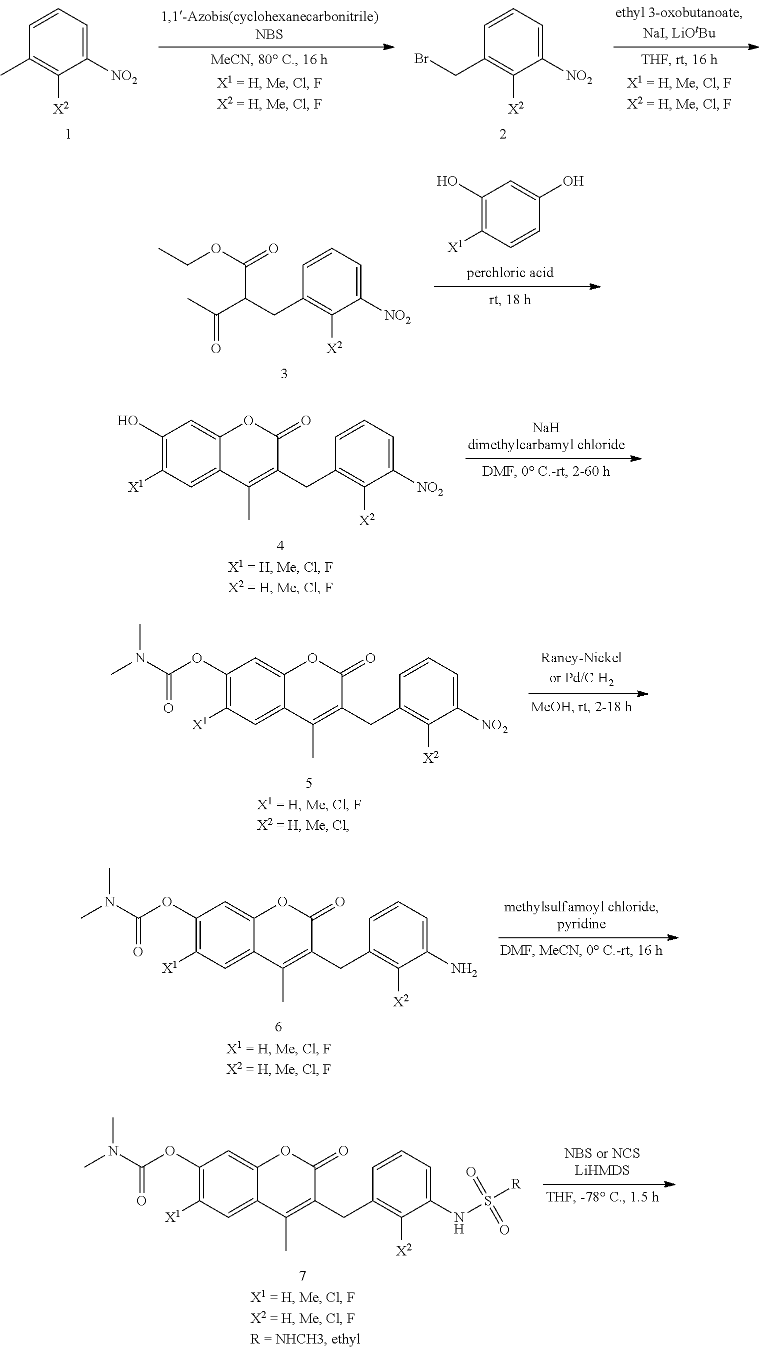

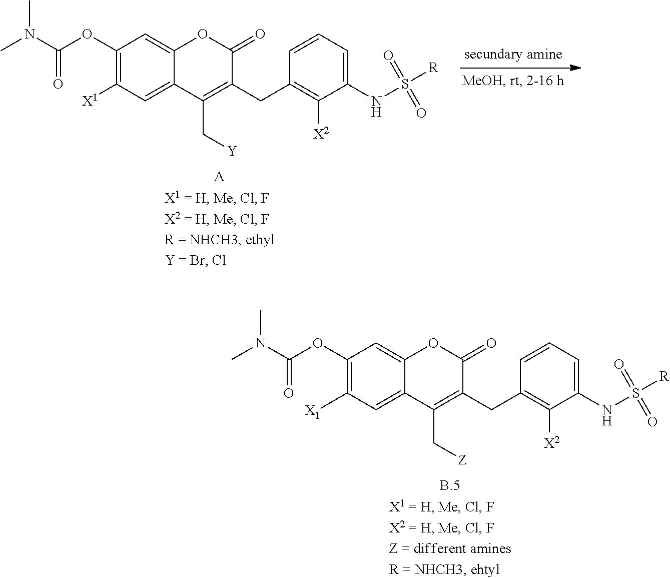

Compound 2: To a solution of compound 1 and NBS (57.8 g, 321 mmol, 1.20 eq.) in MeCN (1340 mL) under nitrogen atmosphere was added 1,1-azobis(cyclohexanecarbonitrile) (0.12 eq.). The formed reaction mixture was stirred at 80° C. for 16 h. The reaction mixture was allowed to cool to rt and concentrated under reduced pressure to give an orange suspension. Et₂O was added and the formed suspension was stirred for 18 hours at rt. The suspension was filtered and the residue was washed with some extra Et₂O. Combined organic layers were washed with aqueous saturated NaHCO₃ and brine, dried over Na₂SO₄, filtered and concentrated under reduced pressure to obtain benyzle bromide Compound 2 as a dark red oil that crystalized upon standing.

Compound 3: A mixture of Compound 2 (1.0 eq.) and sodium iodide (1.0 eq.) was stirred in THF (dry) for 30 min. In another flask, ethyl 3-oxobutanoate (1.10 eq.) was dissolved in THF (dry) and lithium tert-butoxide (1.10 eq.) was slowly added. The reaction mixture was stirred for 30 min. and was then slowly added to the bromide suspension. The resulting reaction mixture was stirred for 16 h at rt. The reaction mixture was quenched with water and the product was extracted with EtOAc. Combined organic extracts were washed with brine, dried over Na₂SO₄, filtered and concentrated under reduced pressure to obtain a dark brown oil. The oil was coated onto hydro matrix and purified by column chromatography with method ‘flash’ (heptane/EtOAc=1:0→7:3) to obtain Compound 3 as a light yellow oil.

Compound 4: To a solution of Compound 3 (1.0 eq.) in perchloric acid or methane sulfonic acid (10-20 eq.) the resorcinol derivative (1.2 eq.) was added. The reaction mixture was stirred for 1-18 h at rt. Water was added to the reaction mixture and the product was filtered off, washed with water and Et₂O. The residue was dried to obtain the coumarin Compound 4 as a solid.

Compound 5: To a solution of the Compound 4 (1.0 eq.) in DMF (dry) (0.1-0.2 M) at 0° C. under N₂ atmosphere, sodium hydride 60% dispersion on mineral oil (1.60 eq.) was added. The reaction mixture was left to stir for 10 min before dimethyl carbamoyl chloride (1.50-1.60 eq.) was added. The reaction mixture was allowed to warm to room temperature and stirred for an additional 2-60 h. Water was added to quench the reaction mixture. The suspension was filtered, washed with water and Et₂O. The residue was dried to obtain the dimethylcarbamate Compound 5 as a solid.

Compound 5: The dimethylcarbamate (1.0 eq.) was suspended in Methanol (0.2 M), in some cases some CH₂Cl₂ was added to get a solution. Argon was bubbled through the solution for 10 min. Then a 50% Raney®-Nickel slurry in water (1.0 eq.) or 10% palladium on activated carbon (0.05 eq.) was added. The formed reaction mixture was purged with hydrogen and stirred for 2-18 h at rt. The reaction mixture was filtered over kieselguhr and washed with MeCN, CH₂Cl₂ and MeOH. The filtrate was concentrated under reduced pressure to obtain the primary amine as a solid.

Compound 7: To an ice bath cooled (0° C.) suspension of aniline Compound 6 (1.0 eq.) and pyridine (3.0 eq.) in DMF (0.2 M), a solution of methylsulfamoyl chloride (2.5 eq.) in MeCN (anhydrous) (0.2 M) was added dropwise. After complete addition the formed reaction mixture was allowed to warm-up to room temperature and stirred for 1-16 h. Water was added to the reaction mixture and the formed suspension was stirred for 1 hour. The suspension was filtered, washed with water and Et2O. The residue was dried to obtain the sulfamoyl as a solid.

Compound A: A solution of Compound 7 (1.0 eq.) in THF (dry) (0.06-0.10 M) under nitrogen atmosphere was cooled to −78° C. and LiHMDS 1M in THF (3.0 eq.) was slowly added. After full addition the formed reaction mixture was in some cases diluted with some extra tetrahydrofuran (dry) and stirred for 30 min, allowed to warm to 0° C. This was added to a cooled (−78° C.) solution of NCS or NBS (1.2 eq.) in THF (dry) (0.04 M), drop-wise via a cannula over 15 minutes. The formed reaction mixture was stirred for 1 hour at −78° C. At −78° C. the reaction mixture was quenched with HCl 1M and allowed to warm to rt. Some extra water was added and the product was extracted with EtOAc. Combined organic layers were washed with brine, dried over Na₂SO₄, filtered and concentrated under reduced pressure. The impure product Compound 7 was used as such.

Compound B.5: Compound A (1.0 eq.) was suspended in MeOH (0.10-0.20 M). The amine (1-10 eq.) was added. Optionally 2-5 eq. of Et₃N were added and the reaction mixture was stirred for 2-16 h at rt. The reaction mixture was filtered and purified by preparative HPLC (method: prep acid or prep base) to obtain the desired amine B.5 after freeze drying or Genevac™ as a solid.

### Example 2

### Synthesis of Compound 244

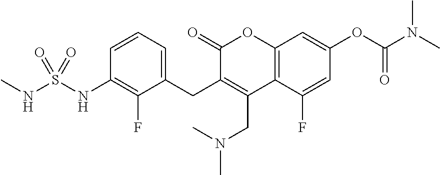

Compound 244 was prepared in 1 step:

Step 1: Starting with 4-(chloromethyl)-5-fluoro-3-(2-fluoro-3-((N-methylsulfamoyl)amino)benzyl)-2-oxo-2H-chromen-7-yl dimethylcarbamate and dimethylamine 2.0 M in MeOH, following the general synthesis of Compound B.5. The product was combined with another batch and purified by column chromatography to obtain the title compound after freeze drying as a white solid.

4-((dimethylamino)methyl)-5-fluoro-3-(2-fluoro-3-((N-methylsulfamoyl)amino)benzyl)-2-oxo-2H-chromen-7-yl dimethylcarbamate Analysis: LCMS (Method T): t_{R}=1.52 min; m/z calculated for [M+H]⁺=525.2, found=525.2; ¹H NMR (400 MHz, DMSO) δ 9.37 (s, 1H), 7.26 (t, J=7.9 Hz, 1H), 7.18-7.08 (m, 2H), 6.94 (s, 1H), 6.76 (s, 1H), 4.02 (s, 2H), 3.59 (s, 2H), 3.05 (s, 3H), 2.93 (s, 3H), 2.18 (s, 6H).

### Example 3

### Synthesis of Compound 245

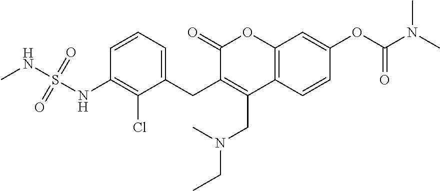

Compound 245 was prepared in 1 step:

Step 1: Starting with 4-(bromomethyl)-3-(2-chloro-3-((N-methylsulfamoyl)amino)benzyl)-2-oxo-2H-chromen-7-yl dimethylcarbamate and N-ethylmethylamine, in DCMDCM following procedure the general synthesis of Compound B.5. After full conversion the reaction was concentrated under reduced pressure. The impure product was purified by column chromatography to obtain the title compound as a white solid.

3-(2-chloro-3-((N-methylsulfamoyl)amino)benzyl)-4-((ethyl(methyl)amino)methyl)-2-oxo-2H-chromen-7-yl dimethylcarbamate Analysis: LCMS (Method T): t_{R}=1.75 min; m/z calculated for [M+H]⁺=537.0/539.0, found=537.2/539.2; 1H NMR (400 MHz, DMSO) δ 9.02 (s, 1H), 8.12 (d, J=8.8 Hz, 1H), 7.38 (d, J=8.0 Hz, 1H), 7.29-7.08 (m, 4H), 6.78-6.68 (m, 1H), 4.09 (s, 2H), 3.60 (s, 2H), 3.07 (s, 3H), 2.94 (s, 3H), 2.55 (d, J=4.2 Hz, 3H), 2.43 (q, J=7.1 Hz, 2H), 2.10 (s, 3H), 0.98 (t, J=7.1 Hz, 3H).

### Example 4

### Synthesis of Compound 246

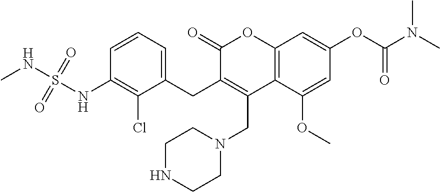

Compound 246 was prepared in 2 steps:

Step 1: Starting with 4-(bromomethyl)-3-(2-chloro-3-((N-methylsulfamoyl)amino)benzyl)-2-oxo-2H-chromen-7-yl dimethylcarbamate and N-Boc piperazine, following the general synthesis of Compound B.5. The product was purified by prep basic. Desired fractions were combined and concentrated under reduced pressure to obtain the amine as a colorless oil.

Step 2: The amine was dissolved in 1,4-dioxane (3 mL) and HCl in dioxane (4M, 16.7 eq,) was added and stirred for 1 hour at rt. The reaction mixture was concentrated under reduced pressure and the twice co-evaporated with DCMDCM. The residue was dissolved in MeCN/water and lyophilized to obtain the title compound as a white solid.

3-(2-chloro-3-((N-methylsulfamoyl)amino)benzyl)-2-oxo-4-(piperazin-1-ylmethyl)-2H-chromen-7-yl dimethylcarbamate hydrochloride Analysis: LCMS (Method T): t_{R}=1.40 min; m/z calculated for [M+H]⁺=564.1/566.1, found=564.2/566.2; 1H NMR (400 MHz, DMSO) δ 9.00 (s, 1H), 8.73-8.68 (m, 1H), 8.09 (d, J=8.9 Hz, 1H), 7.39 (dd, J=8.1, 1.5 Hz, 1H), 7.30-7.24 (m, 2H), 7.20-7.12 (m, 2H), 6.77 (d, 1H), 4.07 (s, 2H), 3.75 (s, 2H), 3.07 (s, 3H), 3.00-2.85 (m, 7H), 2.72-2.62 (m, 4H), 2.57 (d, J=4.7 Hz, 3H).

### Example 5

### Synthesis of Compound 249

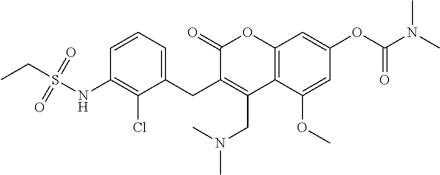 [SMILES: CCS(=O)(=O)Nc1cccc(Cc2c(CN(C)C)c3c(OC)cc(OC(=O)N(C)C)cc3oc2=O)c1Cl]

Compound 249 was prepared in 3 steps:

Step 1: Following the procedure of the general synthesis of Compound B.5 starting with the bromine compound and dimethylamine 2.0 M in MeOH. The impure product was purified by column chromatography to obtain the title compound as a white solid.

3-(2-chloro-3-(ethylsulfonamido)benzyl)-4-((dimethylamino)methyl)-2-oxo-2H-chromen-7-yl dimethylcarbamate Analysis: LCMS (Method R): t_{R}=0.95 min; m/z calculated for [M+H]⁺=522.1, found=522.2; ¹H NMR (400 MHz, DMSO) δ 9.69 (s, 1H), 8.09 (d, J=8.9 Hz, 1H), 7.33 (dd, J=8.0, 1.5 Hz, 1H), 7.24 (d, J=2.3 Hz, 1H), 7.20-7.07 (m, 2H), 6.85 (dd, J=7.8, 1.5 Hz, 1H), 4.11 (s, 2H), 3.56 (s, 2H), 3.14 (q, J=7.3 Hz, 2H), 3.07 (s, 3H), 2.94 (s, 3H), 2.18 (s, 6H), 1.29 (t, J=7.3 Hz, 3H).

### Example 6

### Synthesis of Compound 252

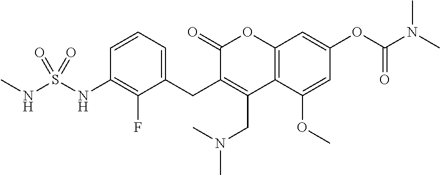

Compound 252 was prepared in 1 step:

Step 1: Starting with 4-(chloromethyl)-3-(2-fluoro-3-((N-methylsulfamoyl)amino)benzyl)-5-methoxy-2-oxo-2H-chromen-7-yl dimethylcarbamate (50 mg, 0.097 mmol) and dimethylamine 2.0 M in MeOH, following the general synthesis of Compound B.5. The product was purified by column chromatography to obtain the title compound after freeze drying as a white solid.

4-((dimethylamino)methyl)-3-(2-fluoro-3-((N-methylsulfamoyl)amino)benzyl)-5-methoxy-2-oxo-2H-chromen-7-yl dimethylcarbamate Analysis: LCMS (Method R): t_{R}=0.89 min; m/z calculated for [M+H]⁺=537.2, found=537.2; ¹H NMR (400 MHz, DMSO) δ 9.38 (s, 1H), 7.27 (td, J=8.1, 1.7 Hz, 1H), 7.16 (s, 1H), 6.99 (t, J=7.9 Hz, 1H), 6.84 (d, J=2.3 Hz, 1H), 6.82-6.75 (m, 2H), 4.03 (s, 2H), 3.89 (s, 3H), 3.74 (s, 2H), 3.06 (s, 3H), 2.93 (s, 3H), 2.16 (s, 6H).

### Example 7

### Synthesis of Compound 253

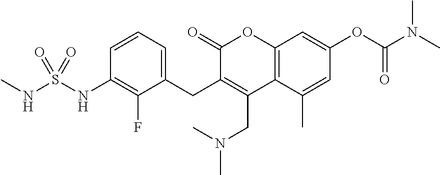

Compound 253 was prepared in 1 step:

Step 1: Starting with 4-(chloromethyl)-3-(2-fluoro-3-((N-methylsulfamoyl)amino)benzyl)-5-methyl-2-oxo-2H-chromen-7-yl dimethylcarbamate and dimethylamine 2.0 M in MeOH, following the general synthesis of Compound B.5. The product was purified by column chromatography to obtain the title compound after freeze drying as a white solid.

4-((dimethylamino)methyl)-3-(2-fluoro-3-((N-methylsulfamoyl)amino)benzyl)-5-methyl-2-oxo-2H-chromen-7-yl dimethylcarbamate Analysis: LCMS (Method R): t_{R}=0.85 min; m/z calculated for [M+H]⁺=521.2, found=521.2; ¹H NMR (400 MHz, DMSO) δ 9.36 (s, 1H), 7.28 (td, J=7.8, 1.6 Hz, 1H), 7.21 (q, J=5.0 Hz, 1H), 7.14 (d, J=1.7 Hz, 1H), 7.00 (t, J=7.9 Hz, 1H), 6.88 (d, J=1.7 Hz, 1H), 6.85-6.77 (m, 1H), 4.07 (s, 2H), 3.63 (s, 2H), 3.12 (s, 3H), 2.93 (s, 3H), 2.52 (d, J=5.0 Hz, 3H), 2.37 (s, 3H), 2.08 (s, 6H).

### Example 8

### Synthesis of Compound 254

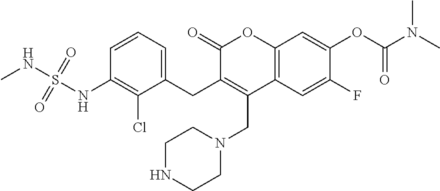

Compound 254 was prepared in 1 step:

Step 1: Starting with 3-(2-chloro-3-((N-methylsulfamoyl)amino)benzyl)-4-(chloromethyl)-6-fluoro-2-oxo-2H-chromen-7-yl dimethylcarbamate and piperazine, following the general synthesis of Compound B.5. The impure product was purified by column chromatography to obtain the title compound after freeze drying as a white solid.

3-(2-chloro-3-((N-methylsulfamoyl)amino)benzyl)-6-fluoro-2-oxo-4-(piperazin-1-ylmethyl)-2H-chromen-7-yl dimethylcarbamate Analysis: LCMS (Method R): t_{R}=0.98 min; m/z calculated for [M+H]⁺=582.2, found=582.2; ¹H NMR (400 MHz, DMSO) δ 8.05 (d, J=11.7 Hz, 1H), 7.48 (d, J=6.8 Hz, 1H), 7.38 (dd, J=8.1, 1.5 Hz, 1H), 7.13 (t, J=7.9 Hz, 1H), 6.74 (dd, J=7.8, 1.5 Hz, 1H), 4.08 (s, 2H), 3.58 (s, 2H), 3.09 (s, 3H), 2.95 (s, 3H), 2.59 (t, J=4.7 Hz, 3H), 2.55 (s, 3H), 2.35 (s, 4H).

### Example 9

### Synthesis of Compound 255

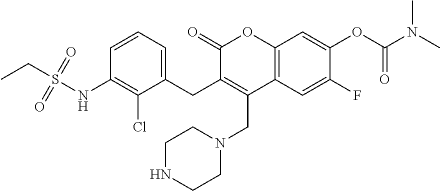

Compound 255 was prepared in 1 step:

Step 1: Following the procedure of the general synthesis of Compound B.5, starting with the bromine using piperazine. The impure product was purified by column chromatography to obtain the title compound as a white solid after lyophilization.

3-(2-chloro-3-(ethylsulfonamido)benzyl)-6-fluoro-2-oxo-4-(piperazin-1-ylmethyl)-2H-chromen-7-yl dimethylcarbamate Analysis: LCMS (Method R): t_{R}=1.02 min; m/z calculated for [M+H]⁺=581.2, found=581.2; ¹H NMR (400 MHz, DMSO) δ 8.04 (d, J=11.7 Hz, 1H), 7.48 (d, J=6.9 Hz, 1H), 7.29 (dd, J=8.2, 1.5 Hz, 1H), 7.04 (t, J=7.9 Hz, 1H), 6.61 (d, J=7.7 Hz, 1H), 4.07 (s, 2H), 3.58 (s, 2H), 3.09 (s, 3H), 3.01 (q, J=7.3 Hz, 2H), 2.95 (s, 3H), 2.61 (t, J=4.5 Hz, 4H), 2.37 (s, 5H), 1.24 (t, J=7.3 Hz, 3H).

### Example 10

### Synthesis of Compound 256

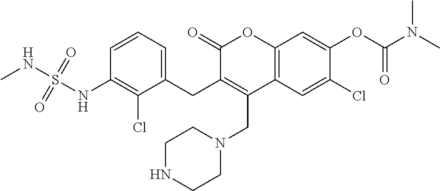

Compound 254 was prepared in 1 step:

Step 1: Starting with 6-chloro-3-(2-chloro-3-((N-methylsulfamoyl)amino)benzyl)-4-(chloromethyl)-2-oxo-2H-chromen-7-yl dimethylcarbamate and piperazine, following the general synthesis of Compound B.5. The impure product was purified by column chromatography to obtain the title compound after freeze drying as a white solid.

6-chloro-3-(2-chloro-3-((N-methylsulfamoyl)amino)benzyl)-2-oxo-4-(piperazin-1-ylmethyl)-2H-chromen-7-yl dimethylcarbamate hydrochloride Analysis: LCMS (Method R): t_{R}=1.02 min; m/z calculated for [M+H]⁺=598.1, found=598.2; ¹H NMR (400 MHz, DMSO) δ 9.05 (s, 1H), 8.66 (s, 2H), 8.21 (s, 1H), 7.53 (s, 1H), 7.40 (d, J=7.9 Hz, 1H), 7.30 (d, J=5.2 Hz, 1H), 7.15 (t, J=7.9 Hz, 1H), 6.80 (d, J=7.8 Hz, 1H), 4.07 (s, 2H), 3.77 (s, 2H), 3.11 (s, 3H), 2.95 (s, 3H), 3.94 (s, 4H) 2.68 (s, 4H), 2.57 (d, J=4.5 Hz, 3H).

### Example 11

### Synthesis of Compound 257

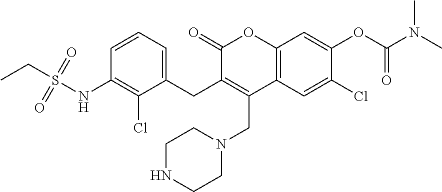

Compound 257 was prepared in 3 steps:

Step 1: Following the procedure of the general synthesis of Compound B.5, starting with the bromine using piperazine. The impure product was purified by column chromatography to obtain the title compound as a white solid after lyophilization.

6-chloro-3-(2-chloro-3-(ethylsulfonamido)benzyl)-2-oxo-4-(piperazin-1-ylmethyl)-2H-chromen-7-yl dimethylcarbamate hydrochloride Analysis: LCMS (Method R): t_{R}=1.05 min; m/z calculated for [M+H]⁺=597.1, found=597.2; ¹H NMR (400 MHz, DMSO) δ 9.48 (s, 1H), 9.04 (s, 2H), 8.23 (s, 1H), 7.53 (s, 1H), 7.34 (d, J=8.0 Hz, 1H), 7.17 (t, J=7.9 Hz, 1H), 6.89 (d, J=7.7 Hz, 1H), 4.09 (s, 2H), 3.82 (s, 2H), 3.16 (q, J=7.4 Hz, 2H), 3.11 (s, 3H), 2.95 (d, J=5.5 Hz, 7H), 2.74 (s, 4H), 1.30 (t, J=7.3 Hz, 3H).

### Example 12

### Synthesis of Compound 258

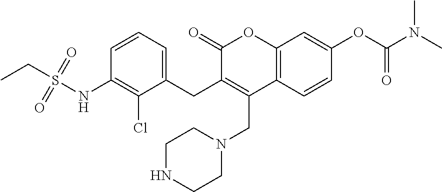

Compound 258 was prepared in 3 steps:

Step 1: Following the procedure of the general synthesis of Compound B.5, starting with the bromine using piperazine. The impure product was purified by column chromatography to obtain the title compound as a white solid after lyophilization.

3-(2-chloro-3-(ethylsulfonamido)benzyl)-2-oxo-4-(piperazin-1-ylmethyl)-2H-chromen-7-yl dimethylcarbamate Analysis: LCMS (Method R): t_{R}=1.00 min; m/z calculated for [M+H]⁺=563.2, found=563.2; ¹H NMR (400 MHz, DMSO) δ 8.26 (s, 1H), 8.10 (d, J=8.9 Hz, 1H), 7.31 (d, J=8.1 Hz, 1H), 7.25 (d, J=2.3 Hz, 1H), 7.17 (dd, J=8.8, 2.4 Hz, 1H), 7.11 (t, J=7.9 Hz, 1H), 6.74 (d, J=7.7 Hz, 1H), 4.08 (s, 2H), 3.62 (s, 2H), 3.08 (d, J=3.5 Hz, 5H), 2.94 (s, 3H), 2.63 (s, 4H), 2.40 (s, 4H), 1.27 (t, J=7.3 Hz, 3H).

### Example 13

### Synthesis of Compound 261

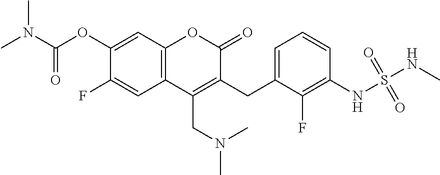

Compound 261 was prepared in 1 step:

Step 1: Starting with 4-(bromomethyl)-6-fluoro-3-(2-fluoro-3-((N-methylsulfamoyl)amino)benzyl)-2-oxo-2H-chromen-7-yl dimethylcarbamate and N-Methylethylamine, following the general synthesis of Compound B.5, The product was purified by column chromatography to obtain the title compound after freeze drying as a white solid.

4-((ethyl(methyl)amino)methyl)-6-fluoro-3-(2-fluoro-3-((N-methylsulfamoyl)amino)benzyl)-2-oxo-2H-chromen-7-yl dimethylcarbamate Analysis: LCMS (Method T): t_{R}=1.73 min; m/z calculated for [M+H]⁺=538.6, found=539.2; ¹H NMR (400 MHz, CDCl3) δ 7.96 (d, J=11.2 Hz, 1H), 7.40 (td, J=7.8, 1.5 Hz, 1H), 7.19 (d, J=6.7 Hz, 1H), 7.01 (t, J=8.0 Hz, 1H), 6.87 (d, J=7.7 Hz, 1H), 6.60 (s, 1H), 4.42 (d, J=5.6 Hz, 1H), 4.15 (s, 2H), 3.63 (s, 2H), 3.15 (s, 3H), 3.05 (s, 3H), 2.76 (d, J=5.3 Hz, 3H), 2.48 (s, 2H), 2.17 (s, 3H), 1.25 (s, 1H), 1.10 (s, 3H)

### Example 14

### Synthesis of Compound 262

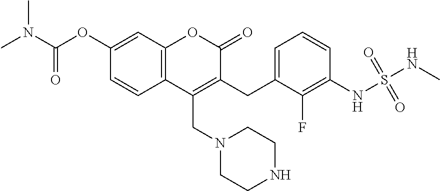

Compound 262 was prepared in 2 steps:

Step 1: Starting with 4-(bromomethyl)-3-(2-fluoro-3-((N-methylsulfamoyl)amino)benzyl)-2-oxo-2H-chromen-7-yl dimethylcarbamate (and N-Boc piperazine, following the general synthesis of Compound E.5. The product was purified by prep basic. Desired fractions were combined and concentrated under reduced pressure to obtain the amine (as a colorless oil.

Step 2: The amine was dissolved in 1,4-dioxane (3 mL) and HCl in dioxane (4M, 16.7 eq.) was added and stirred for 1 hour at rt. The reaction mixture was concentrated under reduced pressure and the twice co-evaporated with DCMDCM. The residue was dissolved in MeCN/water and lyophilized to obtain the title compound as a white solid.

Analysis: LCMS (Method S): t_{R}=1.00 min; m/z calculated for [M+H]⁺=548.2, found=548.2; 1H NMR (400 MHz, DMSO) δ 9.37 (s, 1H), 8.88 (s, 2H), 8.09 (d, J=8.8 Hz, 1H), 7.32-7.19 (m, 3H), 7.17 (dd, J=8.9, 2.4 Hz, 1H), 7.00 (t, J=7.8 Hz, 1H), 6.86-6.78 (m, 1H), 4.04 (s, 2H), 3.86 (s, 2H), 3.07 (s, 3H), 2.95 (d, J=14.4 Hz, 7H), 2.73 (s, 4H), 2.54 (d, J=2.8 Hz, 3H).

### Example 15

### Synthesis of Compound 263

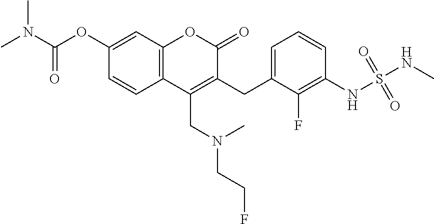

Compound 263 was prepared in 1 step:

Step 1: Starting with 4-(bromomethyl)-3-(2-fluoro-3-((N-methylsulfamoyl)amino)benzyl)-2-oxo-2H-chromen-7-yl dimethylcarbamate (and 2-fluoro-N-methylethan-1-amine, following the general synthesis of Compound B.5. The product was purified by column chromatography to obtain the title compound (after freeze drying as a white solid.

3-(2-fluoro-3-((N-methylsulfamoyl)amino)benzyl)-4-(((2-fluoroethyl)(methyl)amino)methyl)-2-oxo-2H-chromen-7-yl dimethylcarbamate Analysis: LCMS (Method R): t_{R}=1.22 min; m/z calculated for [M+H]⁺=539.2, found=539.2; ¹H NMR (400 MHz, DMSO) δ 8.12 (d, J=8.9 Hz, 1H), 7.31-7.22 (m, 2H), 7.14 (dd, J=8.9, 2.4 Hz, 1H), 6.99 (t, J=7.9 Hz, 1H), 6.81 (s, 1H), 4.59 (t, J=4.7 Hz, 1H), 4.47 (t, J=4.7 Hz, 1H), 4.05 (s, 2H), 3.80 (s, 2H), 3.06 (s, 3H), 2.93 (s, 3H), 2.79 (t, J=4.8 Hz, 1H), 2.71 (t, J=4.8 Hz, 1H), 2.19 (s, 3H).

### Example 16

### Synthesis of Compound 264

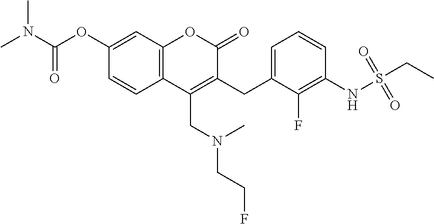 [SMILES: CCS(=O)(=O)Nc1cccc(Cc2c(CN(C)CCF)c3ccc(OC(=O)N(C)C)cc3oc2=O)c1F]

Compound 264 was prepared in 3 steps:

Step 1: Following the procedure of the general synthesis of Compound B.5, starting with the bromine and 2-fluoro-N-methylethan-1-amine. After full conversion the impure product was purified by column chromatography to obtain the title compound as an off-white solid.

3-(3-(ethylsulfonamido)-2-fluorobenzyl)-4-(((2-fluoroethyl)(methyl)amino)methyl)-2-oxo-2H-chromen-7-yl dimethylcarbamate Analysis: LCMS (Method R): t_{R}=1.32 min; m/z calculated for [M+H]⁺=538.2, found=538.2; ¹H NMR (400 MHz, DMSO) δ 9.60 (s, 1H), 8.12 (d, J=8.9 Hz, 1H), 7.29-7.18 (m, 2H), 7.14 (dd, J=8.8, 2.3 Hz, 1H), 7.01 (t, J=8.0 Hz, 1H), 6.90 (t, J=7.4 Hz, 1H), 4.58 (t, J=4.8 Hz, 1H), 4.46 (t, J=4.8 Hz, 1H), 4.06 (s, 2H), 3.81 (s, 2H), 3.08 (d, J=13.3 Hz, 5H), 2.93 (s, 3H), 2.78 (t, J=4.7 Hz, 1H), 2.71 (t, J=4.7 Hz, 1H), 2.18 (s, 3H), 1.26 (t, J=7.3 Hz, 3H).

### Example 17

### Synthesis of Compound 265

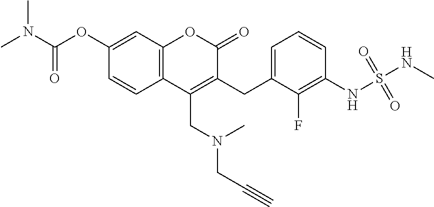

Compound 265 was prepared in 1 step:

Step 1: Starting with 4-(bromomethyl)-3-(2-fluoro-3-((N-methylsulfamoyl)amino)benzyl)-2-oxo-2H-chromen-7-yl dimethylcarbamate and N-methylprop-2-yn-1-amine, following the general synthesis of Compound B.5. The product was purified by column chromatography to obtain the title compound after freeze drying as a white solid.

### Example 18

### Synthesis of Compound 266

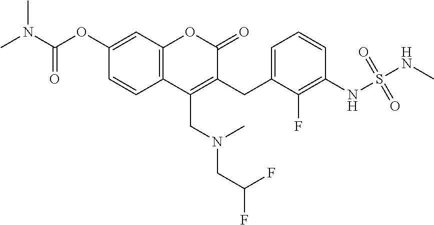

Compound 266 was prepared in 1 step:

Step 1: Starting with 4-(bromomethyl)-3-(2-fluoro-3-((N-methylsulfamoyl)amino)benzyl)-2-oxo-2H-chromen-7-yl dimethylcarbamate and 2,2-difluoro-N-methylethan-1-amine, following the general synthesis of Compound B.5. The product was purified by column chromatography to obtain the title compound after freeze drying as a white solid.

4-(((2,2-difluoroethyl)(methyl)amino)methyl)-3-(2-fluoro-3-((N-methylsulfamoyl)amino)benzyl)-2-oxo-2H-chromen-7-yl dimethylcarbamate Analysis: LCMS (Method R): t_{R}=1.64 min; m/z calculated for [M+H]⁺=557.2, found=557.2; ¹H NMR (400 MHz, DMSO) δ 9.41 (s, 1H), 8.09 (d, J=8.9 Hz, 1H), 7.38-7.23 (m, 2H), 7.14 (dd, J=8.8, 2.4 Hz, 2H), 6.98 (t, J=8.0 Hz, 1H), 6.79 (s, 1H), 6.11 (t, 1H), 4.05 (s, 2H), 3.89 (s, 2H), 3.06 (s, 3H), 2.96-2.80 (m, 5H), 2.24 (s, 3H).

### Example 19

### Synthesis of Compound 267

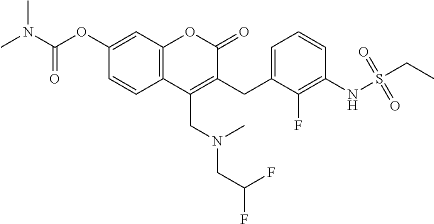 [SMILES: CCS(=O)(=O)Nc1cccc(Cc2c(CN(C)CC(F)F)c3ccc(OC(=O)N(C)C)cc3oc2=O)c1F]

Compound 267 was prepared in 3 steps:

Step 1: Following the procedure of the general synthesis of Compound B.5, starting with 220 mg, 0.384 mmol of the bromine and 2,2-difluoro-N-methylethan-1-amine. After full conversion the impure product was purified by column chromatography to obtain the title compound as an off-white solid.

4-(((2,2-difluoroethyl)(methyl)amino)methyl)-3-(3-(ethylsulfonamido)-2-fluorobenzyl)-2-oxo-2H-chromen-7-yl dimethylcarbamate Analysis: LCMS (Method R): t_{R}=1.69 min; m/z calculated for [M+H]⁺=556.2, found=556.4; ¹H NMR (400 MHz, DMSO) δ 9.62 (s, 1H), 8.09 (d, J=8.9 Hz, 1H), 7.24 (q, J=3.0 Hz, 2H), 7.14 (dd, J=8.9, 2.4 Hz, 1H), 7.01 (t, J=7.9 Hz, 1H), 6.89 (t, J=7.1 Hz, 1H), 6.31-5.93 (m, 1H), 4.06 (s, 2H), 3.90 (s, 2H), 3.17-3.02 (m, 5H), 2.99-2.80 (m, 5H), 2.24 (s, 3H), 1.26 (t, J=7.3 Hz, 3H).

### Example 20

### Synthesis of Compound 268

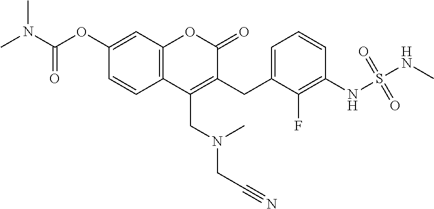

Compound 268 was prepared in 1 step:

Step 1: Starting with 4-(bromomethyl)-3-(2-fluoro-3-((N-methylsulfamoyl)amino)benzyl)-2-oxo-2H-chromen-7-yl dimethylcarbamate (45 mg, 0.075 mmol, py:40%) and 2-(methylamino)acetonitrile, following the general synthesis of Compound B.2, with addition of Net₃ 3.0 eq. The product was purified by prep basic followed by prep acid to obtain the title compound (3.8 mg, 0.007 mmol, y: 21%) after freeze drying as a white solid.

### Example 21

### Synthesis of Compound 269

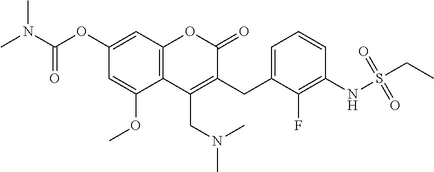 [SMILES: CCS(=O)(=O)Nc1cccc(Cc2c(CN(C)C)c3c(OC)cc(OC(=O)N(C)C)cc3oc2=O)c1F]

Compound 269 was prepared in 1 step:

Step 1: Starting with 4-(chloromethyl)-3-(2-fluoro-3-((N-methylsulfamoyl)amino)benzyl)-5-methoxy-2-oxo-2H-chromen-7-yl dimethylcarbamate and dimethylamine 2.0 M in MeOH, following the general synthesis of Compound B.5. The product was purified by column chromatography to obtain the title compound after freeze drying as a white solid.

### Example 22

### Synthesis of Compound 271

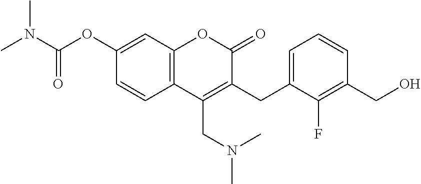 [SMILES: CN(C)Cc1c(Cc2cccc(CO)c2F)c(=O)oc2cc(OC(=O)N(C)C)ccc12]

Compound 271 was prepared in 1 step:

Step 1: To a solution of 3-(3-bromo-2-fluorobenzyl)-4-((dimethylamino)methyl)-2-oxo-2H-chromen-7-yl dimethylcarbamate (2.0 g, 4.19 mmol, 1.0 eq.) and (tributylstannyl)methanol (1.614 g, 5.03 mmol) (1.61 g, 5.03 mmol, 1.2 eq.) in 1,4-dioxane (0.1 M) under inert atmosphere was added Pd(Ph₃p)₄ (0.242 g, 0.209 mmol, 0.05 eq.). The formed reaction mixture was stirred for 18 hours at 100° C. Reaction mixture was filtered and washed with MeCN. The filtrate was concentrated under reduced pressure and the residue was purified by column chromatography with method ‘flash’ (heptane/EtOAc=1:0→2:8). 40 mg of the still impure product was purified by prep basic to obtain the title compound (29 mg, 0.067 mmol, yield: 1.5%) as an off-white solid. Yield: The title compound was isolated as an off white solid (1% over 1 step)

4-((dimethylamino)methyl)-3-(2-fluoro-3-(hydroxymethyl)benzyl)-2-oxo-2H-chromen-7-yl dimethylcarbamate Analysis: LCMS (Method T): t_{R}=1.53 min; m/z calculated for [M+H]⁺=429.2, found=429.2; 1H NMR (400 MHz, DMSO) δ 8.07 (d, J=8.8 Hz, 1H), 7.33-7.27 (m, 1H), 7.22 (d, J=2.3 Hz, 1H), 7.14 (dd, J=8.8, 2.4 Hz, 1H), 7.08-6.96 (m, 2H), 5.23 (t, J=5.7 Hz, 1H), 4.55 (d, J=5.5 Hz, 2H), 4.04 (s, 2H), 3.65 (s, 2H), 3.06 (s, 3H), 2.93 (s, 3H), 2.19 (s, 6H).

### Example 23

### Synthesis of Compound 272

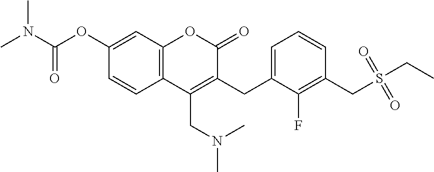 [SMILES: CCS(=O)(=O)Cc1cccc(Cc2c(CN(C)C)c3ccc(OC(=O)N(C)C)cc3oc2=O)c1F]

Compound 272 was prepared in 1 step:

Step 1: Starting with 4-(bromomethyl)-3-(3-((ethylsulfonyl)methyl)-2-fluorobenzyl)-2-oxo-2H-chromen-7-yl dimethylcarbamate (45 mg, 0.075 mmol, py:40%) and dimethylamine, following the general synthesis of Compound B.2, with addition of Net₃ 3.0 eq. The product was purified by prep basic followed by prep acid to obtain the title compound (3.8 mg, 0.007 mmol, y: 21%) after freeze drying as a white solid.

Yield: Compound 272 was isolated as a white solid (21% over 1 step).

### Example 24

### Synthesis of Compound 273

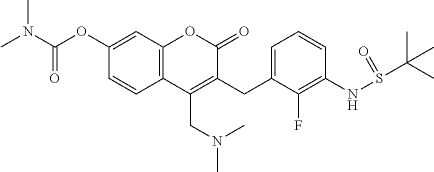 [SMILES: CN(C)Cc1c(Cc2cccc(NS(=O)C(C)(C)C)c2F)c(=O)oc2cc(OC(=O)N(C)C)ccc12]

Compound 273 was prepared in 3 step:

Step 1: To a solution of 3-(3-bromo-2-fluorobenzyl)-4-((dimethylamino)methyl)-2-oxo-2H-chromen-7-yl dimethylcarbamate (2.0 g, 4.19 mmol, 1.0 eq.) and (tributylstannyl)methanol (1.614 g, 5.03 mmol) (1.61 g, 5.03 mmol, 1.2 eq.) in 1,4-dioxane (0.1 M) under inert atmosphere was added Pd(Ph₃p)₄ (0.242 g, 0.209 mmol, 0.05 eq.). The reaction mixture was stirred for 18 hours at 100° C. Reaction mixture was filtered and washed with MeCN. The filtrate was concentrated under reduced pressure and the residue was purified by column chromatography with method ‘flash’ (heptane/EtOAc=1:0→2:8). Desired fraction were combined and concentrated under reduced pressure to obtain 4-((dimethylamino)methyl)-3-(2-fluoro-3-(hydroxymethyl)benzyl)-2-oxo-2H-chromen-7-yl dimethylcarbamate (1.46 g, 3.3 mmol, yield: 79%) as a clear oil.

Step 2: To a solution of 4-((dimethylamino)methyl)-3-(2-fluoro-3-(hydroxymethyl)benzyl)-2-oxo-2H-chromen-7-yl dimethylcarbamate (1.41 g, 3.29 mmol, 1.0 eq.) in DCM (0.23 M) at 0° C. was slowly added a solution of thionyl chloride (0.48 ml, 6.58 mmol, 2.0 eq.) in DCM (2 ml). The formed reaction mixture was stirred for 1 hour allowing to warm to r. The reaction mixture was concentrated under reduced pressure and stripped twice with DCM to obtain 3-(3-(chloromethyl)-2-fluorobenzyl)-4-((dimethylamino)methyl)-2-oxo-2H-chromen-7-yl dimethylcarbamate (1.68 g, 3.60 mmol, yield: 109%) as an off-white solid.

Step 3: To a mixture of 3-(3-(chloromethyl)-2-fluorobenzyl)-4-((dimethylamino)methyl)-2-oxo-2H-chromen-7-yl dimethylcarbamate (0.5 g, 1.12 mmol, 1.0 eq.) in ethanol (Abs) (0.1 M) under N₂ atmosphere was added thiourea (0.102 g, 1.343 mmol, 1.2 eq.). The reaction mixture was stirred for 2 hours at 80° C. and then for 2 days at rt. NaOH 2 N (1.68 ml, 3.36 mmol, 3.0 eq.) was added and stirred for 2 hours at 80° C. The reaction mixture was acidified with 1M HCl. Some extra water was added followed by DCM. The layers were separated by a phase separator and the organic layer was concentrated under reduced pressure to obtain 4-((dimethylamino)methyl)-3-(2-fluoro-3-(mercaptomethyl)benzyl)-2-oxo-2H-chromen-7-yl dimethylcarbamate and its dimer ((((disulfanediylbis(methylene))bis(2-fluoro-3,1-phenylene))bis(methylene))bis(4-((dimethylamino)methyl)-2-oxo-2H-chromene-3,7-diyl) bis(dimethylcarbamate)) (570 mg, 0.67 mmol, yield: 60%) as a yellow solid.

Step 4: (((disulfanediylbis(methylene))bis(2-fluoro-3,1-phenylene))bis(methylene))bis(4-((dimethylamino)methyl)-2-oxo-2H-chromene-3,7-diyl) bis(dimethylcarbamate) (0.57 g, 0.257 mmol, 1.0 eq.) was dissolved in THF (0.11 M)/water (0.11 M) and cooled to 0° C. tri-n-butylphosphine (0.071 ml, 0.283 mmol, 1.1 eq.) was slowly added and stirred for 1 hour at rt. Water and DCM were added and layers were separated by a phase separator. The organic layer was concentrated under reduced pressure and the residue was purified by column chromatography with method ‘flash’ (DCM/MeOH=1:0→98:2). Desired fraction were combined and concentrated under reduced pressure to obtain 4-((dimethylamino)methyl)-3-(2-fluoro-3-(mercaptomethyl)benzyl)-2-oxo-2H-chromen-7-yl dimethylcarbamate (283 mg, 0.618 mmol, yield: 55%) as a clear oil.

Step 5: 4-((dimethylamino)methyl)-3-(2-fluoro-3-(mercaptomethyl)benzyl)-2-oxo-2H-chromen-7-yl dimethylcarbamate (50 mg, 0.112 mmol, 1.0 eq.) was dissolved in DMF (dry) (0.23 M), Cs₂CO₃ (36.6 mg, 0.112 mmol, 1.0 eq.) and iodoethane (10.91 μl, 0.135 mmol, 1.2 eq.) were added and stirred for 1 hour at rt. iodoethane (1.818 μl, 0.022 mmol, 0.2 eq.) was added and stirred for 30 minutest at rt. Water was added to the reaction mixture and the product was extracted with DCM. Combined organic layers were washed with brine, dried over Na₂SO₄, filtered and concentrated under reduced pressure to obtain 4-((dimethylamino)methyl)-3-(3-((ethylthio)methyl)-2-fluorobenzyl)-2-oxo-2H-chromen-7-yl dimethylcarbamate (43 mg, 0.058 mmol, yield: 52%) as a yellow oil.

Step 6: 4-((dimethylamino)methyl)-3-(3-((ethylthio)methyl)-2-fluorobenzyl)-2-oxo-2H-chromen-7-yl dimethylcarbamate (43 mg, 0.091 mmol, 1.0 eq.) was dissolved in MeOH (0.06 M)/Water (0.06 M), oxone, monopersulfate compound (55.9 mg, 0.091 mmol, 1.0 eq.) was added and stirred for 1 hour at rt. Water was added to the reaction mixture and the product was extracted with DCM. Combined organic layers were washed with brine, dried over Na₂SO₄, filtered and concentrated under reduced pressure. The impure product was purified by prep basic followed by prep acid to obtain the title compound (6.5 mg, 0.012 mmol, yield: 14%) as a white solid.

4-((dimethylamino)methyl)-3-(3-((ethylsulfonyl)methyl)-2-fluorobenzyl)-2-oxo-2H-chromen-7-yl dimethylcarbamate Analysis: LCMS (Method T): t_{R}=1.60 min; m/z calculated for [M+H]⁺=505.2, found=505.2; 1H NMR (400 MHz, DMSO) δ 8.08 (d, J=8.8 Hz, 1H), 7.35-7.27 (m, 1H), 7.22 (d, J=2.4 Hz, 1H), 7.18-7.04 (m, 3H), 4.52 (s, 2H), 4.07 (s, 2H), 3.65 (s, 2H), 3.12 (q, J=7.5 Hz, 2H), 3.06 (s, 3H), 2.93 (s, 3H), 2.18 (s, 6H), 1.25 (t, J=7.4 Hz, 3H).

### Example 25

### Synthesis of Compound 274

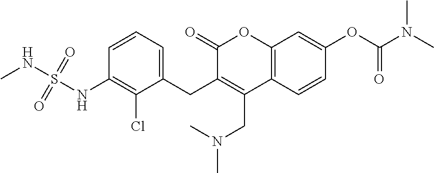

Compound 274 was prepared in 1 step:

Step 1: 3-(3-bromo-2-fluorobenzyl)-4-((dimethylamino)methyl)-2-oxo-2H-chromen-7-yl dimethylcarbamate (50 mg, 0.105 mmol, 1.0 eq.) was dissolved in Toluene (dry) (0.2 M) and flushed with argon. Then, Pd₂(dba)₃ (4.80 mg, 5.24 mol, 0.05 eq.), t-BuXPhos (8.90 mg, 0.021 mmol, 0.2 eq.), K₂CO₃ (29.0 mg, 0.209 mmol, 2.0 eq.) and 2-methylpropane-2-sulfinamide (25.4 mg, 0.209 mmol, 2.0 eq.) were added. The formed reaction mixture was stirred for 17 hours at 50° C. The reaction mixture was flushed with argon and Pd₂(dba)₃ (4.80 mg, 5.24 mol, 0.05 eq.) and t-BuXPhos (8.90 mg, 0.021 mmol, 0.2 eq.) were added. The reaction mixture was stirred for 48 hours at 50° C. The impure product was purified by prep. basic to obtain the title compound (40 mg, 0.076 mmol, yield: 72.7%) as a white solid. Yield: The title compound was isolated as a white solid (73% over 1 step).

3-(3-((tert-butylsulfinyl)amino)-2-fluorobenzyl)-4-((dimethylamino)methyl)-2-oxo-2H-chromen-7-yl dimethylcarbamate Analysis: LCMS (Method T): t_{R}=1.70 min; m/z calculated for [M+H]⁺=518.2, found=518.2; 1H NMR (400 MHz, DMSO) δ 8.08 (d, J=8.8 Hz, 1H), 7.61 (s, 1H), 7.22 (d, J=2.3 Hz, 1H), 7.17-7.06 (m, 2H), 6.97 (t, J=7.8 Hz, 1H), 6.76 (t, J=7.0 Hz, 1H), 4.04 (s, 2H), 3.65 (s, 2H), 3.06 (s, 3H), 2.93 (s, 3H), 2.18 (s, 6H), 1.25 (s, 9H).

### Example 26

### Synthesis of Compound 278

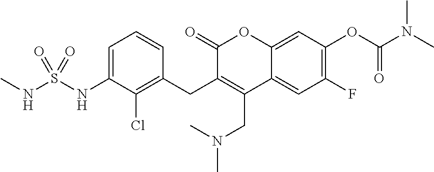

Compound 278 was prepared in 1 step:

Step 1: Starting with 3-(2-chloro-3-((N-methylsulfamoyl)amino)benzyl)-4-(chloromethyl)-6-fluoro-2-oxo-2H-chromen-7-yl dimethylcarbamate and dimethylamine 2.0 M in MeOH, following the general synthesis of Compound B.5. The impure product was purified by prep basic to obtain the title compound after freeze drying as a white solid.

3-(2-chloro-3-((N-methylsulfamoyl)amino)benzyl)-4-((dimethylamino)methyl)-6-fluoro-2-oxo-2H-chromen-7-yl dimethylcarbamate Analysis: LCMS (Method T): t_{R}=2.84 min; m/z calculated for [M+H]⁺=541.0/543.0, found=541.3/543.2; ¹H NMR (400 MHz, DMSO) δ 9.00 (s, 1H), 8.01 (d, J=11.7 Hz, 1H), 7.48 (d, J=6.8 Hz, 1H), 7.41-7.36 (m, 1H), 7.13 (t, J=7.9 Hz, 1H), 6.74 (d, J=7.8 Hz, 1H), 4.09 (s, 2H), 3.54 (s, 2H), 3.09 (s, 3H), 2.95 (s, 3H), 2.57-2.53 (m, 3H), 2.18 (s, 6H).

### Example 27

### Synthesis of Compound 279

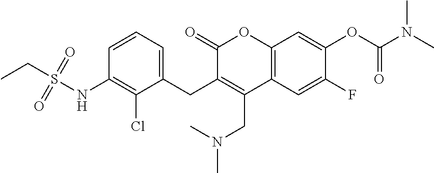 [SMILES: CCS(=O)(=O)Nc1cccc(Cc2c(CN(C)C)c3cc(F)c(OC(=O)N(C)C)cc3oc2=O)c1Cl]

Compound 279 was prepared in 1 step:

Step 1: Following the procedure of the general synthesis of Compound B.5, starting with the bromine using dimethylamine 2.0 M in MeOH. The impure product was purified by column chromatography to obtain the title compound as a white solid after lyophilization.

3-(2-chloro-3-(ethylsulfonamido)benzyl)-4-((dimethylamino)methyl)-6-fluoro-2-oxo-2H-chromen-7-yl dimethylcarbamate Analysis: LCMS (Method T): t_{R}=3.03 min; m/z calculated for [M+H]⁺=540.0/542.0, found=540.2/542.2; ¹H NMR (400 MHz, DMSO) δ 9.45 (s, 1H), 8.01 (d, J=11.7 Hz, 1H), 7.48 (d, J=6.9 Hz, 1H), 7.33 (dd, J=8.0, 1.5 Hz, 1H), 7.15 (t, J=7.9 Hz, 1H), 6.84 (d, J=7.7 Hz, 1H), 4.10 (s, 2H), 3.55 (s, 2H), 3.13 (q, J=7.2 Hz, 2H), 3.09 (s, 3H), 2.95 (s, 4H), 2.17 (s, 7H), 1.28 (t, J=7.3 Hz, 4H).

### Example 28

### Synthesis of Compound 280

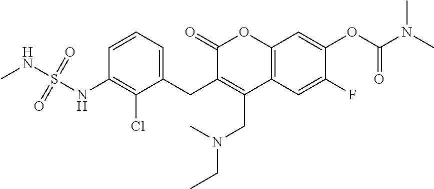

Compound 280 was prepared in 1 step:

Step 1: Starting with 3-(2-chloro-3-((N-methylsulfamoyl)amino)benzyl)-4-(chloromethyl)-6-fluoro-2-oxo-2H-chromen-7-yl dimethylcarbamate and N-ethylmethylamine, following the general synthesis of Compound B.5. The impure product was purified by column chromatography to obtain the title compound after freeze drying as a white solid.

3-(2-chloro-3-((N-methylsulfamoyl)amino)benzyl)-4-((ethyl(methyl)amino)methyl)-6-fluoro-2-oxo-2H-chromen-7-yl dimethylcarbamate Analysis: LCMS (Method M): t_{R}=3.06 min; m/z calculated for [M+H]⁺=555.0/557.0, found=555.3/557.3; ¹H NMR (400 MHz, DMSO) δ 9.01 (s, 1H), 8.05 (d, J=11.7 Hz, 1H), 7.48 (d, J=6.9 Hz, 1H), 7.38 (d, 1H), 7.12 (t, J=7.9 Hz, 1H), 6.72 (s, 1H), 4.09 (s, 2H), 3.60 (s, 2H), 3.09 (s, 3H), 2.95 (s, 3H), 2.54 (d, J=3.6 Hz, 3H), 2.43 (q, J=7.2 Hz, 2H), 2.10 (s, 3H), 0.98 (t, J=7.1 Hz, 3H).

### Example 29

### Synthesis of Compound 281

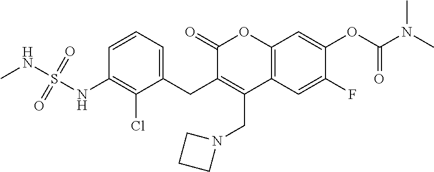

Compound 281 was prepared in 1 step:

Step 1: Starting with 3-(2-chloro-3-((N-methylsulfamoyl)amino)benzyl)-4-(chloromethyl)-6-fluoro-2-oxo-2H-chromen-7-yl dimethylcarbamate and azetidine, following the general synthesis of Compound B.5. The impure product was purified by column chromatography to obtain the title compound after freeze drying as a white solid.

4-(azetidin-1-ylmethyl)-3-(2-chloro-3-((N-methylsulfamoyl)amino)benzyl)-6-fluoro-2-oxo-2H-chromen-7-yl dimethylcarbamate Analysis: LCMS (Method T): t_{R}=2.97 min; m/z calculated for [M+H]⁺=553.0/555.0, found=553.2/555.2; ¹H NMR (400 MHz, DMSO) δ 9.02 (s, 1H), 8.02 (d, J=11.6 Hz, 1H), 7.47 (d, J=6.9 Hz, 1H), 7.38 (d, 1H), 7.12 (t, J=8.0 Hz, 1H), 6.70 (s, 1H), 4.12 (s, 2H), 3.72 (s, 2H), 3.14 (t, J=6.9 Hz, 4H), 3.09 (s, 3H), 2.95 (s, 3H), 2.55 (d, J=3.8 Hz, 3H), 2.07 (s, 2H), 1.90 (p, 2H).

### Example 30

### Synthesis of Compound 282

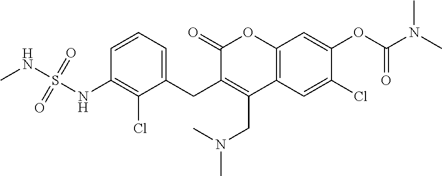

Compound 282 was prepared in 1 step:

Step 1: Starting with 6-chloro-3-(2-chloro-3-((N-methylsulfamoyl)amino)benzyl)-4-(chloromethyl)-2-oxo-2H-chromen-7-yl dimethylcarbamate and dimethylamine 2.0 M in MeOH, following the general synthesis of Compound B.5. The impure product was purified by column chromatography to obtain the title compound) after freeze drying as a white solid.

6-chloro-3-(2-chloro-3-((N-methylsulfamoyl)amino)benzyl)-4-((dimethylamino)methyl)-2-oxo-2H-chromen-7-yl dimethylcarbamate Analysis: LCMS (Method T): t_{R}=3.14 min; m/z calculated for [M+H]⁺=557.4/559.4, found=557.2/559.2; ¹H NMR (400 MHz, DMSO) δ 9.00 (s, 1H), 8.22 (s, 1H), 7.50 (s, 1H), 7.39 (dd, J=8.0, 1.5 Hz, 1H), 7.13 (t, J=7.9 Hz, 1H), 6.77 (s, 1H), 4.09 (s, 2H), 3.56 (s, 2H), 3.11 (s, 3H), 2.95 (s, 3H), 2.55 (d, J=4.3 Hz, 3H), 2.18 (s, 6H).

### Example 31

### Synthesis of Compound 283

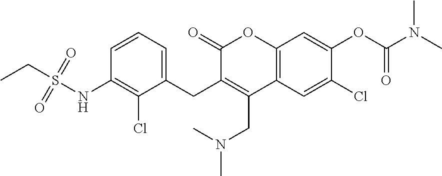 [SMILES: CCS(=O)(=O)Nc1cccc(Cc2c(CN(C)C)c3cc(Cl)c(OC(=O)N(C)C)cc3oc2=O)c1Cl]

Compound 283 was prepared in 1 step1:

Step 1: Following the procedure of the general synthesis of Compound B.5, starting with the bromine using dimethylamine 2.0 M in MeOH. The impure product was purified by column chromatography to obtain the title compound as a white solid after lyophilization.

6-chloro-3-(2-chloro-3-(ethylsulfonamido)benzyl)-4-((dimethylamino)methyl)-2-oxo-2H-chromen-7-yl dimethylcarbamate Analysis: LCMS (Method T): t_{R}=3.18 min; m/z calculated for [M+H]⁺=556.5/558.5, found=556.2/558.2; ¹H NMR (400 MHz, DMSO) δ 9.57 (s, 1H), 8.22 (s, 1H), 7.50 (s, 1H), 7.33 (dd, J=8.0, 1.5 Hz, 1H), 7.15 (t, J=7.9 Hz, 1H), 6.85 (d, J=7.8 Hz, 1H), 4.10 (s, 2H), 3.57 (s, 2H), 3.14 (t, J=7.3 Hz, 2H), 3.11 (s, 3H), 2.95 (s, 3H), 2.18 (s, 6H), 1.28 (t, J=7.3 Hz, 3H).

### Example 32

### Synthesis of Compound 284

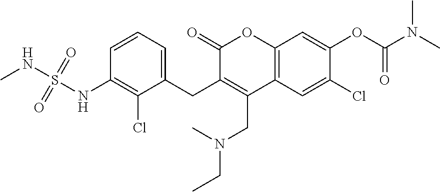

Compound 284 was prepared in 1 step:

Step 1: Starting with 6-chloro-3-(2-chloro-3-((N-methylsulfamoyl)amino)benzyl)-4-(chloromethyl)-2-oxo-2H-chromen-7-yl dimethylcarbamate and N-ethylmethylamine, following the general synthesis of Compound B.5. The impure product was purified by column chromatography to obtain the title compound (after freeze drying as a white solid.

6-chloro-3-(2-chloro-3-((N-methylsulfamoyl)amino)benzyl)-4-((ethyl(methyl)amino)methyl)-2-oxo-2H-chromen-7-yl dimethylcarbamate Analysis: LCMS (Method T): t_{R}=3.37 min; m/z calculated for [M+H]⁺=571.5/573.5, found=571.2/573.2; ¹H NMR (400 MHz, DMSO) δ 9.00 (s, 1H), 8.28 (s, 1H), 7.50 (s, 1H), 7.41-7.37 (m, 1H), 7.23 (s, 1H), 7.14 (t, J=7.9 Hz, 1H), 6.77 (d, J=7.8 Hz, 1H), 4.09 (s, 2H), 3.62 (s, 2H), 3.12 (s, 1H), 3.11 (s, 3H), 2.97 (s, 1H), 2.95 (s, 3H), 2.55 (d, J=4.5 Hz, 4H), 2.44 (q, J=7.1 Hz, 3H), 2.10 (s, 3H), 1.00 (t, J=7.1 Hz, 3H).

### Example 33

### Synthesis of Compound 285

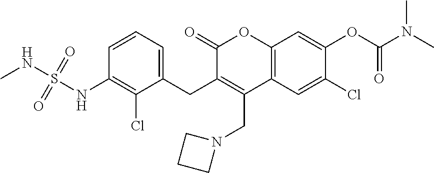

Compound 285 was prepared in 1 step:

Step 1: Starting with 6-chloro-3-(2-chloro-3-((N-methylsulfamoyl)amino)benzyl)-4-(chloromethyl)-2-oxo-2H-chromen-7-yl dimethylcarbamate and azetidine, following the general synthesis of Compound B.5. The impure product was purified by column chromatography to obtain the title compound after freeze drying as a white solid.

4-(azetidin-1-ylmethyl)-6-chloro-3-(2-chloro-3-((N-methylsulfamoyl)amino)benzyl)-2-oxo-2H-chromen-7-yl dimethylcarbamate Analysis: LCMS (Method T): t_{R}=3.13 min; m/z calculated for [M+H]⁺=569.5/571.5, found=569.2/571.2; ¹H NMR (400 MHz, DMSO) δ 9.01 (s, 1H), 8.20 (s, 1H), 7.49 (s, 1H), 7.38 (dd, J=8.2, 1.5 Hz, 1H), 7.13 (t, J=7.9 Hz, 1H), 6.72 (s, 1H), 4.12 (s, 2H), 3.74 (s, 2H), 3.15 (t, J=6.9 Hz, 4H), 3.11 (s, 3H), 2.96 (s, 3H), 2.55 (d, J=3.8 Hz, 3H), 1.90 (p, J=6.8 Hz, 2H).

### Example 34

### Synthesis of Compound 286

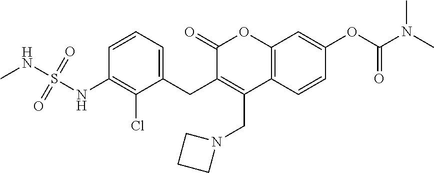

Compound 286 was prepared in 1 step:

Step 1: Starting with 3-(2-chloro-3-((N-methylsulfamoyl)amino)benzyl)-4-(chloromethyl)-2-oxo-2H-chromen-7-yl dimethylcarbamate and azetidine, following the general synthesis of Compound B.5. The impure product was purified by column chromatography to obtain the title compound after freeze drying as a white solid.

4-(azetidin-1-ylmethyl)-3-(2-chloro-3-((N-methylsulfamoyl)amino)benzyl)-2-oxo-2H-chromen-7-yl dimethylcarbamate Analysis: LCMS: t_{R}=1.60 min; m/z calculated for [M+H]⁺=535.1, found=535.2; ¹H NMR (400 MHz, DMSO) δ 8.35 (s, 1H), 8.07 (d, J=8.8 Hz, 1H), 7.39 (dd, J=8.2, 1.5 Hz, 1H), 7.29-7.20 (m, 2H), 7.20-7.07 (m, 2H), 6.73 (dd, J=7.8, 1.5 Hz, 1H), 4.12 (s, 2H), 3.72 (s, 2H), 3.15 (t, J=6.9 Hz, 4H), 3.07 (s, 3H), 2.94 (s, 3H), 2.56 (s, 3H), 1.89 (p, J=6.8 Hz, 2H).

### Example 35

### Materials & Methods

Media components, reagents and buffers for Western Blot: All cell culture media components were obtained from ThermoFisher Scientific. Cell lysis/Protein Extraction Reagent (Cell Signal Technology, Cat No: 9803). 20 mM Tris-HCl (pH 7.5), 150 mM NaCl, 1 mM Na₂EDTA, 1 mM EGTA, 1% Triton, 2.5 mM sodium pyrophosphate, 1 mM beta-glycerophosphate, 1 mM Na₃VO₄, 1 μg/ml leupeptin, Protease inhibitors (Roche, Cat no.11873580001), Phosphatase inhibitors (Cell Signaling Technologies, Cat No. 5870). Coomassie protein assay reagent (ThermoFisher Scientific, Cat. No. 1856209). Laemmli sample loading 4× buffer (ThermoFisher Scientific, Cat No. NP0007). MOPS/SDS electrophoresis running buffer (GenScript, Cat No. M00138). Tris-buffered saline with Tween 20 (TBST buffer): 20 mM Tris-HCl (pH 7.5), 150 mM NaCl, 0.1% Tween 20 NuPAGE gels, 4-12% (ThermoFisher Scientific, Cat No. NP0322BOX). iBLOT nitrocellulose transfer kit (ThermoFisher Scientific Cat No. IB301002). Blocking Buffer (LICOR Cat No. 927-50000).

Antibodies: Phospho-STAT3 (S727), mouse polyclonal antibodies were obtained from BD Biosciences (Cat No. 612542), following 5 antibodies were obtained from Cell Signaling Technologies. Anti-STAT3, rabbit monoclonal antibodies (Cat No. 12640), Anti-phospho-MEK1/2 (S218/S222), rabbit polyclonal antibodies (Cat No: 9121), Anti MEK-1/2, rabbit monoclonal antibodies (Cat No: 9122), Anti-ERK, mouse monoclonal antibodies (Cat No: 9107), and Anti-phospho-ERK, rabbit monoclonal antibodies (Cat No. 4377).

Secondary antibodies: IRDye 800CW goat anti-rabbit antibodies (LICOR Cat No. 926-32211), IRDye 680RD goat anti-rabbit antibodies (LICOR Cat No. 926-68071), IRDye 800CW goat anti-mouse antibodies (LICOR Cat No. 926-32210) and IRDye 680RD goat anti-mouse antibodies (LICOR Cat No. 926-68070).

Tumor Cell Lines: Cell Lines and Tissue Culture conditions: The A549 (Cat No. CCL-185) cell line was obtained from American Type Culture Collection (ATCC) and grown in T75 flasks in DMEM containing 10% FBS and Pen-Strep at 37° C. in a humidified, 5% CO₂ incubator.

The Colon26 syngeneic adenocarcinoma cell line was obtained from the National Cancer Institute. Colon26 tumor cells were maintained as exponentially growing cultures in RPMI-1640 medium containing 10% fetal bovine serum, 2 mM glutamine, 100 units/mL penicillin G sodium, 100 μg/mL streptomycin sulfate, 25 μg/mL gentamicin, 10 mM HEPES, and 0.075% sodium bicarbonate. The tumor cells were grown in tissue culture flasks in a humidified incubator at 37° C., in an atmosphere of 5% CO2 and 95% air.

Subculture conditions: Adherent cells were grown to approximately 90% confluency, culture medium was aspirated and the cell layer was rinsed with PBS. Two mL trypsin solution (0.25%) was added to the flask and observed under an inverted microscope until cell layer is dispersed. Eight mL media was added, cells were spun down at 1000×g for 5 minutes. Cell pellet was re-suspended in 10 mL media and an appropriate volume was inoculated into a new culture flask.

Compound (drug) treatment: Cells were plated in a 6-well plate at a density of 250,000-300,000 cells/well in 3 mL media and incubated 37° C. in a humidified, 5% CO₂ incubator. Next day, 10 mM stock solutions of compounds were diluted 10- and 100-fold in DMSO to yield 100 and 10 uM solutions, respectively. These solutions were added to the cells (3 uL/well), mixed by swirling the plate, and incubated at 37° C. in a humidified, 5% CO₂ incubator for 2 hours.

Cell lysis and protein estimation: Cells were washed with PBS, and scraped in 50 μL of lysis buffer containing protease and phosphatase inhibitors. Cell lysates were stored at −20° C. Cell lysates were thawed and spun at 12,000 rpm for one minute, 3 ul of the supernatant was added to 500 μL of Coomassie blue reagent following by 500 μL of water. Absorbance was read at 595 nm after 10 minutes of incubation. Protein standards were used (0-20 mg/mL) to calculate protein concentrations of test samples.

Western Blotting: For electrophoresis 20 μg of protein was mixed with 5 ul of 4× Laemmle's sample buffer and 1 ul of 0.4 M DTT in a volume of 20 ul made up with lysis buffer. All samples were heated at 95° C. for 5 minutes, cooled to room temperature and spun down. Protein samples were loaded onto 4-12% polyacrylamide gels and run at 100V for approximately 1.5 hours till the blue dye reached the bottom. After the run, gel was removed and protein transfer was done using iBlot for 7 minutes, as per manufacturer's recommendations. After the transfer, nitrocellulose membrane was incubated on a shaker in 5 mL of blocking buffer at room temperature for 1 hr. The blot was then incubated overnight on a shaker in 5 ml of blocking buffer containing 0.2% Tween-20 and primary antibody, at room temperature. Anti-phospho-STAT3 antibody was used at a dilution of 1:500, the other 3 primary antibodies were used at a dilution of 1:1000.

Next day, the blot was washed 3 times for 10 min each with 10 mL of TBST followed by incubation on a shaker in 5 ml of blocking buffer containing 0.2% Tween-20 and 0.5 ul of the IRDye labeled secondary antibodies, diluted 1:10000, at room temperature for 1 hr. The blot was then washed 3 times for 10 min each with 10 mL of TBST and dried between sheets of paper towels. Imaging was done using LICOR's Odyssey imaging system, quantitation was done using their software, Image Studio version 3.1.

Animal Studies: Female BALB/c mice (BALB/cAnNCrl, Charles River) were eleven weeks old on Day 1 of the study and had a body weight (BW) range of 15.8 to 21.4 g. The animals were fed ad libitum water (reverse osmosis, 1 ppm Cl) and NIH 31 Modified and Irradiated Lab Diet® consisting of 18.0% crude protein, 5.0% crude fat, and 5.0% crude fiber. The mice were housed on irradiated Enrich-o'Cobs™ bedding in static microisolators on a 12-hour light cycle at 20-22° C. (68-72° F.) and 40-60% humidity. Charles River Discovery Services North Carolina (CR Discovery Services) specifically complies with the recommendations of the Guide for Care and Use of Laboratory Animals with respect to restraint, husbandry, surgical procedures, feed and fluid regulation, and veterinary care. The animal care and use program at CR Discovery Services is accredited by the Association for Assessment and Accreditation of Laboratory Animal Care International (AAALAC), which assures compliance with accepted standards for the care and use of laboratory animals.

In Vivo Implantation and Growth: Colon26 tumor cells used for implantation were harvested during log phase growth and re-suspended at a concentration of 1×10⁷ cells/mL in cold phosphate buffered saline (PBS). Each mouse was injected subcutaneously in the right flank with 1×10⁶ tumor cells (in a 0.1 mL cell suspension). The tumors were measured with a caliper in two dimensions to monitor size as the mean volume approached the desired 80 to 120 mm3 range. Tumor size was calculated using the formula: Tumor volume (mm³)=(w²·l)/2, where w=width and l=length, in mm, of a tumor. Tumor weight may be estimated with the assumption that 1 mg is equivalent to 1 mm, of tumor volume. Tumors were measured with a caliper twice weekly for the duration of the study.

Treatment: Tumor bearing BALB/c mice were randomized into treatment groups once target range was reached (n=5 per group). All treatments were administered orally (p.o.) once a day for fourteen days (QD×14) in a volume of 10 mL/kg (0.2 mL per 20 g mouse), adjusted to the BW of each animal.

Sampling for Pharmacokinetic analysis: Blood, skeletal muscle, livers and tumors were collected from three animals each from the designated groups two hours after animals received a single dose. Full blood volume was collected by terminal cardiac puncture under isoflurane anesthesia, processed for plasma and the presence of K2EDTA anti-coagulant and stored at −80° C. Skeletal muscle groups composed of right and left gastrocnemius, tibialis and soleus muscles were collected as a unit, snap frozen and stored at −80° C. Livers were collected were snap frozen and shipped to CRL-Worcester for bioanalytical analysis. Sample inventories are appended.

Data Analysis: Tumors were measured using calipers twice per week with the data being expressed as either median+/−interquartile range or as individual plots in days. Tumor growth inhibition (TGI) was calculated as follows: % TGI=1−(T/C)×100, where: T=median Tumor volume for a treatment group, and C=median Tumor volume for the designated control group.

### Example 36

### Pharmacokinetic Properties

### Physicochemical Properties: Absorption & Efflux

Materials and Methods: Stock solutions of Test Article (TA) was prepared at 50 mM in DMSO and was further diluted to 10 mM using DMSO. Control inhibitor stock solutions were prepared at concentrations that were 1000× the final assay concentration. Individual stocks for controls ranitidine, warfarin and talinolol were prepared at 10 mM in DMSO and verapamil stock solution was prepared at 25 mM in DMSO. Caco-2 cells, obtained from ATCC, clone C2BBe1, were grown in 96-well Transwell plates, cultured for 27 days in Dulbecco's modified Eagle's medium supplemented with 10% fetal bovine serum, 1% L-glutamax,1% penicillin-streptomycin (pen-strep) and 10 mM HEPES (2-[4-(2-hydroxyethyl) piperazin-1-yl]ethane sulfonic acid) and incubated at 37° C., 5% CO2, and humidified.

On the day of the assay, solutions for test article and control substrates were prepared by diluting each of the stock solutions 500-fold (to get 2× the final assay concentrations) with transport buffer (HBSS: Hank's balanced salt solution; Corning catalog #21-023-CV). To these assay solutions, test article (10 mM for conditions to be tested for test article as inhibitor) and control inhibitor (for conditions to be tested for test article and control articles as substrates) stock solutions were diluted 500-fold to get 2× the final assay concentrations). To the solutions where no inhibitor is added, blank DMSO was added to normalize DMSO percentage in the assay system. Solutions were incubated at 37° C. for at least 15 minutes. The basal assay plates were prepared by filling apical-to-basolateral wells of a 96-well sterile plate with 250 μL of Transport Buffer and basolateral-to-apical wells with 130 μL of 2× assay solutions prepared above and 130 μL of HBSS. Samples (10 μL) were collected each from basal compartment of basolateral-to-apical wells for time zero (T0) samples and were diluted with 4 volumes of transport buffer (HBSS). Monolayer integrity was assessed for the assay plate before and after the assay by taking measurements of Transepithelial Electrical Resistance (TEER) of cell monolayer in each assay well. Caco-2 cell plates were prepared for the bidirectional assay by exchanging the cell culture medium (Dulbecco's modified Eagle's medium with supplements) in the apical wells of the plate three times with 85 μL transport buffer (HBSS). After the final wash, 52.5 μL of the buffer was removed from the apical wells (leaving 62.5 L buffer in the wells) and replaced with 62.5 μL of 2× assay solutions prepared above for apical-to-basolateral wells or 52.5 μL of fresh Transport Buffer for basolateral-to-apical wells. The total volume in the apical wells at this point is assumed to be 125 μL for apical-to-basolateral wells and 115 μL for basolateral-to-apical wells. Samples (10 μL) were collected from each apical compartment of apical-to-basolateral wells for time zero (T0) samples and were diluted with 4 volumes of transport buffer (HBSS). The final nominal concentrations for the assay were 10 M for TA as substrate and as inhibitor, 10 μM for all control substrates, 10 μM for valspodar, Ko143, ranitidine, talinolol and warfarin and 25 μM for verapamil. The apical section of the Caco-2 plate was transferred to the basal plate and incubated at 37° C. for 2 hours; the assay was performed in triplicate. Following the 2-hour incubation period, samples (10 μL each) were collected from all apical compartments, samples (50 μL each) were collected from apical-to-basolateral basal compartments, and samples (10 μL each) were collected from basolateral-to-apical basal compartments. The 10-μL samples from both the apical and basolateral sides of the cell monolayer were diluted with 4 volumes of transport buffer (HBSS). The samples were quenched with 100 μL ice-cold acetonitrile containing internal standards at 250 ng/mL concentration. An aliquot (50 μL) of quenched sample was removed and diluted with 100 μL Milli-Q water. Samples were stored refrigerated until analysis.

Bioanalysis: Test Article and control articles, the LC system used a Waters XSELECT HSS T3 2.5 μm, 30×2.1 mm column, with a gradient (0.9 mL/min flow rate) starting at 99% mobile phase A (0.1% formic acid in water) to 95% mobile phase B (0.1% formic acid in acetonitrile). The column was set to a temperature of 55° C. Analytes and internal standards were detected using an Applied Biosystems Sciex API-5500 triple quadrupole mass spectrometer with Agilent 1260 Infinity Binary Pump and Apricot Designs ADDA High-Speed Dual Arm Autosampling System. The instrument was equipped with an electrospray ionization source (600° C.) operated in the positive-ion mode. Analytes and internal standards were monitored in the multiple-reaction-monitoring (MRM) scan mode.

### Data Analysis and Calculations:

Data were captured and processed using Analyst version 1.6. Data were analyzed, and results were calculated using Microsoft Excel.

Mean peak area ratios were used to calculate following parameters:

### Absorption:

$$\% Absorption ( A - B ) = \frac{Post - assay basal mean area ratio}{Pre - assay apical mean area ratio} \times 100$$

$$\% Absorption ( B - A ) = \frac{Post - assay apical mean area ratio}{Pre - assay basal mean area ratio} \times 100$$

### Recovery:

$$\% Recovery ( A - B ) = \frac{\begin{matrix}[ Post - assay apical mean area ratio \times \\ Apical volume ( μL ) at timepoint ] + \\ [ Post - assay basal mean area ratio \times \\ Basal volume ( μL ) at timepoint ]\end{matrix}}{\begin{matrix}Pre - assay apical mean area ratio \times \\ Initial apical volume ( μL )\end{matrix}} \times 100$$

$$\% Recovery ( B - A ) = \frac{\begin{matrix}[ Post - assay basal mean area ratio \times \\ Basal volume ( μL ) at timepoint ] + \\ [ Post - assay apical mean area ratio \times \\ Apical volume ( μL ) at timepoint ]\end{matrix}}{\begin{matrix}Pre - assay basal mean area ratio \times \\ Initial basal volume ( μL )\end{matrix}} \times 100$$

### Apparent Permeability:

$${P}_{app} ( A - B ) in cm / s = \frac{\% Absorption ( A - B ) \times Basal volume ( mL ) at timepoint}{Time ( s ) \times Surface area ( 0.143 {cm}^{2} )}$$

$${P}_{app} ( B - A ) in cm / s = \frac{\% Absorption ( B - A ) \times Apical volume ( mL ) at timepoint}{Time ( s ) \times Surface area ( 0.143 {cm}^{2} )}$$

$$Efflux Ratio ( ER ) of {P}_{app} = \frac{{P}_{app} ( B - A )}{{P}_{app} ( A - B )}$$

$$An Efflux Ratio > 2 is considered active efflux .$$

$$\% {Inhibition}_{n} = ( ( {ER}_{control} - {ER}_{n} ) / ( {ER}_{control} ) ) * 100$$

Physicochemical Properties: hERG Channel

Materials and Methods: CHO cells (transformed with adenovirus 5 DNA; Stably transfected with full length hERG cDNA) were cultured in Ham's F-12 supplemented with 10% fetal bovine serum, 100 U/mL penicillin G sodium, 100 μg/mL streptomycin sulfate and 400 μg/mL Zeocin. Before testing, cells in culture dishes were rinsed with Hank's Balanced Salt Solution, detached with accutase. Immediately before use in the SyncroPatch® 384PE system, the cells were washed in HB-PS to remove the accutase and re-suspended in 15 mL of HB-PS.

The Test Article (TA) effects were evaluated using SyncroPatch® 384PE systems (SP384PE; Nanion Technologies, Livingston, NJ). HEPES-buffered intracellular solution (Charles River proprietary) for whole cell recordings was loaded into the intracellular compartment of the SP384PE. Extracellular buffer (HB-PS) and Cell suspension (in HB-PS) were pipetted into the extracellular compartment of the SP384PE chip. After establishment of a whole-cell configuration, membrane currents were recorded using patch clamp amplifier in the SP384PE system.

Test article (TA) concentrations (eight (8) concentrations of each test article were evaluated) were applied to naïve cells (n=4, where n=replicates/concentration). Each application consisted of addition of 40 μL of 2× concentrated test article solution to the total 80 μL of final volume of the extracellular well of the SP384PE chip. Duration of exposure to each compound concentration was five (5) minutes. Cisapride (hERG positive control): eight (8) concentrations ranging from 0.003-3 M were used to determine the dose response of the block of hERG current.

hERG current was measured using stimulus voltage patterns with fixed amplitudes: activation pre-pulse (TP1) to +40 mV for 2 s and test pulse (TP2) to −40 mV for 2 s from a holding potential of −80 mV. hERG current was measured as the outward peak current at TP2 (tail current). The stimulation was repeated with 0.1 Hz frequency during 2 min as baseline and 5 min after TA application.

Data acquisition and analyses were performed using the SP384PE system operation software. The decrease in current amplitude after TA application was used to calculate the percent block relative to control. Results for each TA concentration (n≥2) were averaged; the mean and standard error values were calculated, and used to generate dose-response curves.

Tonic Block effect was calculated as:

$$\% Block ( Tonic ) = ( 1 - {I}_{TP 2 , TA} / {I}_{TP 2 , Baseline} ) \times 100 \% ,$$

where I_{TP2, baseline} and I_{TP2, TA} were the outward peak K⁺ currents elicited by the TP2 before application and 5 min after application of a test article, respectively.

The data were corrected for run-down:

$$\% {Block}^{′} = 100 \% - ( ( \% Block - \% PC ) * ( 100 \% / ( \% VC - \% PC ) ) ,$$

where % VC and % PC were the mean values of the current block with the vehicle and positive controls, respectively.

Concentration-response data were fitted to an equation of the following form:

$$\% Block = \% VC + \{ ( \% PC - \% VC ) / [ 1 + {( [ Test ] / {IC}_{50} )}^{N} ] \} ,$$

where [Test] was the concentration of test article, IC₅₀ was the concentration of the test article producing half-maximal block, N was the Hill coefficient, % VC was the mean current block at the vehicle control and % Block was the percentage of ion channel current inhibited at each concentration of a test article. Nonlinear least squares fits were solved with the XLfit add-in for Excel (Microsoft, Redmond, WA).

### Physicochemical Properties: Microsome Stability

Materials and Methods: Individual stock solutions of Test Articles (TA) were prepared at 30 mM in DMSO and were further diluted to 2 mM in DMSO. A stock solution of verapamil (control article) was prepared at 2 mM in DMSO. Prior to use in the assay, the 2 mM stock solutions were diluted 10-fold with acetonitrile. The acetonitrile diluted stocks were diluted 1:50 into warm (37° C.) 0.1 M potassium phosphate, pH 7.4, containing 2.0 mM NADPH (2× final assay concentration). Frozen liver microsomes were quick thawed in a 37° C. water bath and then placed on ice. The liver microsomes (20 mg protein/mL) were diluted in 0.1 M potassium phosphate buffer, pH 7.4, (warmed to 37° C.) to a concentration of 1 mg protein/mL (2× final assay concentration). To initiate the reaction, 75 μL of diluted 2× microsomes were added to an equal volume of 2× compound/NADPH solution in a polypropylene 96-well microtiter plate. The plate was incubated with gentle shaking at 37° C. Duplicate 30 μL aliquots were removed immediately after compound addition (To: time zero) and at 60 minutes. At each timepoint, control and test article samples were quenched with 180 μL of ice-cold acetonitrile containing internal standards. The quenched samples were gently mixed and were stored in a −20° C. freezer for at least 30 minutes. After the final time point (60 minutes), the quenched samples were vortexed (10 minutes) and then centrifuged at 3100 rpm for 10 minutes at 4° C. Supernatant (50 μL) was removed, transferred to a new 96-well plate, and diluted with 100 μL of water. The sample plate was sealed, mixed, and then stored refrigerated until analysis.

### Bioanalysis:

For the analysis of all test articles and verapamil (control), a Waters XSELECT HSS T3 2.5 μm, 30×2.1 mm column was used with a gradient (0.9 mL/min flow rate) starting at 99% mobile phase A (0.1% formic acid in water) to 95% mobile phase B (0.1% formic acid in acetonitrile). The column oven was set to a temperature of 55° C.

Analytes and internal standards were detected using an Applied Biosystems Sciex API 5500 triple quadrupole mass spectrometer with Sound Analytics ADDA Autosampler. The instruments were equipped with an electrospray ionization source (600° C.) operated in the positive ion.

Data Collection and Calculations: Analyst (AB Sciex) and Acquity (Waters) were used for data acquisition. Results were calculated using Microsoft Excel.

Peak area ratios for IS-normalized test article counts were used to calculate the percent remaining relative to the time zero (T₀) value. As appropriate, results were plotted and further analyzed to calculate half-life (T_{1/2}) and CLᵢₙₜ values.

Mean peak area ratios were used to calculate the percent remaining relative to the T₀ value:

$$\% Remaining at {Time}_{x} = ( mean peak ratio {T}_{x} / Mean peak area ratio {T}_{0} ) \times 100$$

Results were plotted [ln (% remaining) vs. incubation time] and further analyzed to calculate half-life (T_{1/2}) values:

$${T}_{1 / 2} = - 0.693 / Slope$$

$${CL}_{int} = ( ln 2 / {T}_{1 / 2} ) \times ( 1 / protein conc ) \times 1000$$

Clᵢₙₜ is intrinsic clearance calculated in the units of mL/min/kg of protein
CD-1 mouse
CD-1 mouse: 45 mg microsomes/g liver; 87.5 g liver/kg body weight
Sprague-Dawley rat: 45 mg microsomes/g liver; 45 g liver/kg body weight
Beagle dog: 45 mg microsomes/g liver; 25 g liver/kg body weight
Human: 45 mg microsomes/g liver; 20 g liver/kg body weight

Physicochemical Properties: Plasma Stability

Materials and Methods: Stock solutions of Test Article (TA) were prepared at a concentration of 50 mM in DMSO and was further diluted to 2 mM with DMSO. Individual stock solutions of propantheline and lovastatin were prepared at 10 mM in DMSO and were further diluted to 2 mM with DMSO.

Frozen matrices (Balb/c mouse, Wistar Han rat, Beagle dog, and human plasma containing K₂EDTA as anti-coagulant) were thawed and centrifuged at 3100 rpm for 10 minutes at 4° C. to remove particulates. The lipid layer was then removed, and the supernatant was transferred to a new tube without disturbing the pellet. After the matrices were incubated at 37° C. for at least 10 minutes, the pH of each matrix was adjusted to pH 7.4, using 10% phosphoric acid or 1N sodium hydroxide as necessary. Test and control article stock solutions were spiked (2 μL) into matrix (1.998 mL) and mixed to final assay concentration of 2 M. Duplicate 30-μL aliquots of spiked plasma were transferred to matrix tubes in 96-well plate immediately after spiking for timepoints of 0, 30, 60, 120, 240, and 360 minutes. These tubes were then incubated at 37° C. with shaking and at each timepoint, the corresponding tubes containing samples were quenched with 180 μL of cold acetonitrile containing the internal standards. The quenched samples were vortex-mixed briefly and stored refrigerated. After the final time point (360 minutes), the plates with all quenched samples were vortex-mixed for 10 minutes and then centrifuged at 3100 rpm for 10 minutes at 4° C. to sediment the precipitated protein. The supernatant (50 μL) was transferred to a clean 96-well plate and diluted with 100 μL of water. The plates were stored refrigerated until analysis.

Bioanalysis: For the analysis of TA and Propantheline, a Waters XSELECT HSS T3 2.5 μm, 30×2.1 mm column was used with a gradient (0.9 mL/min flow rate) starting at 99% mobile phase A (0.1% formic acid in water) to 95% mobile phase B (0.1% formic acid in acetonitrile). The column was set to a temperature of 55° C.

For the analysis of Lovastatin, a Waters XSELECT HSS T3 2.5 lm, 30×2.1 mm column was used with a gradient (0.9 mL/min flow rate) starting at 70% mobile phase A (0.1% formic acid in water) to 95% mobile phase B (0.1% formic acid in acetonitrile). The column was set to a temperature of 55° C.

All analytes and internal standards were detected using an Applied Biosystems Sciex API-5500 triple quadrupole mass spectrometer with Agilent 1260 Infinity Binary Pump and Apricot Designs ADDA High-Speed Dual Arm Autosampling System. The instrument was equipped with an electrospray ionization source (500° C.) operated in the positive-ion mode.

Data Analysis: Peak area ratios for IS-normalized test article and control article counts were used to calculate the percent remaining relative to the time zero (T₀) value. As appropriate, results were plotted and further analyzed to calculate half-life (T_{1/2}) values.

Mean peak area ratios were used to calculate the % remaining relative to the T₀ value:

$$\% Remaining at {Time}_{x} = ( mean peak area ratio {T}_{x} / mean peak area ratio {T}_{0} ) \times 100$$

Results were plotted [ln (% remaining) vs. incubation time] and further analyzed to calculate half-life (T_{1/2}) values: T_{1/2}=−0.693/Slope

Physicochemical Properties: CYP450 Panel

Materials and Methods: Stock solutions of Test Article (TA) were prepared at 50 mM in DMSO, followed by eight serial dilutions in water down to 0.00545 μM. Each TA solution and DMSO alone were diluted 3.33-fold in acetonitrile followed by a further 100-fold dilution into 100 mM potassium phosphate buffer, pH 7.4 with and without 3 mM NADPH (to make 3× compound solutions). Positive control inhibitors (fluvoxamine, ticlopidine, quercetin, sulfaphenazole, omeprazole, paroxetine and mifepristone) were dissolved at 50 mM in DMSO followed by eight serial dilutions in DMSO down to 0.00545 mM. Each inhibitor solution and DMSO alone were diluted 3.33-fold in acetonitrile followed by a further 100-fold dilution into 100 mM potassium phosphate buffer, pH 7.4 with and without 3 mM NADPH (to make 3× compound solutions). Pooled human liver microsomes were submerged in a 37° C. water bath until just thawed and then placed on ice. The human liver microsomes were diluted in 100 mM potassium phosphate buffer (warmed), pH 7.4 to 0.3 mg/mL (3× concentration) directly before use. The substrates were used at approximately their Kₘ concentration for the respective isoenzyme, which were expected to fall within the linear range of CYP450-mediated metabolism. The substrates in DMSO (40 mM phenacetin, 25 mM bupropion, 1 mM amodiaquine, 10 mM diclofenac, 40 mM mephenytoin, 10 mM dextromethorphan, and 125 mM testosterone) and in methanol (3.06 mM midazolam) were added to a 3 mM NADPH solution prepared in 100 mM potassium phosphate (KPhos) buffer, pH 7.4 (buffer) to make a (3×) buffer/cofactor/substrate (BCS) solution with concentrations ranging from 3 μM to 150 μM. Similar dilution of substrates was done in 100 mM KPhos buffer, pH 7.4 not containing any NADPH to make 3× buffer/substrate (BS) solution.

To initiate the reactions (in duplicate), beginning with the longest preincubation timepoint, 50-μL of diluted 3× compound solutions were added to an equal volume of 3× microsomes in a 96-well polypropylene plate and incubated at 37° C. This step was done for each preincubation timepoint. Once the appropriate preincubation timepoint was finished, 50 μL of diluted 3× substrate solutions were added to the preincubation reaction mixture prepared above. The 3× substrate solutions with 3 mM NADPH in 100 mM KPhos buffer, pH 7.4 were added to the plate containing compound solutions with no NADPH in 100 mM KPhos buffer, pH 7.4 and vice versa; the final concentrations of DMSO and ACN were less than 1%. For the test article and the positive control inhibitors, final concentrations were 0.00, 0.00545, 0.0218, 0.0870, 0.348, 1.39, 5.55, 22.2, 33.3 and 50.0 μM.

For the T₀ control condition, 50 μL aliquots (from the samples containing 0.0 μM and the highest concentration of the test or control article) were immediately removed and quenched with 200 μL of ice-cold acetonitrile containing internal standards. The assay plates were sealed and incubated at 37° C. with very gentle agitation. After a 30-minute incubation period, 50 μL aliquots were removed and quenched with 200 μL of ice-cold acetonitrile containing internal standards at 250 ng/mL concentration. The quenched samples were stored refrigerated.

Quenched samples were centrifuged at 3100 rpm for 5 minutes at 4° C. Supernatants from 1A2, 2B6 and 2C8 assays were pooled (50 μL per sample). Supernatants from 2D6, 3A4 (midazolam substrate) and 3A4 (testosterone substrate) assays were pooled (50 μL per sample). Pooled samples were diluted with 300 μL water prior to analysis. For unpooled 2C9 and 2C19 assay samples, supernatant (50 μL) was diluted with 100 μL water prior to analysis.

Bioanalysis: For TA, a Waters XSELECT HSS T3 2.5 μm, 30×2.1 mm column was used with a gradient (1.0 mL/min flow rate) starting at 99% mobile phase A (0.1% formic acid in water) to 95% mobile phase B (0.1% formic acid in acetonitrile). The column was set to a temperature of 55° C.

For CYP specific metabolites, a Waters XSELECT HSS T3 2.5 μm, 50×2.1 mm column was used with a gradient (0.8 mL/min flow rate) starting at 100% mobile phase A (0.1% formic acid in water) to 95% mobile phase B (0.1% formic acid in acetonitrile). The column was set to a temperature of 55° C.

TA, CYP specific metabolites and internal standards were detected using an Applied Biosystems Sciex API-5500 triple quadrupole mass spectrometer with Waters Acquity UPLC System. The instrument was equipped with an electrospray ionization source (600° C.) operated in the positive-ion mode except CYP2C9 samples were monitored in negative-ion mode.

Data Collection and Calculations: Data were captured and processed using Analyst Version 1.6.2. Data were analyzed, and results were calculated using Microsoft Excel 2010 and GraphPad Prism v.5.02 (for curve fitting and IC₅₀ calculations).

Percent inhibition was calculated by the following equation:

$$\% {Inhibition}_{n} = 100 - ( ( Area {Ratio}_{n} - No Activity Background ) / Net Signal * 100 )$$

Where:
No Activity Background (i.e., full inhibition) is the T0 quenched condition
Full Activity is T30 without inhibitor (i.e., non-inhibited) condition
Net Signal=Full Activity−No Activity Background
Percent Activity=100%−% Inhibition

TABLE 2
Physicochemical Properties: Absorption & Efflux

| | Mean Pₐₚₚ A- | Mean Pₐₚₚ B- | Mean (B-A/A- | A-B |
|---|---|---|---|---|
| Compound | B | A | B) | Permeability |
| (10 μM; Caco-2; 2 hrs) | (10⁻⁶ cm/s) | (10⁻⁶ cm/s) | (Efflux Ratio)^{∧} | Ranking* |
| Reference-1 | 8.83 | 28.2 | 3.19 | High |
| Reference-2 | 18.1 | 20.9 | 1.16 | High |

Controls

| | | | | |
|---|---|---|---|---|
| Ranitidine | 0.347 | 1.41 | 4.06 | Low |
| Talinolal | 0.375 | 8.20 | 21.8 | Effluxed |
| Talinolal + Verapamil | 0.766 | 3.07 | 4.01 | Inhibited |
| (2.5 uM) | | | | Efflux |
| Warfarin | 36.6 | 26.6 | 0.726 | High |

Reference 1 is Selumetinib.
Reference 2 is 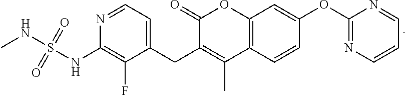 [SMILES: CNS(=O)(=O)Nc1nccc(Cc2c(C)c3ccc(Oc4ncccn4)cc3oc2=O)c1F]
^{∧}An efflux ratio >2 indicates potential for the compound to be a substrate for Pgp or other active transporter.
*Permeability Ranking: lower is <1 x 10 = 6 cm/s; higher is >1 x 10 = 6cm/s

TABLE 3
Physicochemical Properties: hERG Channel

| Compound | |
|---|---|
| (30 to 0.01 μM dose range) | hERG IC50 [μM] |
| Reference-1 | >30 |
| Reference-2 | >30 |
| Controls | |
| Cisapride | 0.01 |

Reference 1 is Selumetinib.
Reference 2 is

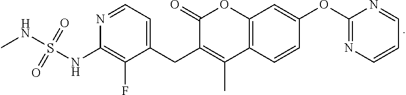 [SMILES: CNS(=O)(=O)Nc1nccc(Cc2c(C)c3ccc(Oc4ncccn4)cc3oc2=O)c1F]

TABLE 4
Physicochemical Properties: Microsome Stability

| Com- | Human | Beagle Dog | SD Rat | CD-1 Mouse |
|---|---|---|---|---|
| pound | (% Reamining | (% Reamining | (% Remaining | (% Reamining |
| [2 μM] | @1 hour) | @1 hour) | @1 hour) | @1 hour) |
| Refer- | 75.8 | 97.5 | 54.6 | 13.9 |
| ence-1 | | | | |
| Refer- | 107.1 | 111.6 | 102.0 | 96.0 |
| ence-2 | | | | |

Controls

| | | | | |
|---|---|---|---|---|
| Vera- | 5.8 | 6.0 | 0.7 | 0.8 |
| pamil | | | | |

Reference 1 is Selumetinib.
Reference 2 is

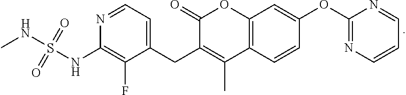 [SMILES: CNS(=O)(=O)Nc1nccc(Cc2c(C)c3ccc(Oc4ncccn4)cc3oc2=O)c1F]

TABLE 5
Physicochemical Properties: Plasma Stability

| | | | | CD-1 |
|---|---|---|---|---|
| | | | SD Rat | Mouse |
| | Human | Beagle Dog | (% | (% |
| Compound | (% Reamining | (% Reamining | Remaining | Reamining |
| [2 μM] | @2 hour) | @2 hour) | @2 hour) | @2 hour) |
| Reference-1 | 98.1 | 100.4 | 94.9 | 104.8 |
| Reference-2 | 91.1 | 99.8 | 99.8 | 92.9 |

Controls

| | | | | |
|---|---|---|---|---|
| Propantheline | 5.7 | 58.4 | 92.6 | 27.1 |
| Lovastatin | 81.8 | 94.1 | 0.6 | 0.6 |

Reference 1 is Selumetinib.
Reference 2 is

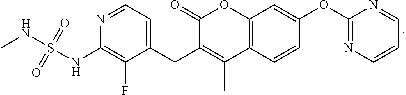 [SMILES: CNS(=O)(=O)Nc1nccc(Cc2c(C)c3ccc(Oc4ncccn4)cc3oc2=O)c1F]

TABLE 6
Physicochemical Properties: CYP450 Panel

| Compound | | | | | | | | |
|---|---|---|---|---|---|---|---|---|
| [10 μM] | 1A2* | 2B6* | 2C8* | 2C9* | 2C19* | 2D6* | 3A4* | 3A4^{∧} |
| Reference-1 | 8.3 | 30.2 | 18.9 | 14.1 | 13.9 | 5.0 | 25.9 | 21.4 |
| Reference-2 | 0.8 | 13.6 | 41.5 | −1.6 | −0.9 | 3.4 | 16.6 | 15.7 |

Controls

| | | | | | | | | |
|---|---|---|---|---|---|---|---|---|
| Fluvoxamine | 100.2 | — | — | — | — | — | — | — |
| Ticlopidine | — | 101.5 | — | — | — | — | — | — |
| Quercetin | — | — | 93.1 | — | — | — | — | — |
| Sulfa- | — | — | — | 101.2 | — | — | — | — |
| phenazole | | | | | | | | |
| Omeprazole | — | — | — | — | 61.6 | — | — | — |
| Quinidine | — | — | — | — | — | 99.7 | — | — |
| Ketoconazole | — | — | — | — | — | — | 110.1 | — |
| Ketoconazole | — | — | — | — | — | — | — | 105.5 |

Reference 1 is Selumetinib.
Reference 2 is 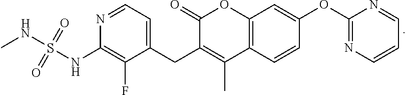 [SMILES: CNS(=O)(=O)Nc1nccc(Cc2c(C)c3ccc(Oc4ncccn4)cc3oc2=O)c1F]
*1A2 (phenacetin), 2B6 (bupropion), 2C8 (amodiaquine), 2C9 (Diclofenac), 2C19 (mephenytoin), 2D6 (dextromethophan), 3A4 (midazolam)
^{∧}3A4 (testosterone)

### Example 37

### Cell-Based pERK Dose Response Study

Materials and Methods: A549 or A375 cells were seeded in 6-well plates at an appropriate seeding density on day 0. On day 2 or 3, after checking the health and confluency of cells, the media was aspirated and replaced with 1 mL media containing a predetermined concentration of compound and incubated for 2 hours. After 2 hours, the media was aspirated, and the cells washed 2 times with cold PBS. Cells were lysed on ice, with 50 uL 1×CST lysis buffer +1 mM PMSF for 5 minutes. After 5 minutes, cells were removed using a scraper and transferred to cold 1.5 mL tubes, and centrifuged for 10 min, 4° C., 14,000×g. Supernatant was gently removed and snap frozen in liquid nitrogen. Protein concentration of the lysate was determined using Bradford Reagent (analyzed using SpectraMax M2E) and diluted to 1 mg/mL or pERK or 1.5 mg/mL for pMEK analysis. Cell lysates were then analyzed for phosphor-ERK/total-ERK levels on a Jess system (ProteinSimple; Cat #JS3346) using the following antibodies: tERK1/2 (CST 4696; 1:50) and pERK1/2 (CST 4377; 1:50). For pMEK/tMEK, the following antibodies were used: tMEK1/2 (CST 4694; 1:15) and pMEK1/2 (CST 9154; 1:400). All dilutions were in Milk-free antibody diluent.

Mouse & Human Microsome (t_{1/2} Min), Mouse & Human Clᵢₙₜ (μl/min/mg)

Test compounds were dissolved in DMSO to a concentration of 10 mM and further diluted to 100 μM using acetonitrile. Liver microsomes from selected species were incubated in duplicate with the test compound at a final concentration of 1 μM in 0.1 M potassium phosphate buffer (pH 7.4) containing 3.3 mM MgCl₂, 0.5 mg/ml microsomal protein, in the presence or absence of NADPH (1 mM). Incubations were performed at 37° C. in a total volume of 500 μl. Control incubations with reference substances were included for each experiment.

At different time points (t=0, 5, 15, 30, 45 min), 50 μl of the incubation mixture was transferred into a quench plate containing acetonitrile and internal standard (200 nM labetalol) cooled to 4° C. After the last time point, the quench plates were mixed thoroughly and centrifuged for 15 minutes at 3700 rpm and 10° C. (Eppendorf 5804R). The supernatant was transferred to new 96 well plates and subjected to LC-MS analysis. The disappearance of the parent compound was determined.

All sample analysis was performed using a Vanquish Horizon UHPLC-system equipped with an autosampler, a binary pump, a column compartment and a diode array detector coupled to a Q Exactive focus hybrid quadrupole-Orbitrap mass spectrometer (Thermo Fisher Scientific) equipped with heated electrospray ion source.

The percentage of test compound remaining was defined as the ratio of test compound peak area at a specific time point and the peak area in the t=0 min samples multiplied by 100%.

The metabolic stability was evaluated by plotting the natural logarithm of the percentage test compound remaining versus time and performing linear regression. Using this graph, the following parameters were calculated: elimination constant k (min⁻¹)=−slope, in vitro half-life (t_{1/2})=ln(2)/k, in vitro intrinsic clearance Clᵢₙₜ(in μl/min/mg protein)=[ln(2)×incubation volume in μl/mg protein]/t_{1/2}.

Kinetic Solubility (PBS pH=7.4; 4 hr)

Test compounds were dissolved in DMSO to a concentration of 10 mM and further diluted to 100 μM in buffer (10 mM PBS, pH 7.4) in a 96 well plate at a final DMSO concentration of 1%. The plates were shaken for 4 h at room temperature in an Eppendorf Thermomixer. After incubation, the plates were centrifuged for 20 minutes at 4680 rpm. From the supernatant, 150 μL was transferred to a new 96 well plate and 50 μL DMSO was added to ensure continued dissolution. Samples were measured on LC-UV at injection volumes of 1 and 8 μL. Peak areas were determined and compared to peak areas obtained using calibration curves of the test compounds in DMSO. All sample analysis was performed using an Agilent 1290 HPLC-system equipped with an autosampler, a binary pump, a column compartment and a diode array detector.

e Log D (lipophilicity; pH=7.4)

Test compounds were dissolved in DMSO at a concentration of 10 mM and further diluted with methanol:water 1:1. Samples were analyzed using gradient HPLC with three different isocratic mobile phases of 0.25% octanol in methanol (60, 65 and 70%) and 20 mM MOPS buffer (pH 7.4) with decylamine. If needed, (for low E log Doct compounds) the isocratic mobile phases were adjusted to e.g. 40, 45 and 50% methanol. Peaks were detected using a diode array at absorbance 220-320 nm.

Capacity factors data (k′_=(tr−_t0)/t0) obtained at various amounts of methanol were extrapolated to 0% methanol and k′w values are determined using a linear procedure. E log Doct (7.4) is calculated using a series of reference standards with known Log D values. Each experiment was performed in triplicate.

All sample analysis was performed using an Agilent HPLC-system equipped with an autosampler, a binary pump, a column compartment and a diode array detector.

PAMPA (pH=7.4; Papp 10-6 cm/s)

PAMPA studies were conducted using the PAMPA Explorer kit (pION Inc.) and the double sink protocol (Avdeef, 2005). Stock solutions of all test compounds were dissolved in DMSO to a concentration of 10 mM. Each stock solution was diluted to 50 M in pH 7.4 Prisma HT buffer (pION) and 200 μl was added to each well of the donor plate in triplicate. The polyvinylidene fluoride (PVDF, 0.45 μm) filter membrane on the acceptor plate was coated with 5 μl gastrointestinal tract lipid formulation (GIT-0, pION) and to each well of the acceptor plate, 200 μl of acceptor sink buffer (pION) was added. The acceptor filter plate was then carefully placed on top of the donor plate to form a sandwich. The sandwich was incubated at 25° C. for 4 h without stirring. UV-vis spectra of the solutions in the blank, reference, acceptor and donor plates were measured using a microplate reader (Tecan Infinite 200PRO M Nano Plus). Permeability values were calculated using the PAMPA explorer software v. 3.8.0.2 (pION). Control incubations with ketoprofen (low permeability) and verapamil (high permeability) were included in each experiment.

Table 7 illustrates a A549 (KRAS G12S) pERK Dose Response study.

TABLE 7

| Attribute | Reference 1 | |
|---|---|---|
| pERK IC50 (A549) [12-point dose] | 7.2 | nM |
| A549 pERK:tERK ratio [10 nM; 2 hr] | 0.56 | |
| A375 pERK:tERK ratio [10 nM; 2 hr] | 0.59 | |
| CRAF A549 pMEK:tMEK [100 nM; 2 hr] | 2.09 | |
| Mouse Microsome (t_{1/2} min) | 18 | min |
| Mouse Clᵢₙₜ (μl/min/mg) | 78 | |
| Human Microsome (t_{1/2} min) | >90 | min |
| Kinetic Solubility (PBS pH = 7.4; 4 hr) | 36 | μM |

| | |
|---|---|
| eLogD (lipophilicity; pH = 7.4) | 3.6 |
| PAMPA (pH = 7.4; Pₐₚₚ 10⁻⁶ cm/s) | 29 |

Reference 1 is Selumetinib

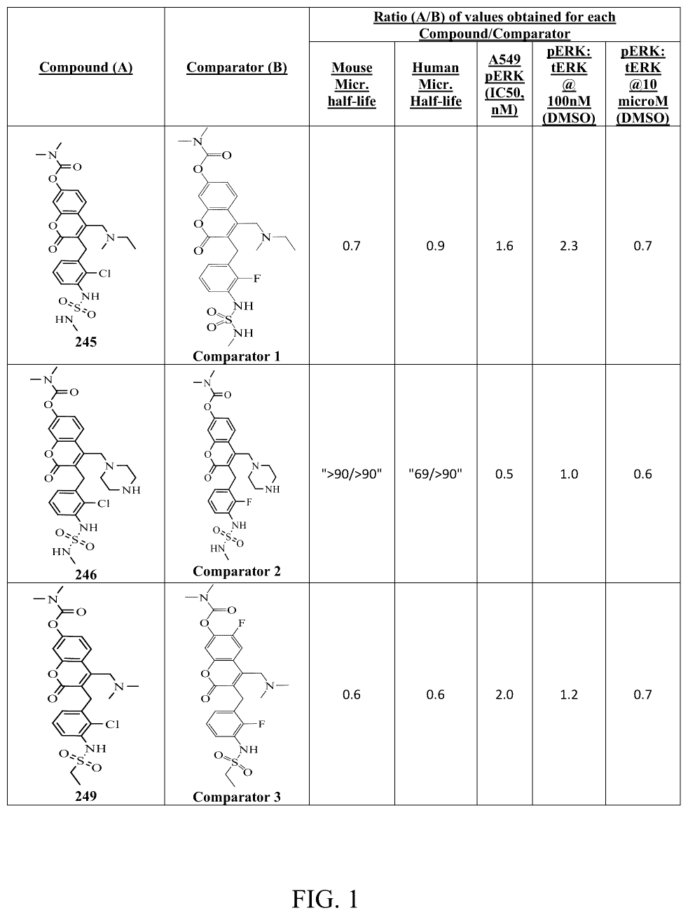

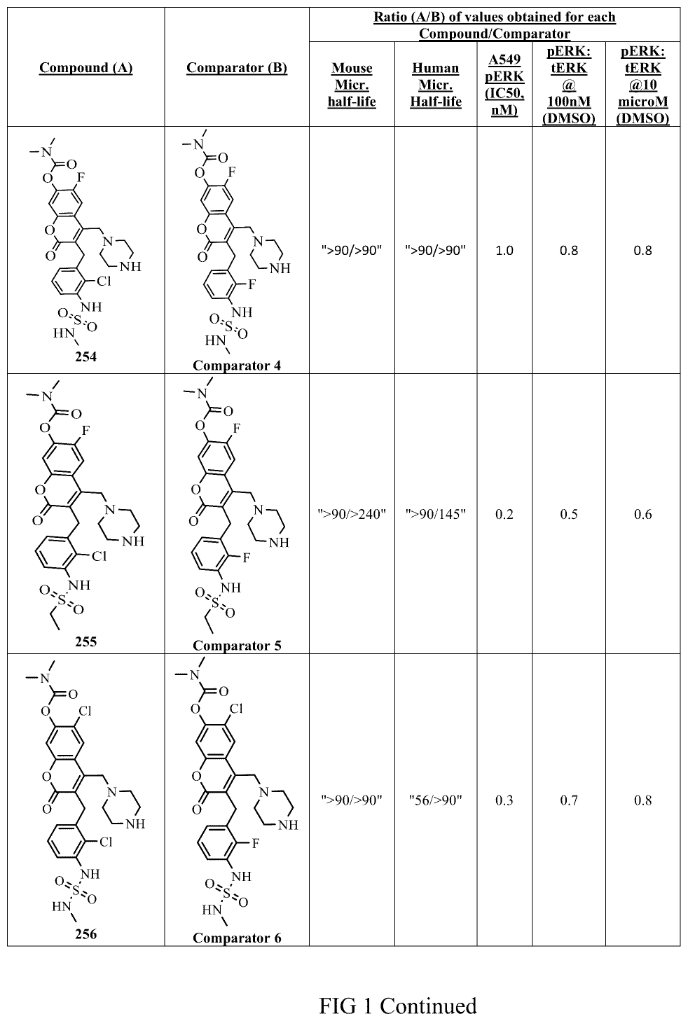

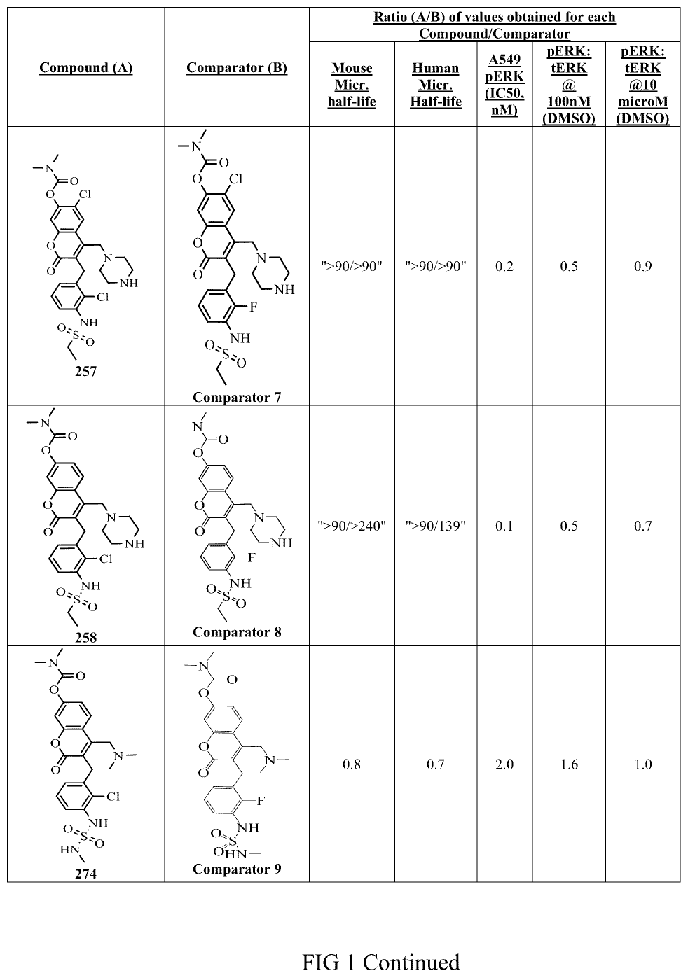

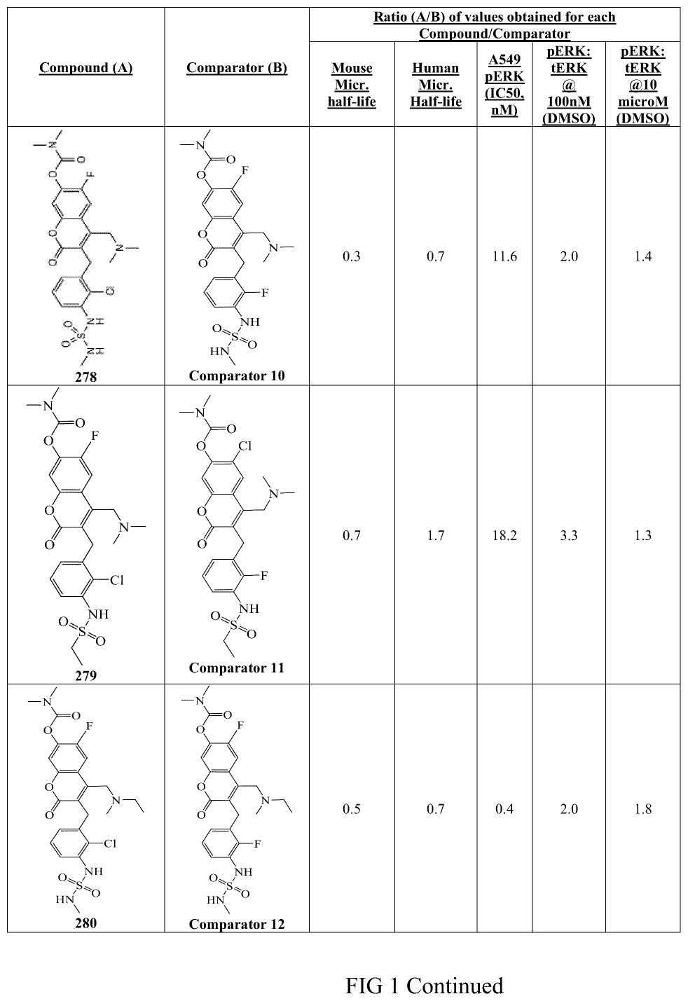

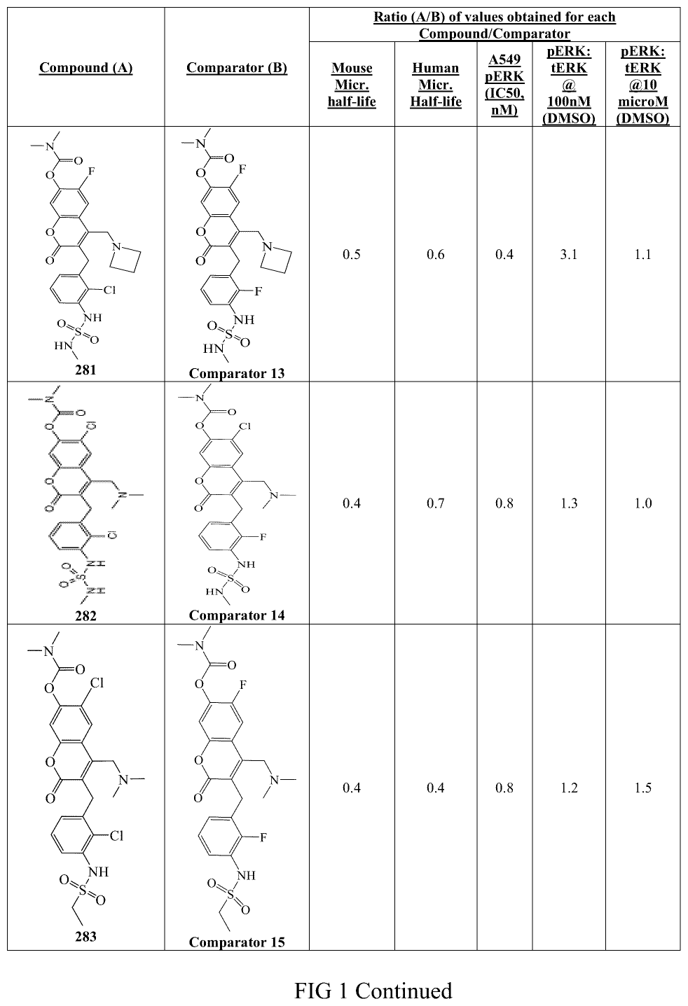

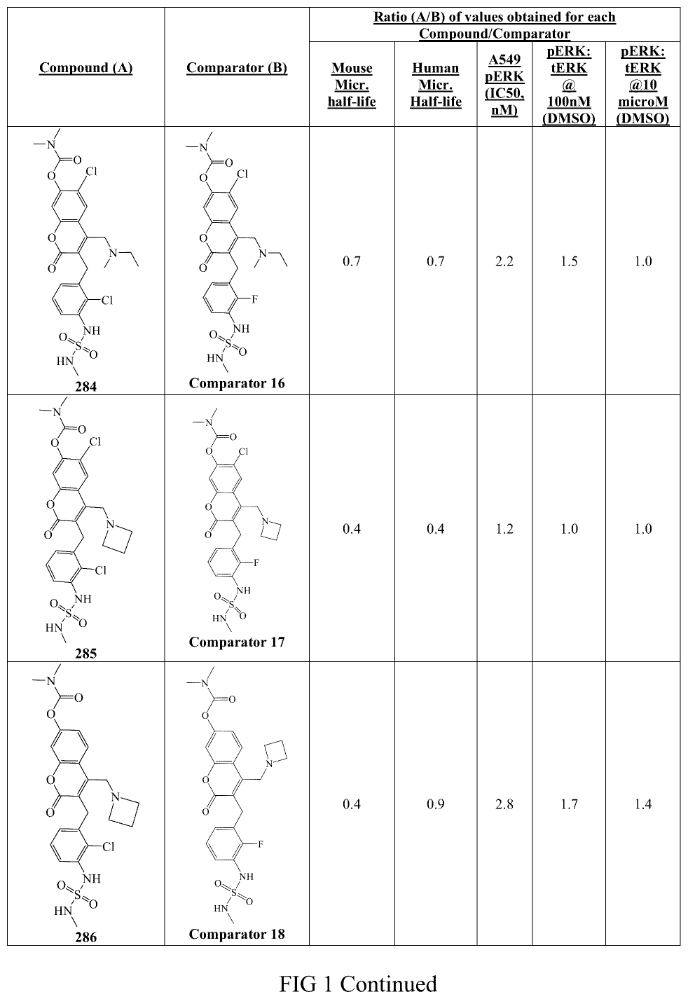

The results of a phospo ERK Screen in A549 (KRAS G12S) study is described in FIG. 1, together with the Mouse and Human Microsomal stability and the phosphorylated-ERK IC50 values in A549 cells.

### Example 38

### Drug Profiles

Table 8 describes the attributes of two compounds and a reference compound.

TABLE 8

| Attribute | Reference 2 | | Compound 9 | | Compound 19 | |
|---|---|---|---|---|---|---|
| pERK IC50 (A549) [12-point dose] | 7.2 | nM | 19.8 | nM | 40-50 | nM |
| A549 pERK:tERK ratio [10 nm; 2 hr] | 0.56 | | 0.62 | | n.t. | |
| A375 pERK:tERK ratio [10 nM; 2 hr] | 0.59 | | 0.71 | | n.t. | |
| CRAF A549 pMEK:tMEK [100 nM; 2 hr] | 2.09 | | 0.47 | | n.t. | |
| Mouse Microsome (t_{1/2} min) | 18 | min | 44 | min | 53 | min |
| Mouse Clint (μl/min/mg) | 78 | | 32 | | 26 | |
| Human Microsome (t_{1/2} min) | >90 | min | 35 | min | 38 | min |
| Human Clint (μl/min/mg) | <15 | | 40 | | 37 | |
| Kinetic Solubility (PBS pH = 7.4; 4 hr) | 36 | uM | >80 | uM | >80 | uM |

| | | | |
|---|---|---|---|
| eLogD (lipophilicity; pH = 7.4) | 3.6 | 3.0 | 2.9 |
| PAMPA (pH = 7.4; Papp 10 − 6 cm/s) | 29 | 37 | 51 |

### Example 39

### A549-pERK 10 nM

A549 (Cat No. CCL-185) cell line was obtained from American Type Culture Collection (ATCC). They were grown in T75 flasks in DMEM containing 10% FBS and Pen-Strep. at 37° C. in a humidified, 5% CO₂ incubator. The adherent cells were grown to about 90% confluency, culture medium was aspirated, and the cell layer was rinsed with PBS. Two mL trypsin solution (0.25%) was added to the flask and observed under an inverted microscope until cell layer is dispersed. Eight mL media was added, cells were spun down at 1000 g for 5 minutes. Cell pellet was re-suspended in 10 mL media and an appropriate volume was inoculated into a new culture flask. Cells were plated in a 6-well plate at a density of 250,000-300,000 cells/well in 3 mL media and incubated 37° C. in a humidified, 5% CO₂ incubator. Next day, 10 mM stock solutions of compounds were diluted 10- and 100-fold in DMSO to yield 100 and 10 μM solutions, respectively. These solutions were added to the cells (3 μL/well), mixed by swirling the plate, and incubated at 37° C. in a humidified, 5% CO₂ incubator for 2 hours. Cells were washed with PBS and scraped in 50 L of lysis buffer containing protease and phosphatase inhibitors. Cell lysates were stored at −20° C. Cell lysates were thawed and spun at 12,000 rpm for one minute, 3 μl of the supernatant was added to 500 μL of Coomassie blue reagent following by 500 μL of water. Absorbance was read at 595 nm after 10 minutes of incubation. Protein standards were used (0-20 mg/mL) to calculate protein concentrations of test samples. For electrophoresis 20 μg of protein was mixed with 5 μl of 4× Laemmle's sample buffer and 1 μl of 0.4 M DTT in a volume of 20 μl made up with lysis buffer. All samples were heated at 95° C. for 5 minutes, cooled to room temperature and spun down. Protein samples were loaded onto 4-12% polyacrylamide gels and run at 100V for about 1.5 hours till the blue dye reached the bottom. After the run, gel was removed and protein transfer was done using iBlot for 7 minutes, as per manufacturer's recommendations. After the transfer, nitrocellulose membrane was incubated on a shaker in 5 mL of blocking buffer at room temperature for 1 hr. The blot was then incubated overnight on a shaker in 5 ml of blocking buffer containing 0.2% Tween-20 and primary antibody, at room temperature. Anti-phospho-STAT3 antibody was used at a dilution of 1:500, the other 3 primary antibodies were used at a dilution of 1:1000. Next day, the blot was washed 3 times for 10 min each with 10 mL of TBST followed by incubation on a shaker in 5 ml of blocking buffer containing 0.2% Tween-20 and 0.5 μl of the IRDye labeled secondary antibodies, diluted 1:10000, at room temperature for 1 hr. The blot was then washed 3 times for 10 min each with 10 mL of TBST and dried between sheets of paper towels.

Antibodies: Phospho-STAT3 (S727), mouse polyclonal antibodies were obtained from BD Biosciences (Cat No. 612542), following 3 antibodies were obtained from Cell Signaling Technologies. Anti-STAT3, rabbit monoclonal antibodies (Cat No. 12640), Anti-ERK, mouse monoclonal antibodies (Cat No: 9107), and Anti-phospho-ERK, rabbit monoclonal antibodies (Cat No. 4377).

Secondary antibodies: IRDye 800CW goat anti-rabbit antibodies (LICOR Cat No. 926-32211), IRDye 680RD goat anti-rabbit antibodies (LICOR Cat No. 926-68071), IRDye 800CW goat anti-mouse antibodies (LICOR Cat No. 926-32210) and IRDye 680RD goat anti-mouse antibodies (LICOR Cat No. 926-68070)

Imaging was done using LICOR's Odyssey imaging system, quantitation was done using their software, Image Studio version 3.1.

A549 or 375 cells were treated in duplicate for two hours with compounds at varying concentrations. After two hours, the cells were lysed, snap frozen, and stored at −80° C. After storage, the lysate was quantified with the Bradford assay. Before the lysates were prepared to run on the Jess, the lysates were diluted to 1 mg/mL for pERK analysis and 1.5 mg/mL pMEK analysis using quantitative Western blotting. ERK and MEK phosphorylation levels were estimated by taking the ratio of phospho-protein to total protein and normalizing to the DMSO control. The results of this study are described in Tables 9-11.

TABLE 9
A549-pERK 10 nM, 2 h

| | A549-pERK | |
|---|---|---|
| Compound | (10 nM, 2 h) | |
| Compound 9 | 63.0 | (mean, n = 20) |
| Compound 10 | 27.7 | (mean, n = 2) |
| Compound 14 | 64 | |
| Compound 15 | 73.8 | |
| Compound 16 | 91.6 | |
| Compound 19 | 59.9 | |
| Compound 21 | 20.4 | (mean, n = 2) |
| Compound 24 | 42.4 | (mean, n = 3) |
| Compound 25 | 66.2 | (mean, n = 2) |
| Compound 26 | 94.2 | |
| Compound 29 | 96 | |
| Compound 30 | 54.3 | |
| Compound 31 | 66.7 | |
| Compound 33 | 55.4 | |
| Compound 48 | 94.3 | |
| Compound 50 | 103 | |
| Compound 56 | 92.9 | |
| Compound 57 | 106 | |
| Compound 57 | 119 | |
| Compound 58 | 98.7 | |
| Compound 58 | 108 | |
| Compound 59 | 105 | |
| Compound 60 | 91.1 | |
| Compound 62 | 73 | |
| Compound 63 | 88 | |
| Compound 64 | 73 | |
| Compound 65 | 100 | |
| Compound 66 | 104 | |
| Compound 67 | 112 | |
| Compound 68 | 68.9 | |
| Compound 69 | 101 | |
| Compound 70 | 103 | |
| Compound 71 | 99.4 | |
| Compound 72 | 85.4 | |
| Compound 73 | 77.7 | |
| Compound 74 | 75.3 | |
| Compound 75 | 69.3 | |
| Compound 76 | 62.1 | |
| Compound 77 | 67.2 | |
| Compound 78 | 78.6 | |
| Compound 79 | 49.4 | |
| Compound 79 | 75.6 | (mean, n = 2) |
| Compound 80 | 56.4 | |
| Compound 81 | 57.6 | |
| Compound 82 | 64.9 | |
| Compound 83 | 59.5 | |
| Compound 84 | 83.2 | |
| Compound 86 | 96.4 | |
| Compound 87 | 63.8 | |
| Compound 88 | 142 | |
| Compound 89 | 83.7 | |
| Compound 90 | 72.4 | (mean, n = 2) |
| Compound 91 | 74.6 | |
| Compound 92 | 93.8 | |
| Compound 93 | 108 | |
| Compound 94 | 93.5 | |
| Compound 95 | 80.2 | |
| Compound 96 | 102 | |
| Compound 97 | 56.5 | (mean, n = 2) |
| Compound 99 | 98.9 | |
| Compound 100 | 63.1 | (mean, n = 2) |
| Compound 104 | 77.3 | |
| Compound 107 | 18.0 | (mean, n = 2) |
| Compound 108 | 5.78 | (mean, n = 2) |
| Compound 109 | 4.51 | (mean, n = 2) |
| Compound 110 | 5.92 | (mean, n = 2) |
| Compound 111 | 4.63 | (mean, n = 2) |
| Compound 112 | 46.3 | (mean, n = 2) |
| Compound 113 | 24.7 | (mean, n = 2) |
| Compound 114 | 36.6 | (mean, n = 2) |
| Compound 115 | 13.5 | (mean, n = 2) |
| Compound 116 | 52.8 | (mean, n = 2) |
| Compound 117 | 58.6 | (mean, n = 3) |
| Compound 118 | 21.1 | (mean, n = 2) |
| Compound 119 | 64.8 | (mean, n = 2) |
| Compound 120 | 77.2 | |
| Compound 121 | 92.5 | |
| Compound 122 | 116 | |
| Compound 124 | 94.6 | |
| Compound 125 | 54.4 | (mean, n = 2) |
| Compound 126 | 91.4 | |
| Compound 127 | 110 | |
| Compound 128 | 66.3 | (mean, n = 2) |
| Compound 129 | 92.2 | |
| Compound 130 | 10.7 | (mean, n = 2) |
| Compound 132 | 62.6 | |
| Compound 133 | 88.1 | |
| Compound 134 | 33.1 | (mean, n = 2) |
| Compound 136 | 50.7 | (mean, n = 2) |
| Compound 137 | 69.3 | |
| Compound 138 | 96.9 | |
| Compound 139 | 53.5 | (mean, n = 2) |
| Compound 140 | 65.0 | (mean, n = 2) |
| Compound 141 | 77.8 | (mean, n = 2) |
| Compound 142 | 94.6 | |
| Compound 143 | 104 | |
| Compound 144 | 129 | |
| Compound 145 | 104 | |
| Compound 146 | 114 | |
| Compound 147 | 21.7 | |
| Compound 148 | 49.6 | |
| Compound 149 | 69.1 | |
| Compound 150 | 47.4 | |
| Compound 151 | 54.2 | |
| Compound 152 | 70.5 | |
| Compound 153 | 50.2 | |
| Compound 154 | 64 | |
| Compound 155 | 84.9 | |
| Compound 156 | 94.4 | |
| Compound 157 | 63.9 | |
| Compound 158 | 80 | |
| Compound 159 | 82.7 | |
| Compound 160 | 72.9 | |
| Compound 161 | 79.6 | |
| Compound 162 | 72.9 | |
| Compound 163 | 104 | |
| Compound 164 | 102 | |
| Compound 165 | 104 | |
| Compound 166 | 60.9 | |
| Compound 172 | 111 | |
| Compound 173 | 97.7 | |
| Compound 180 | 75 | |
| Compound 181 | 100 | |
| Compound 182 | 117 | |
| Compound 183 | 32.4 | (mean, n = 2) |
| Compound 187 | 85.9 | (mean, n = 2) |
| Compound 190 | 107 | |
| Compound 186 | 80.1 | (mean, n = 2) |
| Compound 191 | 131 | |
| Compound 193 | 90.7 | |
| Compound 194 | 83 | |
| Compound 195 | 113 | |
| Compound 196 | 106 | |
| Compound 197 | 31.7 | (mean, n = 2) |

| | |
|---|---|
| Compound 198 | 109 |
| Compound 199 | 118 |
| Compound 200 | 112 |
| Compound 201 | 119 |
| Compound 202 | 119 |
| Compound 203 | 115 |
| Compound 204 | 83.4 |
| Compound 205 | 73.7 |
| Compound 207 | 97.4 |
| Compound 208 | 105 |
| Compound 209 | 78.5 |
| Compound 210 | 81.9 |
| Compound 211 | 121 |
| Compound 212 | 100 |
| Compound 213 | 100 |
| Compound 214 | 98.6 |
| Compound 215 | 100 |
| Compound 216 | 89.5 |
| Compound 217 | 96.7 |
| Compound 218 | 113 |
| Compound 219 | 85.9 |
| Compound 220 | 109 |
| Compound 221 | 114 |
| Compound 222 | 150 |
| Compound 223 | 119 |
| Compound 224 | 119 |

TABLE 10
A549-pERK 10 μM, 2 h

| Compound | A549-pERK 10 uM, 2 h |
|---|---|
| Compound 9 | 1.205 (mean, n = 2) |
| Compound 57 | 7.33 |
| Compound 100 | 0.82 |
| Compound 105 | 7.39 |
| Compound 106 | 27.88 |
| Compound 133 | 23.41 |
| Compound 181 | 22.23 |
| Compound 182 | 6.77 |
| Compound 183 | 0.82 |
| Compound 186 | 0.74 |
| Compound 187 | 0.91 |
| Compound 190 | 15.28 |
| Compound 191 | 8.6 |
| Compound 207 | 11.35 |
| Compound 208 | 23.64 |
| Compound 209 | 34.23 |
| Compound 210 | 78.26 |
| Compound 211 | 70.75 |
| Compound 212 | 13.03 |
| Compound 213 | 70.14 |
| Compound 214 | 7.34 |
| Compound 215 | 11.77 |
| Compound 216 | 4.51 |
| Compound 217 | 11.2 |
| Compound 218 | 9.46 |
| Compound 219 | 13.94 |
| Compound 220 | 4.28 |
| Compound 221 | 17.24 |
| Compound 222 | 9.32 |
| Compound 223 | 3.04 |
| Compound 224 | 28.27 |

TABLE 11
A549-CAR BP 100 nM, 2 h

| | A549-CRAF BP |
|---|---|
| Compound | 100 nM, 2 h |
| Compound 9 | 44.9 (mean, n = 8) |
| Compound 10 | 42.2 |
| Compound 14 | 59.1 |
| Compound 19 | 51.9 |
| Compound 21 | 47.9 (mean, n = 2) |
| Compound 24 | 55.9 (mean, n = 2) |
| Compound 25 | 71 |
| Compound 33 | 51.4 |
| Compound 73 | 33.9 |
| Compound 74 | 59.2 |
| Compound 75 | 66.5 |
| Compound 76 | 53.9 |
| Compound 77 | 78 |
| Compound 78 | 64.2 |
| Compound 79 | 45.2 |
| Compound 79 | 64.9 |
| Compound 80 | 56.1 |
| Compound 81 | 78.3 |
| Compound 82 | 72.3 |
| Compound 83 | 68.4 |
| Compound 84 | 87.3 |
| Compound 86 | 53.2 |
| Compound 87 | 95.8 |
| Compound 88 | 53.6 |
| Compound 90 | 71 |
| Compound 91 | 84.2 |
| Compound 97 | 52.1 (mean, n = 2) |
| Compound 100 | 65.3 |
| Compound 107 | 39.9 |
| Compound 108 | 47.9 (mean, n = 2) |
| Compound 109 | 41.9 (mean, n = 2) |
| Compound 110 | 56.6 (mean, n = 2) |
| Compound 111 | 48.6 (mean, n = 2) |
| Compound 112 | 52.9 (mean, n = 2) |
| Compound 113 | 48.0 (mean, n = 2) |
| Compound 114 | 59.9 (mean, n = 3) |
| Compound 115 | 39.2 (mean, n = 2) |
| Compound 116 | 53.9 (mean, n = 2) |
| Compound 117 | 56.8 (mean, n = 2) |
| Compound 118 | 39.1 (mean, n = 2) |
| Compound 119 | 66.1 |
| Compound 125 | 41.7 |
| Compound 128 | 37.7 |
| Compound 130 | 36.9 |
| Compound 132 | 57.8 |
| Compound 134 | 26.9 |
| Compound 136 | 44.4 (mean, n = 2) |
| Compound 139 | 62.1 |
| Compound 140 | 70.3 |
| Compound 144 | 80.3 |
| Compound 147 | 46.4 |
| Compound 148 | 56.5 |
| Compound 150 | 90.2 |
| Compound 151 | 70.9 |
| Compound 153 | 54.1 |
| Compound 183 | 65.9 (mean, n = 2) |
| Compound 197 | 48.5 (mean, n = 2) |

### Example 40

### A549-pERK 10 nM

A549 or 375 cells were treated in duplicate for two hours with compounds at varying concentrations. After two hours, the cells were lysed, snap frozen, and stored at −80° C. After storage, the lysate was quantified with the Bradford assay. Before the lysates were prepared to run on the Jess, the lysates were diluted to 1 mg/mL for pERK analysis and 1.5 mg/mL pMEK analysis using quantitative Western blotting. ERK and MEK phosphorylation levels were estimated by taking the ratio of phospho-protein to total protein and normalizing to the DMSO control. The results of this study are described in Tables 12-13.

TABLE 12
A549-pERK 10 nM, 2 h

| | A549-pERK |
|---|---|
| Compound | (10 nM, 2 h) |
| Compound 236 | 94.1 |
| Compound 237 | 114 |
| Compound 238 | 68.4 |
| Compound 239 | 96.1 |
| Compound 240 | 66.0 |
| Compound 241 | 96.6 |
| Compound 242 | 69.7 |
| Compound 243 | 86.7 |
| Compound 244 | 44.7 |
| Compound 245 | 68.8 |
| Compound 246 | 19.0 |
| Compound 247 | 105 |
| Compound 248 | 80.9 |
| Compound 249 | — |
| Compound 250 | — |
| Compound 251 | — |
| Compound 252 | — |
| Compound 253 | — |
| Compound 254 | 26.8 |
| Compound 255 | 37.1 |
| Compound 256 | — |
| Compound 257 | — |
| Compound 258 | — |

TABLE 13
A549-pMEK 100 nM, 2 h

| | A549-pMEK |
|---|---|
| Compound | 100 nM, 2 h |
| Compound 236 | — |
| Compound 237 | — |
| Compound 238 | — |
| Compound 239 | — |
| Compound 240 | 34.2 |
| Compound 241 | — |
| Compound 242 | 33.2 |
| Compound 243 | — |
| Compound 244 | 26.1 |
| Compound 245 | — |
| Compound 246 | — |
| Compound 247 | — |
| Compound 248 | — |
| Compound 249 | — |
| Compound 250 | — |
| Compound 251 | — |
| Compound 252 | — |
| Compound 253 | — |
| Compound 254 | — |
| Compound 255 | — |
| Compound 256 | — |
| Compound 257 | — |
| Compound 258 | — |

### Example 41

### Cell-Based pERK and pMEK Dose Response Study

Materials and Methods: Are described in EXAMPLE 35.

Table 14 illustrates the results of a PAMPA study.

| | PAMPA (pH = |
|---|---|
| Compound | 7.4; Pₐₚₚ 10⁻⁶ cm/s) |
| Compound 236 | 52 |
| Compound 237 | 50 |
| Compound 238 | 62 |
| Compound 239 | 0.8 |
| Compound 240 | 62 |
| Compound 241 | 69 |
| Compound 242 | 72 |
| Compound 243 | 48 |
| Compound 244 | 8 |
| Compound 245 | 53 |
| Compound 246 | 2 |
| Compound 247 | 7 |
| Compound 248 | 60 |
| Compound 249 | 64 |
| Compound 250 | — |
| Compound 251 | — |
| Compound 252 | 15 |
| Compound 253 | 48 |
| Compound 254 | 2 |
| Compound 255 | 5 |
| Compound 256 | 4 |
| Compound 257 | 8 |
| Compound 258 | 1 |

The results of a phospho-ERK Screen in A549 (KRAS G12S) study is described in Table 15 below.

TABLE 15
Phospho-ERK Screen in A549 (KRAS G12S): 10 μM for 2 hours

| Compound | pERK:tERK (10 nM 2 h A549) |
|---|---|
| 245 | 69 |
| 246 | 19 |
| 248 | 81 |
| 254 | 27 |
| 255 | 37 |
| 256 | 14 |
| 257 | 17 |
| 136 | 39 (n = 3) |
| 147 | 28 (n = 2) |
| 167 | 27 (n = 2) |
| 168 | 50 (n = 2) |
| 25 | 66 (n = 2) |
| 261 | 68 |
| 168 | 53 (n = 2) |
| 9 | 65 (n = 27) |

### Example 42

### pERK:Total-ERK Dose Response

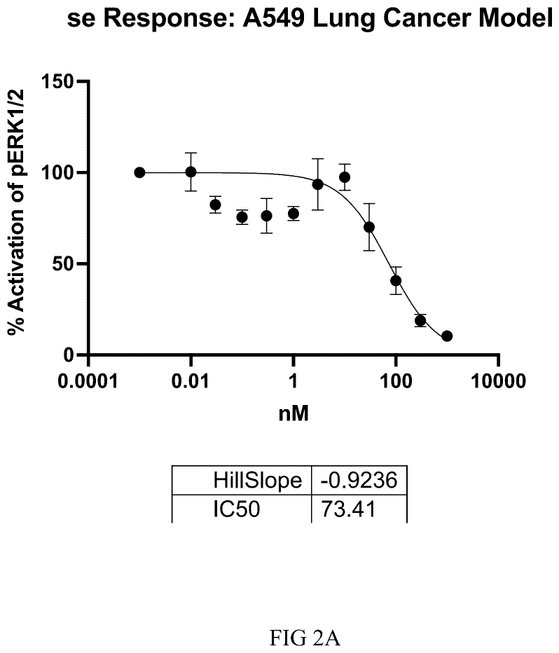

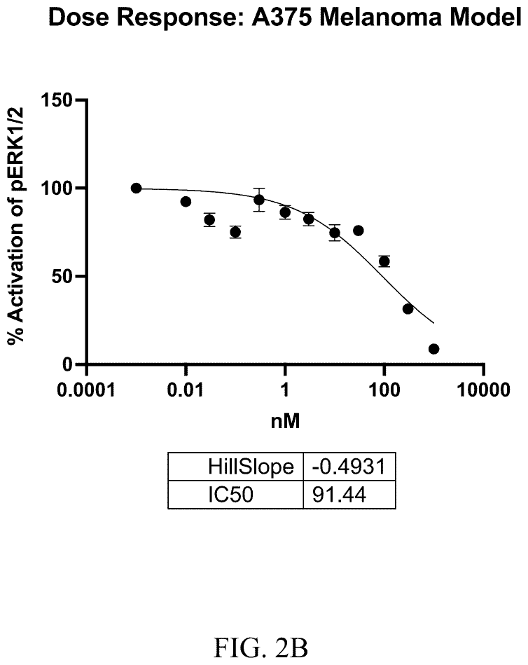

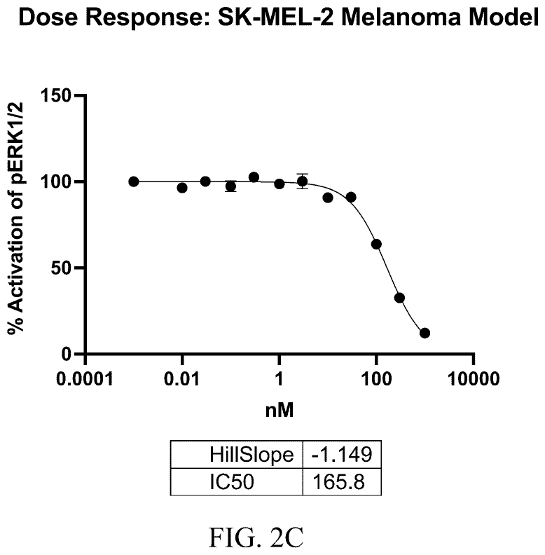

A549, A375 or SK-MEL-2 Melanoma Model cells were treated in duplicate for two hours with compounds at varying concentrations. After two hours, the cells were lysed, snap frozen, and stored at −80° C. After storage, the lysate was quantified with the Bradford assay. Before the lysates were prepared to run on the Jess, the lysates were diluted to 1 mg/mL for pERK analysis and 1.5 mg/mL pMEK analysis using quantitative Western blotting. ERK and MEK phosphorylation levels were estimated by taking the ratio of phospho-protein to total protein and normalizing to the DMSO control. The results of this study are described in FIG. 2A-2C.

### Example 43

### pERK:Total ERK Ratio and pMEK:Total MEK Ratio

Compound 274 was evaluated at 100 nM doses for impact on pERK:tERK and pMEK:tMEK levels across a panel of 9 melanoma tumor models compared to Selumetinib and Binimetinib. The nine models are described in Table 16. Materials and methods are described for in EXAMPLE 35 with the tumor models detailed in Table 16.

TABLE 16

| Model | HRAS | NRAS | BRAF | NF1 |
|---|---|---|---|---|
| SK-MEL-2 | | p.Q61R | | |
| MM127 | | p.G13R | p.G464E | |
| MM415 | | p.Q61L | | |
| MEL-JUSO | p.G13D | p.Q61L | | |
| SK-MEL-30 | | p.Q61K | | |
| Hs852T | | p.G12V | | |
| MeWo | | | | LoF |
| A375 | | | p.V600E | |
| SK-MEL-28 | | | p.V600E | |

The results are presented in Tables 17-8 below. The results show that Compound 274 is a dual-MEK inhibitor due to the reductions in both pERK and pMEK observed across RAS mutant and NF-1 loss of function (LoF) tumor models.

TABLE 17
pERK/tERK ratio normalized to DMSO

| Cell line | Binimetinib | Selumetinib | Compound 274 |
|---|---|---|---|
| SK-MEL-2 | 0.125 | 0.191 | 0.439 |
| MM127 | 0.199 | 0.255 | 0.533 |
| MM415 | 0.056 | 0.114 | 0.423 |
| MEL-JUSO | 0.139 | 0.179 | 0.631 |
| SK-MEL-30 | 0.082 | 0.163 | 0.521 |
| Hs852.T | 0.203 | 0.293 | 0.731 |
| MeWo | 0.161 | 0.196 | 0.519 |
| A375 | 0.136 | 0.199 | 0.477 |
| SK-MEL-28 | 0.079 | 0.152 | 0.745 |

TABLE 18
pMEK/tMEK ratio normalized to DMSO

| Cell line | Binimetinib | Selumetinib | Compound 274 |
|---|---|---|---|
| SK-MEL-2 | 2.587 | 2.055 | 0.518 |
| MM127 | 3.009 | 2.341 | 0.773 |
| MM415 | 3.055 | 2.386 | 0.58 |
| MEL-JUSO | 3.291 | 2.988 | 0.548 |
| SK-MEL-30 | 2.649 | 2.177 | 0.609 |
| Hs852.T | 6.489 | 4.945 | 0.672 |
| MeWo | 7.42 | 5.483 | 0.904 |
| A375 | 1.062 | 0.962 | 0.585 |
| SK-MEL-28 | 1.055 | 0.984 | 0.763 |

### Example 44

### Mouse Pharmacokinetics (PK)

Female mice (BALC/c) received a single dose (3 mice/treatment) of vehicle (10% 1N HCl:90% [20% Captisol in saline pH adjusted to 5.0±0.1] adjusted to pH 3.0±0.1) containing 5× compounds at 10 mg/kg by gavage (p.o.) and at 0.5, 1, 2, 4, 8, 12 and 24-hour post dose humanely euthanized.

Blood was obtained through cardiac puncture into K+EDTA tubes, mixed by inversion and centrifuged to obtain the plasma. Blood will be kept on ice or at approximately 4° C. prior. Centrifugation will take place within 30 minutes of blood collection affording plasma samples. Plasma samples will be frozen as soon as possible after centrifugation and stored at −80° C. until analysis. All samples will be analysed by means of LC-MS/MS. Method development for bioanalysis will be performed by pharmacokinetics Department of Crown Bioscience (Taicang) Inc. using LC-MS/MS analysis (using Waters HSS T3 2.1×50 mm (1.8 um) LC column, and an API-4000 Electrospray MS unit). The results of a kinetic solubility (free base) study are represented in Table 19.

TABLE 19

| Compound ID | Kinetic Solubility [μM] |
|---|---|
| 9 | >80 |
| 10 | >80 |
| 24 | 57 |
| 25 | >80 |
| 120 | >80 |
| 146 | >80 |
| 161 | >80 |
| 166 | 80 |
| 238 | 40 |
| 240 | 67 |
| 245 | 28 |
| 248 | >80 |
| 256 | >80 |
| 257 | >80 |
| 274 | 78 |

The results of the pharmacokinetics (oral cassette dosing) study are represented in Table 20.

TABLE 20

| Compound | T_{1/2} | Tₘₐₓ | Cₘₐₓ | AUC0-t | AUC0-∞ |
|---|---|---|---|---|---|
| ID | (hr) | (hr) | (ng/ml) | (ng*hr/ml) | (ng*hr/ml) |
| 9 | 1.1 | 0.083 | 725 | 441 | 445 |
| 10 | — | 8 | 3.6 | 23.1 | — |
| 24 | — | 1 | 422.7 | 850.1 | — |
| 25 | 1.1 | 0.083 | 1037 | 1336 | 1426 |
| 120 | 0.3 | 0.083 | 1422.7 | 669.3 | 673.2 |
| 146 | — | 0.5 | 4.9 | 23.2 | — |
| 161 | 0.9 | 0.083 | 857 | 416 | 434 |
| 166 | — | 2 | 9.1 | 11.8 | — |
| 238 | 1.0 | 0.083 | 857 | 416 | 434 |
| 240 | — | 0.083 | 865 | 290 | — |
| 245 | — | 0.083 | 384 | 154 | — |
| 248 | 5.2 | 0.5 | 8.3 | 19.5 | 28.3 |
| 256 | — | 0.5 | 8.3 | 19.5 | 28.3 |
| 257 | — | 4 | 20.3 | 103 | — |
| 274 | 0.3 | 0.083 | 795.7 | 392 | 395.8 |

BALB/c immunocompetent mice 0.083, 0.5, 1, 2, 4, 6, 12, 24 hour after 5 mg/kg p.o. dose

### Example 45

### Colon-26 Syngeneic Colorectal Tumor Pharmacology Study

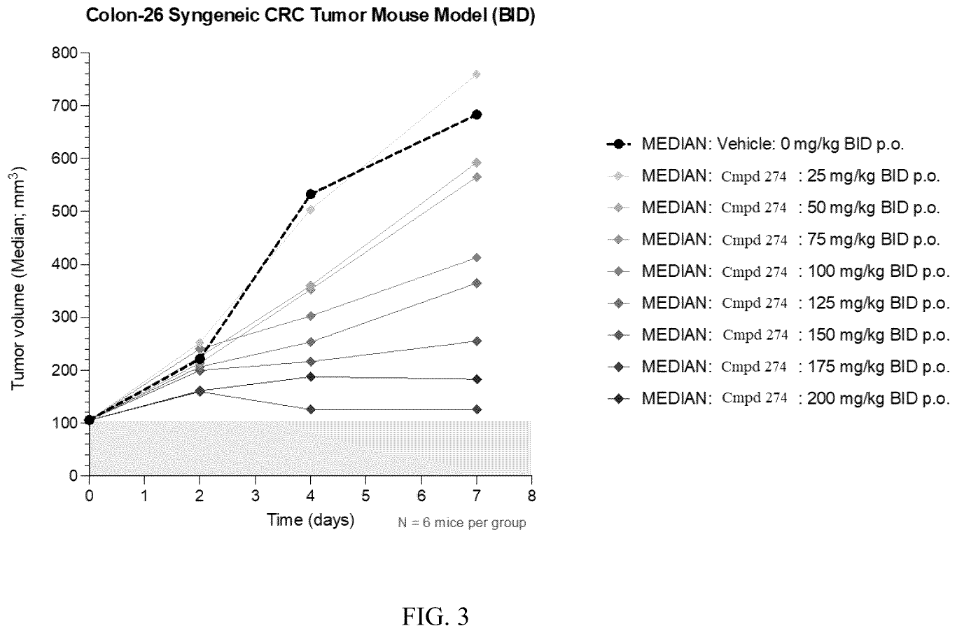

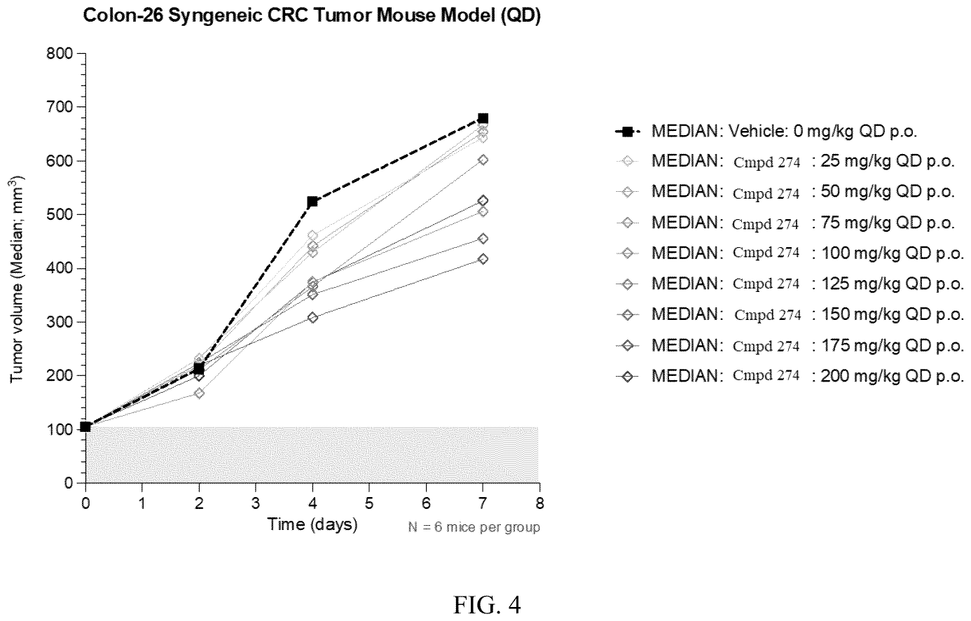

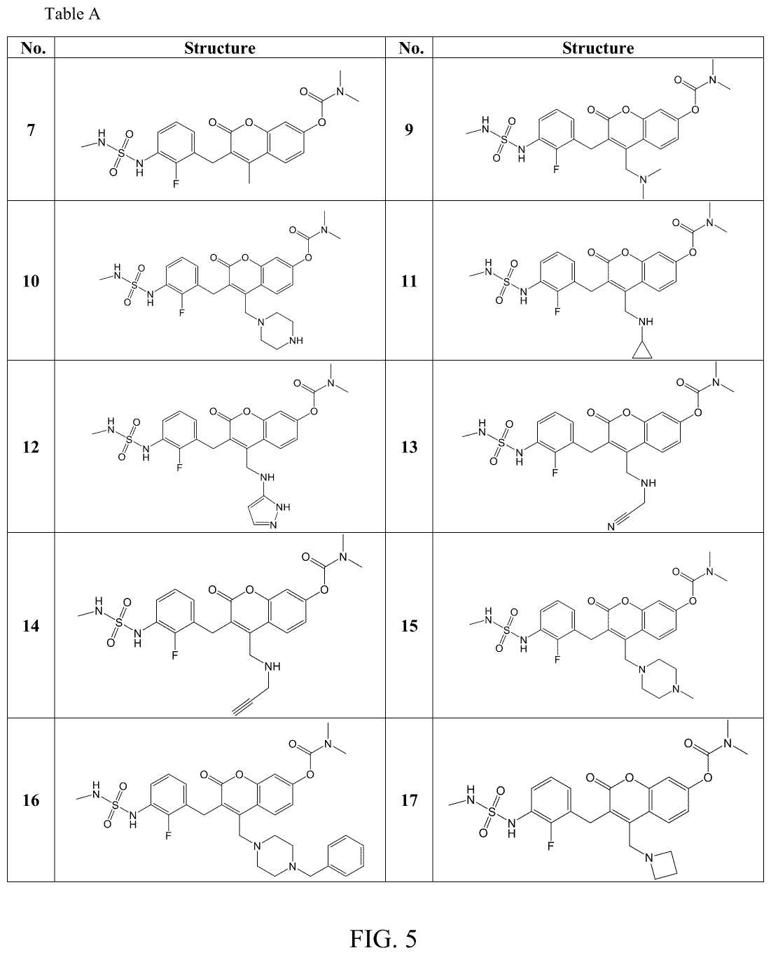

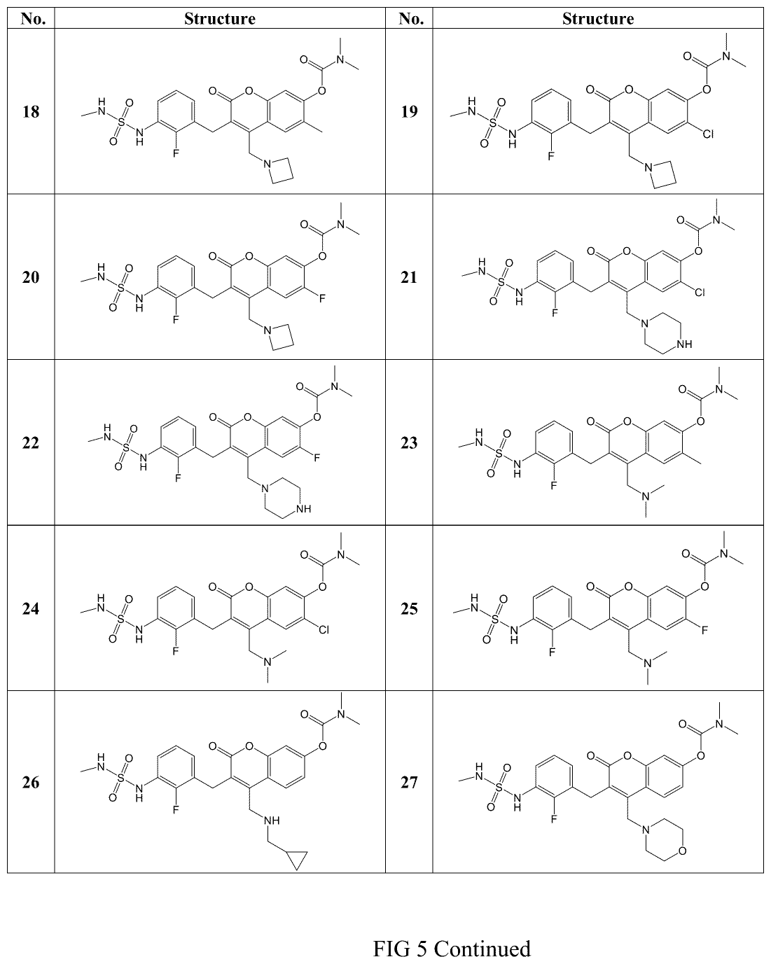

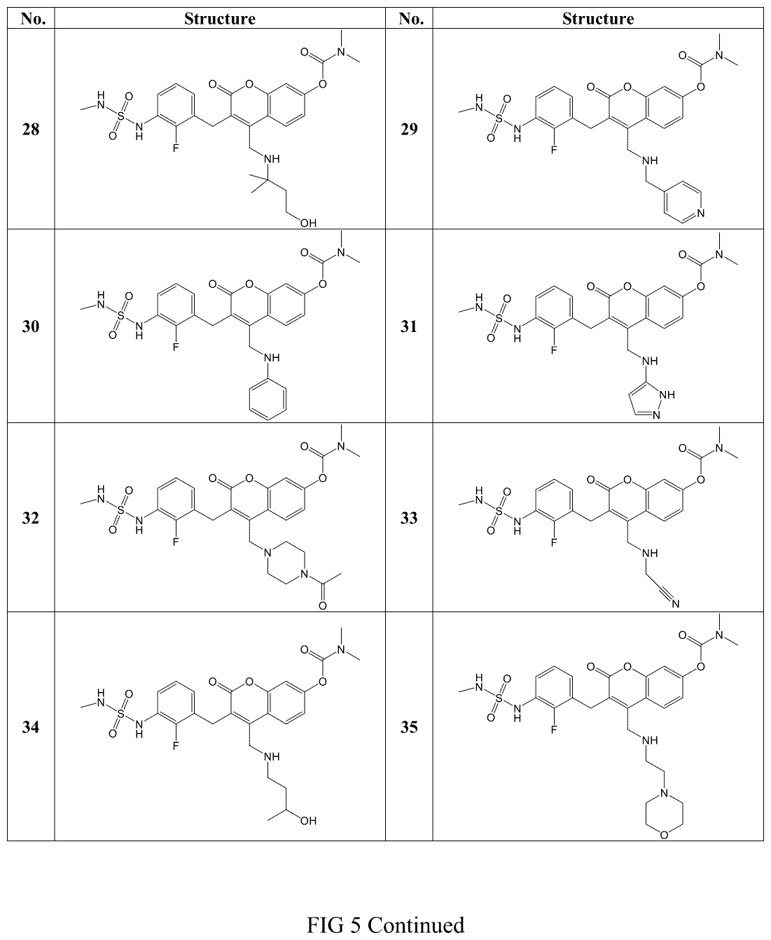

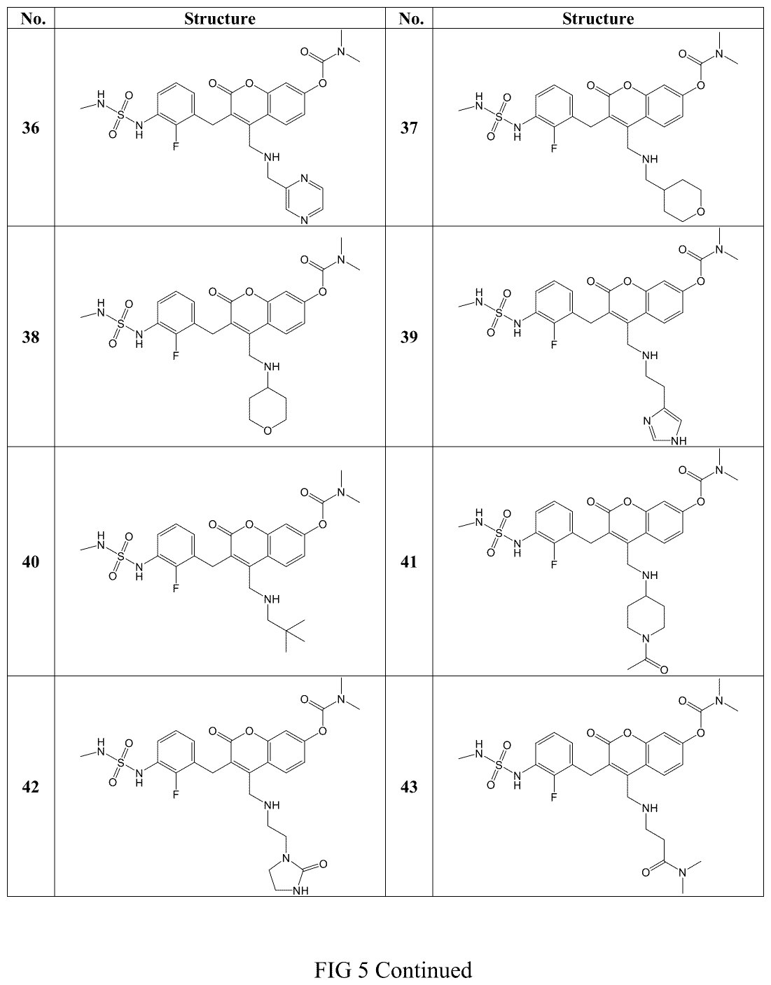

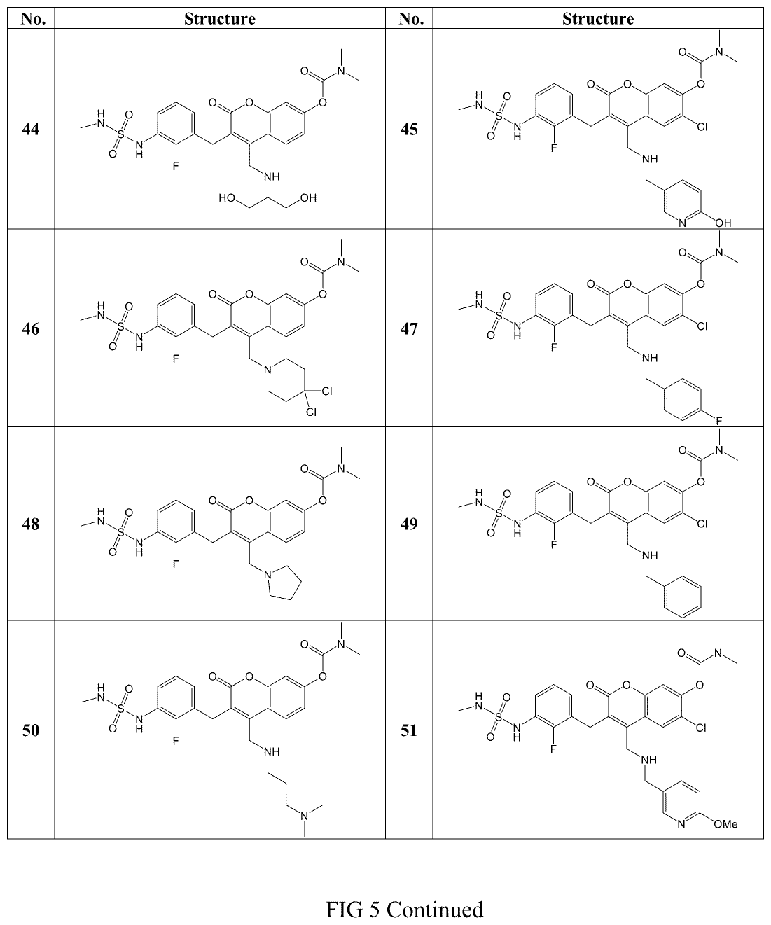

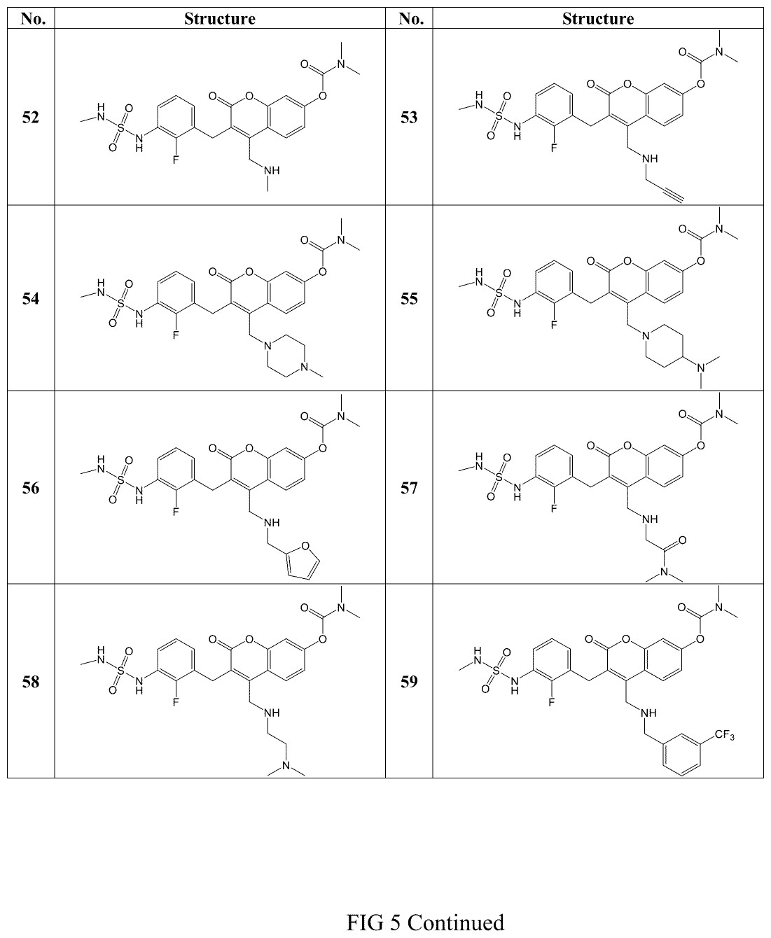

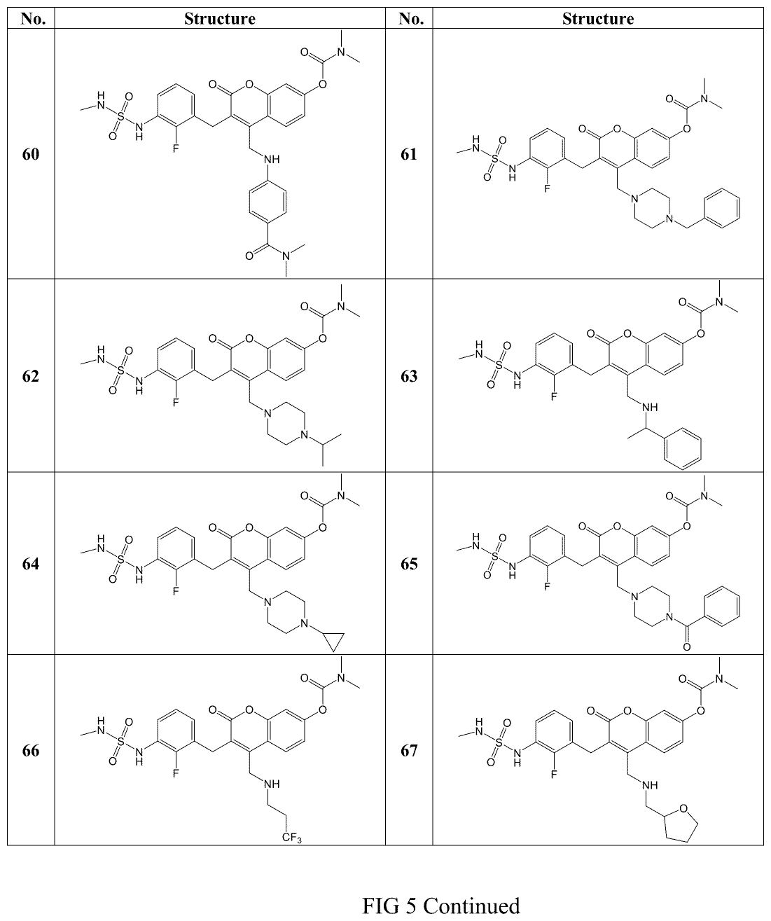

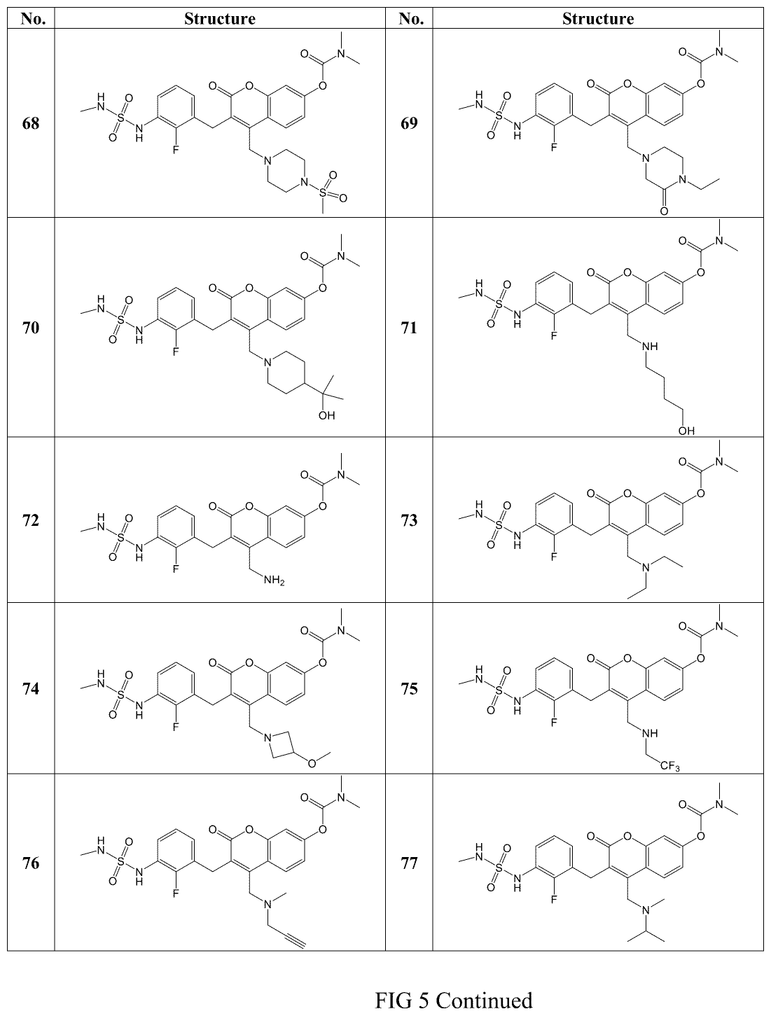

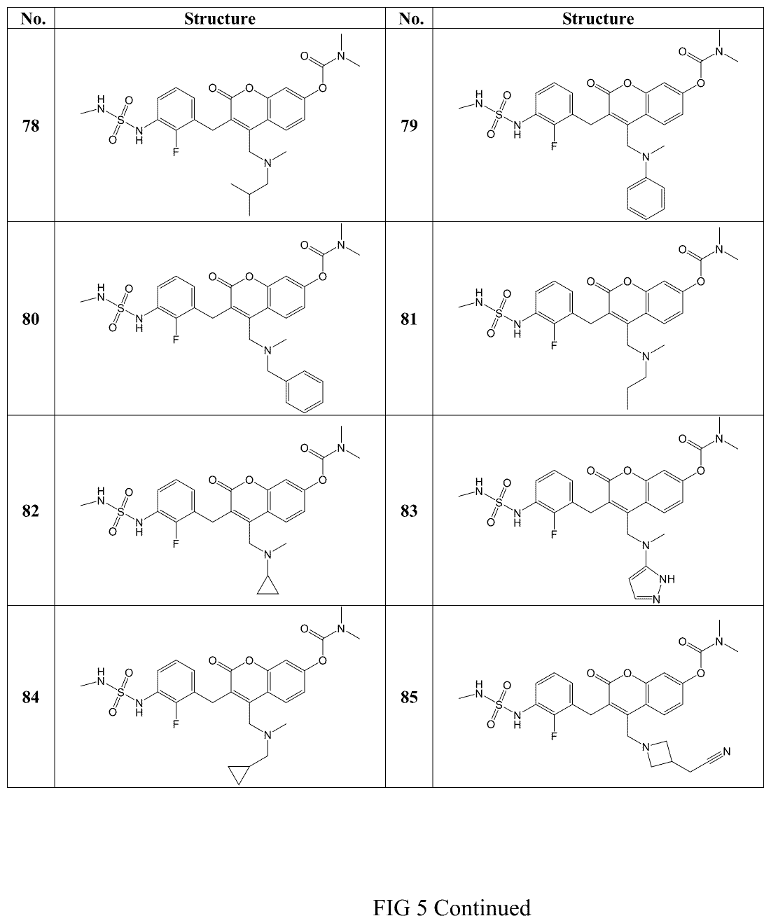

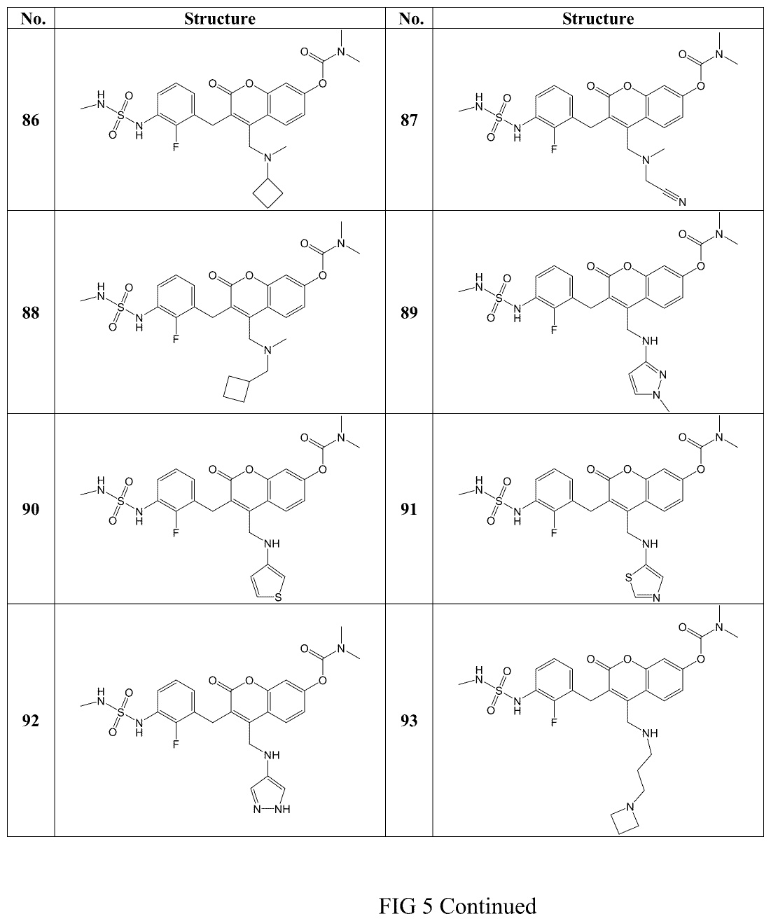

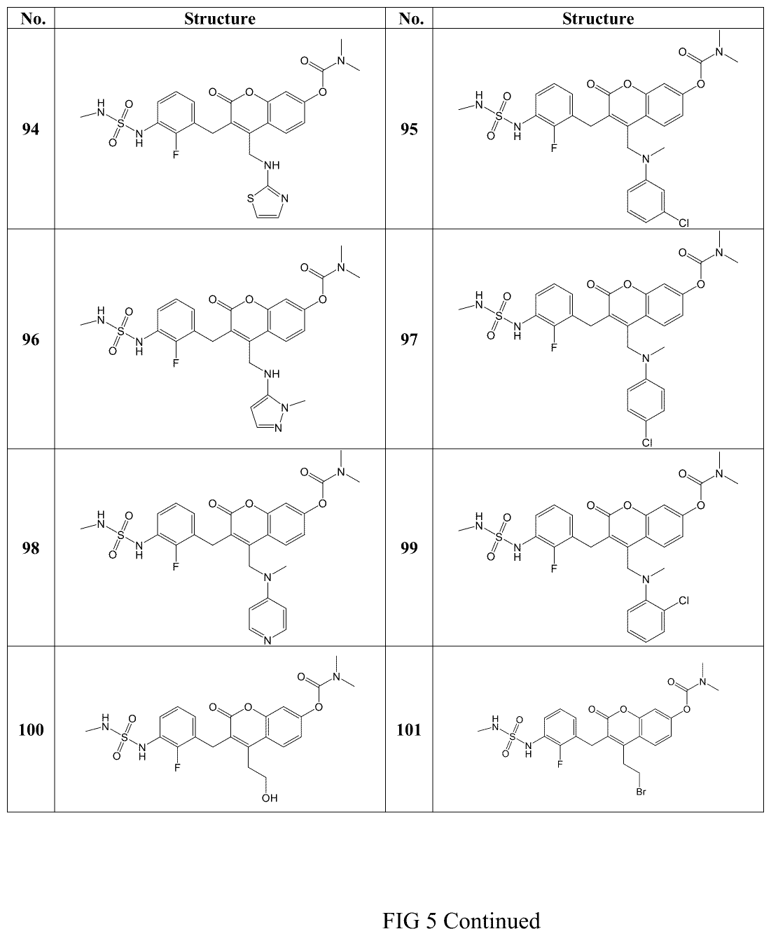

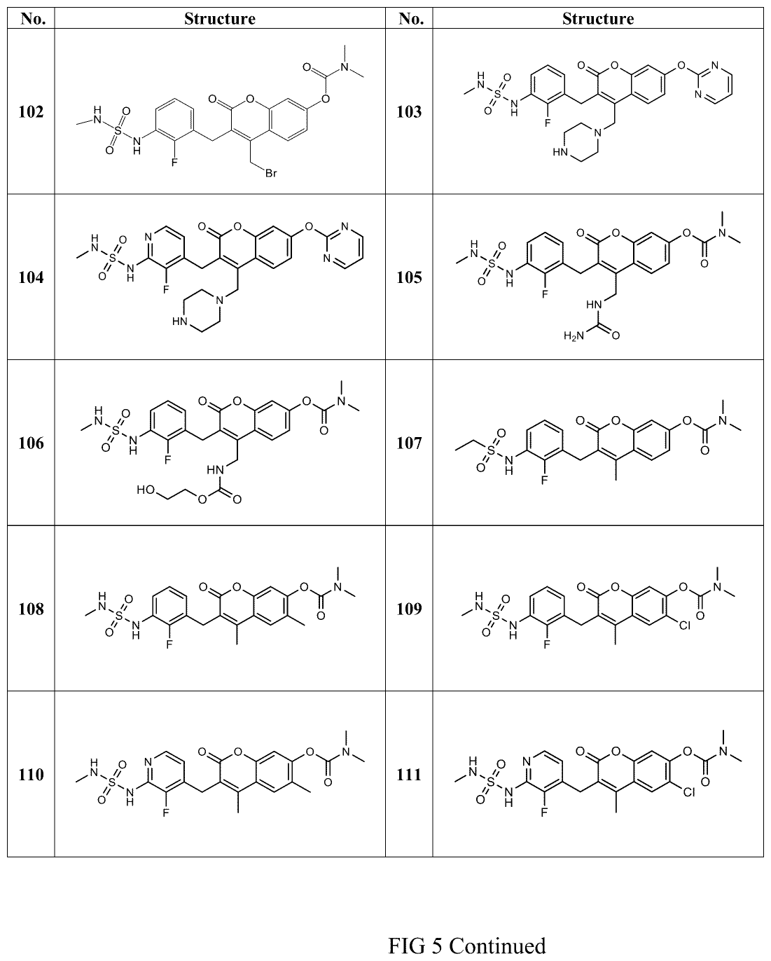

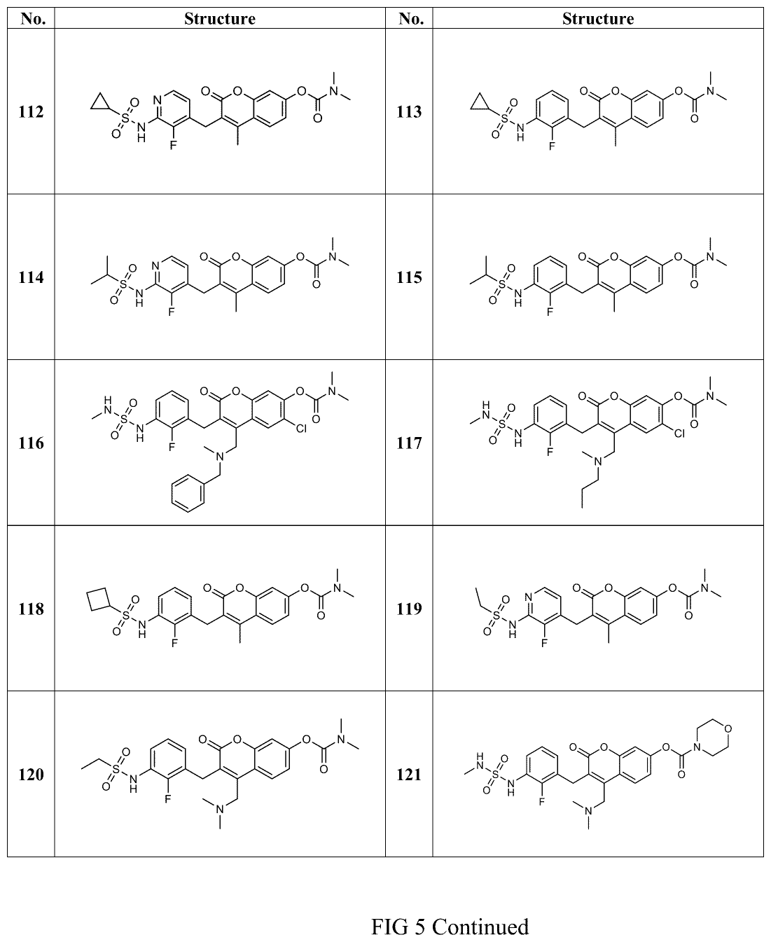

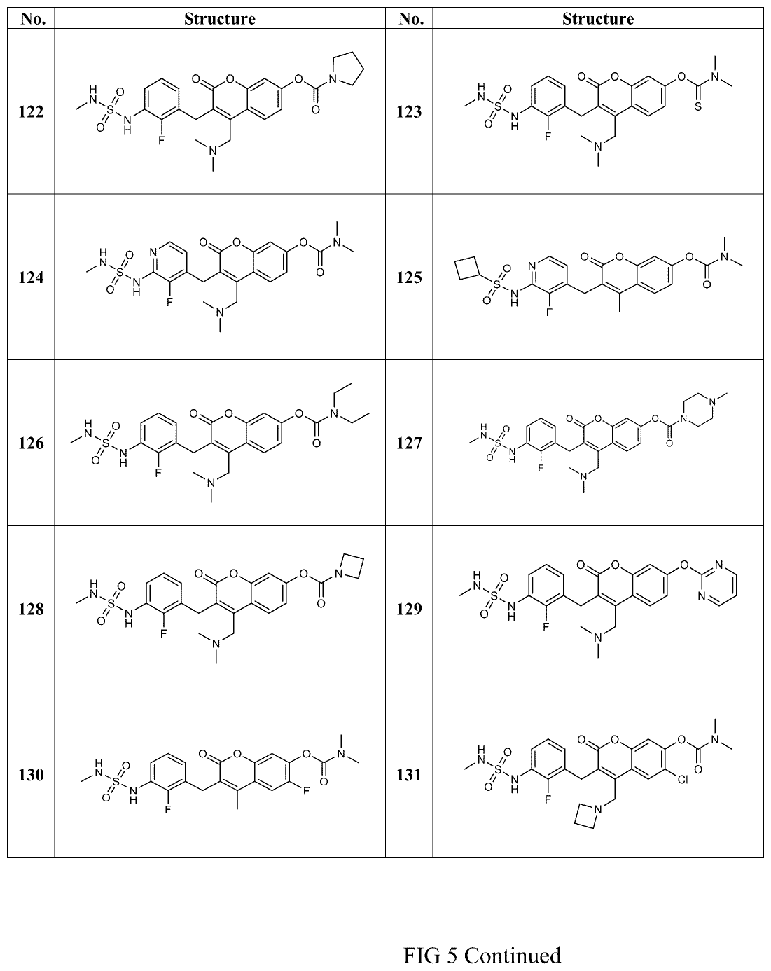

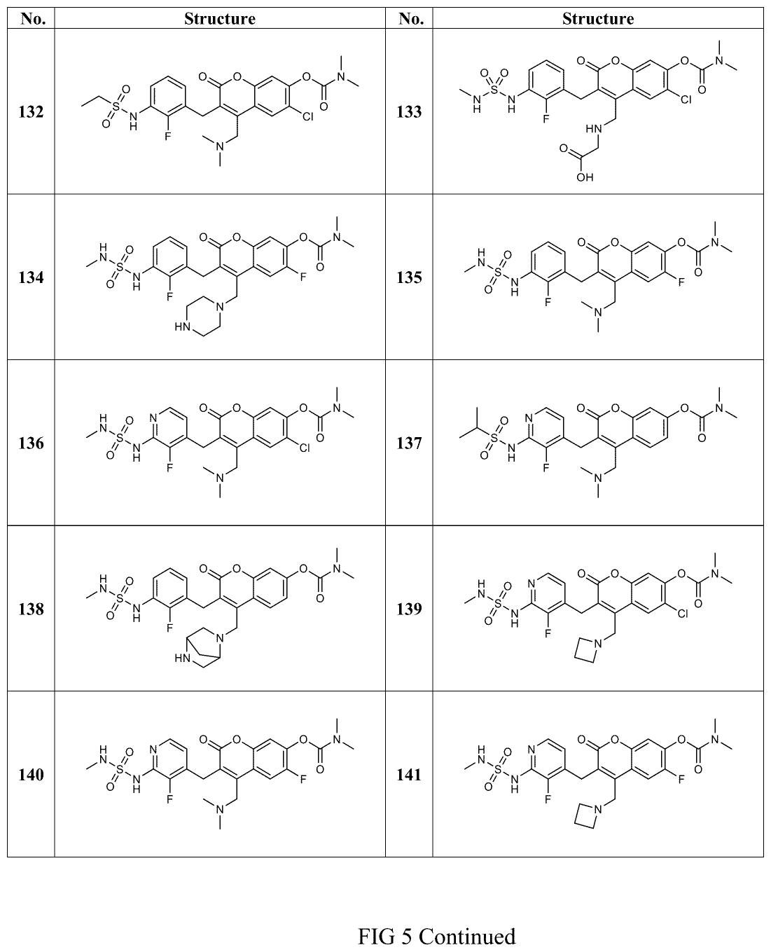

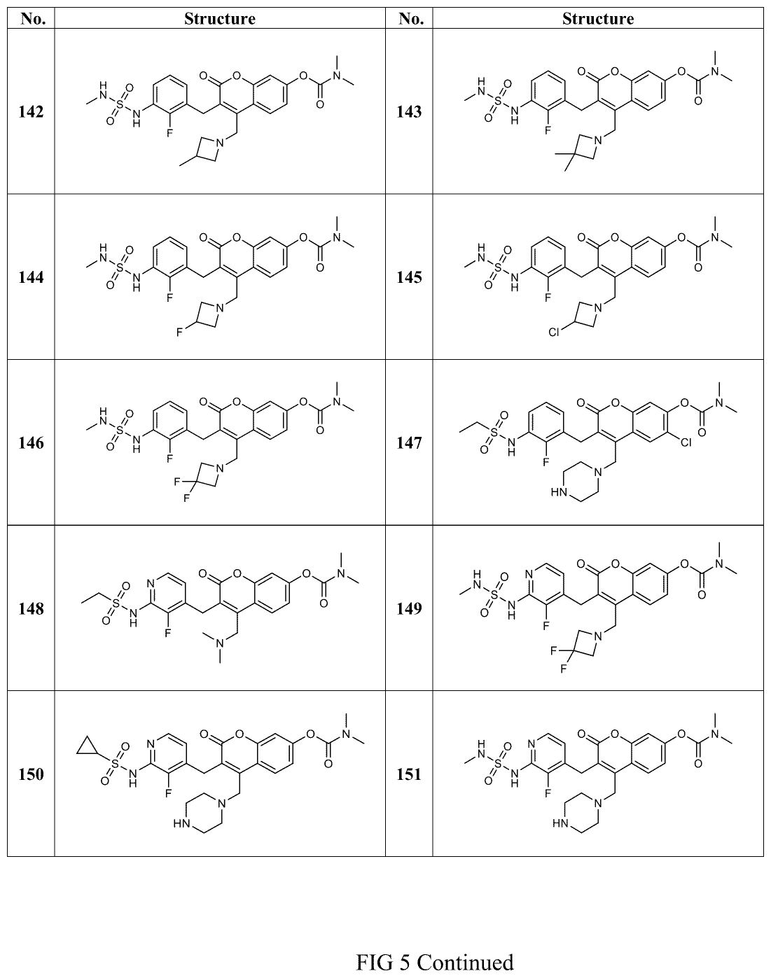

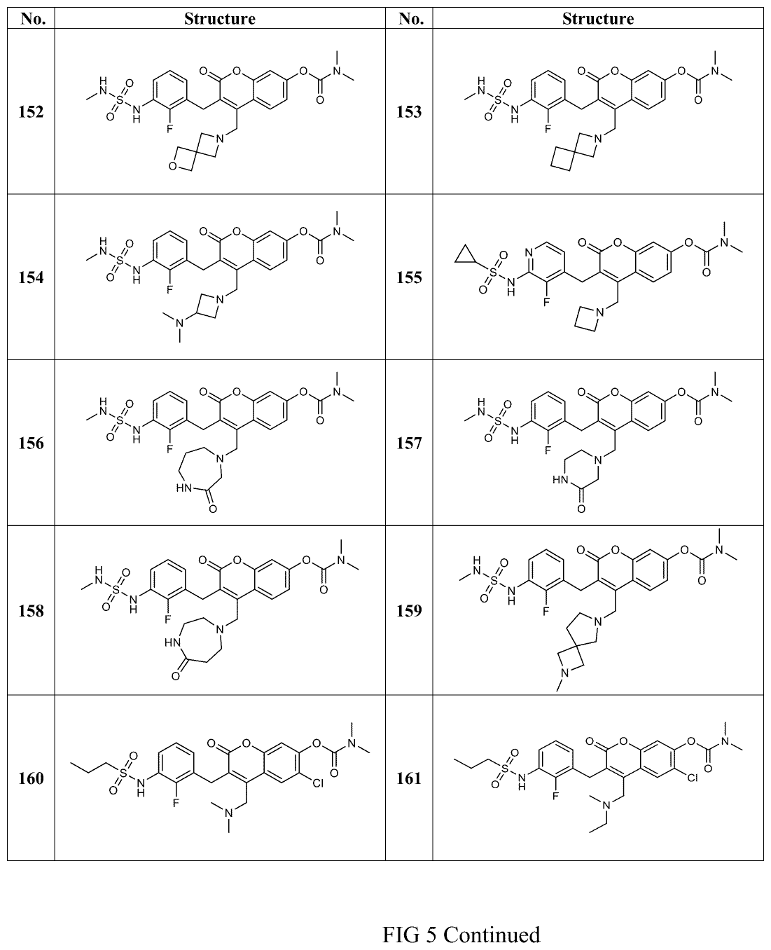

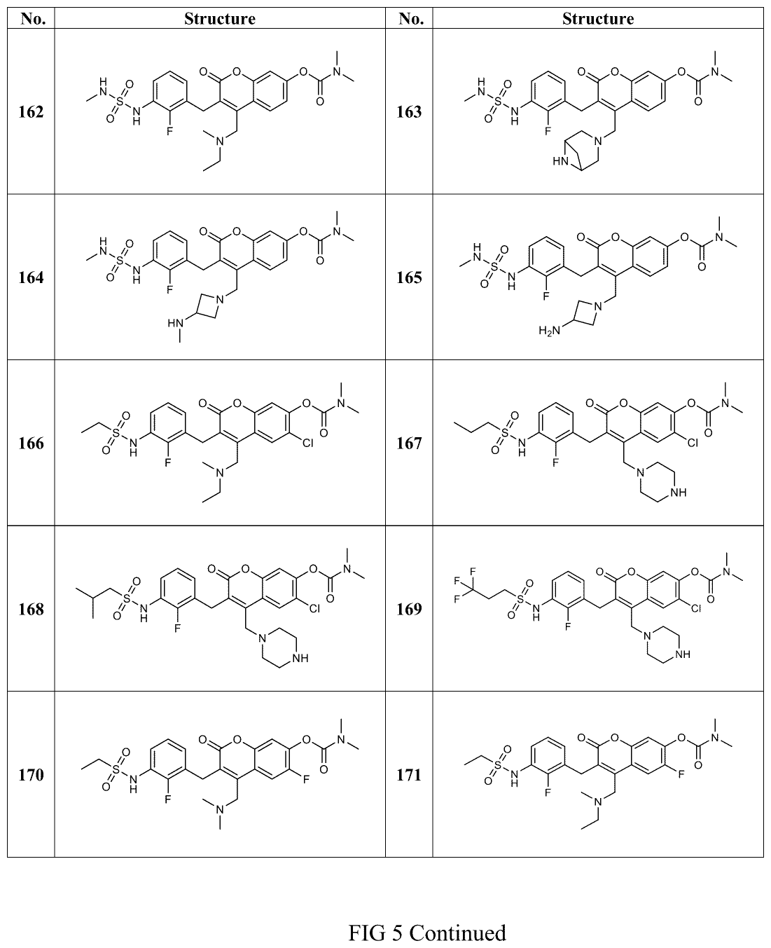

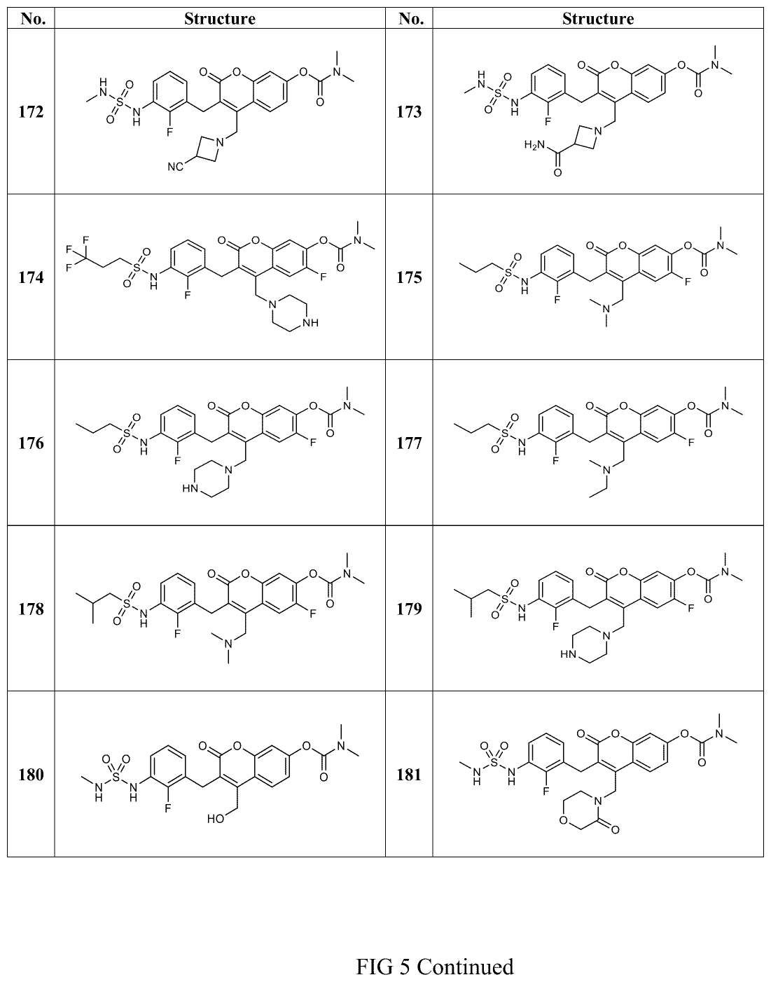

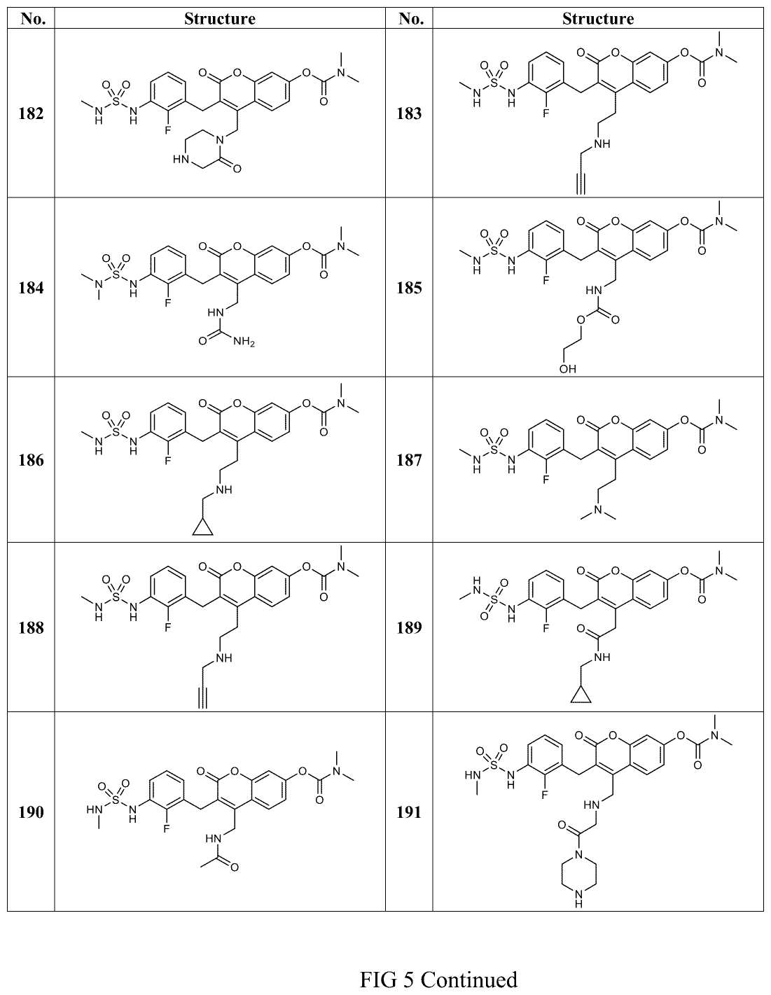

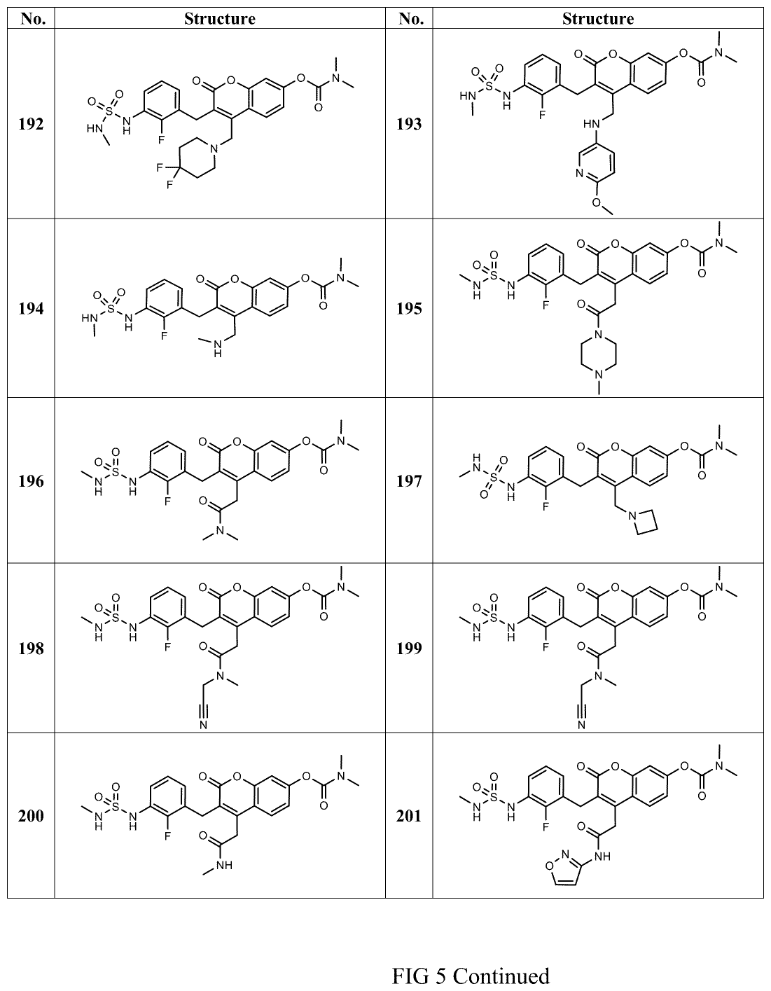

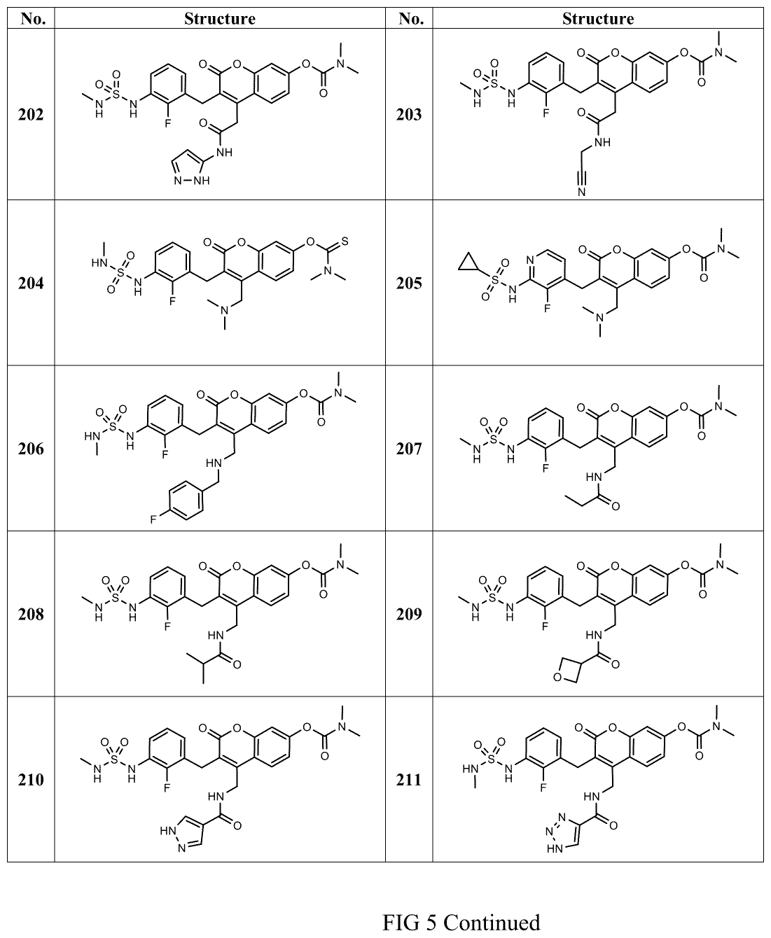

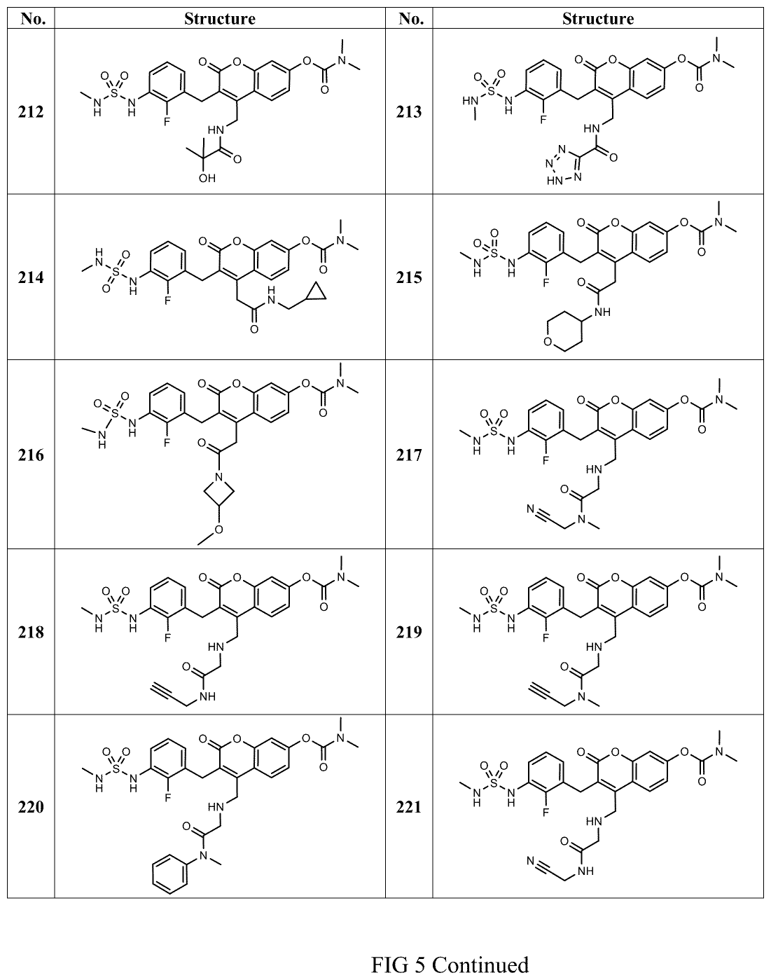

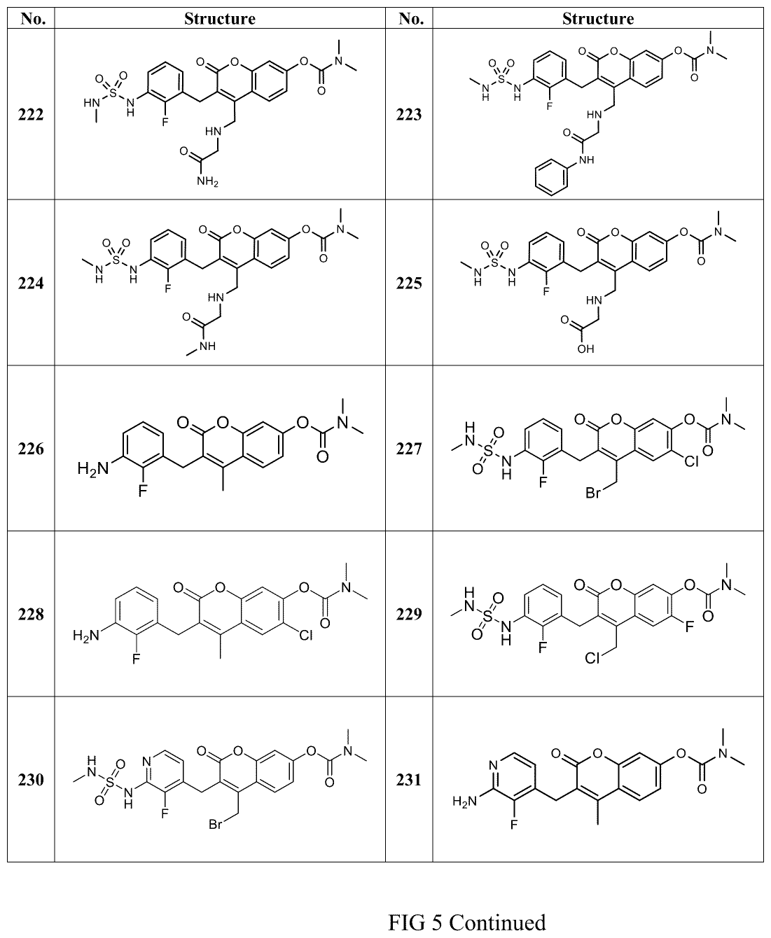

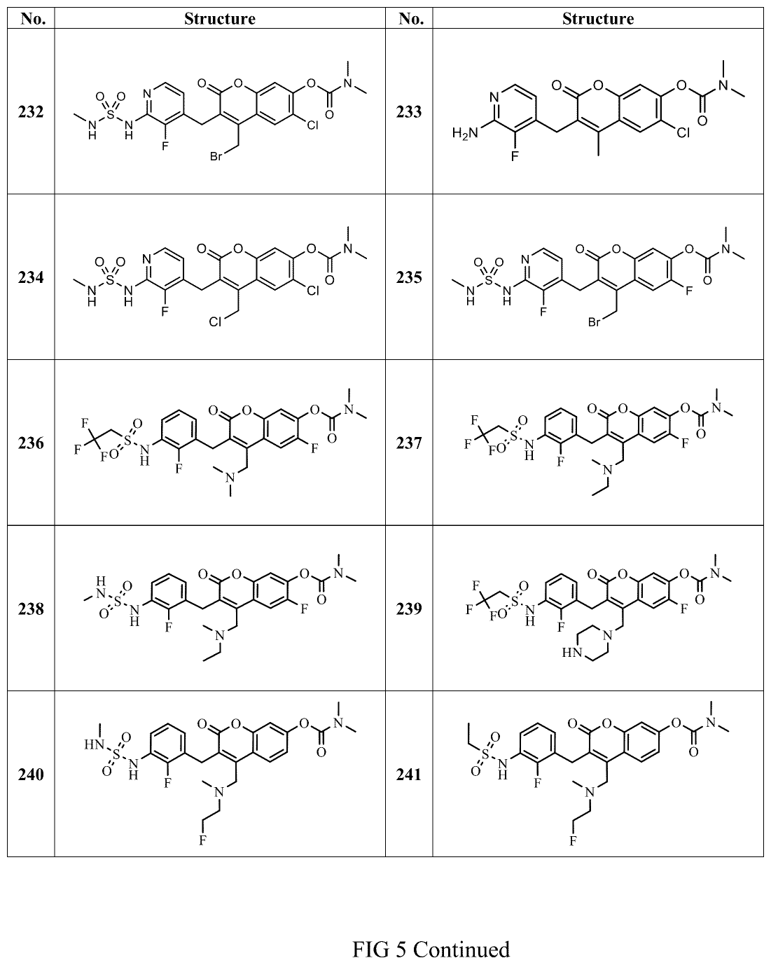

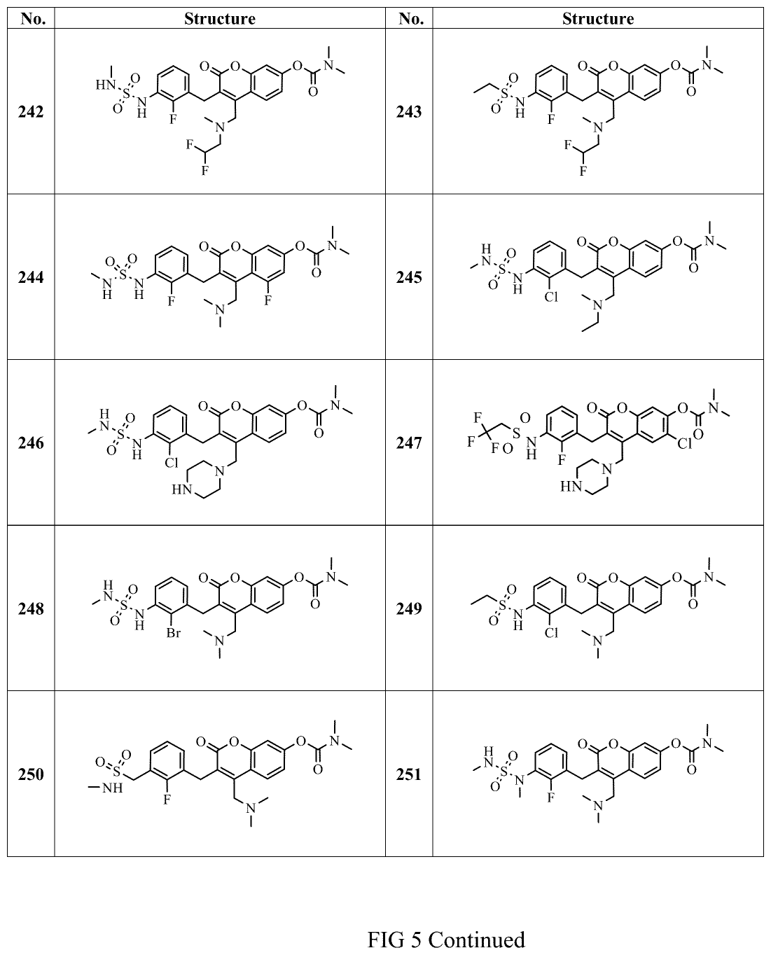

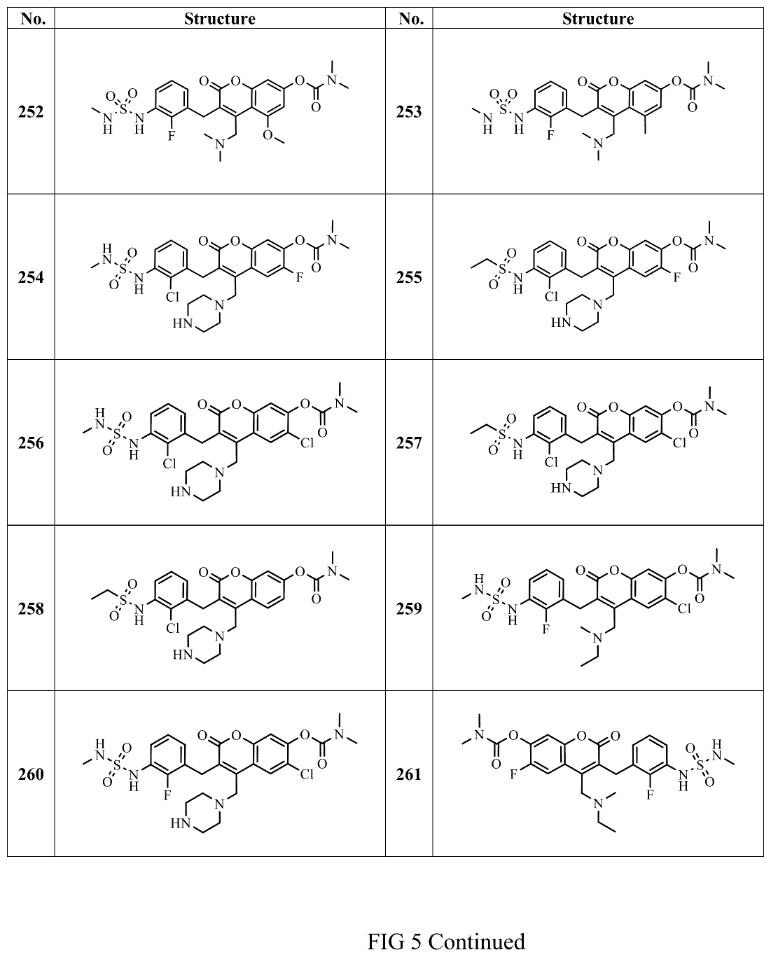

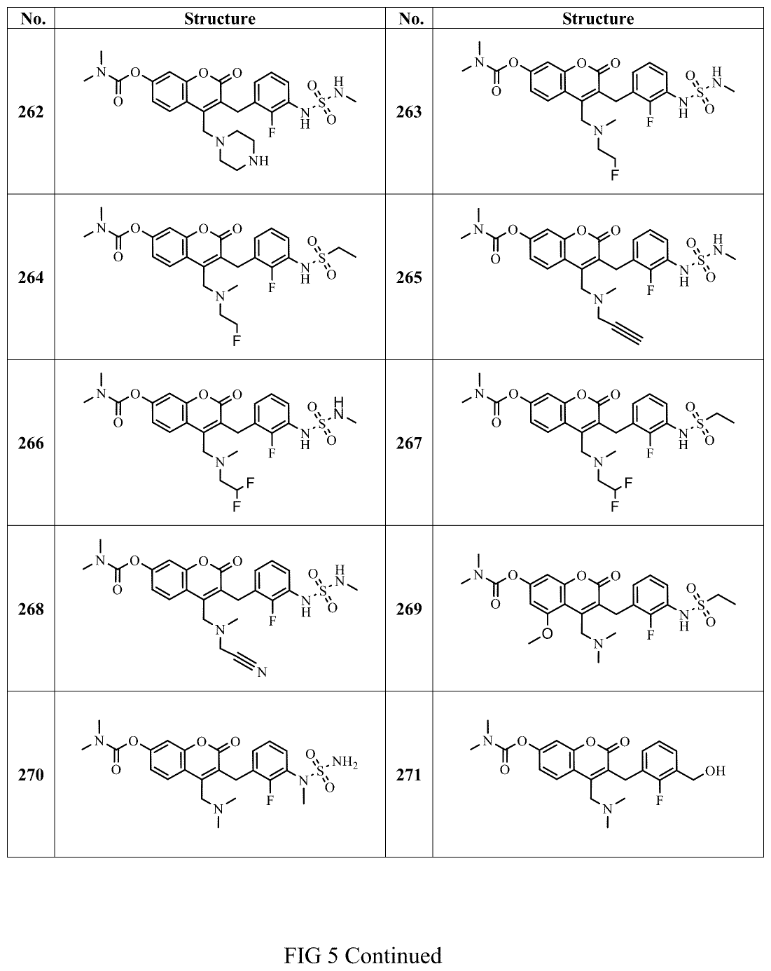

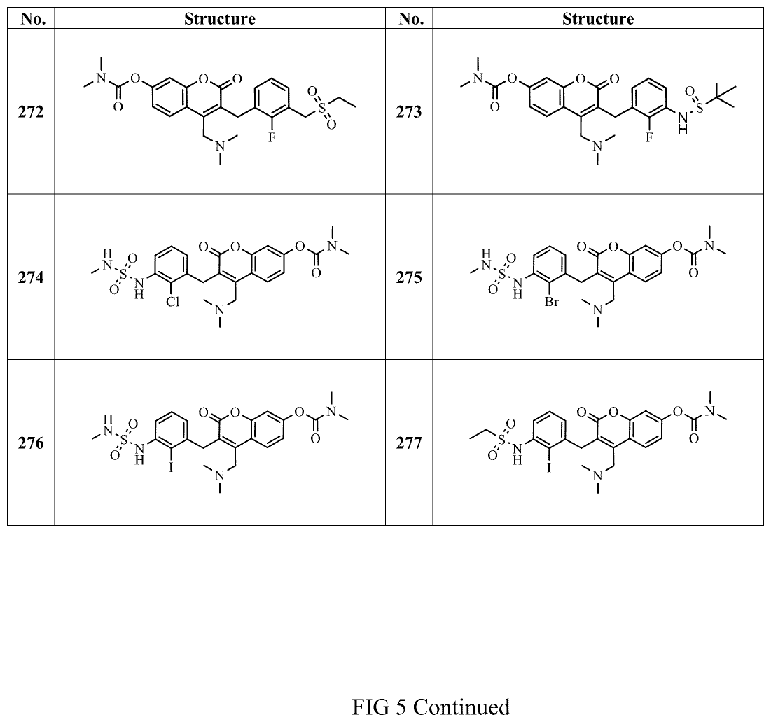

Female mice (either athymic BALC/c nude) were inoculated with a single subcutaneous injection of 0.1 ml of mouse Colon-26 cell suspension (1×10⁵ cells per animal) in the inguinal region. Colon26 (C26) tumor bearing mice. Mice received either vehicle (10% 1N HCl:90% [20% Captisol in saline pH adjusted to 5.0±0.1] adjusted to pH 3.0±0.1), or vehicle containing compounds at 25, 50, 75, 100, 125, 150, 175 and 200 mg/kg by gavage (p.o.) either QD or BID for 8 days. Tumor growth inhibition (TGI) was calculate and represented in FIGS. 3 and 4.

### Example 46

### Human Ether-a-go-go Related Gene (hERG) Channel

In this study, hERG Ion channel block of IC50 values were determined. The values are described in Table 21. In brief, CHO cells were cultured in Ham's F-12 supplemented with 10% fetal bovine serum, 100 U/mL penicillin G sodium, 100 μg/mL streptomycin sulfate and 400 μg/mL Zeocin. Before testing, cells in culture dishes were rinsed with Hank's Balanced Salt Solution, detached with accutase. Immediately before use in the SyncroPatch® 384PE system, the cells were washed in HB-PS to remove the accutase and re-suspended in 15 mL of HB-PS. Test article (TA) concentrations were applied to naïve cells (n=4, where n=replicates/concentration). Each application consisted of addition of 40 μL of 2× concentrated test article solution to the total 80 μL of final volume of the extracellular well of the SP384PE chip. Duration of exposure to each compound concentration was five (5) minutes. hERG current was measured using stimulus voltage patterns with fixed amplitudes: activation pre-pulse (TP1) to +40 mV for 2 s and test pulse (TP2) to −40 mV for 2 s from a holding potential of −80 mV. hERG current was measured as the outward peak current at TP2 (tail current). The stimulation was repeated with 0.1 Hz frequency during 2 min as baseline and 5 min after TA application. Data acquisition and analyses were performed using the SP384PE system operation software. The decrease in current amplitude after TA application was used to calculate the percent block relative to control. Results for each TA concentration (n≥2) were averaged; the mean and standard error values were calculated, and used to generate dose-response curves.

TABLE 21

| Compound ID | IC50 (μM) |
|---|---|
| 9 | 22.4 |
| 10 | >30 |
| 124 | 4.2 |
| 146 | 13.1 |
| 161 | 5.6 |
| 167 | 1.5 |
| 238 | 19.4 |
| 245 | 16.1 |
| 246 | >30 |
| 254 | >30 |
| 255 | 18.1 |
| 256 | 21.6 |
| 257 | 8.4 |
| 274 | >30 |
| Cisapride (control | 0.02 |

### Example 47

### Membrane Permeability (Caco-2)

In this study, membrane permeability (Caco-2) was determined. The results of this study are described in Table 22.

TABLE 22

| | Mean Pₐₚₚ | Mean Pₐₚₚ | Mean | A − B |
|---|---|---|---|---|
| | A − B | B − A | Efflux | Permeability |
| Compound ID | (10⁻⁶ cm/s) | (10⁻⁶ cm/s) | Ratio | Ranking |
| 9 | 16.4 | 21.7 | 1.32 | Higher |
| 10 | 0.00 | 2.43 | 0.00 | Lower |
| 25 | 4.39 | 16.7 | 3.82 | Higher |
| 120 | 21.9 | 12.4 | 0.567 | Higher |
| 124 | 3.51 | 18.0 | 5.12 | Higher |
| 134 | 3.99 | 8.08 | 2.01 | Higher |
| 146 | 0.240 | 8.54 | 35.5 | Lower |
| 161 | 20.5 | 16.7 | 0.813 | Higher |
| 167 | 0.845 | 19.6 | 23.2 | Lower |
| 238 | 14.2 | 13.5 | 0.956 | Higher |
| 245 | 11.9 | 6.87 | 0.578 | Higher |
| 246 | 4.16 | 6.99 | 1.68 | Higher |
| 256 | 3.59 | 13.4 | 3.73 | Higher |
| 257 | 4.08 | 22.1 | 5.41 | Higher |
| 274 | 16.8 | 8.95 | 0.533 | Higher |
| Talinolo NI | 0.412 | 6.54 | 15.9 | Lower |
| Talinolol VERA | 0.940 | 2.39 | 2.55 | Lower |
| Warfarin | 40.5 | 20.8 | 0.513 | Higher |
| Ranitidine NI | 0.407 | 1.18 | 2.89 | Lower |

### Example 48

### Hepatocyte Stability

In this study, human, cynomolgus monkey, beagle dog, wistar hannover rat, and balb/c mouse hepatocyle stability was determined. Table 23 describes the results of the human hepatocyte stability at 2 μM concentration and 0.5 million cells/ml for 120 min. Table 24 describes the results of the cynomolgus monkey hepatocyte stability at 2 μM concentration and 0.5 million cells/ml. Table 25 describes the results of the beagle dog hepatocyte stability at 2 μM concentration and 0.5 million cells/ml. Table 26 describes the results of the wistar hannover rat hepatocyte stability at 2 μM concentration and 0.5 million cells/ml. Table 26 describes the results of the balb/c mouse hepatocyte stability at 2 μM concentration and 0.5 million cells/ml.

TABLE 23

| | | % | | | % cell |
|---|---|---|---|---|---|
| | T_{1/2} | Remaining | CLᵢₙₜ | CLᵢₙₜ | viability |
| Cmpd No. | (min) | At T₁₂₀ | (mL/min/kg) | (L/hr/kg) | (initial) |
| 9 | 116 | 48.9 | 32.2 | 1.93 | 89.0 |
| 10 | 8179 | 99.0 | 0.458 | 0.0275 | 89.0 |
| 25 | 103228 | 99.9 | 0.0363 | 0.00218 | 89.0 |
| 120 | 111 | 47.4 | 33.6 | 2.02 | 89.0 |
| 124 | 236 | 70.3 | 15.9 | 0.953 | 89.0 |
| 134 | 1521 | 94.7 | 2.46 | 0.148 | 89.0 |
| 146 | 2120 | 96.2 | 1.77 | 0.106 | 89.0 |
| 161 | 73.3 | 32.2 | 51.0 | 3.06 | 89.0 |
| 167 | 134 | 53.8 | 27.9 | 1.67 | 89.0 |
| 238 | 64.0 | 27.3 | 58.4 | 3.51 | 89.0 |
| 245 | 42.2 | 13.9 | 88.7 | 5.32 | 89.0 |
| 246 | 1725 | 95.3 | 2.17 | 0.130 | 89.0 |
| 256 | 927 | 91.4 | 4.04 | 0.242 | 89.0 |
| 257 | 719 | 89.1 | 5.21 | 0.312 | 89.0 |
| 274 | 112 | 47.5 | 33.5 | 2.01 | 89.0 |
| Verapamil | 44.4 | 15.4 | 84.2 | 5.05 | 89.0 |

TABLE 24

| | | % | | | % cell |
|---|---|---|---|---|---|
| | T_{1/2} | Remaining | CLᵢₙₜ | CLᵢₙₜ | viability |
| Cmpd No. | (min) | At T₁₂₀ | (mL/min/kg) | (L/hr/kg) | (initial) |
| 9 | 102 | 44.3 | 54.9 | 3.30 | 77.8 |
| 10 | >120 | 102.3 | <46.8 | <2.81 | 77.8 |
| 25 | 380 | 80.4 | 14.8 | 0.886 | 77.8 |
| 120 | 72.1 | 31.6 | 77.8 | 4.67 | 77.8 |
| 124 | 117 | 49.2 | 47.9 | 2.87 | 77.8 |
| 134 | 344 | 78.5 | 16.3 | 0.980 | 77.8 |
| 146 | 276 | 74.0 | 20.3 | 1.22 | 77.8 |
| 161 | 41.8 | 13.7 | 134 | 8.05 | 77.8 |
| 167 | 67.1 | 29.0 | 83.7 | 5.02 | 77.8 |
| 238 | 57.2 | 23.3 | 98.2 | 5.89 | 77.8 |
| 245 | 24.4 | 3.3 | 230 | 13.8 | 77.8 |
| 246 | 405 | 81.4 | 13.9 | 0.832 | 77.8 |
| 256 | 135 | 53.9 | 41.7 | 2.50 | 77.8 |
| 257 | 179 | 62.8 | 31.4 | 1.88 | 77.8 |
| 274 | 59.1 | 24.5 | 94.9 | 5.7 | 77.8 |
| Verapamil | 20.5 | 1.7 | 274 | 16.5 | 77.8 |

TABLE 25

| | | % | | | % cell |
|---|---|---|---|---|---|
| | T_{1/2} | Remaining | CLᵢₙₜ | CLᵢₙₜ | viability |
| Cmpd No. | (min) | At T₁₂₀ | (mL/min/kg) | (L/hr/kg) | (initial) |
| 9 | 202 | 66.2 | 36.9 | 2.22 | 73.1 |
| 10 | 414 | 81.8 | 18.0 | 1.08 | 73.1 |
| 25 | 178 | 62.7 | 41.9 | 2.51 | 73.1 |
| 120 | 118 | 49.5 | 63.0 | 3.78 | 73.1 |
| 124 | 78.0 | 34.4 | 95.6 | 5.73 | 73.1 |
| 134 | 285 | 74.7 | 26.1 | 1.57 | 73.1 |
| 146 | 128 | 52.1 | 58.4 | 3.50 | 73.1 |
| 161 | 95.5 | 41.9 | 78.0 | 4.68 | 73.1 |
| 167 | 48.0 | 17.7 | 155 | 9.31 | 73.1 |
| 238 | 71.2 | 31.1 | 105 | 6.28 | 73.1 |
| 245 | 57.2 | 23.4 | 130 | 7.81 | 73.1 |
| 246 | 364 | 79.6 | 20.5 | 1.23 | 73.1 |
| 256 | 179 | 62.8 | 41.7 | 2.50 | 73.1 |
| 257 | 113 | 47.8 | 66.1 | 3.96 | 73.1 |
| 274 | 127 | 51.9 | 58.7 | 3.52 | 73.1 |
| Verapamil | 43.8 | 15.0 | 170 | 10.2 | 73.1 |

TABLE 26

| | | % | | | % cell |
|---|---|---|---|---|---|
| | T_{1/2} | Remaining | CLᵢₙₜ | CLᵢₙₜ | viability |
| Cmpd No. | (min) | At T₁₂₀ | (mL/min/kg) | (L/hr/kg) | (initial) |
| 9 | 79.0 | 34.9 | 47.3 | 2.84 | 80.6 |
| 10 | 255 | 72.1 | 14.7 | 0.882 | 80.6 |
| 25 | 163 | 60.0 | 23.0 | 1.38 | 80.6 |
| 120 | 73.1 | 32.0 | 51.2 | 3.07 | 80.6 |
| 124 | 73.4 | 32.2 | 51.0 | 3.06 | 80.6 |
| 134 | 162 | 59.8 | 23.1 | 1.39 | 80.6 |
| 146 | 115 | 48.6 | 32.5 | 1.95 | 80.6 |
| 161 | 65.2 | 27.9 | 57.4 | 3.44 | 80.6 |
| 167 | 71.1 | 31.0 | 52.6 | 3.16 | 80.6 |
| 238 | 56.6 | 23.0 | 66.1 | 3.97 | 80.6 |
| 245 | 44.9 | 15.7 | 83.4 | 5.00 | 80.6 |
| 246 | 180 | 63.0 | 20.8 | 1.25 | 80.6 |
| 256 | 105 | 45.7 | 35.3 | 2.12 | 80.6 |
| 257 | 105 | 45.4 | 35.5 | 2.13 | 80.6 |
| 274 | 79.0 | 34.9 | 47.3 | 2.84 | 80.6 |
| Verapamil | 36.5 | 10.2 | 103 | 6.15 | 80.6 |

TABLE 27

| | | % | | | % cell |
|---|---|---|---|---|---|
| | T_{1/2} | Remaining | CLᵢₙₜ | CLᵢₙₜ | viability |
| Cmpd No. | (min) | At T₁₂₀ | (mL/min/kg) | (L/hr/kg) | (initial) |
| 9 | 274 | 73.8 | 59.7 | 3.58 | 58.9 |
| 10 | 316 | 76.9 | 51.8 | 3.11 | 58.9 |
| 25 | 253 | 72.0 | 64.7 | 3.88 | 58.9 |
| 120 | 247 | 71.4 | 66.3 | 3.98 | 58.9 |
| 124 | 140 | 55.1 | 117 | 7.04 | 58.9 |
| 134 | 235 | 70.2 | 69.7 | 4.18 | 58.9 |
| 146 | 281 | 74.4 | 58.3 | 3.50 | 58.9 |
| 161 | 143 | 56.0 | 114 | 6.86 | 58.9 |
| 167 | 129 | 52.5 | 127 | 7.61 | 58.9 |
| 238 | 149 | 57.3 | 110 | 6.57 | 58.9 |
| 245 | 130 | 52.8 | 126 | 7.54 | 58.9 |
| 246 | 272 | 73.6 | 60.3 | 3.62 | 58.9 |
| 256 | 197 | 65.6 | 83.0 | 4.98 | 58.9 |
| 257 | 206 | 66.7 | 79.6 | 4.78 | 58.9 |
| 274 | 188 | 64.3 | 87.1 | 5.23 | 58.9 |
| Verapamil | 57.6 | 23.6 | 284 | 17.1 | 58.9 |

### Example 49

### Plasma Stability

In this study, human, cynomolgus monkey, beagle dog, wistar hannover rat, and balb/c plasma stability was determined. Table 28 describes the results of the human plasma stability at 2 μM concentration and anti-coagulant K2-EDTA from 10 to 120 min. Table 29 describes the results of the cynomolgus monkey plasma stability at 2 μM concentration and anti-coagulant K2-EDTA. Table 30 describes the results of the beagle dog plasma stability at 2 μM concentration and anti-coagulant K2-EDTA. Table 31 describes the results of the wistar hannover rat plasma stability at 2 μM concentration and anti-coagulant K2-EDTA. Table 32 describes the results of the balb/c plasma stability at 2 μM concentration and anti-coagulant K2-EDTA.

TABLE 28

| Compound ID | T_{1/2} (min) | % Remaining at T120 |
|---|---|---|
| 9 | >120 | 99.8 |
| 10 | >102 | 97.8 |
| 25 | >120 | 112.1 |
| 120 | >120 | 123.8 |
| 124 | 631 | 85.0 |
| 134 | >120 | 107.8 |
| 146 | >120 | 95.4 |
| 161 | 3541 | 97.0 |
| 167 | 948 | 84.6 |
| 238 | 4114 | 95.7 |
| 245 | >120 | 106.8 |
| 246 | >120 | 105.7 |
| 256 | 841 | 84.8 |
| 257 | 10367 | 96.3 |
| 274 | >120 | 121.2 |
| Propantheline | 56.6 | 20.8 |

TABLE 29

| Compound ID | T_{1/2} (min) | % Remaining at T120 |
|---|---|---|
| 9 | >120 | 110.4 |
| 10 | 435 | 85.2 |
| 25 | >120 | 107.3 |
| 120 | >120 | 107.3 |
| 124 | 1279 | 95.9 |
| 134 | 946 | 93.0 |
| 146 | 603 | 89.2 |
| 161 | >120 | 98.4 |
| 167 | >120 | 117.3 |
| 238 | >120 | 101.1 |
| 245 | 1379 | 96.5 |
| 246 | >120 | 104.5 |
| 256 | 1711 | 95.0 |
| 257 | 1207 | 98.0 |
| 274 | >120 | 104.5 |
| Propantheline | 450 | 84.6 |

TABLE 30

| Compound ID | T_{1/2} (min) | % Remaining at T120 |
|---|---|---|
| 9 | >120 | 100.5 |
| 10 | 353 | 83.4 |
| 25 | 593 | 90.1 |
| 120 | >120 | 100.9 |
| 124 | 5649 | 101.5 |
| 134 | 627 | 89.9 |
| 146 | 3540 | 95.0 |
| 161 | >120 | 102.6 |
| 167 | >120 | 106.2 |
| 238 | 1395 | 93.8 |
| 245 | >120 | 104.1 |
| 246 | 3629 | 100.2 |
| 256 | >120 | 99.4 |
| 257 | >120 | 100.9 |
| 274 | >120 | 100.5 |
| Propantheline | 292 | 71.0 |

TABLE 31

| Compound ID | T_{1/2} (min) | % Remaining at T120 |
|---|---|---|
| 9 | 1180 | 93.4 |
| 10 | 2738 | 91.8 |
| 25 | 3326 | 95.5 |
| 120 | 2561 | 96.8 |
| 124 | >120 | 100.3 |
| 134 | 1269 | 95.2 |
| 146 | 3941 | 97.9 |
| 161 | 8518 | 98.1 |
| 167 | >120 | 106.9 |
| 238 | 1785 | 95.5 |
| 245 | 1935 | 96.6 |
| 246 | >120 | 99.3 |
| 256 | 10732 | 97.2 |
| 257 | 1011 | 91.5 |
| 274 | 2536 | 97.7 |
| Propantheline | >120 | 115.2 |

TABLE 32

| Compound ID | T_{1/2} (min) | % Remaining at T120 |
|---|---|---|
| 9 | 527 | 86.0 |
| 10 | >120 | 114.7 |
| 25 | >120 | 99.9 |
| 120 | 14321 | 94.2 |
| 124 | >120 | 105.4 |
| 134 | 2811 | 100.2 |
| 146 | 747 | 89.8 |
| 161 | 317 | 74.9 |
| 167 | >120 | 100.5 |
| 238 | 160 | 57.5 |
| 245 | 479 | 81.9 |
| 246 | 1108 | 94.5 |
| 256 | 586 | 86.8 |
| 257 | 586 | 87.5 |
| 274 | 7583 | 92.7 |
| Propantheline | 101 | 37.3 |

### Example 50

### Human Liver Microsomes (MLM) CYP Inhibition

In this study, human liver microsomes as a mean percentage CYP inhibition at 10 μM were determined. The results of this study are depicted in Table 33.

TABLE 33

| Cmpd | CYP-1A2 | CYP-2B6 | CYP-2C8 | CYP-2C9 | CYP-2C19 | CYP-2D6 | CYP-3A4 | CYP-3A4 |
|---|---|---|---|---|---|---|---|---|
| ID | (Phenacetin) | (Bupropion) | (Amodiaquine) | (Diclofenac) | (Mephenytoin) | (Dextromethorphan) | (Midazolam) | (Testosterone) |
| 9 | 2.8 | −2.5 | 15.5 | 13.4 | 17.4 | 4.4 | 9.6 | 30.9 |
| 10 | 3.1 | 19.3 | 19.4 | 25.4 | 23.2 | 8.9 | 13.7 | 19.2 |
| 25 | 7.9 | 16.0 | 77.2 | 28.0 | 26.5 | 5.7 | 48.5 | 47.2 |
| 120 | −3.7 | 13.4 | 21.9 | 11.1 | 14.2 | −1.9 | 11.4 | 12.6 |
| 124 | 9.9 | 25.5 | 59.0 | 65.5 | 50.6 | 10.8 | 63.2 | 62.6 |
| 134 | −0.9 | 10.3 | 78.8 | 20.5 | 20.6 | 0.5 | 44.5 | 52.6 |
| 146 | 8.2 | 2.0 | 59.6 | 32.0 | 36.9 | 11.1 | 28.4 | 32.0 |
| 161 | −3.1 | −2.3 | 7.1 | 17.1 | 19.1 | 8.3 | 13.5 | 38.8 |
| 167 | 10.8 | 19.8 | 61.8 | 71.8 | 67.1 | 10.1 | 54.8 | 51.2 |
| 245 | −0.3 | 6.0 | 30.4 | 25.8 | 29.0 | 13.7 | 42.3 | 72.8 |
| 246 | −3.7 | 29.7 | 10.5 | 19.4 | 22.0 | 5.1 | 16.2 | 24.6 |
| 256 | 6.2 | 1.9 | 97.0 | 36.5 | 40.8 | 7.1 | 61.8 | 69.2 |
| 257 | 9.9 | 13.7 | 97.5 | 53.7 | 47.5 | 10.1 | 67.4 | 66.0 |
| 274 | −3.4 | −0.6 | 11.9 | 17.1 | 15.1 | 2.5 | 20.1 | 50.4 |

Controls

| | | | | | | | |
|---|---|---|---|---|---|---|---|
| Fluvoxamine | Ticlopidine | Quercetin | Sulfaphenazole | Omeprazole | Quinidine | Ketoconazole | Ketoconazole |

Dose

| 10 μM | 10 μM | 10 μM | 10 μM | 10 μM | 1 μM | 10 μM | 10 μM |
|---|---|---|---|---|---|---|---|
| 98.5 | 99.0 | 88.3 | 95.6 | 60.7 | 93.1 | 106.6 | 102.8 |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1-53. (canceled)

54. A compound having the chemical structure of Formula (IV) 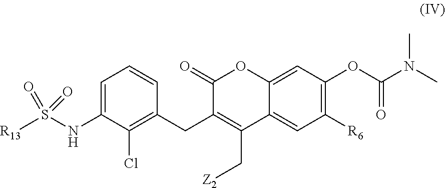
or a pharmaceutically acceptable salt thereof, wherein
R₆ is hydrogen, fluoro or chloro;
R₁₃ is ethyl or —NR_{A}R_{B} wherein R_{A} is hydrogen and R_{B} is methyl;
Z₂ is —NR⁵R^{5′}, 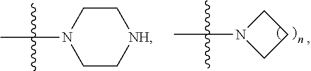
wherein n is 1, 2, 3 or 4;
R⁵ is C₁ to C₆ alkyl; and
R^{5′} is C₁ to C₆ alkyl.

55. The compound of claim 54, wherein n is 1.

56. The compound of claim 54, wherein Z₂ is —NR⁵R^{5′}.

57. The compound of claim 56, wherein R⁵ is methyl, and R^{5′} is methyl or ethyl.

58. The compound of claim 57, wherein R₁₃ is —NR_{A}R_{B}.

59. The compound of claim 57, wherein R₁₃ is ethyl.

60. The compound of claim 54, wherein Z₂ is

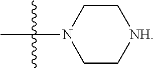 [SMILES: *N1CCNCC1]

61. The compound of claim 60, wherein R₁₃ is —NR_{A}R_{B}.

62. The compound of claim 60, wherein R₁₃ is ethyl.

63. The compound of claim 54, wherein R₁₃ is —NR_{A}R_{B}.

64. The compound of claim 54, wherein R₁₃ is ethyl.

65. The compound of claim 54, wherein compound having the chemical structure of Formula (IV) is selected from the group consisting of: 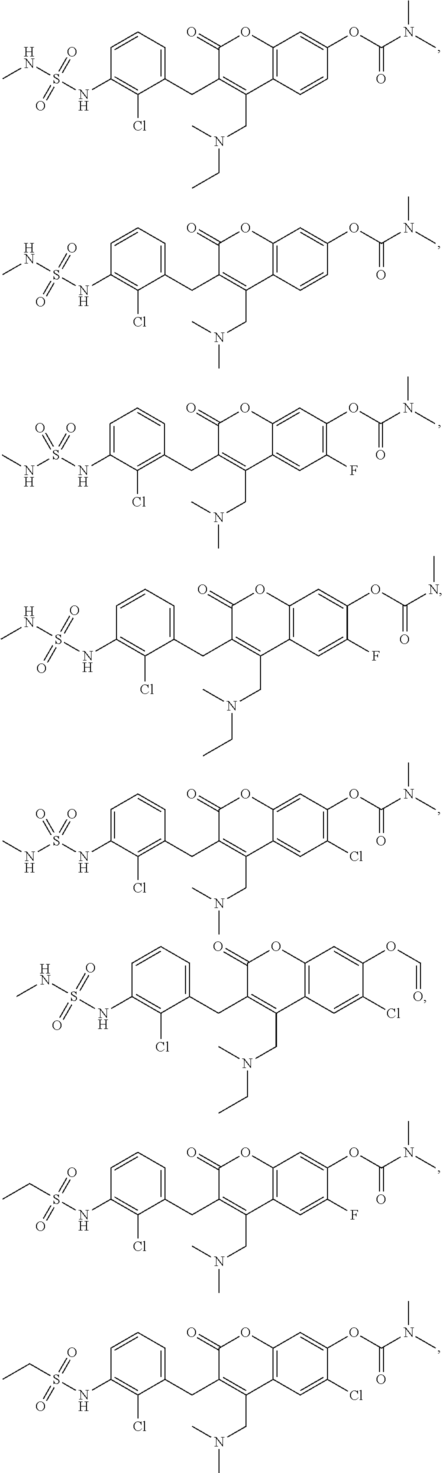 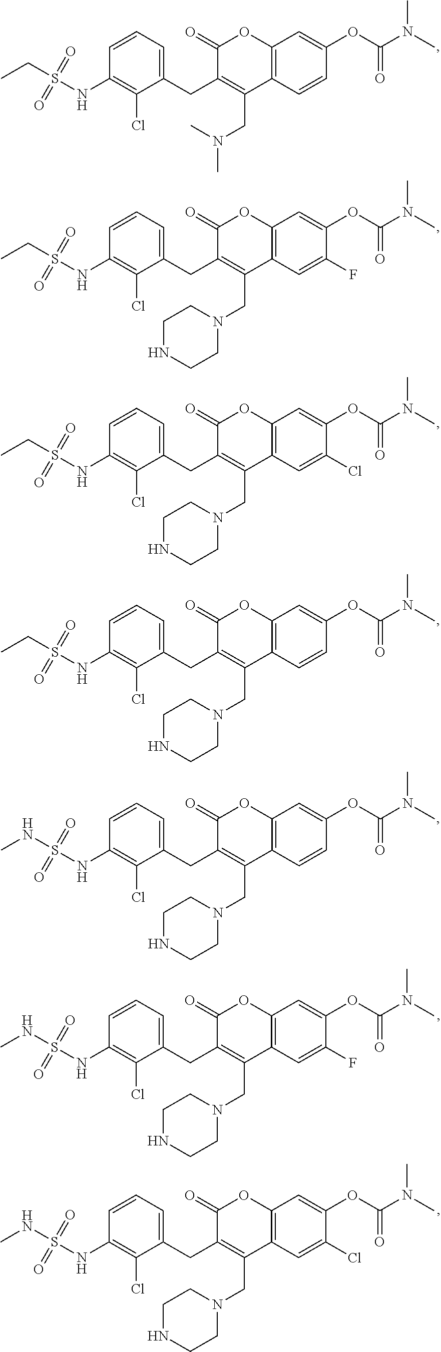 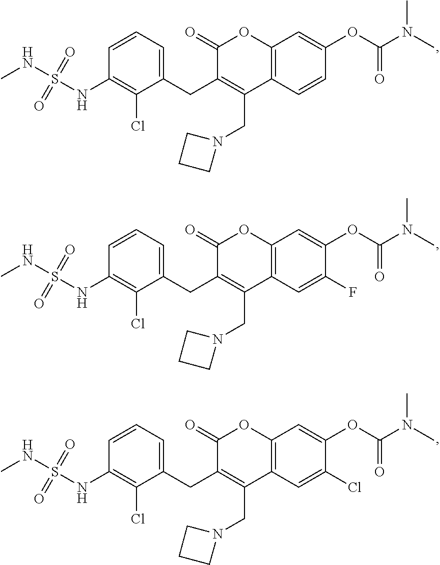
or a pharmaceutically acceptable salt thereof.

66. The compound of claim 54, wherein the compound is 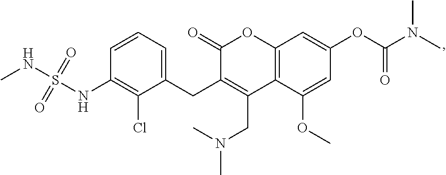
or a pharmaceutically acceptable salt thereof.

67. The compound of claim 54, wherein the compound is 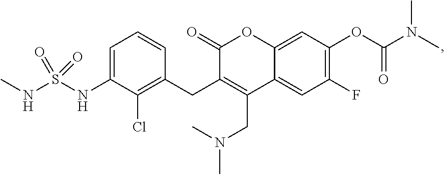
or a pharmaceutically acceptable salt thereof.

68. The compound of claim 54, wherein the compound is 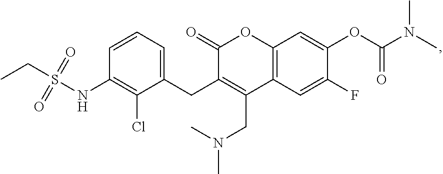 [SMILES: CCS(=O)(=O)Nc1cccc(Cc2c(CN(C)C)c3cc(F)c(OC(=O)N(C)C)cc3oc2=O)c1Cl]
or a pharmaceutically acceptable salt thereof.

69. The compound of claim 54, wherein the compound is 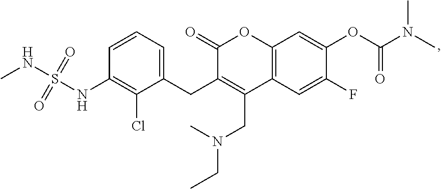
or a pharmaceutically acceptable salt thereof.

70. The compound of claim 54, wherein the compound is 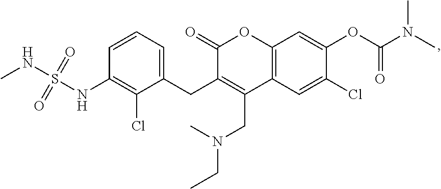
or a pharmaceutically acceptable salt thereof.

71. The compound of claim 54, wherein the compound is 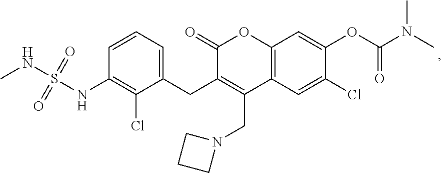
or a pharmaceutically acceptable salt thereof.

72. The compound of claim 54, wherein the compound is 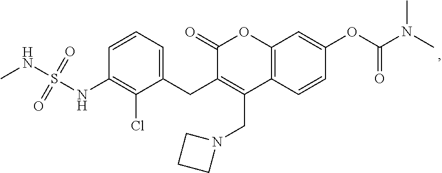
or a pharmaceutically acceptable salt thereof.

73. A method of treating cancer, comprising administering to a subject in need thereof an effective amount of a compound of claim 54 or a pharmaceutical composition thereof.
